(19) United States
(12) Patent Application Publication
(10) Pub. No.: US 2026/0124205 A1
(43) Pub. Date: May 7, 2026

(54) MODULATORS OF BETA CATENIN AND USES THEREOF

(71) Applicant: Dewpoint Therapeutics, Inc., Boston, MA (US)

(72) Inventors: Adam Talbot, Boston, MA (US); Thomas Durand-Reville, Boston, MA (US)

(21) Appl. No.: 19/364,191

(22) Filed: Oct. 21, 2025

Related U.S. Application Data
(60) Provisional application No. 63/710,194, filed on Oct. 22, 2024.

(51) Int. Cl. A61K 31/506; A61K 31/428; A61K 31/4439; A61K 31/444; A61K 31/5383; A61K 31/5386; A61P 35/00; C07B 59/00; C07D 417/12; C07D 417/14; C07D 471/10; C07D 487/10; C07D 491/107; C07D 495/10; C07D 519/00

(52) U.S. Cl. CPC A61K 31/506; A61K 31/428; A61K 31/4439; A61K 31/444; A61K 31/5383; A61K 31/5386; A61P 35/00; C07B 59/002; C07D 417/12; C07D 417/14; C07D 471/10; C07D 487/10; C07D 491/107; C07D 495/10; C07D 519/00

(57) ABSTRACT

Provided are compounds of the Formula I:

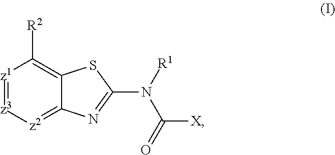

or pharmaceutically acceptable salts thereof, which are useful for the modulation of beta catenin condensates and in the treatment of a variety of conditions or diseases associated therewith.

### RELATED APPLICATIONS

This application claims the benefit of priority to U.S. Provisional Application No. 63/710,194, filed Oct. 22, 2024, the entire contents of which are incorporated herein by reference.

### BACKGROUND

Biomolecular condensates are ubiquitous spatiotemporal organizers of biology, compartmentalizing and integrating a wide variety of regulatory pathways within cells. See Banani, S. F. et al. Nat Rev Mol Cell Bio 18, 285-298 (2017); and Hyman, A. A. et al. Annu Rev Cell Dev Bi 30, 39-58 (2014). Aberrant condensates, or condensatopathies, act as central nodes of dysfunction in disease, representing a new class of targets for drug discovery. See Alberti, S. et al. Nat Rev Mol Cell Bio 22, 196-213 (2021); Boija, A. et al. Cancer Cell 39, 174-192 (2021); Mitrea, D. M. et al. Nat Rev Drug Discov 1-22 (2022); Martin, E. W. et al. J. Mol. Biol. 168380 (2023); and Patel, A. et al. Frontiers Mol Biosci 9, 1007744 (2022). This new perspective expands the target space and enables new strategies for pursuing targets historically considered ‘undruggable’; these strategies include but are not limited to modifying the condensates these targets associate to, or targeting structural states that are selectively adopted by the ‘undruggable’ target inside the condensate. See Mitrea, D. et al. Nat Rev Drug Discov 1-22 (2022). Due to their integrative role, dysfunction of condensates leads to complex defects in multiple biological processes, explaining the root cause of complex diseases, such as cancer and neurodegeneration. This central role in pathophysiology also opens opportunities to discover broad-acting drugs that are active across large patient populations with a shared condensatopathy but of diverse genetic background.

Beta catenin is a validated anti-neoplastic target, known to be a driver of over-proliferation and aberrant transcriptional programming in a broad spectrum of cancers, including, but not limited to colorectal, breast, skin and pancreatic cancer. See Liu, J. et al. Signal Transduct Target Ther. (2022); and Zhang, Y. et al. J Hematol Oncol 13, 165 (2020). The cancer-driving activity of beta catenin is attributed to its constitutively active transcriptional promoting function, which results from several mutations and other aberrations along the dysregulated Wnt-pathway. Transcriptionally active beta catenin drives the expression of dozens of genes including NOTUM, MYC, AXIN2 and LEF1. See Nusse, R. et al. Nature 519, 163 (2015) and Herbst, A. et al. BMC Genomics 15, 74 (2014). Thus, what is needed is novel small molecules that modulate beta catenin's aberrant transcriptional activation and cell proliferation through its sequestration into nuclear condensates.

### SUMMARY

Provided herein are compounds having the Formula I:

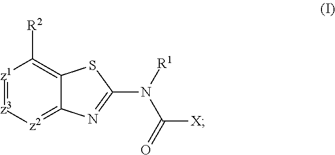

and pharmaceutically acceptable salts and compositions thereof, wherein R¹, R², X, z¹, z², and z³ are as described herein. In one aspect, the described compounds of Formula I and pharmaceutically acceptable salts thereof modulate beta catenin (e.g., sequester beta catenin in nuclear condensates), and are useful in a variety of therapeutic applications such as, for example, in treating cancer. See for example the cancer cell-line derived xenograft study and related data shown in the exemplification section below.

Pharmaceutical compositions comprising the described compounds and pharmaceutically acceptable salts of the described compounds, as well as methods for their preparation are also included.

### BRIEF DESCRIPTION OF THE DRAWINGS

FIG. 1 shows the beta catenin condensate modulation in DLD-1 cells treated with Example 1 or DMSO.

FIG. 2 shows tumor volumes for mice treated with Example 1 and mice provided vehicle.

FIG. 3 shows the gene expression changes in DLD-1 cells treated with Example 1 or DMSO.

### DETAILED DESCRIPTION

### 1. General Description of Compounds

In a first embodiment, provided herein are compounds having the Formula I:

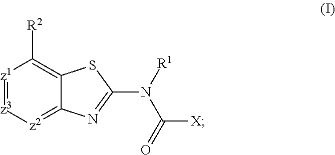

or a pharmaceutically acceptable salt thereof, wherein:
z¹ is N or —CR³;
z² is N or —CR⁵;
z³ is N or —CR⁴;
R¹ is hydrogen, (C₁-C₄)alkyl, hydroxy(C₁-C₄)alkyl, (C₁-C₄)alkylene(C₁-C₄)alkoxy, or (C₁-C₄)alkylene[OP(O)(OH)₂];
R² is aryl, heteroaryl, heterocyclyl, or cycloalkyl, each of which are optionally substituted with 1 to 3 groups selected from R^{1A};
R³ and R⁴ are each independently hydrogen, halo, cyano, (C₁-C₄)alkyl, halo(C₁-C₄)alkyl, (C₁-C₄)alkoxy, halo(C₁-C₄)alkoxy, (C₂-C₄)alkenyl, (C₁-C₄)alkylene[heterocyclyl], (C₁-C₄)alkylene[aryl], (C₁-C₄)alkylene[heteroaryl], (C₁-C₄)alkylene[cycloalkyl], —S(O)₂(C₁-C₄)alkyl, or —S(O)(C₁-C₄)alkyl, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano;
R⁵ is hydrogen, halo, or (C₁-C₄)alkyl;
X is —OR⁶ or —NR⁷R⁸;
R⁶ is cycloalkyl, heterocyclyl, (C₁-C₄)alkylene(C₁-C₄alkoxy), (C₁-C₄)alkylene[aryl], (C₁-C₄)alkylene[heteroaryl], (C₁-C₄)alkylene[heterocyclyl], or (C₁-C₄)alkylene[cycloalkyl], wherein the aryl, heteroaryl, and cycloalkyl are each optionally substituted with 1 to 3 groups selected from R^{2A};
R⁷ and R⁸ are taken together to form a heterocyclyl optionally substituted with 1 to 3 groups selected from R^{2A};
each R^{1A} is independently selected from halo, cyano, (C₁-C₄)alkyl, hydroxy(C₁-C₄)alkyl, hydroxy[halo(C₁-C₄)alkyl], cyano(C₁-C₄)alkyl, halo(C₁-C₄)alkyl, (C₁-C₄)alkoxy, deuterated(C₁-C₄)alkoxy, (C₁-C₄)alkoxyO(C₁-C₄)alkyl, (C₁-C₄)alkoxyO[deuterated(C₁-C₄)alkyl], halo(C₁-C₄)alkoxy, (C₁-C₄)alkoxyO[halo(C₁-C₄)alkyl], oxo, hydroxy, —O[hydroxy(C₁-C₄)alkyl], —O[cycloalkyl], —O[heterocyclyl], —O[aryl], —O[heteroaryl], (C₁-C₄)alkylene[cycloalkyl], (C₁-C₄)alkylene[aryl], (C₁-C₄)alkylene[heterocyclyl], (C₁-C₄)alkylene[heteroaryl], —O(C₁-C₄)alkylene[NR^{a}R^{b}], —O(C₁-C₄)alkylene[NR^{a}C(O)R^{b}], —O(C₁-C₄)alkylene[cycloalkyl], —O(C₁-C₄)alkylene[aryl], —O(C₁-C₄)alkylene[heterocyclyl], —O(C₁-C₄)alkylene[heteroaryl], cycloalkyl, heterocyclyl, aryl, heteroaryl, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], (C₁-C₄)alkoxyO[cycloalkyl], (C₁-C₄)alkoxyO[heterocyclyl], (C₁-C₄)alkoxyO[heteroaryl], (C₁-C₄)alkoxyO[phenyl], —C(O)[heterocyclyl], —SR^{a}, —S(O)R^{a}, —SO₂R^{a}, —S(O)(NR^{a})R^{b}, —NR^{b}SO₂R^{b}, —SO₂NR^{a}, —C(O)R^{a}, —C(O)OR^{a}, —C(O)NR^{a}R^{b}, —NR^{a}C(O)R^{b}, —NR^{a}C(O)NR^{b}, —NR^{a}R^{b}, —NH(C₁-C₄)alkylene[heterocyclyl], and (C₁-C₄)alkyleneNR^{a}R^{b}, where each occurrence of cycloalkyl, heterocyclyl, aryl, and heteroaryl alone, or as part of another group, is optionally substituted with 1 to 3 groups selected from halo, oxo, (C₁-C₄)alkyl, halo(C₁-C₄)alkyl, hydroxy(C₁-C₄)alkyl, (C₁-C₄)alkoxy, halo(C₁-C₄)alkoxy, hydroxy, —NH₂, —NH(C₁-C₄)alkyl, —N((C₁-C₄)alkyl)₂, cycloalkyl, heterocyclyl, aryl, heteroaryl, and cyano;
each R^{2A} is independently selected from halo, (C₁-C₄)alkyl, halo(C₁-C₄)alkyl, hydroxy(C₁-C₄)alkyl, deuterated(C₁-C₄)alkyl, (C₁-C₄)alkoxy, halo(C₁-C₄)alkoxy, deuterated(C₁-C₄)alkoxy, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], oxo, —O[cycloalkyl], cyano, hydroxy, S(O)(C₁-C₄)alkyl, —S(O)₂(C₁-C₄)alkyl, hydroxy, cycloalkyl, heterocyclyl, aryl, heteroaryl, (C₁-C₄)alkylene[cycloalkyl], (C₁-C₄)alkylene[aryl], (C₁-C₄)alkylene[heterocyclyl], (C₁-C₄)alkylene[heteroaryl], —C(O)R^{a1}, —C(O)OR^{a1}, —C(O)NR^{a1}R^{b1}, —NR^{a1}C(O)R^{b1}, —NR^{a1}C(O)NR^{b1}, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano; and
each R^{a}, R^{a1}, R^{b}, and R^{b1} is each independently selected from hydrogen, (C₁-C₄)alkyl, halo(C₁-C₄)alkyl, (C₁-C₄)alkylene(C₁-C₄)alkoxy, (C₁-C₄)alkyleneNH₂, (C₁-C₄)alkyleneNH((C₁-C₄alkyl), (C₁-C₄)alkyleneN((C₁-C₄alkyl)₂, cycloalkyl, and heterocyclyl, wherein the cycloalkyl and heterocyclyl are each optionally substituted with (C₁-C₄)alkyl, hydroxy, or hydroxy(C₁-C₄)alkyl;
provided that:
   (i) if R⁷ and R⁸, taken together with the N to which they are attached, form pyrrolidinyl, then R^{2A} is not —C(O)NH₂;
   (ii) if R⁷ and R⁸, taken together with the N to which they are attached, form piperidinyl, then R^{2A} is not benzyl; and
   (iii) if R⁷ and R⁸, taken together with the N to which they are attached, form 6- to 12-membered heterocyclyl, then R² is substituted by 1 to 3 groups selected from R^{1A}.

### 2. Definitions

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

As used in the structure herein a hyphen (−) or squiggly line “ [symbol] ” indicates the point of attachment of the particular depicted structure or substituent group to the appropriate atom(s) in the remainder of the molecule.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The term “alkyl,” when used alone or as part of a substituent group, refers to a straight- or branched-chain hydrocarbon group having from 1 to 12 carbon atoms (“C₁-C₁₂”), for example 1 to 6 carbons atoms (“C₁-C₆”), or 1 to 4 carbons atoms (“C₁-C₄”) in the group. Examples of alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentyl, amyl, neopentyl, 3-methyl-2-butanyl, tertiary amyl), hexyl (C₆) (e.g., n-hexyl), heptyl (C₇) (e.g., n-heptyl), octyl (C₈) (e.g., n-octyl), and the like. In some embodiments, the alkyl group is a C₁-C₆alkyl; in other embodiments, it is a C₁-C₄alkyl; and in other embodiments, it is a C₁-C₃alkyl.

As used herein, the term “alkenyl” refers to a straight- or branched-chain group having from 2 to 12 carbon atoms (“C₂-C₁₂”) in the group, wherein the group includes at least one carbon-carbon double bond. Examples of alkenyl groups include vinyl (—CH═CH₂; C₂alkenyl), allyl (—CH₂—CH═CH₂; C₃alkenyl), propenyl (—CH═CHCH₃; C₃alkenyl), isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (—CH═CHCH₂CH₂CH₃; C₅alkenyl), and the like.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “cycloalkyl” when used alone or as part of a substituent group refers to cyclic-containing, saturated or partially unsaturated hydrocarbon groups having from 3 to 10 carbon atoms (“C₃-C₁₀”), for example from 3 to 7 carbon atoms (“C₃-C₇”). Examples of cycloalkyl groups include cyclopropyl (C₃), cyclobutyl (C₄), cyclopentyl (C₅), cyclohexyl (C₆), cycloheptyl (C₇), and the like. In some embodiments, the cycloalkyl group is a C₃₋₄cycloalkyl; in other embodiments, it is a C₃-C₆cycloalkyl; and in other embodiments, it is C₃-C₈cycloalkyl.

The term “alkylene” refers to a straight- or branched-chain group having from 2 to 12 carbon atoms (“C₂-C₁₂”) in the group which is further substituted by a separate group. Examples of alkylene include (C₁-C₄)alkylene[heterocyclyl], which is a C₁-C₄alkyl substituted with a heterocyclyl, (C₁-C₄)alkylene[aryl], which is a C₁-C₄alkyl substituted with an aryl, (C₁-C₄)alkylene[cycloalkyl], which is a C₁-C₄alkyl substituted with a cycloalkyl, and (C₁-C₄)alkylene[heteroaryl], which is a C₁-C₄alkyl substituted with a heteroaryl.

The term “halo” or “halogen,” as used by itself or as part of another group refers to a fluorine, chlorine, bromine, or iodine atom.

As used herein, the term “haloalkyl” and “haloalkenyl” refer to an alkyl or alkenyl group, respectively, wherein one or more of the hydrogen atoms has been replaced with one or more halogen atoms which may be the same or different.

The term “alkoxy” as used by itself or as part of another group refers to an oxygen radical attached to an alkyl group by a single bond. Examples of alkoxyl groups include methoxy (OCH₃), ethoxy (OCH₂CH₃), propoxy (e.g., —OⁿPr, —OⁱPr), or butoxy (e.g., —OₙBu, —OⁱBu, —O^{s}Bu, —O^{t}Bu), and the like. In other embodiments, the alkoxy group is a C₁₋₆alkoxy. In further embodiments, the alkoxy group is a C₁₋₄alkoxy. In further embodiments, the alkoxy group is a C₁₋₃alkoxy.

As used herein, the term “haloalkoxy” refers to an alkoxy group wherein one or more of the hydrogen atoms has been replaced with one or more halogen atoms which may be the same or different.

A “deuterated” alkyl or alkoxy group means that one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of deuterium atoms at all sites of enrichment with the number of hydrogen plus deuterium atoms at all of the sites of enrichment. For example, a compound with two deuterated methyl groups has a 98.0% enrichment when 98.0% of the hydrogen atoms on the two methyl groups have been replaced with deuterium.

The term “aryl” used alone or as part of a larger moiety refers to, unless otherwise specified, a 6- or 10-membered aromatic hydrocarbon ring. An aryl group may be mono- or bi-cyclic, e.g., phenyl or naphthyl.

The term “heteroaryl” used alone or as part of a larger moiety refers to, unless otherwise specified, a 5- to 12-membered aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position.

The term “heterocyclyl” means, unless otherwise specified, a 5- to 12-membered saturated or partially unsaturated heterocyclic ring containing 1 to 4 heteroatoms independently selected from N, O, and S. It can be monocyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position.

The term “spiro” refers to two rings that shares one ring atom (e.g., carbon).

The term “fused” refers to two rings that share two adjacent ring atoms with one another.

The term “bridged” refers to two rings that share three ring atoms with one another.

The term “optionally substituted,” as used herein to describe a chemical moiety defined herein, means that the moiety may, but is not required to be, substituted with one or more suitable functional groups or other substituents as provided herein.

As used herein, the term “oxo” refers to a “═O” functional group.

As used herein, the term “substituted” means that an atom or group of atoms has replaced hydrogen as the substituent attached to another group.

The term “about” when used in combination with a numeric value or range of values means the value or range of values may deviate to an extent deemed reasonable to one of ordinary skill in the art.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including supercritical fluid chromatography (SFC), chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S.H. Tables of Resolving Agents and Optical Resolutions p. 268 (E. L. Eliel, Ed., Univ. of Notre Dame Press, Notre Dame, IN 1972).

Exemplary compounds of the disclosure including a chiral center may be depicted herein as having particular stereochemistries, but for which absolute stereochemistry has not been obtained. Absolute configurations can be obtained using methods known in the art.

As used herein, the term “stereoisomers” refers to compounds which have identical chemical constitution and connectivity but differ with regard to the arrangement of the atoms or groups in space, e.g., enantiomers or diastereomers.

When the stereochemical configuration at a chiral center in a compound having one or more chiral centers is depicted by its chemical name (e.g., where the configuration is indicated in the chemical name by “R” or “S”) or structure (e.g., the configuration is indicated by dashed or wedge bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

It will be understood that certain compounds disclosed herein may exist in tautomeric forms. Such forms are included as part of the present disclosure. Thus, when a compound herein is represented by a structural formula or designated by a chemical name herein, all tautomeric forms which may exist for the compound are encompassed by the structural formula.

When the compounds described herein contain olefinic double bonds or other centers of geometric asymmetry, and unless specified otherwise, it is intended that they include both E and Z geometric isomers.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

In some embodiments, the pharmaceutically acceptable salt of a compound described herein is a mono or di-sodium salt e.g., when R¹ is (C₁-C₄)alkylene[OP(O)(OH)₂].

“Pharmaceutically acceptable” means approved or approvable by a regulatory agency of the Federal or a state government or the corresponding agency in a country other than the United States, or that is listed in the U.S. Pharmacopoeia or other generally recognized pharmacopoeia for use in animals, e.g., in humans.

The term “effective amount” or “therapeutically effective amount” refers to an amount of a compound described herein that is sufficient to achieve the desired therapeutic effect (such as treatment of a condition recited herein) under the conditions of administration.

The use of any and all examples, or exemplary language (e.g., “such as” and “e.g.”) provided herein, is intended to better illustrate the disclosure and is not a limitation on the scope of the disclosure unless otherwise claimed. Phrases such as “in one aspect”, “in one embodiment”, “in another aspect”, “in another embodiment”, “in embodiments”, and the like should not be construed as indicating that such elements occur or exist in isolation or that such elements are not shared by other aspects or embodiments of the disclosure. Rather, it should be understood that all aspects and embodiments may be freely combined with any and all other aspects and embodiments of the disclosure as described herein. No language in the specification should be construed as indicating that any non-claimed element is essential to the practice of the disclosure.

### 3. Compounds

As part of a second embodiment, for the compounds of Formula I, or a pharmaceutically acceptable salt thereof, z³ is —CR⁴; and each R^{2A} is independently selected from halo, (C₁-C₄)alkyl, halo(C₁-C₄)alkyl, hydroxy(C₁-C₄)alkyl, deuterated(C₁-C₄)alkyl, (C₁-C₄)alkoxy, halo(C₁-C₄)alkoxy, deuterated(C₁-C₄)alkoxy, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], oxo, cyano, hydroxy, S(O)(C₁-C₄)alkyl, —S(O)₂(C₁-C₄)alkyl, hydroxy, cycloalkyl, heterocyclyl, aryl, heteroaryl, (C₁-C₄)alkylene[cycloalkyl], (C₁-C₄)alkylene[heterocyclyl], (C₁-C₄)alkylene[heteroaryl], —C(O)R^{a1}, —C(O)OR^{a1}, —NR^{a1}C(O)R^{b1}, —NR^{a1}C(O)NR^{b1}, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano, and wherein the remaining variables are as described above for Formula I.

As part of a third embodiment, for the compounds of Formula I, or a pharmaceutically acceptable salt thereof, (i) z¹ is N, z² is —CR⁵, and z³ is —CR⁴; (ii) z¹ is —CR³, z³ is —CR⁴, and z² is N; or (iii) z¹ is —CR³, z² is —CR⁵, and z³ is —CR⁴, wherein the remaining variables are as described above for Formula I or the second embodiment. Alternatively, as part of the third embodiment, for the compounds of Formula I, or a pharmaceutically acceptable salt thereof, z¹ is —CR³, z² is —CR⁵, and z³ is —CR, and wherein the remaining variables are as described above for Formula I or the second embodiment.

As part of a fourth embodiment, for the compounds of Formula I, or a pharmaceutically acceptable salt thereof, R³ is hydrogen, halo, (C₁-C₄)alkyl, halo(C₁-C₄)alkyl, (C₂-C₄)alkenyl, (C₁-C₄)alkoxy, cyano, —S(O)₂(C₁-C₄)alkyl, or (C₁-C₄)alkylene[heterocyclyl], wherein said heterocyclyl is optionally substituted by 1 halo, and wherein the remaining variables are as described above for Formula I or any one of the second or third embodiments. Alternatively, as part of the fourth embodiment, for the compounds of Formula I, or a pharmaceutically acceptable salt thereof, R³ is hydrogen, —F, —Cl, —CH₃, —CF₃, —CH═CH₂, —OCH₃, cyano, —S(O)₂CH₃

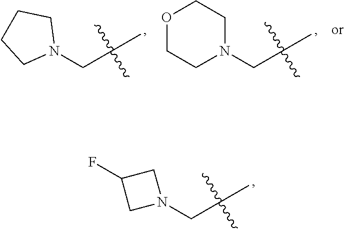 [SMILES: *CN1CCCC1 ; *CN1CCOCC1 ; *CN1CC(F)C1]

and wherein the remaining variables are as described above for Formula I or any one of the second or third embodiments.

As part of a fifth embodiment, for the compounds of Formula I, or a pharmaceutically acceptable salt thereof, R⁴ is hydrogen, halo, or (C₁-C₄)alkyl, and wherein the remaining variables are as described above for Formula I or any one of the second to fourth embodiments. Alternatively, as part of the fifth embodiment, for the compounds of Formula I, or a pharmaceutically acceptable salt thereof, R⁴ is hydrogen, —F, or —CH₃, and wherein the remaining variables are as described above for Formula I or any one of the second to fourth embodiments.

As part of a sixth embodiment, for the compounds of Formula I, or a pharmaceutically acceptable salt thereof, R⁵ is hydrogen, halo, or (C₁-C₄)alkyl, and wherein the remaining variables are as described above for Formula I or any one of the second to fifth embodiments. Alternatively, as part of the sixth embodiment, for the compounds of Formula I, or a pharmaceutically acceptable salt thereof, R⁵ is hydrogen, —F, or —CH₃, and wherein the remaining variables are as described above for Formula I or any one of the second to fifth embodiments.

As part of a seventh embodiment, for the compounds of Formula I, or a pharmaceutically acceptable salt thereof, R¹ is hydrogen or (C₁-C₄)alkylene[OP(O)(OH)₂], and wherein the remaining variables are as described above for Formula I or any one of the second to sixth embodiments. Alternatively, as part of the seventh embodiment, for the compounds of Formula I, or a pharmaceutically acceptable salt thereof, R¹ is hydrogen, and wherein the remaining variables are as described above for Formula I or any one of the second to sixth embodiments.

As part of an eighth embodiment, the compounds of Formula I have the structural Formula II:

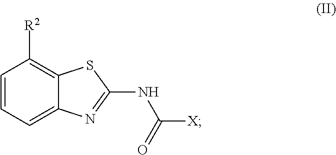

or a pharmaceutically acceptable salt thereof, and wherein the remaining variables are as described above for Formula I.

As part of a ninth embodiment, for the compounds of Formula I or II, or a pharmaceutically acceptable salt thereof, R² is phenyl, cyclohexyl, azetidinyl, morpholinyl, tetrahydropyridinyl, tetrahydropyranyl, dihydropyranyl, piperdinyl, azaspirohexanyl, azaspiroheptanyl, oxaazaspiroheptanyl, oxaazaspirooctanyl, oxaazaspirononanyl, pyrazolyl, triazolyl, pyridinyl, pyrimidinyl, pyrazinyl, pyridazinyl, isoindolinyl, dihydropyrazolooxazinyl, dihydrodioxinopyridinyl, or hexahydropyrazinopyridooxazinyl, each of which is optionally substituted with 1 to 3 groups selected from R^{1A}, and wherein the remaining variables are as described above for Formula I or II or any one of the second to eighth embodiments. Alternatively, as part of the ninth embodiment, for the compounds of Formula I or II, or a pharmaceutically acceptable salt thereof, R² is:

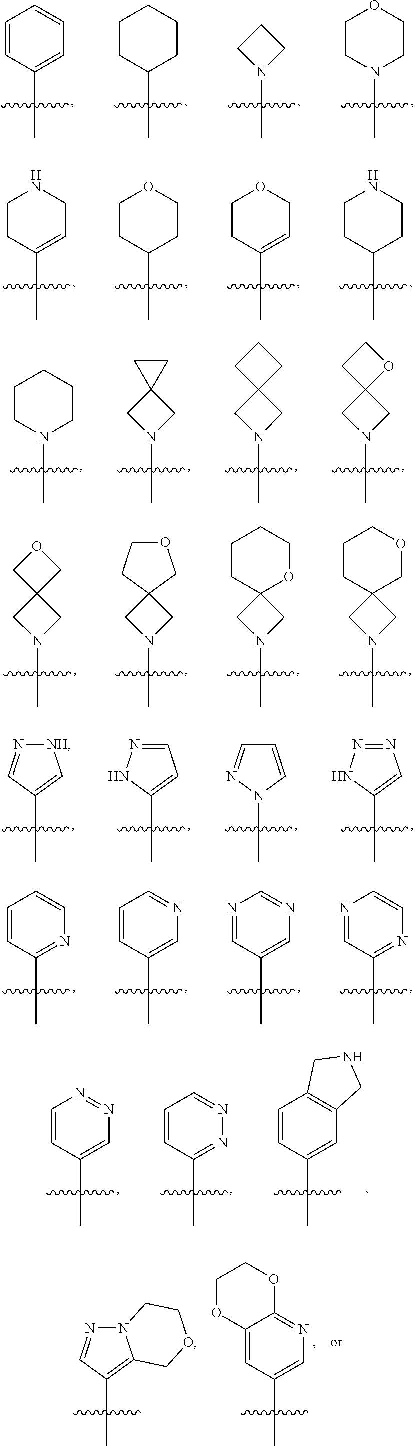

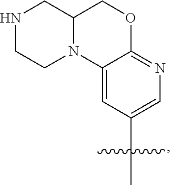 [SMILES: *c1cnc2c(c1)N1CCNCC1CO2]

each of which is optionally substituted with 1 or 2 groups selected from R^{1A}, and wherein the remaining variables are as described above for Formula I or II or any one of the second to eighth embodiments. Alternatively, as part of the ninth embodiment, for the compounds of Formula I or II, or a pharmaceutically acceptable salt thereof, R² is:

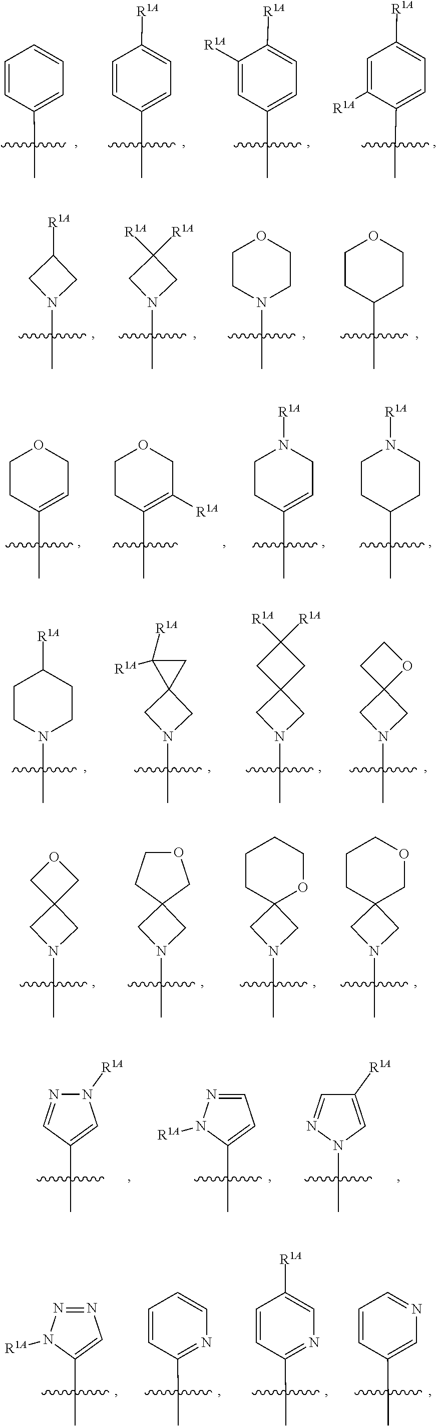

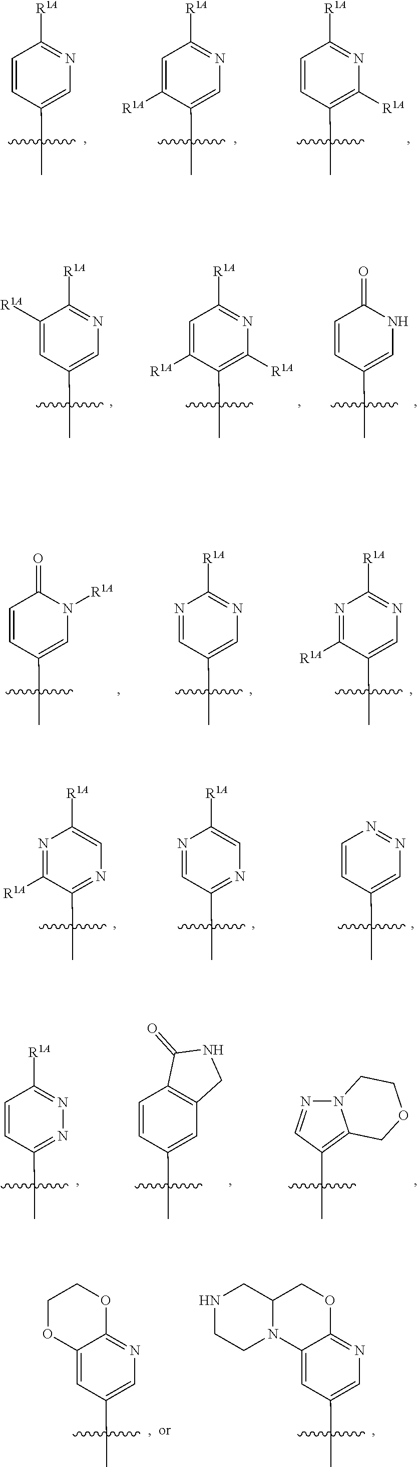

and wherein the remaining variables are as described above for Formula I or II or any one of the second to eighth embodiments.

As part of a tenth embodiment, for the compounds of Formula I or II, or a pharmaceutically acceptable salt thereof, each R^{IA} is independently halo, (C₁-C₄)alkyl, halo(C₁-C₄)alkyl, hydroxy(C₁-C₄)alkyl, hydroxy[halo(C₁-C₄)alkyl], (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], hydroxy, (C₁-C₄)alkoxy, halo(C₁-C₄)alkoxy, (C₁-C₄)alkoxyO(C₁-C₄)alkyl, (C₁-C₄)alkoxyO[deuterated(C₁-C₄)alkyl], (C₁-C₄)alkoxyO[halo(C₁-C₄)alkyl], —O(C₁-C₄)alkylene[NR^{a}R^{b}], —NR^{a}R^{b}, —NH(C₁-C₄)alkylene[heterocyclyl], cyano, —C(O)R^{a}, —C(O)NR^{a}R^{b}, —SO₂R^{a}, cycloalkyl, heterocyclyl, (C₁-C₄)alkylene[heterocyclyl], —O(C₁-C₄)alkylene[cycloalkyl], —O(C₁-C₄)alkylene[heterocyclyl], (C₁-C₄)alkoxyO[cycloalkyl], —O[cycloalkyl], or —O[heterocyclyl], where each occurrence of cycloalkyl and heterocyclyl, alone, or as part of another group, is optionally substituted with 1 to 3 groups selected from oxo, (C₁-C₄)alkyl, hydroxy, —NH(C₁-C₄alkyl), and heterocyclyl, and wherein the remaining variables are as described above for Formula I or II or any one of the second to ninth embodiments.

As part of an eleventh embodiment, for the compounds of Formula I or II, or a pharmaceutically acceptable salt thereof, R^{a} and R^{b} are each independently hydrogen, (C₁-C₄)alkyl, (C₁-C₄)alkylene(C₁-C₄)alkoxy, cycloalkyl, or heterocyclyl, wherein the cycloalkyl and heterocyclyl are each optionally substituted with (C₁-C₄)alkyl, hydroxy, or hydroxy(C₁-C₄)alkyl, and wherein the remaining variables are as described above for Formula I or II or any one of the second to tenth embodiments.

As part of a twelfth embodiment, for the compounds of Formula I or II, or a pharmaceutically acceptable salt thereof, each R^{1A} is independently —F, —Cl, —CH₃, —CH(CH₃)₂, —CH₂F, —CHF₂, —CF₃, —CF₂CH₃, —CH₂CF₃, —CF₂CH₂OH, —CH₂OCH₃, —CH₂OCD₃, —CH₂OCHF₂, —CH₂OCF₃, —CH₂CH₂OCH₃, —C(OH)(CH₃)₂, —CH₂C(OH)(CH₃)₂, hydroxy, —OCH₃, —OCH₂CH₃, —OCH(CH₃)₂, —OCHF₂, —OCF₃, —OCH₂CH₂OCH₃, —OCH₂CH₂OCD₃, —OCH₂CH₂OCHF₂, —OCH₂CH₂N(CH₃)₂, —NH₂, —NHCH₃, —N(CH₃)₂, cyano, —C(O)CH₃, —C(O)NH₂, —C(O)NHCH₃, —C(O)N(CH₃)₂, —C(O)NHCH₂CH₂OCH₃, —S(O)₂CH₃, cyclopropyl,

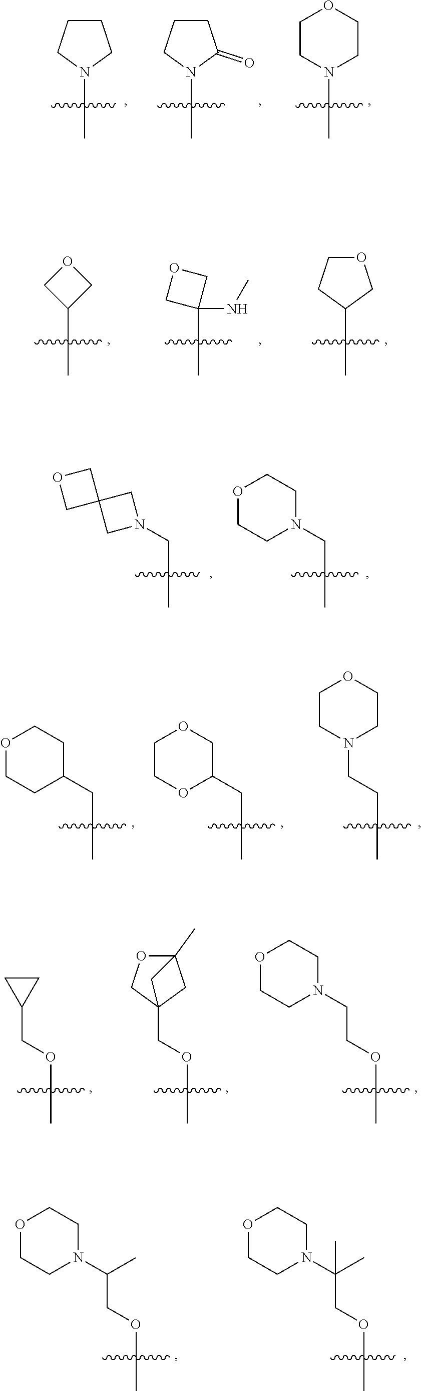

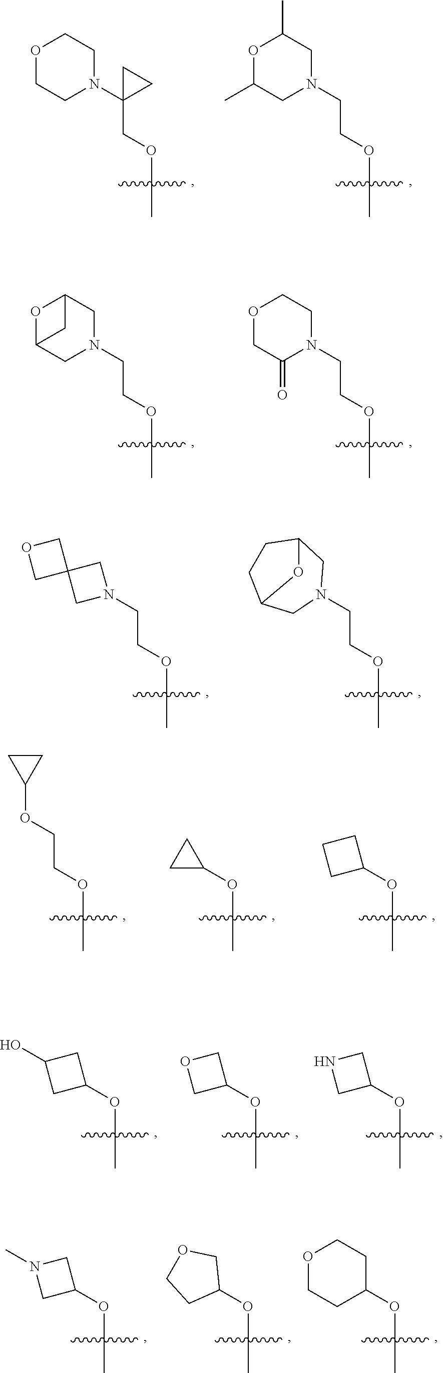

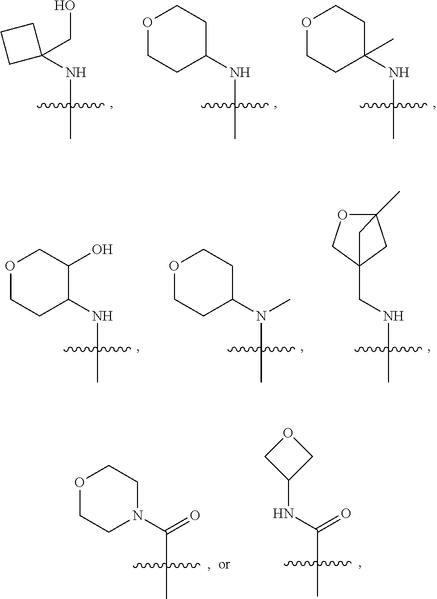

and wherein the remaining variables are as described above for Formula I or II or any one of the second to eleventh embodiments.

As part of a thirteenth embodiment, for the compounds of Formula I or II, or a pharmaceutically acceptable salt thereof, X is —OR⁶, and wherein the remaining variables are as described above for Formula I or II or any one of the second to twelfth embodiments.

As part of a fourteenth embodiment, for the compounds of Formula I or II, or a pharmaceutically acceptable salt thereof, R⁶ is (C₁-C₄)alkylene[heteroaryl], and wherein the remaining variables are as described above for Formula I or II or any one of the second to thirteenth embodiments. Alternatively, as part of the fourteenth embodiment, for the compounds of Formula I or II, or a pharmaceutically acceptable salt thereof, R⁶ is

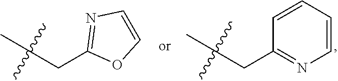 [SMILES: *Cc1ncco1 ; *Cc1ccccn1]

and wherein the remaining variables are as described above for Formula I or II or any one of the second to thirteenth embodiments.

As part of a fifteenth embodiment, for the compounds of Formula I or II, or a pharmaceutically acceptable salt thereof, X is —NR⁷R⁸, and wherein the remaining variables are as described above for Formula I or II or any one of the second to twelfth embodiments.

As part of a sixteenth embodiment, for the compounds of Formula I or II, or a pharmaceutically acceptable salt thereof, R⁷ and R⁸, together with the N to which they are attached, form azetidinyl, azaspirohexanyl, azaspiroheptanyl, oxaazaspiroheptanyl, thiaazaspiroheptanyl, diazaspirooctanyl, oxaazaspirooctanyl, oxaazaspirononanyl, diazaspirononanyl, oxadiazaspirononanyl, or dioxaazaspirononanyl, each of which are optionally substituted with 1 to 3 groups selected from R^{2A}, and wherein the remaining variables are as described above for Formula I or II or any one of the second to twelfth or fifteenth embodiments. Alternatively, as part of the sixteenth embodiment, for the compounds of Formula I or II, or a pharmaceutically acceptable salt thereof, R⁷ and R⁸, together with the N to which they are attached, form:

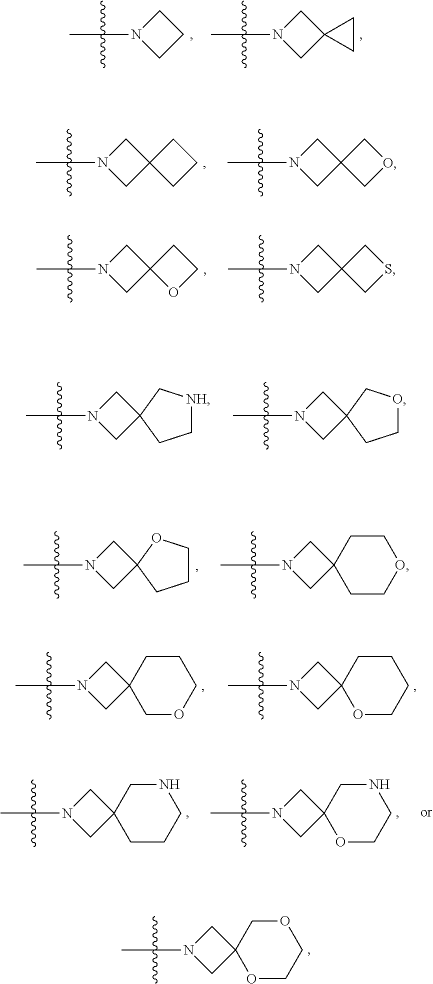

each of which are optionally substituted with 1 to 3 groups selected from R^{2A}, and wherein the remaining variables are as described above for Formula I or II or any one of the second to twelfth or fifteenth embodiments. Alternatively, as part of the sixteenth embodiment, for the compounds of Formula I or II, or a pharmaceutically acceptable salt thereof, R⁷ and R⁸, together with the N to which they are attached, form:

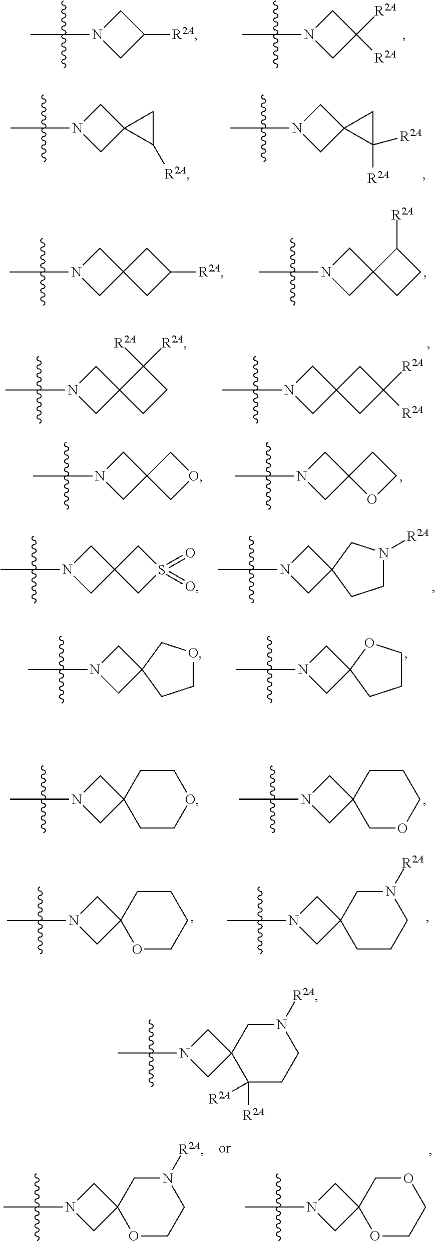

each of which are optionally substituted with 1 to 3 groups selected from R^{2A}, and wherein the remaining variables are as described above for Formula I or II or any one of the second to twelfth or fifteenth embodiments.

As part of a seventeenth embodiment, for the compounds of Formula I or II, or a pharmaceutically acceptable salt thereof, each R^{2A} is independently halo, (C₁-C₄)alkyl, deuterated(C₁-C₄)alkyl, halo(C₁-C₄)alkyl, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], hydroxy(C₁-C₄)alkyl, hydroxy, (C₁-C₄)alkoxy, deuterated(C₁-C₄)alkoxy, —S(O)₂(C₁-C₄)alkyl, cyano, or cycloalky, and wherein the remaining variables are as described above for Formula I or II or any one of the second to twelfth, fifteenth, or sixteenth embodiments. Alternatively, as part of the seventeenth embodiment, for the compounds of Formula I or II, or a pharmaceutically acceptable salt thereof, R^{2A} is independently —F, —CH₃, —CD₃, —CH₂F, —CF₃, —CH₂CF₃, —CH₂OCH₃, —CH₂OCH₂CH₃, —CH₂OCD₃, —CH₂OCHF₂, —CH₂OCF₃, —C(OH)(CH₃)₂, —C(CH₃)₂OCH₃, hydroxy, —OCH₃, —OCD₃, —OCH₂CH₃, —OCH(CH₃)₂, —S(O)₂CH₃, cyano, or cyclopropyl, and wherein the remaining variables are as described above for Formula I or II or any one of the second to twelfth, fifteenth, or sixteenth embodiments.

As part of an eighteenth embodiment, the compounds of Formula I have the structural Formula III:

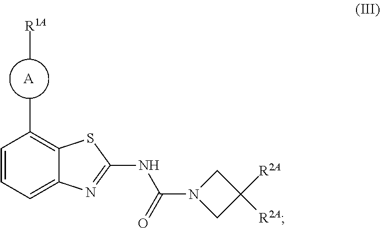

or a pharmaceutically acceptable salt thereof, wherein Ring A is a 5- to 6-membered nitrogen containing heteroaryl, and wherein the remaining variables are as described above for Formula I.

As part of a nineteenth embodiment, for the compounds of Formula I, II, or III, or a pharmaceutically acceptable salt thereof, Ring A is pyrazolyl, pyridinyl, or pyrimidinyl, and wherein the remaining variables are as described above for Formula I, II, or III or the eighteenth embodiment. Alternatively, as part of the nineteenth embodiment, for the compounds of Formula I, II, or III, or a pharmaceutically acceptable salt thereof, Ring A is:

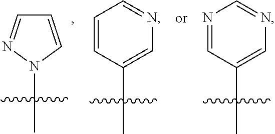

each of which is substituted by R^{1A}, and wherein the remaining variables are as described above for Formula I, II, or III or the eighteenth embodiment. Alternatively, as part of the nineteenth embodiment, for the compounds of Formula I, II, or III, or a pharmaceutically acceptable salt thereof, Ring A is:

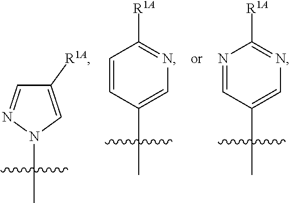

and wherein the remaining variables are as described above for Formula I, II, or III or the eighteenth embodiment.

As part of a twentieth embodiment, for the compounds of Formula I, II, or III, or a pharmaceutically acceptable salt thereof, R^{1A} is (C₁-C₄)alkoxy, halo(C₁-C₄)alkoxy, (C₁-C₄)alkoxyO(C₁-C₄)alkyl, —O[cycloalkyl], or —O[heterocyclyl], and wherein the remaining variables are as described above for Formula I, II, or III or the eighteenth or nineteenth embodiment. Alternatively, as part of the twentieth embodiment, for the compounds of Formula I, II, or III, or a pharmaceutically acceptable salt thereof, R^{1A} is —OCH₃, —OCHF₂, —OCH₂CH₃, —OCH(CH₃)₂, —O-cyclopropyl, —OCH₂CH₂OCH₃, or

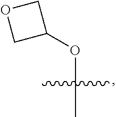 [SMILES: *OC1COC1]

and wherein the remaining variables are as described above for Formula I, II, or III or the eighteenth or nineteenth embodiment.

As part of a twenty-first embodiment, for the compounds of Formula I, II, or III, or a pharmaceutically acceptable salt thereof, R^{2A} is independently halo, (C₁-C₄)alkyl, (C₁-C₄)alkylene(C₁-C₄)alkoxy, (C₁-C₄)alkylene[deuterated(C₁-C₄)alkoxy], (C₁-C₄)alkoxy, or deuterated(C₁-C₄)alkoxy, and wherein the remaining variables are as described above for Formula I, II, or III or one of the eighteenth to twentieth embodiments. Alternatively, as part of the twenty-first embodiment, for the compounds of Formula I, II, or III, or a pharmaceutically acceptable salt thereof, R^{2A} is —F, —CH₃, —CH₂OCH₃, —CH₂OCH₂CH₃, —CH₂OCD₃, or —OCD₃, and wherein the remaining variables are as described above for Formula I, II, or III or one of the eighteenth to twentieth embodiments.

As part of a twenty-second embodiment, for the compounds of Formula I, II, or III, or a pharmaceutically acceptable salt thereof, R^{1A} is (C₁-C₄)alkoxy; and each R^{2A} is independently halo, (C₁-C₄)alkylene(C₁-C₄)alkoxy, or (C₁-C₄)alkylene[deuterated(C₁-C₄)alkoxy], and wherein the remaining variables are as described above for Formula I, II, or III or the eighteenth embodiment. Alternatively, as part of the twenty-second embodiment, for the compounds of Formula I, II, or III, or a pharmaceutically acceptable salt thereof, R^{1A} is —OCH₃ or —OCH₂CH₃; and each R^{2A} is independently —F, —CH₂OCH₃, or —CH₂OCD₃, and wherein the remaining variables are as described above for Formula I, II, or III or the eighteenth embodiment.

Additional compounds are described and exemplified herein, and are included in the present disclosure. Pharmaceutically acceptable salts thereof as well as the neutral forms of such compounds are included.

### 4. Uses, Formulation and Administration

The compounds and compositions described herein are generally useful for modulating the activity of beta catenin. In some aspects, the compounds, pharmaceutical acceptable salts, and pharmaceutical compositions described herein sequester beta catenin in nuclear condensates. In some aspects, the compounds, pharmaceutical acceptable salts, and pharmaceutical compositions described herein modulate beta catenin condensates.

In some aspects, the compounds and pharmaceutical compositions described herein are useful in treating a disease or condition associated with beta catenin function. Thus, provided herein are methods of treating a disease or condition associated with beta catenin function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. In some aspects, the compounds and pharmaceutical compositions described herein are useful in treating a disease or condition associated with the modulation of beta catenin condensates. Thus, provided herein are methods of treating a disease or condition associated with the modulation of beta catenin condensates, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a disease or condition associated with beta catenin function. Also provided is a compound described herein, or a pharmaceutically acceptable salt thereof, or a pharmaceutical composition comprising a disclosed compound or pharmaceutically acceptable salt thereof, for use in treating a disease or condition associated with beta catenin. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disease or condition associated with the modulation of beta catenin condensates. Also provided is a compound described herein, or a pharmaceutically acceptable salt thereof, or a pharmaceutical composition comprising a disclosed compound or pharmaceutically acceptable salt thereof, for use in treating a disease or condition associated with the modulation of beta catenin condensates.

In one aspect, the disease or condition associated with beta catenin or the modulation of beta catenin condensates is cancer. In embodiments, the cancer is bladder cancer, breast cancer, colorectal cancer, endometrial cancer, gastric cancer, head and neck cancer, leukemia, liver cancer, lung cancer, ovarian cancer, pancreatic cancer, or a solid tumor. In embodiments, the breast cancer is triple negative breast cancer. In embodiments, the leukemia is chronic lymphocytic leukemia or acute myeloid leukemia. In embodiments, the lung cancer is non-small cell lung cancer.

In some aspects, the compounds, pharmaceutical acceptable salts, and pharmaceutical compositions described herein modulate the function of beta catenin. In some aspects, modulating beta catenin comprises modulating beta catenin partitioning into condensates. In some aspects, modulating beta catenin comprises modulating transcription of beta catenin-dependent genes. In some aspects, modulating beta catenin comprises modulating beta catenin association with chromatin. In some aspects, modulating beta catenin comprises modulating beta catenin interaction network. In some aspects, modulating beta catenin comprises modulating beta catenin posttranscriptional modification status. In some aspects, modulating beta catenin comprises modulating direct and indirect regulators of beta catenin and the WNT pathway. In some aspects, modulating beta catenin comprises modulating the cell cycle.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

While there is described herein a number of embodiments, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Synthetic Procedure A

### Example 1: 3-Fluoro-3-(methoxymethyl)-N-(7-(2-methoxypyrimidin-5-yl)benzo[d]thiazol-2-yl)azetidine-1-carboxamide

### Step 1

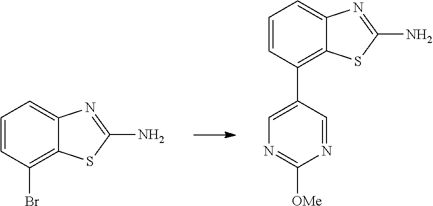 [SMILES: Nc1nc2cccc(Br)c2s1 ; COc1ncc(-c2cccc3nc(N)sc23)cn1]

A mixture of 7-bromobenzo[d]thiazol-2-amine (50.0 g, 218 mmol), (2-methoxypyrimidin-5-yl)boronic acid (40.3 g, 261 mmol), dioxane (750 mL) and water (250 mL) was degassed and purged with nitrogen three times. SPhos Pd G3 (13.6 g, 17.4 mmol) and potassium phosphate (139 g, 654 mmol) were added and the reaction mixture was stirred at 100° C. for 12 h under a nitrogen atmosphere. Additional water (50 mL) was added and stirring continued at room temperature for 30 min. The precipitate was filtered and then triturated with dichloromethane (1 L, 25° C., 2 h) to afford 7-(2-methoxypyrimidin-5-yl)benzo[d]thiazol-2-amine (41.0 g, 158 mmol, 73%) as a gray solid; δ_{H} (400 MHz; DMSO-d6) 8.86 (2H, s), 7.59 (2H, s), 7.41-7.31 (2H, m), 7.14 (1H, dd, J 7.2, 1.2 Hz), 3.99 (3H, s); ES-MS [M+H]⁺: 259.0.

### Step 2

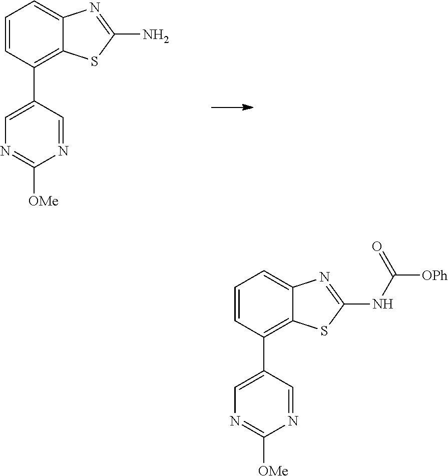 [SMILES: COc1ncc(-c2cccc3nc(N)sc23)cn1 ; COc1ncc(-c2cccc3nc(NC(=O)Oc4ccccc4)sc23)cn1]

Phenyl chloroformate (40.8 mL, 325 mmol) was added to a solution of 7-(2-methoxypyrimidin-5-yl)benzo[d]thiazol-2-amine (56.0 g, 216 mmol) and pyridine (35.0 mL, 433 mmol) in THF (900 mL) at 25° C. The resulting solution was stirred at 25° C. for 12 h. The precipitate was filtered and then triturated with acetonitrile (2×500 mL, 25° C., 2 h) to afford phenyl (7-(2-methoxypyrimidin-5-yl)benzo[d]thiazol-2-yl)carbamate (61.0 g, 157 mmol, 73%) as a gray solid; δ_{H} (400 MHz; DMSO-d6) 12.82 (1H, br s), 8.98 (2H, s), 7.86 (1H, d, J 8.00 Hz), 7.63 (1H, t, J 8.00 Hz), 7.57-7.46 (3H, m), 7.42-7.26 (3H, m), 4.04 (3H, s); ES-MS [M+H]⁺: 379.0.

### Step 3

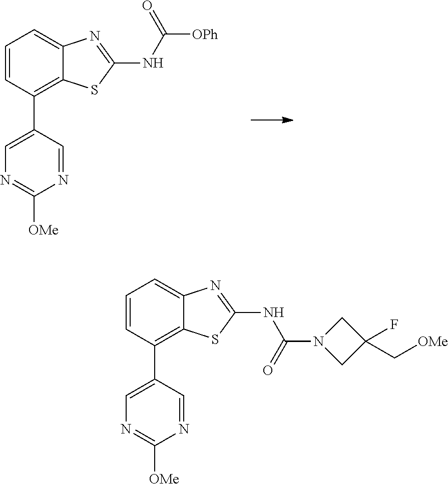 [SMILES: COc1ncc(-c2cccc3nc(NC(=O)Oc4ccccc4)sc23)cn1 ; COCC1(F)CN(C(=O)Nc2nc3cccc(-c4cnc(OC)nc4)c3s2)C1]

Triethylamine (66.2 mL, 475 mmol) was added to a solution of phenyl (7-(2-methoxypyrimidin-5-yl)benzo[d]thiazol-2-yl)carbamate (60.0 g, 158 mmol) and 3-fluoro-3-(methoxymethyl)azetidine hydrochloride (37.0 g, 238 mmol) in dichloromethane (1.2 L). The resulting solution was stirred at 25° C. for 12 h, then filtered and concentrated in vacuo. Trituration was first carried out with a mixture of water (1.8 L) and acetonitrile (600 mL), then ethanol (1 L). Dichloromethane (2 L) and Silicycle SiliaMetS thiol metal scavenger (30.0 g) were added and the resulting mixture was stirred at 30° C. for 30 min, then filtered. Additional scavenger (30.0 g) was added to the filtrate and the resulting mixture was stirred at 30° C. for 30 min, filtered, then concentrated in vacuo. The residue was recrystallized from PE:EtOAc (5:1, 300 mL). Acetonitrile (20 mL) and water (200 mL) were added and the resulting mixture was stirred for 10 min. Concentration in vacuo and lyophilization afforded 3-fluoro-3-(methoxymethyl)-N-(7-(2-methoxypyrimidin-5-yl)benzo[d]thiazol-2-yl)azetidine-1-carboxamide (30.0 g, 77.2 mmol, 49%) as a white solid; δ_{H} (400 MHz; DMSO-d6); 11.69-11.43 (1H, m), 8.93 (2H, s), 7.70 (1H, s), 7.52 (1H, t, J 7.80 Hz), 7.38 (1H, d, J 7.20 Hz), 4.30-4.04 (4H, m), 4.01 (3H, s), 3.78-3.64 (2H, m), 3.34 (3H, s); δ_{F} (400 MHz; DMSO-d6) −155.48; ES-MS [M+H]⁺: 404.0.

Other examples prepared using procedures similar to synthetic procedure A are shown in the following table. All intermediates are commercially available unless noted (see ‘Intermediate synthesis’ section).

| | | | Intermediates | ES- |
|---|---|---|---|---|
| Ex. | | | requiring | MS |
| No. | Name | Structure | synthesis | [M + H]⁺ |
| 2 | 3-(methoxymethyl)-N-(7- phenylbenzo[d]thiazol-2- yl)azetidine-1-carboxamide | 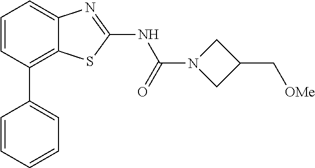 [SMILES: COCC1CN(C(=O)Nc2nc3cccc(-c4ccccc4)c3s2)C1] | n/a | 354.1 |

| | | | | |
|---|---|---|---|---|
| 3 | 3-(methoxymethyl)-N-(7- (6-methoxypyridin-3- yl)benzo[d]thiazol-2- yl)azetidine-1-carboxamide | 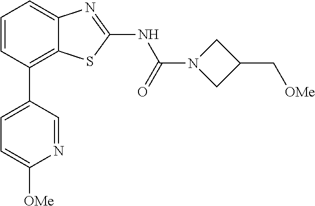 [SMILES: COCC1CN(C(=O)Nc2nc3cccc(-c4ccc(OC)nc4)c3s2)C1] | n/a | 385.1 |

| | | | | |
|---|---|---|---|---|
| 4 | 3-methoxy-3- (methoxymethyl)-N-(7-(6- methoxypyridin-3- yl)benzo[d]thiazol-2- yl)azetidine-1-carboxamide | 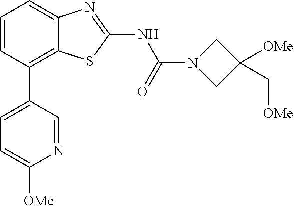 [SMILES: COCC1(OC)CN(C(=O)Nc2nc3cccc(-c4ccc(OC)nc4)c3s2)C1] | n/a | 415.1 |

| | | | | |
|---|---|---|---|---|
| 5 | N-(7-(6-methoxypyridin-3- yl)benzo[d]thiazol-2-yl)-3- ((trifluoromethoxy)methyl) azetidine-1-carboxamide | 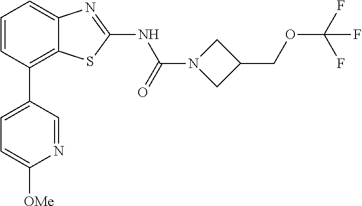 [SMILES: COc1ccc(-c2cccc3nc(NC(=O)N4CC(COC(F)(F)F)C4)sc23)cn1] | n/a | 439.1 |

| | | | | |
|---|---|---|---|---|
| 6 | 3-(2-hydroxypropan-2-yl)- N-(7-(6-methoxypyridin-3- yl)benzo[d]thiazol-2- yl)azetidine-1-carboxamide | 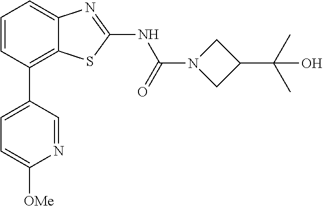 [SMILES: COc1ccc(-c2cccc3nc(NC(=O)N4CC(C(C)(C)O)C4)sc23)cn1] | n/a | 399.1 |

| | | | | |
|---|---|---|---|---|
| 7 | 3-(2-methoxypropan-2-yl)- N-(7-(6-methoxypyridin-3- yl)benzo[d]thiazol-2- yl)azetidine-1-carboxamide | 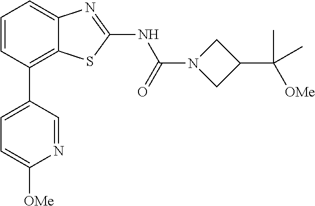 [SMILES: COc1ccc(-c2cccc3nc(NC(=O)N4CC(C(C)(C)OC)C4)sc23)cn1] | n/a | 413.1 |

| | | | | |
|---|---|---|---|---|
| 8 | N-(7-(6-methoxypyridin-3- yl)benzo[d]thiazol-2-yl)-2- oxa-6-azaspiro[3.3]heptane- 6-carboxamide | 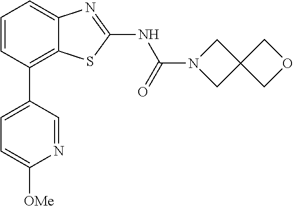 [SMILES: COc1ccc(-c2cccc3nc(NC(=O)N4CC5(COC5)C4)sc23)cn1] | n/a | 383.2 |

| | | | | |
|---|---|---|---|---|
| 9 | N-(7-(6-methoxypyridin-3- yl)benzo[d]thiazol-2-yl)-3- (methylsulfonyl)azetidine- 1-carboxamide | 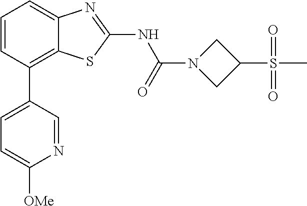 [SMILES: COc1ccc(-c2cccc3nc(NC(=O)N4CC(S(C)(=O)=O)C4)sc23)cn1] | n/a | 419.1 |

| | | | | |
|---|---|---|---|---|
| 10 | N-(7-(6-methoxypyridin-3- yl)benzo[d]thiazol-2-yl)-3- (2,2,2- trifluoroethyl)azetidine-1- carboxamide | 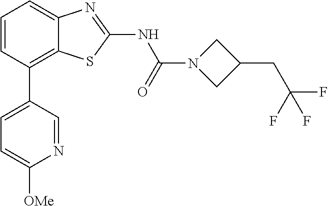 [SMILES: COc1ccc(-c2cccc3nc(NC(=O)N4CC(CC(F)(F)F)C4)sc23)cn1] | n/a | 423.0 |

| | | | | |
|---|---|---|---|---|
| 11 | 3-(methoxy-d3)-N-(7-(6- methoxypyridin-3- yl)benzo[d]thiazol-2-yl)-3- methylazetidine-1- carboxamide | 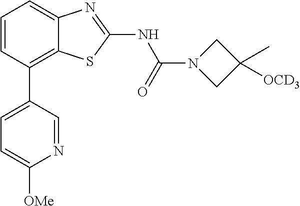 | 3-(methoxy-d3)- 3- methylazetidine | 388.1 |

| | | | | |
|---|---|---|---|---|
| 12 | 3-(methoxymethyl)-N-(7- (6-methoxypyridin-3- yl)benzo[d]thiazol-2-yl)-3- methylazetidine-1- carboxamide | 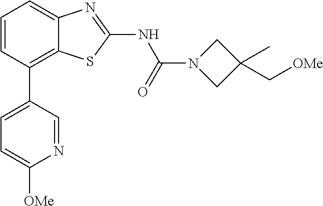 [SMILES: COCC1(C)CN(C(=O)Nc2nc3cccc(-c4ccc(OC)nc4)c3s2)C1] | n/a | 399.1 |

| | | | | |
|---|---|---|---|---|
| 13 | N-(7-(6-methoxypyridin-3- yl)benzo[d]thiazol-2-yl)-8- methyl-5-oxa-2,8- diazaspiro[3.5]nonane-2- carboxamide | 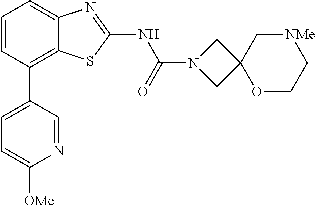 [SMILES: COc1ccc(-c2cccc3nc(NC(=O)N4CC5(CN(C)CCO5)C4)sc23)cn1] | n/a | 426.2 |

| | | | | |
|---|---|---|---|---|
| 14 | 8-cyclopropyl-N-(7-(6- methoxypyridin-3- yl)benzo[d]thiazol-2-yl)-5- oxa-2,8- diazaspiro[3.5]nonane-2- carboxamide | 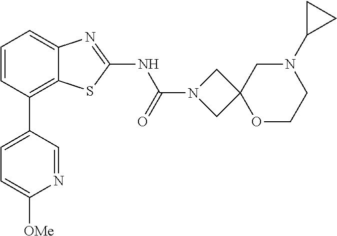 [SMILES: COc1ccc(-c2cccc3nc(NC(=O)N4CC5(C4)CN(C4CC4)CCO5)sc23)cn1] | 8-cyclopropyl-5- oxa-2,8- diazaspiro[3.5] nonane | 452.2 |

| | | | | |
|---|---|---|---|---|
| 15 | N-(7-(6-methoxypyridin-3- yl)benzo[d]thiazol-2-yl)-6- methyl-2,6- diazaspiro[3.5]nonane-2- carboxamide | 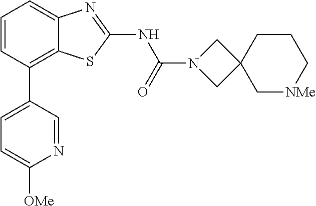 [SMILES: COc1ccc(-c2cccc3nc(NC(=O)N4CC5(CCCN(C)C5)C4)sc23)cn1] | n/a | 424.2 |

| | | | | |
|---|---|---|---|---|
| 16 | N-(7-(6-methoxypyridin-3- yl)benzo[d]thiazol-2-yl)-6- methyl-2,6- diazaspiro[3.4]octane-2- carboxamide | 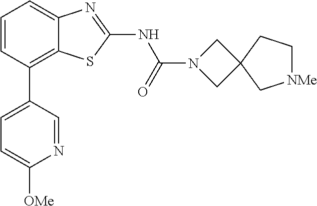 [SMILES: COc1ccc(-c2cccc3nc(NC(=O)N4CC5(CCN(C)C5)C4)sc23)cn1] | n/a | 410.2 |

| | | | | |
|---|---|---|---|---|
| 17 | N-(7-(6-methoxy-2- methylpyridin-3- yl)benzo[d]thiazol-2-yl)-2- oxa-6-azaspiro[3.3]heptane- 6-carboxamide | 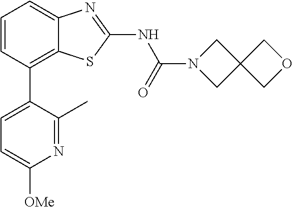 [SMILES: COc1ccc(-c2cccc3nc(NC(=O)N4CC5(COC5)C4)sc23)c(C)n1] | n/a | 397.2 |

| | | | | |
|---|---|---|---|---|
| 18 | 3-(fluoromethyl)-N-(7-(6- methoxypyridin-3- yl)benzo[d]thiazol-2- yl)azetidine-1-carboxamide | 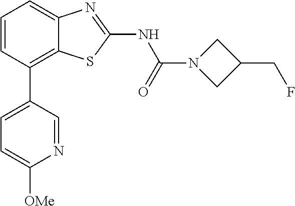 [SMILES: COc1ccc(-c2cccc3nc(NC(=O)N4CC(CF)C4)sc23)cn1] | n/a | 373.1 |

| | | | | |
|---|---|---|---|---|
| 19 | 6-methoxy-N-(7-(6- methoxypyridin-3- yl)benzo[d]thiazol-2-yl)-2- azaspiro[3.3]heptane-2- carboxamide | 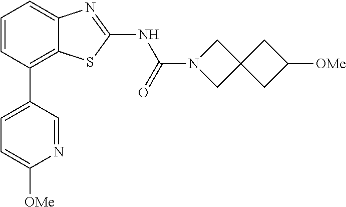 [SMILES: COc1ccc(-c2cccc3nc(NC(=O)N4CC5(CC(OC)C5)C4)sc23)cn1] | n/a | 411.2 |

| | | | | |
|---|---|---|---|---|
| 20 | 6-hydroxy-N-(7-(6- methoxypyridin-3- yl)benzo[d]thiazol-2-yl)-6- methyl-2- azaspiro[3.3]heptane-2- carboxamide | 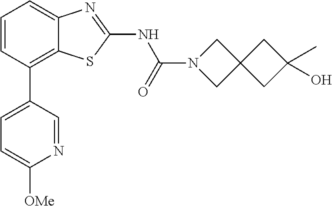 [SMILES: COc1ccc(-c2cccc3nc(NC(=O)N4CC5(C4)CC(C)(O)C5)sc23)cn1] | n/a | 411.2 |

| | | | | |
|---|---|---|---|---|
| 21 | 6,6-difluoro-N-(7-(6- methoxypyridin-3- yl)benzo[d]thiazol-2-yl)-2- azaspiro[3.3]heptane-2- carboxamide | 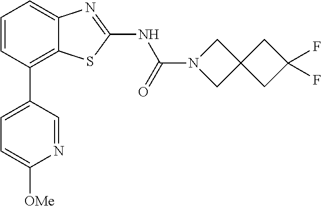 [SMILES: COc1ccc(-c2cccc3nc(NC(=O)N4CC5(C4)CC(F)(F)C5)sc23)cn1] | n/a | 417.1 |

| | | | | |
|---|---|---|---|---|
| 22 | 5,5-difluoro-N-(7-(6- methoxypyridin-3- yl)benzo[d]thiazol-2-yl)-2- azaspiro[3.3]heptane-2- carboxamide | 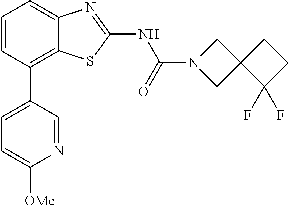 [SMILES: COc1ccc(-c2cccc3nc(NC(=O)N4CC5(CCC5(F)F)C4)sc23)cn1] | n/a | 417.1 |

| | | | | |
|---|---|---|---|---|
| 23 | 6-fluoro-N-(7-(6- methoxypyridin-3- yl)benzo[d]thiazol-2-yl)-2- azaspiro[3.3]heptane-2- carboxamide | 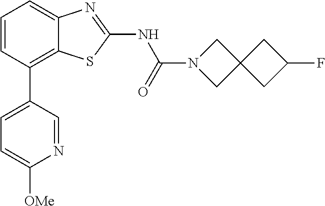 [SMILES: COc1ccc(-c2cccc3nc(NC(=O)N4CC5(CC(F)C5)C4)sc23)cn1] | n/a | 399.1 |

| | | | | |
|---|---|---|---|---|
| 24 | 6-cyano-N-(7-(6- methoxypyridin-3- yl)benzo[d]thiazol-2-yl)-2- azaspiro[3.3]heptane-2- carboxamide | 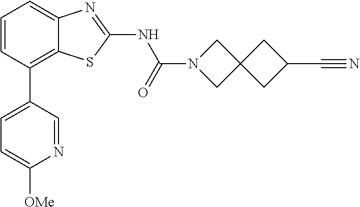 [SMILES: COc1ccc(-c2cccc3nc(NC(=O)N4CC5(CC(C#N)C5)C4)sc23)cn1] | n/a | 406.1 |

| | | | | |
|---|---|---|---|---|
| 25 | 1-cyano-N-(7-(6- methoxypyridin-3- yl)benzo[d]thiazol-2-yl)-5- azaspiro[2.3]hexane-5- carboxamide | 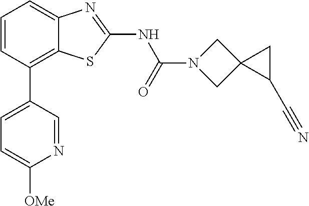 [SMILES: COc1ccc(-c2cccc3nc(NC(=O)N4CC5(CC5C#N)C4)sc23)cn1] | n/a | 392.0 |

| | | | | |
|---|---|---|---|---|
| 26 | 5-methoxy-N-(7-(6- methoxypyridin-3- yl)benzo[d]thiazol-2-yl)-2- azaspiro[3.3]heptane-2- carboxamide | 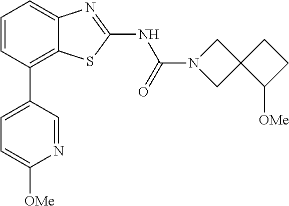 [SMILES: COc1ccc(-c2cccc3nc(NC(=O)N4CC5(CCC5OC)C4)sc23)cn1] | n/a | 411.2 |

| | | | | |
|---|---|---|---|---|
| 27 | 6-(difluoromethyl)-6- hydroxy-N-(7-(6- methoxypyridin-3- yl)benzo[d]thiazol-2-yl)-2- azaspiro[3.3]heptane-2- carboxamide | 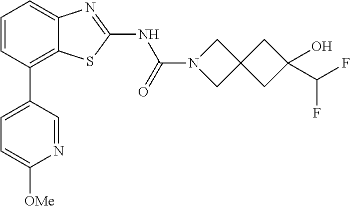 [SMILES: COc1ccc(-c2cccc3nc(NC(=O)N4CC5(C4)CC(O)(C(F)F)C5)sc23)cn1] | n/a | 447.1 |

| | | | | |
|---|---|---|---|---|
| 28 | 3- ((difluoromethoxy)methyl)- 3-fluoro-N-(7-(6- methoxypyridin-3- yl)benzo[d]thiazol-2- yl)azetidine-1-carboxamide | 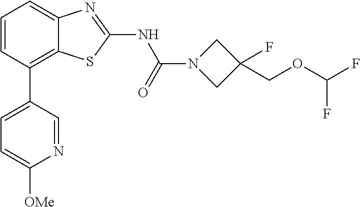 [SMILES: COc1ccc(-c2cccc3nc(NC(=O)N4CC(F)(COC(F)F)C4)sc23)cn1] | 3- ((difluoromethoxy) methyl)-3- fluoroazetidine | 439.1 |

| | | | | |
|---|---|---|---|---|
| 29 | N-(7-(6-methoxypyridin-3- yl)benzo[d]thiazol-2-yl)-6- (trifluoromethyl)-2- azaspiro[3.3]heptane-2- carboxamide | 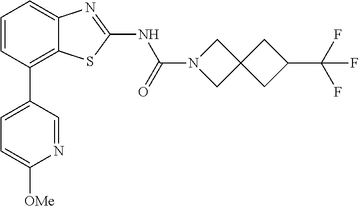 [SMILES: COc1ccc(-c2cccc3nc(NC(=O)N4CC5(CC(C(F)(F)F)C5)C4)sc23)cn1] | n/a | 449.1 |

| | | | | |
|---|---|---|---|---|
| 30 | N-(7-(2-ethoxypyrimidin-5- yl)benzo[d]thiazol-2-yl)-3- fluoro-3-((methoxy- d3)methyl)azetidine-1- carboxamide | 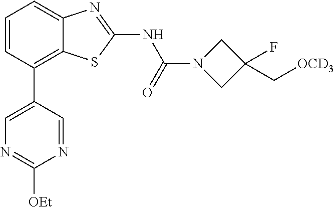 | 3-fluoro-3- ((methoxy- d3)methyl)azetidine | 421.2 |

| | | | | |
|---|---|---|---|---|
| 31 | 9,9-difluoro-N-(7-(6- methoxypyridin-3- yl)benzo[d]thiazol-2-yl)-6- methyl-2,6- diazaspiro[3.5]nonane-2- carboxamide | 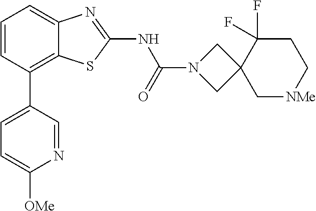 [SMILES: COc1ccc(-c2cccc3nc(NC(=O)N4CC5(CN(C)CCC5(F)F)C4)sc23)cn1] | 9,9-difluoro-6- methyl-2,6- diazaspiro[3.5] nonane | 460.2 |

| | | | | |
|---|---|---|---|---|
| 32 | N-(7-(2-ethoxypyrimidin-5- yl)benzo[d]thiazol-2-yl)-6- oxa-2-azaspiro[3.4]octane- 2-carboxamide | 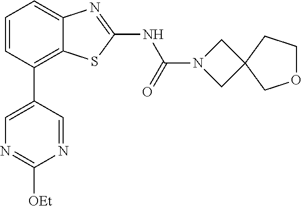 [SMILES: CCOc1ncc(-c2cccc3nc(NC(=O)N4CC5(CCOC5)C4)sc23)cn1] | n/a | 412.2 |

| | | | | |
|---|---|---|---|---|
| 33 | N-(7-(2-methoxypyrimidin- 5-yl)benzo[d]thiazol-2-yl)- 6-oxa-2- azaspiro[3.4]octane-2- carboxamide | 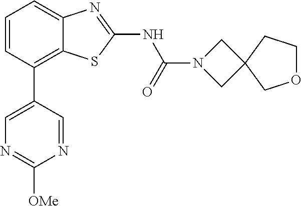 [SMILES: COc1ncc(-c2cccc3nc(NC(=O)N4CC5(CCOC5)C4)sc23)cn1] | n/a | 398.2 |

| | | | | |
|---|---|---|---|---|
| 34 | N-(7-(2-methoxypyrimidin- 5-yl)benzo[d]thiazol-2-yl)- 5-oxa-2- azaspiro[3.4]octane-2- carboxamide | 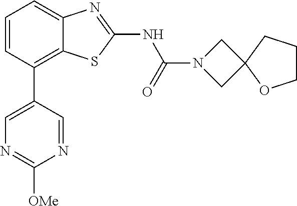 [SMILES: COc1ncc(-c2cccc3nc(NC(=O)N4CC5(CCCO5)C4)sc23)cn1] | n/a | 398.1 |

| | | | | |
|---|---|---|---|---|
| 35 | N-(7-(2-ethoxypyrimidin-5- yl)benzo[d]thiazol-2-yl)-5- oxa-2-azaspiro[3.4]octane- 2-carboxamide | 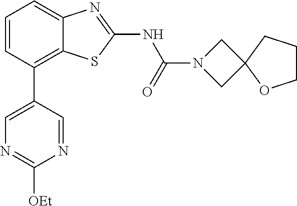 [SMILES: CCOc1ncc(-c2cccc3nc(NC(=O)N4CC5(CCCO5)C4)sc23)cn1] | n/a | 412.2 |

| | | | | |
|---|---|---|---|---|
| 36 | 3-(ethoxymethyl)-N-(7-(2- ethoxypyrimidin-5- yl)benzo[d]thiazol-2-yl)-3- fluoroazetidine-1- carboxamide | 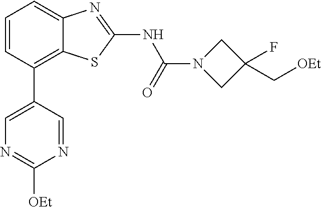 [SMILES: CCOCC1(F)CN(C(=O)Nc2nc3cccc(-c4cnc(OCC)nc4)c3s2)C1] | 3-(ethoxymethyl)- 3-fluoroazetidine | 432.2 |

| | | | | |
|---|---|---|---|---|
| 37 | 3-fluoro-3- (methoxymethyl)-N-(7-(6- methoxypyridin-3- yl)benzo[d]thiazol-2- yl)azetidine-1-carboxamide | 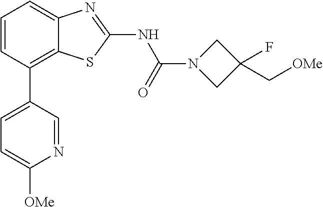 [SMILES: COCC1(F)CN(C(=O)Nc2nc3cccc(-c4ccc(OC)nc4)c3s2)C1] | n/a | 403.2 |

| | | | | |
|---|---|---|---|---|
| 38 | 3-fluoro-3-((methoxy- d3)methyl)-N-(7-(6- methoxypyridin-3- yl)benzo[d]thiazol-2- yl)azetidine-1-carboxamide | 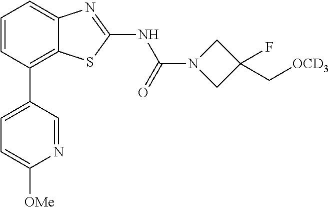 | 3-fluoro-3-((methoxy- d3)methyl)azetidine | 406.1 |

| | | | | |
|---|---|---|---|---|
| 39 | 3-(methoxy-d3)-N-(7-(6- methoxypyridin-3- yl)benzo[d]thiazol-2- yl)azetidine-1-carboxamide | 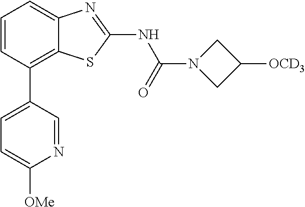 | 3-(methoxy- d3)azetidine | 374.1 |

| | | | | |
|---|---|---|---|---|
| 40 | 3-ethoxy-N-(7-(6- methoxypyridin-3- yl)benzo[d]thiazol-2-yl)-3- methylazetidine-1- carboxamide | 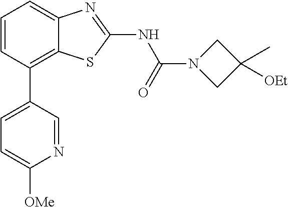 [SMILES: CCOC1(C)CN(C(=O)Nc2nc3cccc(-c4ccc(OC)nc4)c3s2)C1] | n/a | 399.2 |

| | | | | |
|---|---|---|---|---|
| 41 | 3-ethoxy-N-(7-(6- methoxypyridin-3- yl)benzo[d]thiazol-2- yl)azetidine-1-carboxamide | 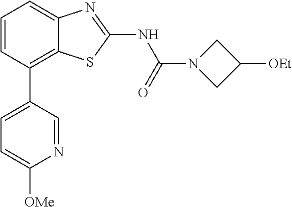 [SMILES: CCOC1CN(C(=O)Nc2nc3cccc(-c4ccc(OC)nc4)c3s2)C1] | n/a | 385.2 |

| | | | | |
|---|---|---|---|---|
| 42 | 3-methoxy-N-(7-(6- methoxypyridin-3- yl)benzo[d]thiazol-2-yl)-3- methylazetidine-1- carboxamide | 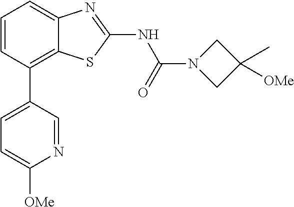 [SMILES: COc1ccc(-c2cccc3nc(NC(=O)N4CC(C)(OC)C4)sc23)cn1] | n/a | 385.2 |

| | | | | |
|---|---|---|---|---|
| 43 | 3-isopropoxy-N-(7-(6- methoxypyridin-3- yl)benzo[d]thiazol-2- yl)azetidine-1-carboxamide | 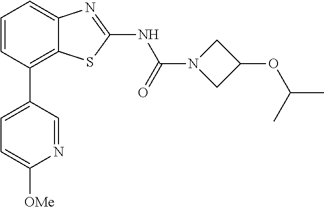 [SMILES: COc1ccc(-c2cccc3nc(NC(=O)N4CC(OC(C)C)C4)sc23)cn1] | n/a | 399.1 |

| | | | | |
|---|---|---|---|---|
| 44 | N-(7-(6-methoxypyridin-3- yl)benzo[d]thiazol-2-yl)-6- oxa-2-azaspiro[3.5]nonane- 2-carboxamide | 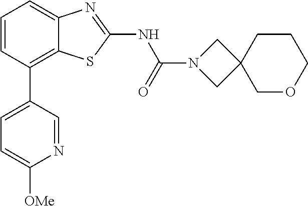 [SMILES: COc1ccc(-c2cccc3nc(NC(=O)N4CC5(CCCOC5)C4)sc23)cn1] | n/a | 411.2 |

| | | | | |
|---|---|---|---|---|
| 45 | N-(7-(6-methoxypyridin-3- yl)benzo[d]thiazol-2-yl)-5- oxa-2-azaspiro[3.5]nonane- 2-carboxamide | 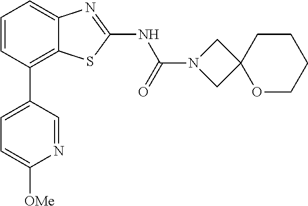 [SMILES: COc1ccc(-c2cccc3nc(NC(=O)N4CC5(CCCCO5)C4)sc23)cn1] | n/a | 411.2 |

| | | | | |
|---|---|---|---|---|
| 46 | N-(7-(6-methoxypyridin-3- yl)benzo[d]thiazol-2-yl)-2- thia-6- azaspiro[3.3]heptane-6- carboxamide2,2-dioxide | 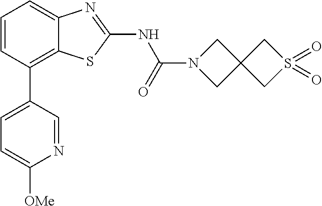 [SMILES: COc1ccc(-c2cccc3nc(NC(=O)N4CC5(C4)CS(=O)(=O)C5)sc23)cn1] | n/a | 431.1 |

| | | | | |
|---|---|---|---|---|
| 47 | 1,1-difluoro-N-(7-(6- methoxypyridin-3- yl)benzo[d]thiazol-2-yl)-5- azaspiro[2.3]hexane-5- carboxamide | 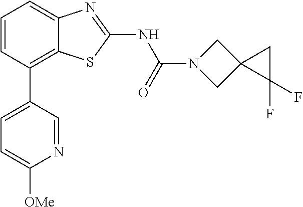 [SMILES: COc1ccc(-c2cccc3nc(NC(=O)N4CC5(C4)CC5(F)F)sc23)cn1] | n/a | 403.1 |

| | | | | |
|---|---|---|---|---|
| 48 | 3-cyclopropyl-3-hydroxy- N-(7-(6-methoxypyridin-3- yl)benzo[d]thiazol-2- yl)azetidine-1-carboxamide | 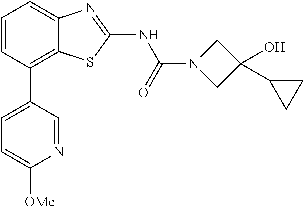 [SMILES: COc1ccc(-c2cccc3nc(NC(=O)N4CC(O)(C5CC5)C4)sc23)cn1] | n/a | 397.2 |

| | | | | |
|---|---|---|---|---|
| 49 | 6-hydroxy-N-(7-(6- methoxypyridin-3- yl)benzo[d]thiazol-2-yl)-6- (trifluoromethyl)-2- azaspiro[3.3]heptane-2- carboxamide | 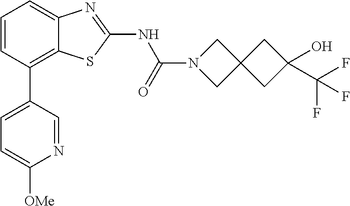 [SMILES: COc1ccc(-c2cccc3nc(NC(=O)N4CC5(C4)CC(O)(C(F)(F)F)C5)sc23)cn1] | n/a | 465.1 |

| | | | | |
|---|---|---|---|---|
| 50 | N-(7-(6-methoxypyridin-3- yl)benzo[d]thiazol-2-yl)-7- oxa-2-azaspiro[3.5]nonane- 2-carboxamide | 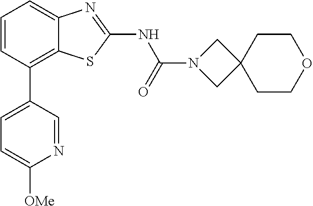 [SMILES: COc1ccc(-c2cccc3nc(NC(=O)N4CC5(CCOCC5)C4)sc23)cn1] | n/a | 411.2 |

| | | | | |
|---|---|---|---|---|
| 51 | 3-hydroxy-N-(7-(6- methoxypyridin-3- yl)benzo[d]thiazol-2-yl)-3- (trifluoromethyl)azetidine- 1-carboxamide | 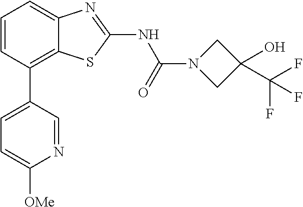 [SMILES: COc1ccc(-c2cccc3nc(NC(=O)N4CC(O)(C(F)(F)F)C4)sc23)cn1] | n/a | 425.1 |

| | | | | |
|---|---|---|---|---|
| 52 | N-(7-(6-methoxypyridin-3- yl)benzo[d]thiazol-2-yl)-1- oxa-6-azaspiro[3.3]heptane- 6-carboxamide | 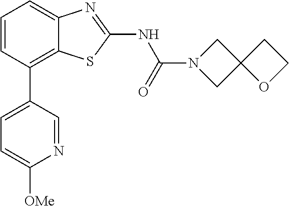 [SMILES: COc1ccc(-c2cccc3nc(NC(=O)N4CC5(CCO5)C4)sc23)cn1] | n/a | 383.1 |

| | | | | |
|---|---|---|---|---|
| 53 | 3- ((difluoromethoxy)methyl)- N-(7-(6-methoxypyridin-3- yl)benzo[d]thiazol-2- yl)azetidine-1-carboxamide | 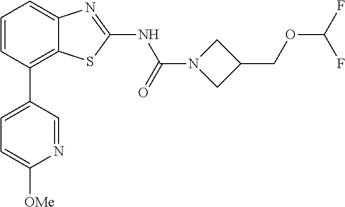 [SMILES: COc1ccc(-c2cccc3nc(NC(=O)N4CC(COC(F)F)C4)sc23)cn1] | n/a | 421.0 |

| | | | | |
|---|---|---|---|---|
| 54 | 3-fluoro-3- (methoxymethyl)-N-(7-(2- methoxypyrimidin-5-yl)-4- methylbenzo[d]thiazol-2- yl)azetidine-1-carboxamide | 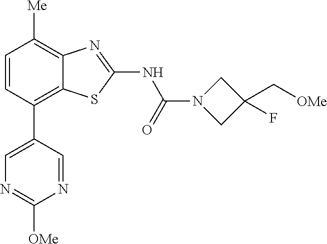 [SMILES: COCC1(F)CN(C(=O)Nc2nc3c(C)ccc(-c4cnc(OC)nc4)c3s2)C1] | n/a | 418.2 |

| | | | | |
|---|---|---|---|---|
| 55 | N-(6-fluoro-7-(6- methoxypyridin-3- yl)benzo[d]thiazol-2-yl)-5- oxa-2-azaspiro[3.4]octane- 2-carboxamide | 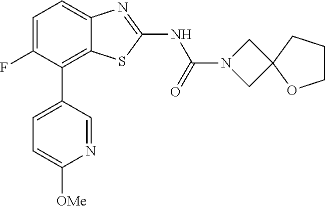 [SMILES: COc1ccc(-c2c(F)ccc3nc(NC(=O)N4CC5(CCCO5)C4)sc23)cn1] | 7-bromo-6- fluorobenzo[d] thiazol-2-amine | 415.1 |

| | | | | |
|---|---|---|---|---|
| 56 | N-(6-fluoro-7-(6- methoxypyridin-3- yl)benzo[d]thiazol-2-yl)-6- oxa-2-azaspiro[3.4]octane- 2-carboxamide | 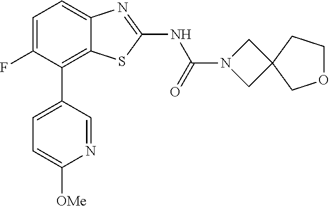 [SMILES: COc1ccc(-c2c(F)ccc3nc(NC(=O)N4CC5(CCOC5)C4)sc23)cn1] | 7-bromo-6- fluorobenzo[d] thiazol-2-amine | 415.1 |

| | | | | |
|---|---|---|---|---|
| 57 | N-(6-chloro-7-(6-(1,1- difluoroethyl)pyridin-3- yl)benzo[d]thiazol-2-yl)-3- (methoxy-d3)-3- methylazetidine-1- carboxamide | 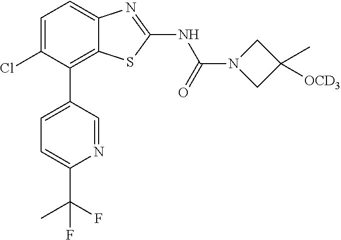 | 3-(methoxy-d3)-3- methylazetidine and 7-bromo-6- chlorobenzo[d] thiazol-2-amine | 456.1 |

| | | | | |
|---|---|---|---|---|
| 58 | N-(6-chloro-7-(6- methoxypyridin-3- yl)benzo[d]thiazol-2-yl)-3- methoxy-3- methylazetidine-1- carboxamide | 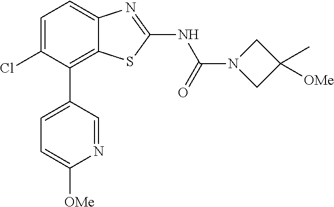 [SMILES: COc1ccc(-c2c(Cl)ccc3nc(NC(=O)N4CC(C)(OC)C4)sc23)cn1] | 7-bromo-6- chlorobenzo[d] thiazol-2-amine | 419.1 |

| | | | | |
|---|---|---|---|---|
| 59 | N-(6-chloro-7-(6-(1,1- difluoroethyl)pyridin-3- yl)benzo[d]thiazol-2-yl)-2- oxa-6-azaspiro[3.3]heptane- 6-carboxamide | 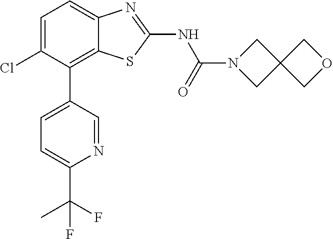 [SMILES: CC(F)(F)c1ccc(-c2c(Cl)ccc3nc(NC(=O)N4CC5(COC5)C4)sc23)cn1] | 7-bromo-6- chlorobenzo[d] thiazol-2-amine | 451.1 |

| | | | | |
|---|---|---|---|---|
| 60 | N-(6-chloro-7-(6- methoxypyridin-3- yl)benzo[d]thiazol-2-yl)-2- oxa-6-azaspiro[3.3]heptane- 6-carboxamide | 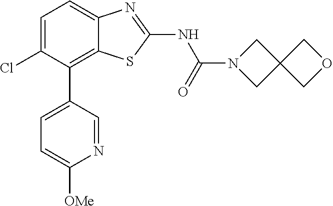 [SMILES: COc1ccc(-c2c(Cl)ccc3nc(NC(=O)N4CC5(COC5)C4)sc23)cn1] | 7-bromo-6- chlorobenzo[d] thiazol-2-amine | 417.2 |

| | | | | |
|---|---|---|---|---|
| 61 | N-(6-chloro-7-(6- methoxypyridin-3- yl)benzo[d]thiazol-2-yl)-3- fluoro-3- (methoxymethyl)azetidine- 1-carboxamide | 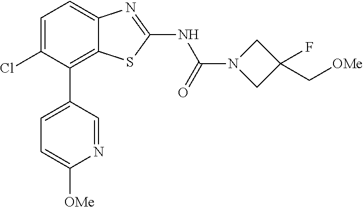 [SMILES: COCC1(F)CN(C(=O)Nc2nc3ccc(Cl)c(-c4ccc(OC)nc4)c3s2)C1] | 7-bromo-6- chlorobenzo[d] thiazol-2-amine | 437.1 |

| | | | | |
|---|---|---|---|---|
| 62 | 3-fluoro-N-(6-methoxy-7- (6-methoxypyridin-3- yl)benzo[d]thiazol-2-yl)-3- (methoxymethyl)azetidine- 1-carboxamide | 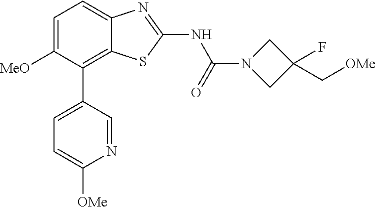 [SMILES: COCC1(F)CN(C(=O)Nc2nc3ccc(OC)c(-c4ccc(OC)nc4)c3s2)C1] | 7-bromo-6- methoxybenzo[d] thiazol-2-amine | 433.2 |

| | | | | |
|---|---|---|---|---|
| 63 | N-(6-methoxy-7-(6- methoxypyridin-3- yl)benzo[d]thiazol-2-yl)-2- oxa-6-azaspiro[3.3]heptane- 6-carboxamide | 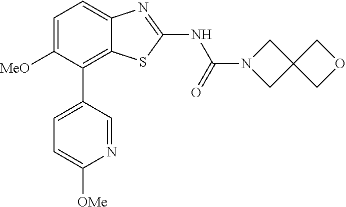 [SMILES: COc1ccc(-c2c(OC)ccc3nc(NC(=O)N4CC5(COC5)C4)sc23)cn1] | 7-bromo-6- methoxybenzo[d] thiazol-2-amine | 413.1 |

| | | | | |
|---|---|---|---|---|
| 64 | N-(7-(6-methoxypyridin-3- yl)-6- methylbenzo[d]thiazol-2- yl)-2-oxa-6- azaspiro[3.3]heptane-6- carboxamide | 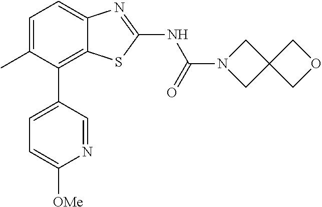 [SMILES: COc1ccc(-c2c(C)ccc3nc(NC(=O)N4CC5(COC5)C4)sc23)cn1] | 7-bromo-6- methylbenzo[d] thiazol-2-amine | 397.2 |

| | | | | |
|---|---|---|---|---|
| 65 | N-(6-chloro-7-(5- methoxypyrazin-2- yl)benzo[d]thiazol-2-yl)-3- (methoxy-d3)-3- methylazetidine-1- carboxamide | 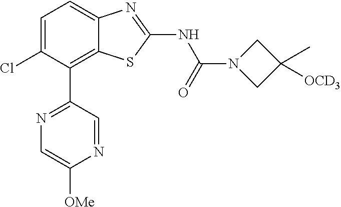 | 3-(methoxy-d3)- 3- methylazetidine and 7-bromo-6- chlorobenzo[d] thiazol-2-amine | 423.3 |
| | | (Stille coupling using 2-methoxy-5- | | |
| | | (tributylstannyl)pyrazine for Step 1: | | |
| | | Pd₂(dba)₃, PCy₃, LiCl, dioxane, | | |
| | | 110° C.) | | |
| 345 | N-(6-fluoro-7-(2- isopropoxypyrimidin-5- yl)benzo[d]thiazol-2-yl)-3- hydroxy-3- (methoxymethyl)azetidine- 1-carboxamide | 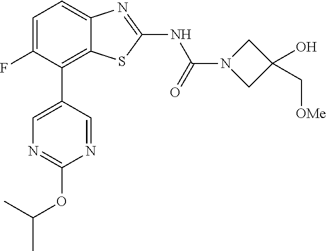 [SMILES: COCC1(O)CN(C(=O)Nc2nc3ccc(F)c(-c4cnc(OC(C)C)nc4)c3s2)C1] | 7-bromo-6- fluorobenzo[d] thiazol-2-amine | 448.2 |

### Synthetic Procedure B

### Example 66: N-(7-(2-ethoxypyrimidin-5-yl)benzo[d]thiazol-2-yl)-3-fluoro-3-(methoxymethyl)azetidine-1-carboxamide

### Step 1

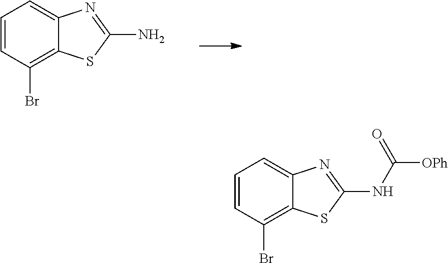 [SMILES: Nc1nc2cccc(Br)c2s1 ; O=C(Nc1nc2cccc(Br)c2s1)Oc1ccccc1]

Phenyl chloroformate (4.11 mL, 32.7 mmol) was added to a solution of 7-bromobenzo[d]thiazol-2-amine (5.00 g, 21.8 mmol) and pyridine (3.52 mL, 43.7 mmol) in THF (100 mL). The resulting mixture was stirred at 20° C. for 2 h and then concentrated in vacuo. Trituration (acetonitrile:water 1:1, 50 mL) afforded phenyl (7-bromobenzo[d]thiazol-2-yl)carbamate (7.00 g, 19.9 mmol, 91%) as an off-white solid; δ_{H} (400 MHz; DMSO-d6) 13.11-12.66 (1H, m), 7.81 (1H, d, J 8 Hz), 7.57-7.44 (4H, m) 7.43-7.33 (3H, m); ES-MS [M+H]⁺: 351.0.

### Step 2

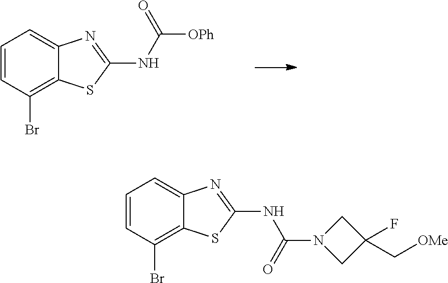 [SMILES: O=C(Nc1nc2cccc(Br)c2s1)Oc1ccccc1 ; COCC1(F)CN(C(=O)Nc2nc3cccc(Br)c3s2)C1]

3-Fluoro-3-(methoxymethyl)azetidine (2.77 g, 11.9 mmol) was added to a solution of phenyl (7-bromobenzo[d]thiazol-2-yl)carbamate (2.00 g, 5.73 mmol) and triethylamine (0.80 mL, 5.73 mmol) in THF (20 mL). The resulting solution was stirred at 20° C. for 2 h, then concentrated in vacuo. Trituration (acetonitrile:water 1:1, 20 mL) afforded N-(7-bromobenzo[d]thiazol-2-yl)-3-fluoro-3-(methoxymethyl)azetidine-1-carboxamide (2.00 g, 5.24 mmol, 91%) as a white solid; δ_{H} (400 MHz; DMSO-d6) 11.84-11.47 (1H, m), 7.69-7.61 (1H, m), 7.45 (1H, d, J 7.75 Hz), 7.38-7.32 (1H, m), 4.27-4.07 (4H, m), 3.75 (1H, s), 3.69 (1H, s), 3.35 (3H, br s); ES-MS [M+H]⁺: 374.0.

### Step 3

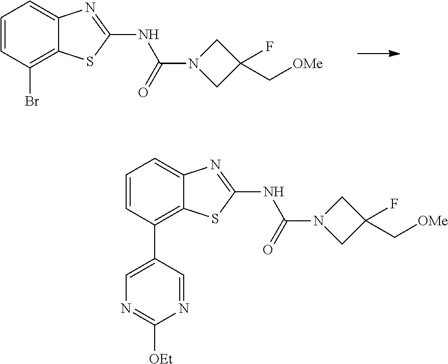 [SMILES: COCC1(F)CN(C(=O)Nc2nc3cccc(Br)c3s2)C1 ; CCOc1ncc(-c2cccc3nc(NC(=O)N4CC(F)(COC)C4)sc23)cn1]

A mixture of N-(7-bromobenzo[d]thiazol-2-yl)-3-fluoro-3-(methoxymethyl)azetidine-1-carboxamide (200 mg, 0.53 mmol), (2-ethoxypyrimidin-5-yl)boronic acid (99 mg, 0.59 mmol), Pd(dppf)Cl₂ (39 mg, 0.05 mmol), Na₂CO₃ (170 mg, 1.60 mmol), dioxane (2 mL) and water (0.5 mL) was degassed and purged with nitrogen three times. The reaction mixture was stirred at 80° C. for 12 h under a nitrogen atmosphere, then cooled, diluted with water (2 mL) and extracted into EtOAc (2×2 mL). The combined organic extracts were dried over anhydrous sodium sulfate and then concentrated in vacuo. Purification by preparative HPLC (Phenomenex Luna C18 150*25 10 μm column; acetonitrile: [water (0.1% formic acid)]; gradient: 37%-67% water (0.1% formic acid) over 10 m afforded N-(7-(2-ethoxypyrimidin-5-yl)benzo[d]thiazol-2-yl)-3-fluoro-3-(methoxymethyl)azetidine-7-carboxamide (66 mg, 0.16 mmol) as a white solid; δ_{H} (400 MHz; CDCl₃) 8.83 (2H, s), 7.74 (1H, d, J 8.19 Hz), 7.54 (1H, m), 7.27 (1H, s), 4.53 (2H, m), 4.27 (4H, m), 3.70 (2H, d, J 17.61 Hz), 3.49 (3H, s), 1.51 (3H, m); δ_{F} (400 MHz; DMSO-d6) −124.8, −155.5; ES-MS [M+H]⁺: 418.2.

Other examples prepared using synthetic procedure B are shown in the following table. All intermediates are commercially available unless noted (see Intermediate synthesis' section).

| | | | Intermediates | |
|---|---|---|---|---|
| Ex. | | | requiring | ES-MS |
| No. | Name | Structure | synthesis | [M + H]⁺ |
| 67 | 3-fluoro-3- (methoxymethyl)-N-(7-(6- ((tetrahydro-2H-pyran-4- yl)amino)pyridin-3- yl)benzo[d]thiazol-2- yl)azetidine-1-carboxamide | 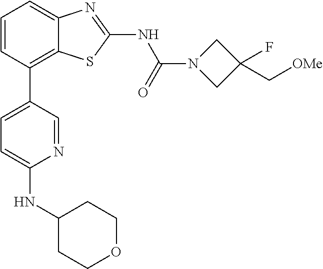 [SMILES: COCC1(F)CN(C(=O)Nc2nc3cccc(-c4ccc(NC5CCOCC5)nc4)c3s2)C1] | N-(tetrahydro-2H- pyran-4-yl)-5- (4,4,5,5- tetramethyl-1,3,2- dioxaborolan-2- yl)pyridin-2-amine | 472.2 |
| 68 | 3-fluoro-3- (methoxymethyl)-N-(7-(6- (methyl(tetrahydro-2H- pyran-4-yl)amino)pyridin- 3-yl)benzo[d]thiazol-2- yl)azetidine-1-carboxamide | 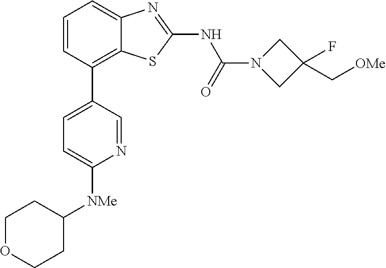 [SMILES: COCC1(F)CN(C(=O)Nc2nc3cccc(-c4ccc(N(C)C5CCOCC5)nc4)c3s2)C1] | N-methyl-N- (tetrahydro-2H- pyran-4-yl)-5- (4,4,5,5- tetramethyl-1,3,2- dioxaborolan-2- yl)pyridin-2-amine | 486.1 |
| 69 | N-(7-(6-(1,1-difluoro-2- hydroxyethyl)pyridin-3- yl)benzo[d]thiazol-2-yl)-3- fluoro-3- (methoxymethyl)azetidine- 1-carboxamide | 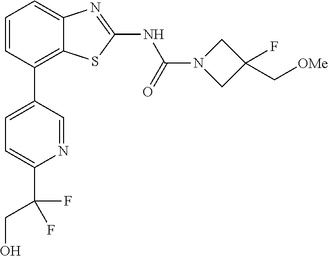 [SMILES: COCC1(F)CN(C(=O)Nc2nc3cccc(-c4ccc(C(F)(F)CO)nc4)c3s2)C1] | 2,2-difluoro-2-(5- (4,4,5,5- tetramethyl-1,3,2- dioxaborolan-2- yl)pyridin-2- yl)ethan-1-ol | 453.1 |
| 70 | 3-fluoro-3- (methoxymethyl)-N-(7-(6- ((4-methyltetrahydro-2H- pyran-4-yl)amino)pyridin- 3-yl)benzo[d]thiazol-2- yl)azetidine-1-carboxamide | 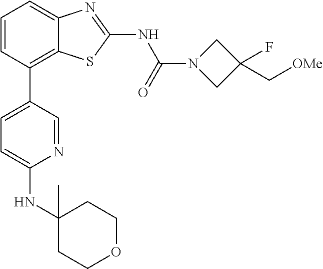 [SMILES: COCC1(F)CN(C(=O)Nc2nc3cccc(-c4ccc(NC5(C)CCOCC5)nc4)c3s2)C1] | N-(4- methyltetrahydro- 2H-pyran-4-yl)-5- (4,4,5,5- tetramethyl-1,3,2- dioxaborolan-2- yl)pyridin-2-amine | 486.1 |
| 71 | N-(7-(6-(oxetan-3- yl)pyridin-3- yl)benzo[d]thiazol-2-yl)-2- oxa-6-azaspiro[3.3]heptane- 6-carboxamide | 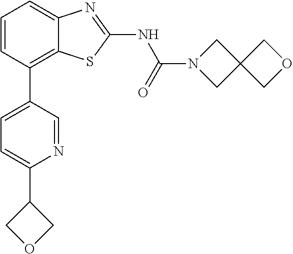 [SMILES: O=C(Nc1nc2cccc(-c3ccc(C4COC4)nc3)c2s1)N1CC2(COC2)C1] | n/a | 409.2 |
| 72 | N-(7-(6-(oxetan-3- yloxy)pyridin-3- yl)benzo[d]thiazol-2-yl)-2- oxa-6-azaspiro[3.3]heptane- 6-carboxamide | 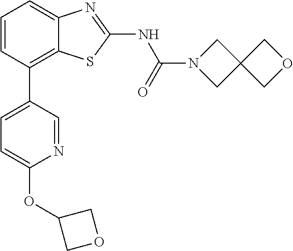 [SMILES: O=C(Nc1nc2cccc(-c3ccc(OC4COC4)nc3)c2s1)N1CC2(COC2)C1] | n/a | 425.1 |
| 73 | N-(7-(6- (difluoromethoxy)pyridin- 3-yl)benzo[d]thiazol-2-yl)- 2-oxa-6- azaspiro[3.3]heptane-6- carboxamide | 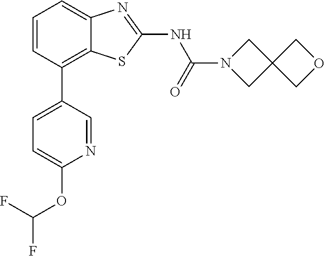 [SMILES: O=C(Nc1nc2cccc(-c3ccc(OC(F)F)nc3)c2s1)N1CC2(COC2)C1] | n/a | 419.1 |
| 74 | N-(7-(2-methoxypyrimidin- 5-yl)benzo[d]thiazol-2-yl)- 2-oxa-6- azaspiro[3.3]heptane-6- carboxamide | 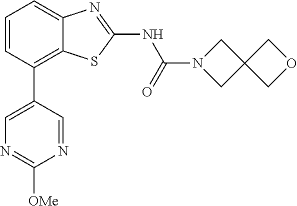 [SMILES: COc1ncc(-c2cccc3nc(NC(=O)N4CC5(COC5)C4)sc23)cn1] | n/a | 384.1 |
| 75 | N-(7-(6- (difluoromethoxy)pyridin- 3-yl)benzo[d]thiazol-2-yl)- 3-fluoro-3- (methoxymethyl)azetidine- 1-carboxamide | 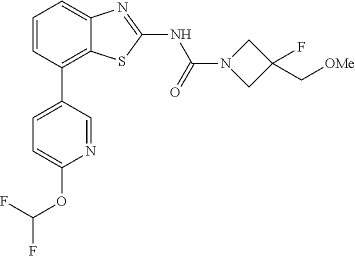 [SMILES: COCC1(F)CN(C(=O)Nc2nc3cccc(-c4ccc(OC(F)F)nc4)c3s2)C1] | n/a | 439.1 |
| 76 | 5-(2-(3-(methoxy-d3)-3- methylazetidine-1- carboxamido)benzo[d] thiazol-7-yl)-N-(oxetan-3- yl)picolinamide | 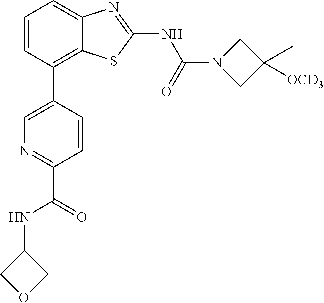 | 3-(methoxy-d3)-3- methylazetidine and N-(oxetan-3-yl)-5- (4,4,5,5- tetramethyl-1,3,2- dioxaborolan-2- yl)picolinamide | 457.3 |
| 77 | 3-(methoxy-d3)-3-methyl- N-(7-(3-methyl-4-(oxetan- 3- ylcarbamoyl)phenyl)benzo [d]thiazol-2-yl)azetidine-1- carboxamide | 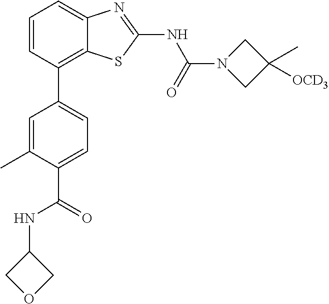 | 3-(methoxy-d3)-3- methylazetidine and 2-methyl-N- (oxetan-3-yl)-4- (4,4,5,5- tetramethyl-1,3,2- dioxaborolan-2- yl)benzamide | 470.1 |
| 78 | (R)-3-(methoxy-d3)-3- methyl-N-(7-(6- ((tetrahydrofuran-3- yl)amino)pyridin-3- yl)benzo[d]thiazol-2- yl)azetidine-1-carboxamide | 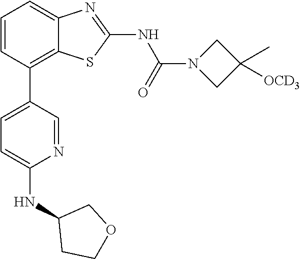 | 3-(methoxy-d3)-3- methylazetidine and (R)-N- (tetrahydrofuran-3- yl)-5-(4,4,5,5- tetramethyl-1,3,2- dioxaborolan-2- yl)pyridin-2-amine | 443.2 |
| 79 | (S)-3-(methoxy-d3)-3- methyl-N-(7-(6- ((tetrahydrofuran-3- yl)amino)pyridin-3- yl)benzo[d]thiazol-2- yl)azetidine-1-carboxamide | 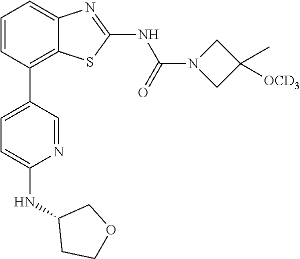 | 3-(methoxy-d3)-3- methylazetidine and (S)-N- (tetrahydrofuran-3- yl)-5-(4,4,5,5- tetramethyl-1,3,2- dioxaborolan-2- yl)pyridin-2-amine | 443.2 |
| 80 | N-(7-(1-(2-hydroxy-2- methylpropyl)-1H-pyrazol- 4-yl)benzo[d]thiazol-2-yl)- 3-(methoxy-d3)-3- methylazetidine-1- carboxamide | 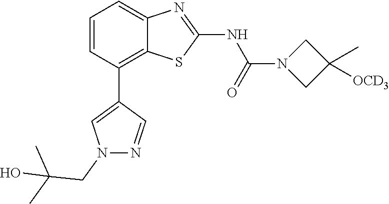 | 3-(methoxy-d3)-3- methylazetidine | 419.3 |
| 81 | 3-fluoro-3-((methoxy- d3)methyl)-N-(7-(2- methoxypyrimidin-5- yl)benzo[d]thiazol-2- yl)azetidine-1-carboxamide | 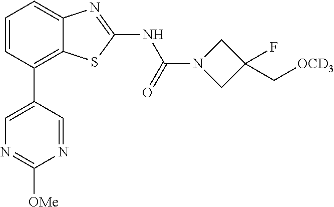 | 3-fluoro-3- ((methoxy- d3)methyl) azetidine | 407.1 |
| 82 | 3-(methoxy-d3)-N-(7-(2- methoxypyrimidin-5- yl)benzo[d]thiazol-2-yl)-3- methylazetidine-1- carboxamide | 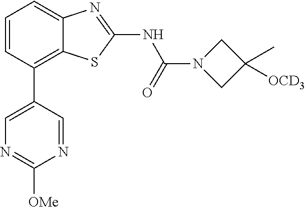 | 3-(methoxy-d3)-3- methylazetidine | 389.1 |
| 83 | N-(7-(6- (difluoromethoxy)pyridin- 3-yl)benzo[d]thiazol-2-yl)- 3-fluoro-3-((methoxy- d3)methyl)azetidine-1- carboxamide | 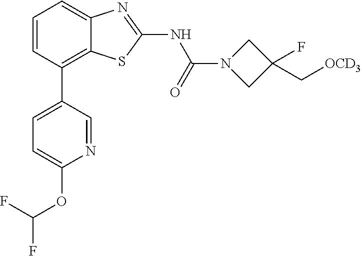 | 3-fluoro-3- ((methoxy- d3)methyl) azetidine | 442.2 |
| 84 | 3-fluoro-N-(7-(1-(2- hydroxy-2-methylpropyl)- 1H-pyrazol-4- yl)benzo[d]thiazol-2-yl)-3- ((methoxy- d3)methyl)azetidine-1- carboxamide | 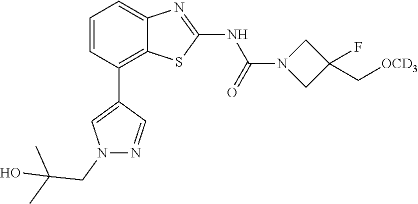 | 3-fluoro-3- ((methoxy- d3)methyl) azetidine | 437.2 |
| 85 | N-(7-(2-(difluoro- methoxy)pyrimidin- 5-yl)benzo[d]thiazol-2- yl)-3-fluoro-3-((methoxy- d3)methyl)azetidine-1- carboxamide | 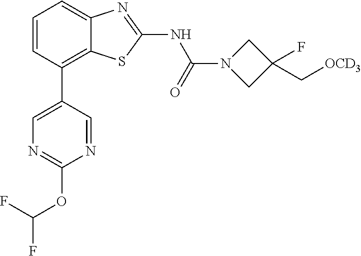 | 3-fluoro-3- ((methoxy- d3)methyl) azetidine | 443.1 |
| 86 | 3-(methoxy-d3)-3-methyl- N-(7-(4-(oxetan-3- ylcarbamoyl)phenyl)benzo [d]thiazol-2-yl)azetidine-1- carboxamide | 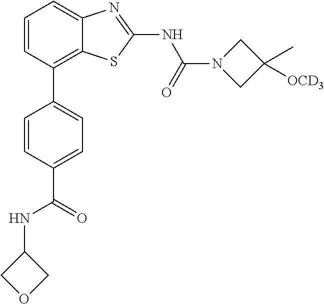 | 3-(methoxy-d3)-3- methylazetidine and N-(oxetan-3-yl)-4- (4,4,5,5- tetramethyl-1,3,2- dioxaborolan-2- yl)benzamide | 456.3 |
| 87 | 3-fluoro-N-(7-(2- isopropoxypyrimidin-5- yl)benzo[d]thiazol-2-yl)-3- ((methoxy- d3)methyl)azetidine-1- carboxamide | 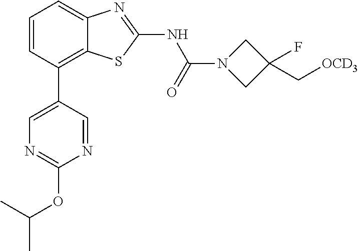 | 3-fluoro-3- ((methoxy- d3)methyl) azetidine | 435.2 |
| 88 | N-(7-(2-ethoxypyrimidin-5- yl)benzo[d]thiazol-2-yl)-3- (methoxy-d3)-3- methylazetidine-1- carboxamide | 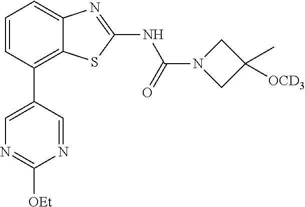 | 3-(methoxy-d3)-3- methylazetidine | 403.2 |
| 89 | N-(7-(2- isopropoxypyrimidin-5- yl)benzo[d]thiazol-2-yl)-3- (methoxy-d3)-3- methylazetidine-1- carboxamide | 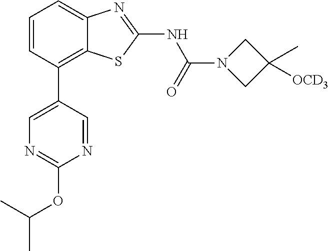 | 3-(methoxy-d3)-3- methylazetidine | 417.2 |
| 90 | N-(7-(4- (difluoromethoxy)phenyl) benzo[d]thiazol-2-yl)-2-oxa- 6-azaspiro[3.3]heptane-6- carboxamide | 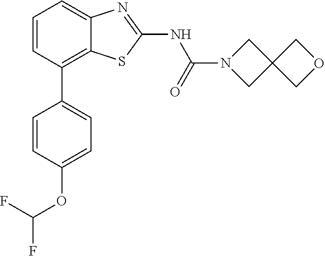 [SMILES: O=C(Nc1nc2cccc(-c3ccc(OC(F)F)cc3)c2s1)N1CC2(COC2)C1] | n/a | 418.1 |
| 91 | N-(7-(2- cyclopropoxypyrimidin-5- yl)benzo[d]thiazol-2-yl)-3- fluoro-3- (methoxymethyl)azetidine- 1-carboxamide | 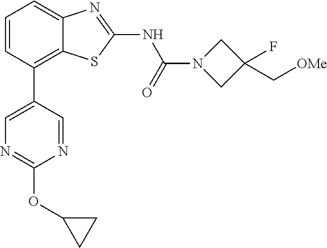 [SMILES: COCC1(F)CN(C(=O)Nc2nc3cccc(-c4cnc(OC5CC5)nc4)c3s2)C1] | 2-cyclopropoxy-5- (4,4,5,5- tetramethyl-1,3,2- dioxaborolan-2- yl)pyrimidine | 430.3 |
| 92 | 3-(methoxy-d3)-3-methyl- N-(7-(2-(oxetan-3-yl)-1- oxoisoindolin-5- yl)benzo[d]thiazol-2- yl)azetidine-1-carboxamide | 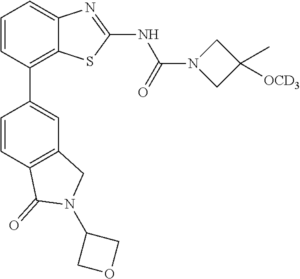 | 3-(methoxy-d3)-3- methylazetidine and 2-(oxetan-3-yl)-5- (4,4,5,5- tetramethyl-1,3,2- dioxaborolan-2- yl)isoindolin-1-one | 468.2 |
| 93 | 3-fluoro-N-(7-(2-(2- methoxyethoxy)pyrimidin- 5-yl)benzo[d]thiazol-2-yl)- 3- (methoxymethyl)azetidine- 1-carboxamide | 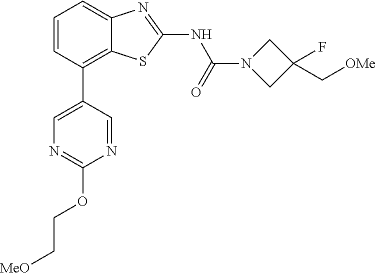 [SMILES: COCCOc1ncc(-c2cccc3nc(NC(=O)N4CC(F)(COC)C4)sc23)cn1] | 2-(2- methoxyethoxy)-5- (4,4,5,5- tetramethyl-1,3,2- dioxaborolan-2- yl)pyrimidine | 448.2 |
| 94 | 3-fluoro-N-(7- (6,6a,7,8,9,10- hexahydropyrazino[1,2- d]pyrido[3,2-b][1,4]oxazin- 3-yl)benzo[d]thiazol-2-yl)- 3- (methoxymethyl)azetidine- 1-carboxamide | 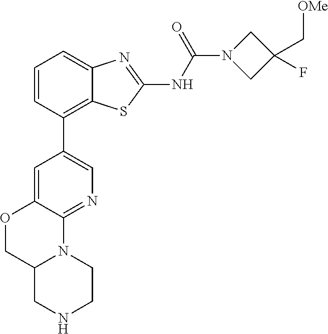 [SMILES: COCC1(F)CN(C(=O)Nc2nc3cccc(-c4cnc5c(c4)OCC4CNCCN54)c3s2)C1] | tert-butyl 3- (4,4,5,5- tetramethyl-1,3,2- dioxaborolan-2-yl)- 6a,7,9,10- tetrahydropyrazino [1,2-d]pyrido[3,2- b][1,4]oxazine- 8(6H)-carboxylate | 485.2 |
| 95 | N-(7-(2-ethoxypyrimidin-5- yl)benzo[d]thiazol-2-yl)- 1,1-difluoro-5- azaspiro[2.3]hexane-5- carboxamide | 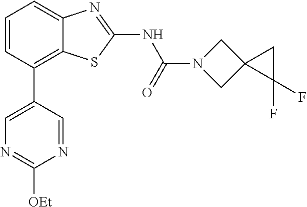 [SMILES: CCOc1ncc(-c2cccc3nc(NC(=O)N4CC5(C4)CC5(F)F)sc23)cn1] | n/a | 418.1 |
| 96 | N-(7-(1-isopropyl-1H- pyrazol-4- yl)benzo[d]thiazol-2-yl)-2- oxa-6-azaspiro[3.3]heptane- 6-carboxamide | 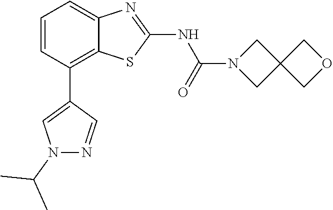 [SMILES: CC(C)n1cc(-c2cccc3nc(NC(=O)N4CC5(COC5)C4)sc23)cn1] | n/a | 384.2 |
| 97 | 3-fluoro-3-((methoxy- d3)methyl)-N-(7-(1- ((tetrahydro-2H-pyran-4- yl)methyl)-1H-pyrazol-4- yl)benzo[d]thiazol-2- yl)azetidine-1-carboxamide | 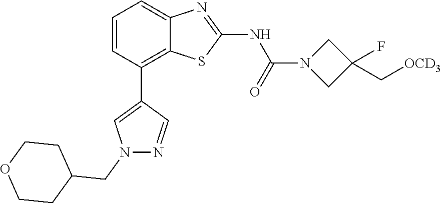 | 3-fluoro-3- ((methoxy- d3)methyl) azetidine | 463.3 |
| 98 | 3-fluoro-3- (methoxymethyl)-N-(7-(1- (2-morpholinoethyl)-1H- pyrazol-4- yl)benzo[d]thiazol-2- yl)azetidine-1-carboxamide | 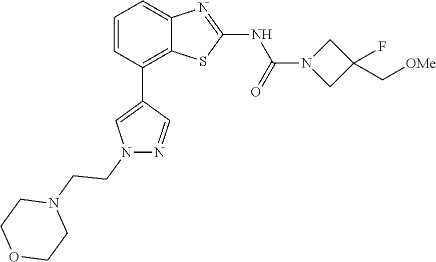 [SMILES: COCC1(F)CN(C(=O)Nc2nc3cccc(-c4cnn(CCN5CCOCC5)c4)c3s2)C1] | n/a | 475.2 |
| 99 | (S)-N-(7-(1-((1,4-dioxan-2- yl)methyl)-1H-pyrazol-4- yl)benzo[d]thiazol-2-yl)-3- fluoro-3- (methoxymethyl)azetidine- 1-carboxamide | 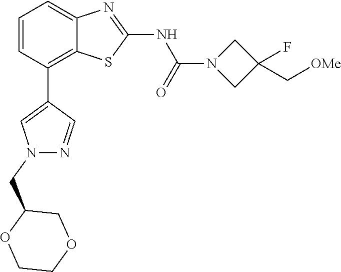 [SMILES: COCC1(F)CN(C(=O)Nc2nc3cccc(-c4cnn(C[C@H]5COCCO5)c4)c3s2)C1] | (S)-1-((1,4-dioxan- 2-yl)methyl)-4- (4,4,5,5- tetramethyl-1,3,2- dioxaborolan-2-yl)- 1H-pyrazole | 462.2 |
| 100 | (R)-N-(7-(1-((1,4-dioxan-2- yl)methyl)-1H-pyrazol-4- yl)benzo[d]thiazol-2-yl)-3- fluoro-3- (methoxymethyl)azetidine- 1-carboxamide | 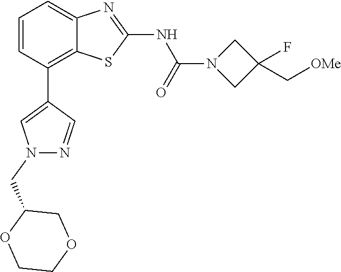 [SMILES: COCC1(F)CN(C(=O)Nc2nc3cccc(-c4cnn(C[C@@H]5COCCO5)c4)c3s2)C1] | (R)-1-((1,4-dioxan- 2-yl)methyl)-4- (4,4,5,5- tetramethyl-1,3,2- dioxaborolan-2-yl)- 1H-pyrazole | 462.2 |
| 101 | N-(7-(1-methyl-1H- pyrazol-5- yl)benzo[d]thiazol-2-yl)-2- oxa-6-azaspiro[3.3]heptane- 6-carboxamide | 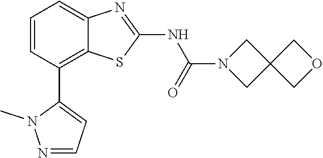 [SMILES: Cn1nccc1-c1cccc2nc(NC(=O)N3CC4(COC4)C3)sc12] | n/a | 356.1 |
| 102 | N-(7-(6,7-dihydro-4H- pyrazolo[5,1-c][1,4]oxazin- 3-yl)benzo[d]thiazol-2-yl)- 2-oxa-6- azaspiro[3.3]heptane-6- carboxamide | 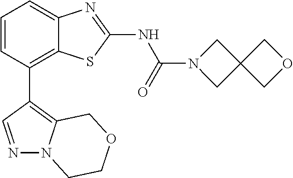 [SMILES: O=C(Nc1nc2cccc(-c3cnn4c3COCC4)c2s1)N1CC2(COC2)C1] | n/a | 398.2 |
| 103 | 1,1-difluoro-N-(7-(1-(2- hydroxy-2-methylpropyl)- 3-methyl-1H-pyrazol-4- yl)benzo[d]thiazol-2-yl)-5- azaspiro[2.3]hexane-5- carboxamide | 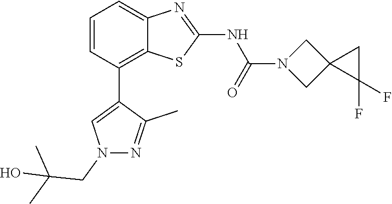 [SMILES: Cc1nn(CC(C)(C)O)cc1-c1cccc2nc(NC(=O)N3CC4(C3)CC4(F)F)sc12] | 2-methyl-1-(3- methyl-4-(4,4,5,5- tetramethyl-1,3,2- dioxaborolan-2-yl)- 1H-pyrazol-1- yl)propan-2-ol | 448.2 |
| 104 | 1,1-difluoro-N-(7-(1-(2- hydroxy-2-methylpropyl)- 1H-pyrazol-4- yl)benzo[d]thiazol-2-yl)-5- azaspiro[2.3]hexane-5- carboxamide | 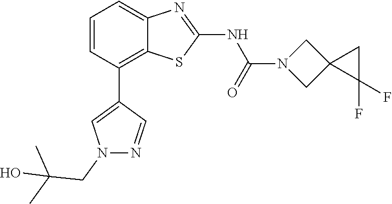 [SMILES: CC(C)(O)Cn1cc(-c2cccc3nc(NC(=O)N4CC5(C4)CC5(F)F)sc23)cn1] | n/a | 434.1 |
| 105 | 5-(2-(3-fluoro-3-((methoxy- d3)methyl)azetidine-1- carboxamido)benzo[d] thiazol-7-yl)-N-(oxetan-3- yl)picolinamide | 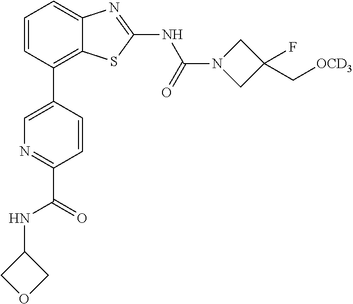 | 3-fluoro-3- ((methoxy- d3)methyl) azetidine and N-(oxetan-3-yl)-5- (4,4,5,5- tetramethyl-1,3,2- dioxaborolan-2- yl)picolinamide | 475.3 |
| 106 | N-(7-(3,6-dihydro-2H- pyran-4-yl)benzo[d]thiazol- 2-yl)-3-fluoro-3- (methoxymethyl)azetidine- 1-carboxamide | 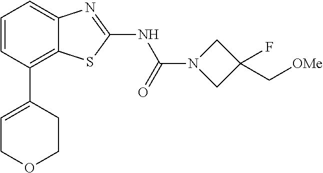 [SMILES: COCC1(F)CN(C(=O)Nc2nc3cccc(C4=CCOCC4)c3s2)C1] | n/a | 378.1 |
| 107 | 3-fluoro-3- (methoxymethyl)-N-(7- (tetrahydro-2H-pyran-4- yl)benzo[d]thiazol-2- yl)azetidine-1-carboxamide | 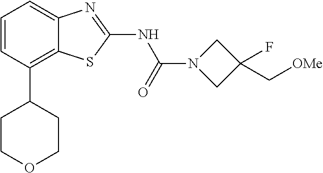 [SMILES: COCC1(F)CN(C(=O)Nc2nc3cccc(C4CCOCC4)c3s2)C1] | n/a | 380.1 |
| | | (Hydrogenation of Example 106: | | |
| | | H₂, 50 PSI, EtOAc) | | |

*Boc removal required as additional final step (5 eq TFA, dichloromethane)

### Synthetic Procedure C

### Example 108: 3-Fluoro-N-(7-(4-methoxy-1H-pyrazol-1-yl)benzo[d]thiazol-2-yl)-3-(methoxymethyl)azetidine-1-carboxamide

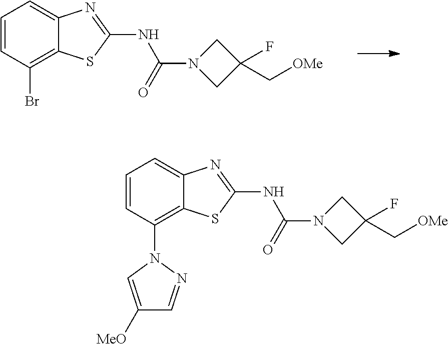 [SMILES: COCC1(F)CN(C(=O)Nc2nc3cccc(Br)c3s2)C1 ; COCC1(F)CN(C(=O)Nc2nc3cccc(-n4cc(OC)cn4)c3s2)C1]

A mixture of N-(7-bromobenzo[d]thiazol-2-yl)-3-fluoro-3-(methoxymethyl)azetidine-1-carboxamide (see B Step 2; 100 mg, 0.27 mmol), DMSO (2 mL), cesium carbonate (435 mg, 1.34 mmol), bis[(tetrabutylammonium iodide)copper(I) iodide](30 mg, 26.7 mmol), 4-methoxy-1H-pyrazole (52 mg, 0.53 mmol) and N,N-dimethylglycine (6 mg, 54 μmol) was stirred at 120° C. for 12 h. Water was added (30 mL) and the mixture was extracted into ethyl acetate (3×30 mL). The combined organic extracts were washed with brine (2×20 mL), dried over anhydrous sodium sulfate and concentrated in vacuo. Preparative HPLC purification (column: Phenomenex Luna C₁₈ 150*25 mm 10 μm; acetonitrile:water (formic acid)]; gradient: 31%-61% B over 10 min) afforded 3-fluoro-N-(7-(4-methoxy-1H-pyrazol-1-yl)benzo[d]thiazol-2-yl)-3-(methoxymethyl)azetidine-1-carboxamide (17 mg, 45 μmol, 17%) as a white solid; δ_{H} (400 MHz; CDCl₃) 7.73 (1H, s), 7.60-7.52 (2H, m), 7.44 (1H, m), 7.29 (1H, d, J 8.1 Hz), 4.29-4.21 (4H, m), 3.86 (3H, s), 3.66 (2H, m), 3.48-3.44 (3H, m); δ_{F} (400 MHz; CDCl₃) −157.4; MS-MS [M+H]⁺: 392.1.

Other examples prepared using synthetic procedure C are shown in the following table. All intermediates are commercially available unless noted (see ‘Intermediate synthesis’ section).

| Ex. | | | Intermediates | ES-MS |
|---|---|---|---|---|
| No. | Name | Structure | requiring synthesis | [M + H]⁺ |
| 109 | 3-fluoro-N-(7-(4-(2- (methoxy-d3)ethoxy)- 1H-pyrazol-1- yl)benzo[d]thiazol-2- yl)-3-((methoxy- d3)methyl)azetidine-1- carboxamide | 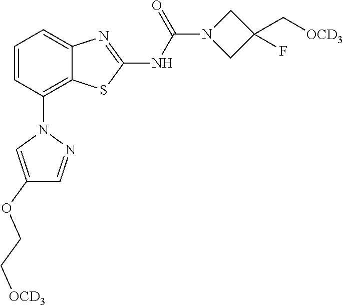 | 3-fluoro-3- ((methoxy- d3)methyl)azetidine and 4-(2-(methoxy- d3)ethoxy)-1H- pyrazole | 442.2 |
| 110 | N-(7-(4-(2- (difluoromethoxy) ethoxy)-1H-pyrazol-1- yl)benzo[d]thiazol-2- yl)-3-fluoro-3- ((methoxy- d3)methyl)azetidine-1- carboxamide | 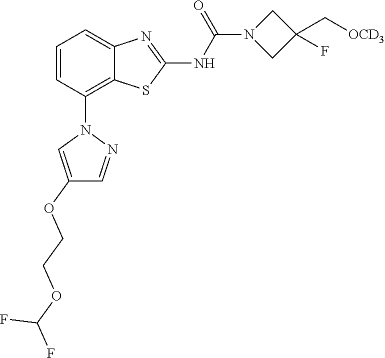 | 3-fluoro-3- ((methoxy- d3)methyl)azetidine and 4-(2- (difluoromethoxy) ethoxy)-1H-pyrazole | 475.2 |
| 111 | 3-fluoro-3-((methoxy- d3)methyl)-N-(7-(4-(2- morpholinoethoxy)-1H- pyrazol-1- yl)benzo[d]thiazol-2- yl)azetidine-1- carboxamide | 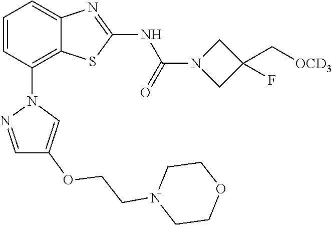 | 3-fluoro-3- ((methoxy- d3)methyl)azetidine and 4-(2-((1H-pyrazol-4- yl)oxy)ethyl) morpholine | 494.1 |
| 112 | 3-fluoro-3- (methoxymethyl)-N-(7- (4-(2- morpholinoethoxy)-1H- pyrazol-1- yl)benzo[d]thiazol-2- yl)azetidine-1- carboxamide | 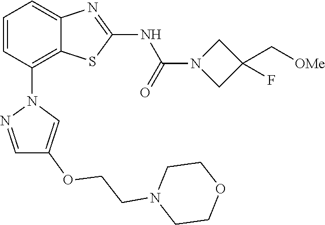 [SMILES: COCC1(F)CN(C(=O)Nc2nc3cccc(-n4cc(OCCN5CCOCC5)cn4)c3s2)C1] | 4-(2-((1H-pyrazol-4- yl)oxy)ethyl) morpholine | 491.2 |
| 113 | N-(7-(4-ethoxy-1H- pyrazol-1- yl)benzo[d]thiazol-2- yl)-3-fluoro-3- (methoxymethyl) azetidine-1-carboxamide | 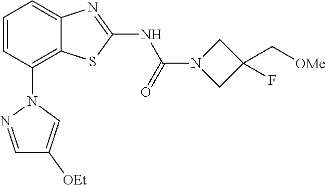 [SMILES: CCOc1cnn(-c2cccc3nc(NC(=O)N4CC(F)(COC)C4)sc23)c1] | n/a | 406.2 |
| 114 | (R)-3-fluoro-3- (methoxymethyl)-N-(7- (4-(2- morpholinopropoxy)- 1H-pyrazol-1- yl)benzo[d]thiazol-2- yl)azetidine-1- carboxamide | 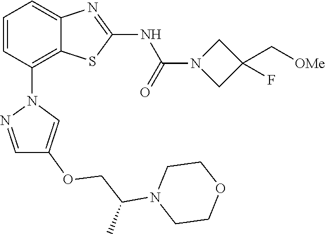 [SMILES: COCC1(F)CN(C(=O)Nc2nc3cccc(-n4cc(OC[C@@H](C)N5CCOCC5)cn4)c3s2)C1] | (R)-4-(1-((1H- pyrazol-4- yl)oxy)propan-2- yl)morpholine | 505.2 |
| 115 | (S)-3-fluoro-3- (methoxymethyl)-N-(7- (4-(2- morpholinopropoxy)- 1H-pyrazol-1- yl)benzo[d]thiazol-2- yl)azetidine-1- carboxamide | 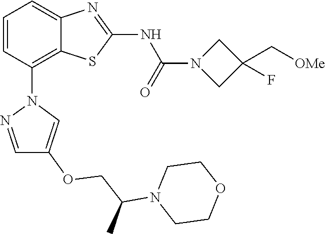 [SMILES: COCC1(F)CN(C(=O)Nc2nc3cccc(-n4cc(OC[C@H](C)N5CCOCC5)cn4)c3s2)C1] | (S)-4-(1-((1H- pyrazol-4- yl)oxy)propan-2- yl)morpholine | 505.2 |
| 116 | 3-fluoro-N-(6-fluoro-7- (4-((tetrahydro-2H- pyran-4-yl)amino)-1H- pyrazol-1- yl)benzo[d]thiazol-2- yl)-3-(methoxy- methyl)azetidine- 1-carboxamide | 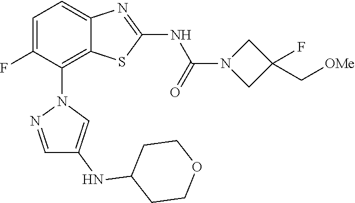 [SMILES: COCC1(F)CN(C(=O)Nc2nc3ccc(F)c(-n4cc(NC5CCOCC5)cn4)c3s2)C1] | N-(7-bromo-6- fluorobenzo[d] thiazol-2-yl)-3-fluoro- 3-(methoxymethyl) azetidine-1- carboxamide and N-(tetrahydro-2H- pyran-4-yl)-1H- pyrazol-4-amine | 479.2 |
| 117 | 3-fluoro-N-(6-fluoro-7- (4-(2-(3- oxomorpholino)ethoxy)- 1H-pyrazol-1- yl)benzo[d]thiazol-2- yl)-3- (methoxymethyl) azetidine-1-carboxamide | 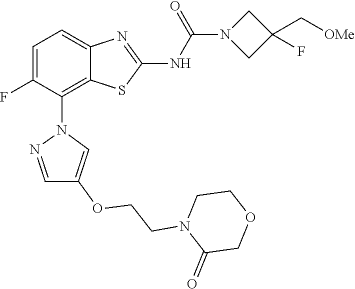 [SMILES: COCC1(F)CN(C(=O)Nc2nc3ccc(F)c(-n4cc(OCCN5CCOCC5=O)cn4)c3s2)C1] | 4-(2-((1H-pyrazol-4- yl)oxy)ethyl) morpholin-3-one and N-(7-bromo-6- fluorobenzo[d] thiazol-2-yl)-3- fluoro-3-(methoxy- methyl)azetidine-1- carboxamide | 523.3 |
| 118 | 3-fluoro-N-(6-fluoro-7- (4-((1-methyl-2- oxabicyclo[2.1.1]hexan- 4-yl)methoxy)-1H- pyrazol-1- yl)benzo[d]thiazol-2- yl)-3- (methoxymethyl) azetidine-1-carboxamide | 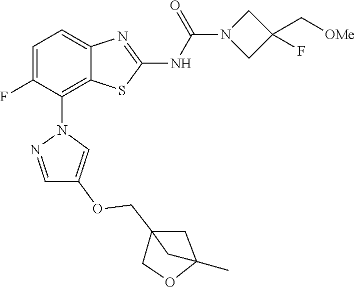 [SMILES: COCC1(F)CN(C(=O)Nc2nc3ccc(F)c(-n4cc(OCC56COC(C)(C5)C6)cn4)c3s2)C1] | 4-((1-methyl-2- oxabicyclo[2.1.1] hexan-4-yl)methoxy)- 1H-pyrazole and N-(7-bromo-6- fluorobenzo[d] thiazol-2-yl)-3- fluoro-3-(methoxy- methyl)azetidine-1- carboxamide | 506.3 |
| 119 | N-(7-(4-ethoxy-1H- pyrazol-1-yl)-6- fluorobenzo[d]thiazol-2- yl)-3-fluoro-3- (methoxymethyl) azetidine-1-carboxamide | 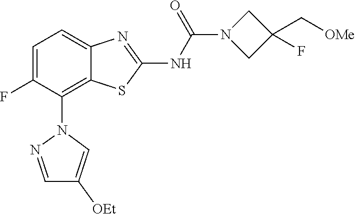 [SMILES: CCOc1cnn(-c2c(F)ccc3nc(NC(=O)N4CC(F)(COC)C4)sc23)c1] | N-(7-bromo-6- fluorobenzo[d] thiazol-2-yl)-3- fluoro-3-(methoxy- methyl)azetidine-1- carboxamide | 424.1 |
| 120 | N-(7-(4-(2-(6-oxa-3- azabicyclo[3.1.1]heptan- 3-yl)ethoxy)-1H- pyrazol-1-yl)-6- fluorobenzo[d]thiazol-2- yl)-3-fluoro-3- ((methoxy- d3)methyl)azetidine-1- carboxamide | 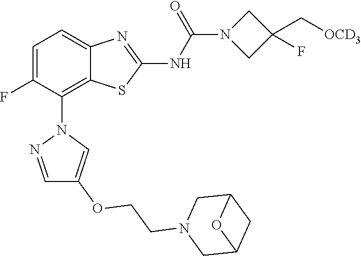 | N-(7-bromo-6- fluorobenzo[d] thiazol-2-yl)-3- fluoro-3-((methoxy- d3)methyl)azetidine- 1-carboxamide and 3-(2-((1H-pyrazol-4- yl)oxy)ethyl)-6-oxa- 3- azabicyclo[3.1.1] heptane | 524.2 |
| 121 | 3-fluoro-N-(6-fluoro-7- (4-methoxy-1H-pyrazol- 1-yl)benzo[d]thiazol-2- yl)-3- (methoxymethyl) azetidine-1-carboxamide | 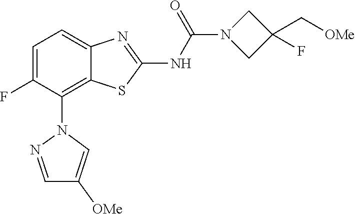 [SMILES: COCC1(F)CN(C(=O)Nc2nc3ccc(F)c(-n4cc(OC)cn4)c3s2)C1] | N-(7-bromo-6- fluorobenzo[d] thiazol-2-yl)-3- fluoro-3-(methoxy- methyl)azetidine- 1-carboxamide | 410.1 |
| 122 | N-(7-(4-(2-(trans-2,6- dimethylmorpholino) ethoxy)-1H-pyrazol-1-yl)- 6-fluorobenzo[d]thiazol- 2-yl)-3-fluoro-3- (methoxymethyl) azetidine-1-carboxamide | 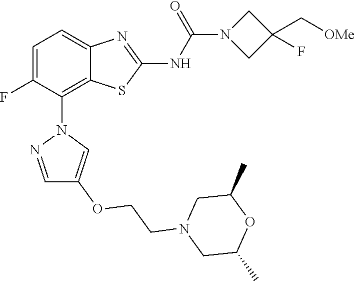 [SMILES: COCC1(F)CN(C(=O)Nc2nc3ccc(F)c(-n4cc(OCCN5C[C@@H](C)O[C@H](C)C5)cn4)c3s2)C1] | trans-4-(2-((1H- pyrazol-4- yl)oxy)ethyl)-2,6- dimethylmorpholine and N-(7-bromo-6- fluorobenzo[d] thiazol-2-yl)-3- fluoro-3-(methoxy- methyl)azetidine- 1-carboxamide | 537.3 |
| 123 | N-(7-(4-(2-(cis-2,6- dimethylmorpholino) ethoxy)-1H-pyrazol-1-yl)- 6-fluorobenzo[d]thiazol- 2-yl)-3-fluoro-3- (methoxymethyl) azetidine-1-carboxamide | 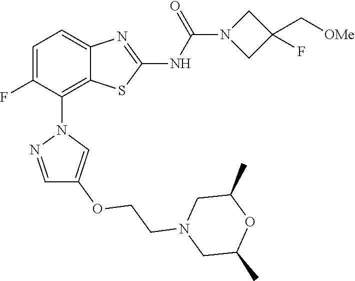 [SMILES: COCC1(F)CN(C(=O)Nc2nc3ccc(F)c(-n4cc(OCCN5C[C@@H](C)O[C@@H](C)C5)cn4)c3s2)C1] | cis-4-(2-((1H- pyrazol-4- yl)oxy)ethyl)-2,6- dimethylmorpholine and N-(7-bromo-6- fluorobenzo[d] thiazol-2-yl)-3- fluoro-3-(methoxy- methyl)azetidine- 1-carboxamide | 537.2 |
| 124 | N-(7-(4-(2-(2-oxa-6- azaspiro[3.3]heptan-6- yl)ethoxy)-1H-pyrazol- 1-yl)-6- fluorobenzo[d]thiazol-2- yl)-3-fluoro-3- (methoxymethyl) azetidine-1-carboxamide | 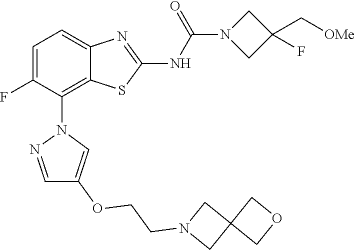 [SMILES: COCC1(F)CN(C(=O)Nc2nc3ccc(F)c(-n4cc(OCCN5CC6(COC6)C5)cn4)c3s2)C1] | 6-(2-((1H-pyrazol-4- yl)oxy)ethyl)-2-oxa- 6- azaspiro[3.3]heptane and N-(7-bromo-6- fluorobenzo[d] thiazol-2-yl)-3- fluoro-3-(methoxy- methyl)azetidine- 1-carboxamide | 521.1 |
| 125 | 3-fluoro-N-(6-fluoro-7- (4-(2-morpholinoethyl)- 1H-pyrazol-1- yl)benzo[d]thiazol-2- yl)-3- (methoxymethyl) azetidine-1-carboxamide | 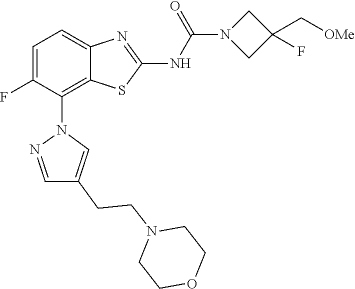 [SMILES: COCC1(F)CN(C(=O)Nc2nc3ccc(F)c(-n4cc(CCN5CCOCC5)cn4)c3s2)C1] | 4-(2-(1H-pyrazol-4- yl)ethyl)morpholine and N-(7-bromo-6- fluorobenzo[d] thiazol-2-yl)-3- fluoro-3-(methoxy- methyl)azetidine- 1-carboxamide | 493.1 |
| 126 | (S)-3-fluoro-N-(6- fluoro-7-(4-(2- morpholinopropoxy)- 1H-pyrazol-1- yl)benzo[d]thiazol-2- yl)-3- (methoxymethyl) azetidine-1-carboxamide | 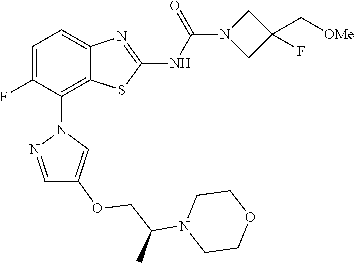 [SMILES: COCC1(F)CN(C(=O)Nc2nc3ccc(F)c(-n4cc(OC[C@H](C)N5CCOCC5)cn4)c3s2)C1] | (S)-4-(1-((1H- pyrazol-4- yl)oxy)propan-2- yl)morpholine and N-(7-bromo-6- fluorobenzo[d] thiazol-2-yl)-3- fluoro-3-(methoxy- methyl)azetidine- 1-carboxamide | 523.2 |
| 127 | (R)-3-fluoro-N-(6- fluoro-7-(4-(2- morpholinopropoxy)- 1H-pyrazol-1- yl)benzo[d]thiazol-2- yl)-3- (methoxymethyl) azetidine-1-carboxamide | 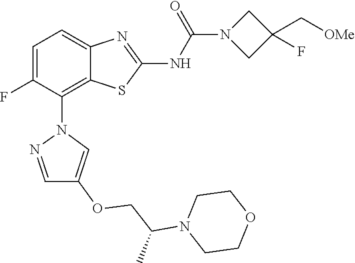 [SMILES: COCC1(F)CN(C(=O)Nc2nc3ccc(F)c(-n4cc(OC[C@@H](C)N5CCOCC5)cn4)c3s2)C1] | (R)-4-(1-((1H- pyrazol-4- yl)oxy)propan-2- yl)morpholine and N-(7-bromo-6- fluorobenzo[d] thiazol-2-yl)-3- fluoro-3-(methoxy- methyl)azetidine- 1-carboxamide | 523.3 |
| 128 | N-(6-fluoro-7-(4-(2- morpholinoethoxy)-1H- pyrazol-1- yl)benzo[d]thiazol-2- yl)-5-oxa-2- azaspiro[3.4]octane-2- carboxamide | 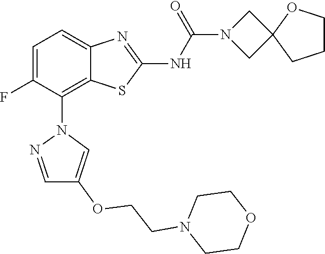 [SMILES: O=C(Nc1nc2ccc(F)c(-n3cc(OCCN4CCOCC4)cn3)c2s1)N1CC2(CCCO2)C1] | N-(7-bromo-6- fluorobenzo[d]thiazol- 2-yl)-5-oxa-2- azaspiro[3.4]octane- 2-carboxamide and 4-(2-((1H-pyrazol-4- yl)oxy)ethyl) morpholine | 503.2 |
| 129 | N-(6-fluoro-7-(4-(2- morpholinoethoxy)-1H- pyrazol-1- yl)benzo[d]thiazol-2- yl)-6-oxa-2- azaspiro[3.4]octane-2- carboxamide | 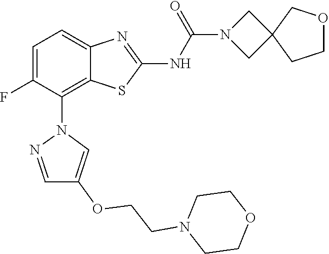 [SMILES: O=C(Nc1nc2ccc(F)c(-n3cc(OCCN4CCOCC4)cn3)c2s1)N1CC2(CCOC2)C1] | N-(7-bromo-6- fluorobenzo[d]thiazol- 2-yl)-6-oxa-2- azaspiro[3.4]octane- 2-carboxamide and 4-(2-((1H-pyrazol-4- yl)oxy)ethyl) morpholine | 503.1 |
| 130 | N-(7-(4-(2-(8-oxa-3- azabicyclo[3.2.1]octan- 3-yl)ethoxy)-1H- pyrazol-1-yl)-6- fluorobenzo[d]thiazol-2- yl)-3-fluoro-3- ((methoxy- d3)methyl)azetidine-1- carboxamide | 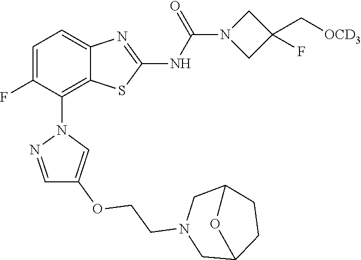 | N-(7-bromo-6- fluorobenzo[d]thiazol- 2-yl)-3-fluoro-3- ((methoxy- d3)methyl)azetidine- 1-carboxamide and 3-(2-((1H-pyrazol-4- yl)oxy)ethyl)-8-oxa- 3- azabicyclo[3.2.1] octane | 538.3 |
| 131 | 3-fluoro-N-(6-fluoro-7- (4-((1- morpholinocyclopropyl) methoxy)-1H-pyrazol-1- yl)benzo[d]thiazol-2- yl)-3- (methoxymethyl) azetidine-1-carboxamide | 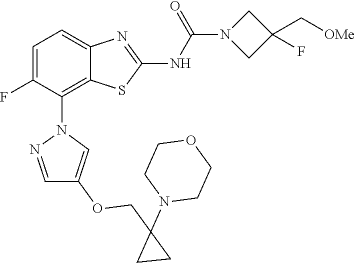 [SMILES: COCC1(F)CN(C(=O)Nc2nc3ccc(F)c(-n4cc(OCC5(N6CCOCC6)CC5)cn4)c3s2)C1] | 4-(1-(((1H-pyrazol- 4-yl)oxy) methyl)cyclopropyl) morpholine | 535.3 |
| 132 | 3-fluoro-N-(6-fluoro-7- (4-(2-methyl-2- morpholinopropoxy)- 1H-pyrazol-1- yl)benzo[d]thiazol-2- yl)-3- (methoxymethyl) azetidine-1-carboxamide | 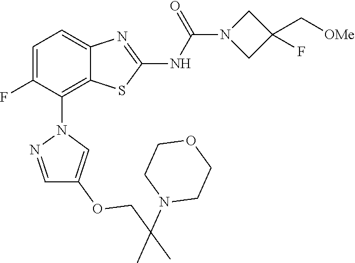 [SMILES: COCC1(F)CN(C(=O)Nc2nc3ccc(F)c(-n4cc(OCC(C)(C)N5CCOCC5)cn4)c3s2)C1] | 4-(1-((1H-pyrazol-4- yl)oxy)-2- methylpropan-2- yl)morpholine | 537.2 |
| 133 | 3-fluoro-N-(6-fluoro-7- (4-(2-methoxyethoxy)- 1H-pyrazol-1- yl)benzo[d]thiazol-2- yl)-3- (methoxymethyl) azetidine-1-carboxamide | 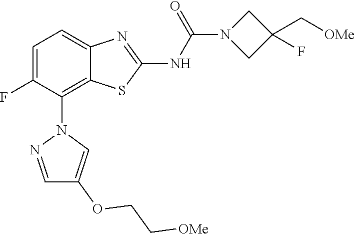 [SMILES: COCCOc1cnn(-c2c(F)ccc3nc(NC(=O)N4CC(F)(COC)C4)sc23)c1] | n/a | 454.1 |
| 134 | 3-fluoro-N-(6-fluoro-7- (4-(2- morpholinoethoxy)-1H- pyrazol-1- yl)benzo[d]thiazol-2- yl)-3- (methoxymethyl) azetidine-1-carboxamide | 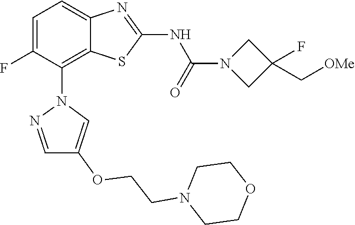 [SMILES: COCC1(F)CN(C(=O)Nc2nc3ccc(F)c(-n4cc(OCCN5CCOCC5)cn4)c3s2)C1] | 4-(2-((1H-pyrazol-4- yl)oxy)ethyl) morpholine | 509.1 |
| 135 | 3-fluoro-N-(6-fluoro-7- (4-(2- morpholinoethoxy)-1H- pyrazol-1- yl)benzo[d]thiazol-2- yl)-3-((methoxy- d3)methyl)azetidine-1- carboxamide | 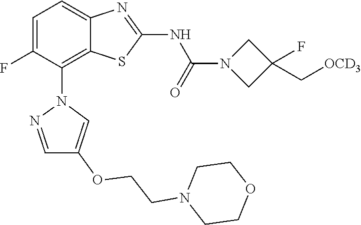 | N-(7-bromo-6- fluorobenzo[d]thiazol- 2-yl)-3-fluoro-3- ((methoxy- d3)methyl)azetidine- 1-carboxamide and 4-(2-((1H-pyrazol-4- yl)oxy)ethyl) morpholine | 512.1 |
| 136 | 3-fluoro-N-(6-fluoro-7- (4-(2-methoxyethoxy)- 1H-pyrazol-1- yl)benzo[d]thiazol-2- yl)-3-((methoxy- d3)methyl)azetidine-1- carboxamide | 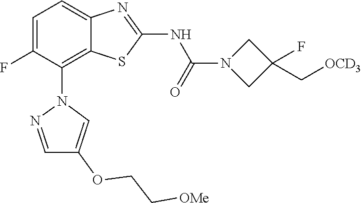 | N-(7-bromo-6- fluorobenzo[d]thiazol- 2-yl)-3-fluoro-3- ((methoxy- d3)methyl)azetidine- 1-carboxamide | 457.2 |
| 137 | 3-fluoro-N-(7-(4-(2- methoxyethoxy)-1H- pyrazol-1- yl)benzo[d]thiazol-2- yl)-3- (methoxymethyl) azetidine-1-carboxamide | 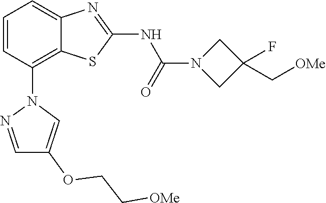 [SMILES: COCCOc1cnn(-c2cccc3nc(NC(=O)N4CC(F)(COC)C4)sc23)c1] | n/a | 436.2 |
| 138 | oxazol-2-ylmethyl (7- (4-morpholino-1H- pyrazol-1- yl)benzo[d]thiazol-2- yl)carbamate | 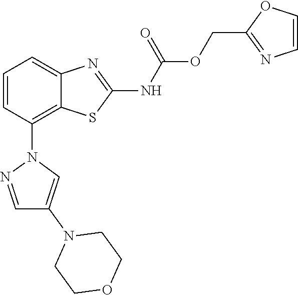 [SMILES: O=C(Nc1nc2cccc(-n3cc(N4CCOCC4)cn3)c2s1)OCc1ncco1] | oxazol-2-ylmethyl (7-bromobenzo [d]thiazol- 2-yl)carbamate | 427.1 |
| 139 | oxazol-2-ylmethyl (7- (4-(2-methoxyethoxy)- 1H-pyrazol-1- yl)benzo[d]thiazol-2- yl)carbamate | 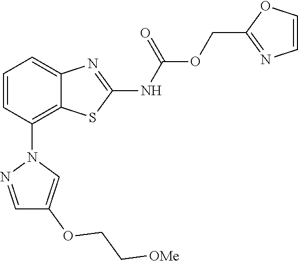 [SMILES: COCCOc1cnn(-c2cccc3nc(NC(=O)OCc4ncco4)sc23)c1] | oxazol-2-ylmethyl (7-bromobenzo [d]thiazol- 2-yl)carbamate | 416.1 |
| 140 | oxazol-2-ylmethyl (7- (4-methoxy-1H-pyrazol- 1-yl)benzo[d]thiazol-2- yl)carbamate | 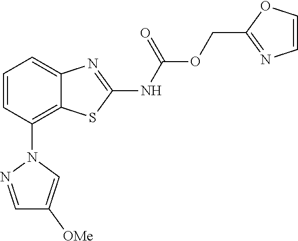 [SMILES: COc1cnn(-c2cccc3nc(NC(=O)OCc4ncco4)sc23)c1] | oxazol-2-ylmethyl (7-bromobenzo [d]thiazol- 2-yl)carbamate | 372.1 |
| 141 | N-(4,6-difluoro-7-(4-(2- morpholinoethoxy)-1H- pyrazol-1- yl)benzo[d]thiazol-2- yl)-3-fluoro-3- (methoxymethyl) azetidine-1-carboxamide | 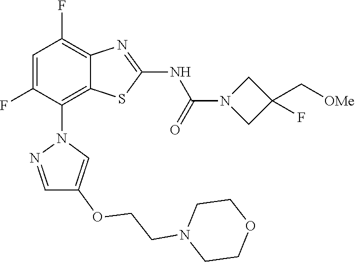 [SMILES: COCC1(F)CN(C(=O)Nc2nc3c(F)cc(F)c(-n4cc(OCCN5CCOCC5)cn4)c3s2)C1] | N-(7-bromo-4,6- difluorobenzo[d] thiazol-2-yl)-3- fluoro-3-(methoxy- methyl)azetidine- 1-carboxamide and 4-(2-((1H-pyrazol-4- yl)oxy)ethyl) morpholine | MISSING |
| 142 | N-(7-(4-(azetidin-3- yloxy)-1H-pyrazol-1- yl)-6- fluorobenzo[d]thiazol-2- yl)-3-fluoro-3- (methoxymethyl) azetidine-1-carboxamide | 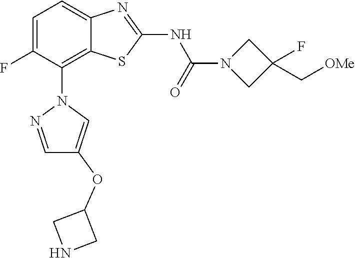 [SMILES: COCC1(F)CN(C(=O)Nc2nc3ccc(F)c(-n4cc(OC5CNC5)cn4)c3s2)C1] | N-(7-bromo-6- fluorobenzo[d] thiazol-2-yl)-3- fluoro-3-(methoxy- methyl)azetidine- 1-carboxamide tert-butyl 3-((1H- pyrazol-4- yl)oxy)azetidine-1- carboxylate | 451.3 |
| | | (Boc removal required as | | |
| | | additional final step (20 eq TFA, | | |
| | | dichloromethane) | | |
| 143 | 3-fluoro-N-(6-fluoro-7- (4-((1-methylazetidin-3- yl)oxy)-1H-pyrazol-1- yl)benzo[d]thiazol-2- yl)-3- (methoxymethyl) azetidine-1-carboxamide | 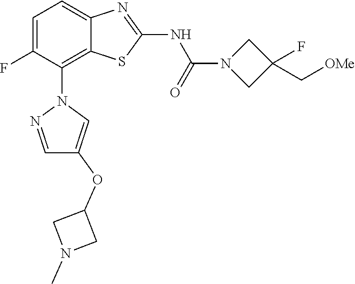 [SMILES: COCC1(F)CN(C(=O)Nc2nc3ccc(F)c(-n4cc(OC5CN(C)C5)cn4)c3s2)C1] | N-(7-bromo-6- fluorobenzo[d]thiazol- 2-yl)-3-fluoro-3- (methoxymethyl) azetidine-1- carboxamide and tert-butyl 3-((1H- pyrazol-4- yl)oxy)azetidine-1- carboxylate | 465.3 |
| | | (Prepared by methylation of | | |
| | | Example 142 using formaldehyde, | | |
| | | NaBH₃CN, AcOH, MeOH, 25° C.) | | |

### Synthetic Procedure D

### Example 144: 3-Fluoro-N-(6-fluoro-7-(6-((tetrahydro-2H-pyran-4-yl)amino)pyridin-3-yl)benzo[d]thiazol-2-yl)-3-((methoxy-d3)methyl)azetidine-1-carboxamide

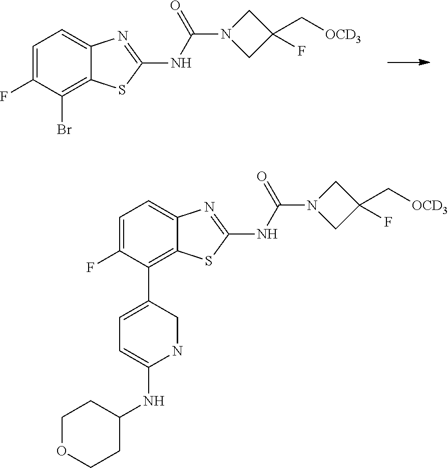

Pd(dppf)Cl₂ (400 mg, 0.55 mmol) was added to a solution of N-(7-bromo-6-fluorobenzo[d]thiazol-2-yl)-3-fluoro-3-((methoxy-d3)methyl)azetidine-1l-carboxamide (2.00 g, 5.06 mmol), N-(tetrahydro-2H-pyran-4-yl)-5-(4,4,5,5-tetramethyl-1,3,2-dioxaborolan-2-yl)pyridin-2-amine (2.3 g, 7.56 mmol), sodium carbonate (1.80 g, 17.0 mmol) in dioxane (20 mL) and water (0.5 mL). The mixture was degassed and purged with nitrogen three times, then stirred at 80° C. for 12 h. Water (40 mL) was added and the mixture was extracted into ethyl acetate (3×30 mL). The combined organic extracts were dried over anhydrous sodium sulfate and concentrated in vacuo. Flash column chromatography on silica (EtOAc:PE, 90:10 to 100:0) then preparative HPLC (column: Phenomenex Luna C₁₈ 150*40 mm 15 μm; acetonitrile:water(formic acid); gradient: 250%-55% B over 10 min) afforded 3-fluoro-N-(6-fluoro-7-(6-((tetrahydro-2H-pyran-4-yl)amino)pyridin-3-yl)benzo[d]thiazol-2-yl)-3-((methoxy-d3)methyl)azetidine-1-carboxamide (702 mg, 1.43 mmol, 28% yield) as a white solid; δ_{H} (400 MHz; DMSO-d6) 11.79-11.17 (1H, m), 8.20 (1H, s), 7.58 (1H, br d, J 8.6 Hz), 7.51 (1H, m), 7.36-7.20 (1H, m), 6.89 (1H, d, J 7.4 Hz), 6.62 (1H, d, J 8.6 Hz), 4.19-3.95 (5H, m), 3.93-3.84 (2H, m), 3.75-3.64 (2H, m), 3.43 (2H, m), 1.92 (2H, m), 1.55-1.37 (2H, m); δ_{F} (400 MHz; DMSO-d6) −125.7, −155.5; ES-MS [M+H]⁺: 493.2.

Other examples prepared using synthetic procedure D are shown in the following table. All intermediates are commercially available unless noted (see ‘Intermediate synthesis’ section).

| | | | Intermediates | |
|---|---|---|---|---|
| Ex. | | | requiring | ES-MS |
| No. | Name | Structure | synthesis | [M + H]⁺ |
| 145 | N-(7-(2- (difluoromethoxy)pyrimidin- 5-yl)-6- fluorobenzo[d]thiazol-2-yl)- 3-fluoro-3-((methoxy- d3)methyl)azetidine-1- carboxamide | 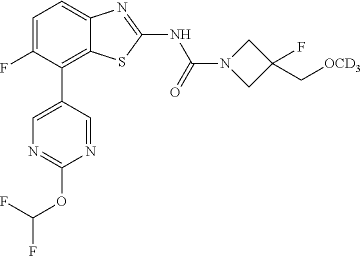 | N-(7-bromo-6- fluorobenzo[d] thiazol-2-yl)-3- fluoro-3-((methoxy- d3)methyl)azetidine- 1-carboxamide | 461.1 |
| 146 | 3-fluoro-N-(6-fluoro-7-(2- ((tetrahydro-2H-pyran-4- yl)amino)pyrimidin-5- yl)benzo[d]thiazol-2-yl)- 3-((methoxy- d3)methyl)azetidine-1- carboxamide | 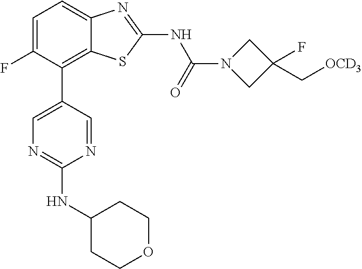 | N-(7-bromo-6- fluorobenzo[d] thiazol-2-yl)-3- fluoro-3-((methoxy- d3)methyl)azetidine- 1-carboxamide and N-(tetrahydro-2H- pyran-4-yl)-5- (4,4,5,5- tetramethyl-1,3,2- dioxaborolan-2- yl)pyrimidin-2- amine | 494.3 |
| 147 | N-(7-(2- (difluoromethoxy) pyrimidin- 5-yl)-6- fluorobenzo[d]thiazol- 2-yl)-3-(methoxy-d3)- 3-methylazetidine-1- carboxamide | 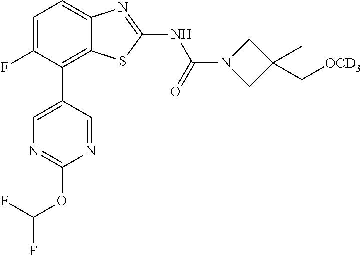 | N-(7-bromo-6- fluorobenzo[d]thia zol-2-yl)-3- (methoxy-d3)-3- methylazetidine-1- carboxamide | 443.2 |
| 148 | N-(6-fluoro-7-(2- ((tetrahydro-2H-pyran-4- yl)amino)pyrimidin-5- yl)benzo[d]thiazol-2-yl)- 3-(methoxy-d3)-3- methylazetidine-1- carboxamide | 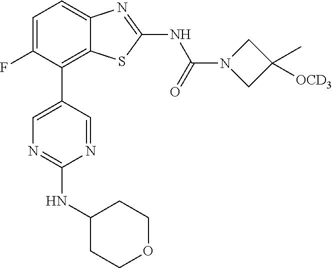 | N-(7-bromo-6- fluorobenzo[d] thiazol-2-yl)-3- (methoxy-d3)-3- methylazetidine-1- carboxamide and N-(tetrahydro-2H- pyran-4-yl)-5- (4,4,5,5- tetramethyl-1,3,2- dioxaborolan-2- yl)pyrimidin-2- amine | 476.3 |
| 149 | 3-fluoro-N-(6-fluoro-7-(6- (morpholinomethyl)pyridin- 3-yl)benzo[d]thiazol-2-yl)- 3-((methoxy- d3)methyl)azetidine-1- carboxamide | 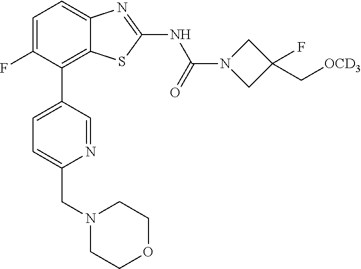 | N-(7-bromo-6- fluorobenzo[d] thiazol-2-yl)-3- fluoro-3-((methoxy- d3)methyl)azetidine- 1-carboxamide | 493.2 |
| 150 | 3-fluoro-N-(6-fluoro-7-(4- (morpholinomethyl)phenyl) benzo[d]thiazol-2-yl)-3- ((methoxy- d3)methyl)azetidine-1- carboxamide | 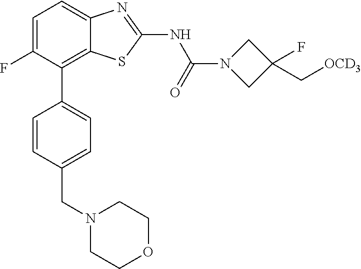 | N-(7-bromo-6- fluorobenzo[d] thiazol-2-yl)-3- fluoro-3-((methoxy- d3)methyl)azetidine- 1-carboxamide | 492.2 |
| 151 | N-(6-fluoro-7-(6- (morpholinomethyl)pyridin- 3-yl)benzo[d]thiazol-2-yl)- 3-(methoxy-d3)-3- methylazetidine-1- carboxamide | 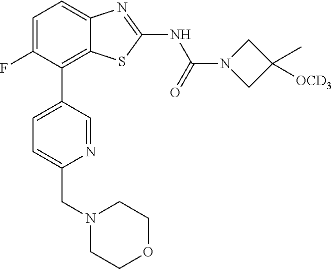 | N-(7-bromo-6- fluorobenzo[d] thiazol-2-yl)-3- (methoxy-d3)-3- methylazetidine-1- carboxamide | 475.2 |
| 152 | N-(6-fluoro-7-(4- (morpholinomethyl)phenyl) benzo[d]thiazol-2-yl)-3- (methoxy-d3)-3- methylazetidine-1- carboxamide | 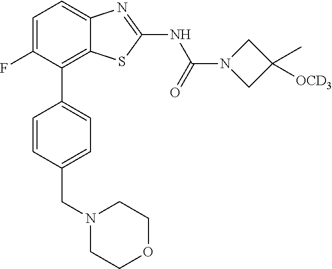 | N-(7-bromo-6- fluorobenzo[d] thiazol-2-yl)-3- (methoxy-d3)-3- methylazetidine-1- carboxamide | 474.2 |
| 153 | 3-fluoro-N-(6-fluoro-7-(2- ((1-(hydroxymethyl) cyclobutyl)amino) pyrimidin-5- yl)benzo[d]thiazol-2-yl)-3- (methoxymethyl)azetidine- 1-carboxamide | 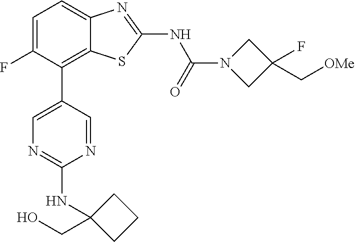 [SMILES: COCC1(F)CN(C(=O)Nc2nc3ccc(F)c(-c4cnc(NC5(CO)CCC5)nc4)c3s2)C1] | N-(7-bromo-6- fluorobenzo[d] thiazol-2-yl)-3-fluoro- 3-(methoxymethyl) azetidine-1- carboxamide and (1-((5-(4,4,5,5- tetramethyl-1,3,2- dioxaborolan-2- yl)pyrimidin-2- yl)amino)cyclobutyl) methanol | 491.2 |
| 154 | N-(6-fluoro-7-(2- isopropoxypyrimidin-5- yl)benzo[d]thiazol-2-yl)-3- methoxy-3-methylazetidine- 1-carboxamide | 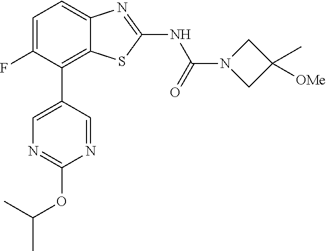 [SMILES: COC1(C)CN(C(=O)Nc2nc3ccc(F)c(-c4cnc(OC(C)C)nc4)c3s2)C1] | N-(7-bromo-6- fluorobenzo[d] thiazol-2-yl)-3- methoxy-3- methylazetidine-1- carboxamide | 432.2 |
| 155 | 3-fluoro-N-(6-fluoro-7-(2- (((3S,4R)-3- hydroxytetrahydro-2H-pyran- 4-yl)amino)pyrimidin-5- yl)benzo[d]thiazol-2-yl)-3- (methoxymethyl)azetidine-1- carboxamide | 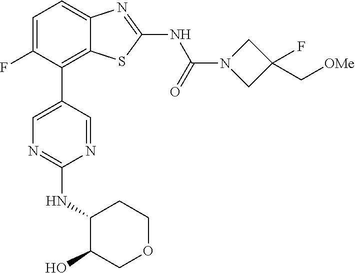 [SMILES: COCC1(F)CN(C(=O)Nc2nc3ccc(F)c(-c4cnc(N[C@@H]5CCOC[C@H]5O)nc4)c3s2)C1] | N-(7-bromo-6- fluorobenzo[d] thiazol-2-yl)- 3-fluoro-3- (methoxymethyl) azetidine-1 - carboxamide and (3S,4R)-4-((5- (4,4,5,5- tetramethyl-1,3,2- dioxaborolan-2- yl)pyrimidin-2- yl)amino)tetrahydro- 2H-pyran-3-ol | 507.1 |
| 156 | 3-fluoro-N-(6-fluoro-7-(2- ((1-methyl-2- oxabicyclo[2.1.1]hexan-4- yl)methoxy)pyrimidin-5- yl)benzo[d]thiazol-2-yl)-3- (methoxymethyl)azetidine-1- carboxamide | 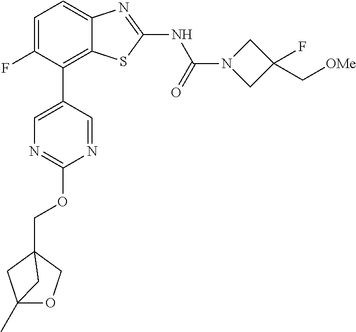 [SMILES: COCC1(F)CN(C(=O)Nc2nc3ccc(F)c(-c4cnc(OCC56COC(C)(C5)C6)nc4)c3s2)C1] | N-(7-bromo-6- fluorobenzo[d] thiazol-2-yl)- 3-fluoro-3- (methoxymethyl) azetidine-1- carboxamide and 2-((1-Methyl-2- oxabicyclo[2.1.1] hexan-4- yl)methoxy)-5- (4,4,5,5- tetramethyl-1,3- dioxolan-2- yl)pyrimidine | 518.4 |
| 157 | 3-fluoro-N-(6-fluoro-7-(2-(2- morpholinoethoxy)pyrimidin- 5-yl)benzo[d]thiazol-2-yl)-3- (methoxymethyl)azetidine-1- carboxamide | 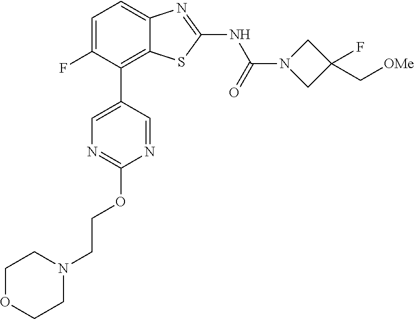 [SMILES: COCC1(F)CN(C(=O)Nc2nc3ccc(F)c(-c4cnc(OCCN5CCOCC5)nc4)c3s2)C1] | N-(7-bromo-6- fluorobenzo[d] thiazol-2-yl)- 3-fluoro-3- (methoxymethyl) azetidine-1- carboxamide and 4-(2-((5-(4,4,5,5- tetramethyl-1,3,2- dioxaborolan-2- yl)pyrimidin-2- yl)oxy)ethyl) morpholine | 521.3 |
| 158 | N-(7-(2- cyclopropoxypyrimidin-5- yl)-6-fluorobenzo[d]thiazol- 2-yl)-3-fluoro-3- (methoxymethyl)azetidine-1- carboxamide | 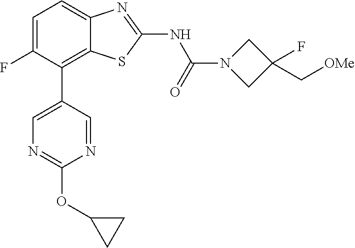 [SMILES: COCC1(F)CN(C(=O)Nc2nc3ccc(F)c(-c4cnc(OC5CC5)nc4)c3s2)C1] | N-(7-bromo-6- fluorobenzo[d] thiazol-2-yl)- 3-fluoro-3- (methoxymethyl) azetidine-1- carboxamide and 2-cyclopropoxy-5- (4,4,5,5- tetramethyl-1,3,2- dioxaborolan-2- yl)pyrimidine | 448.2 |
| 159 | 5-(6-fluoro-2-(3-fluoro-3- (methoxymethyl)azetidine-1- carboxamido)benzo[d] thiazol-7-yl)-N- methylpicolinamide | 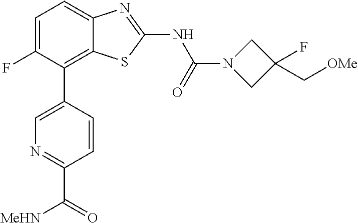 [SMILES: CNC(=O)c1ccc(-c2c(F)ccc3nc(NC(=O)N4CC(F)(COC)C4)sc23)cn1] | N-(7-bromo-6- fluorobenzo[d] thiazol-2-yl)- 3-fluoro-3-(methoxymethyl) azetidine-1- carboxamide | 448.1 |
| 160 | 3-fluoro-N-(6-fluoro-7-(6- (morpholine-4- carbonyl)pyridin-3- yl)benzo[d]thiazol-2-yl)-3- (methoxymethyl)azetidine-1- carboxamide | 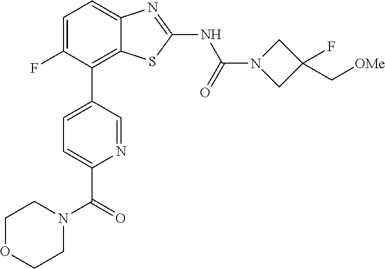 [SMILES: COCC1(F)CN(C(=O)Nc2nc3ccc(F)c(-c4ccc(C(=O)N5CCOCC5)nc4)c3s2)C1] | N-(7-bromo-6- fluorobenzo[d] thiazol-2-yl)- 3-fluoro-3- (methoxymethyl) azetidine-1- carboxamide | 504.2 |
| 161 | 3-fluoro-N-(6-fluoro-7-(2- (morpholine-4- carbonyl)pyrimidin-5- yl)benzo[d]thiazol-2-yl)-3- (methoxymethyl)azetidine-1- carboxamide | 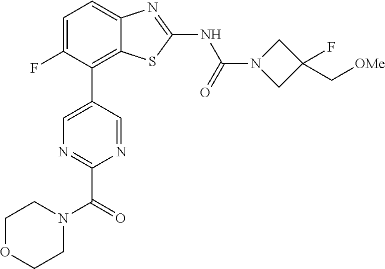 [SMILES: COCC1(F)CN(C(=O)Nc2nc3ccc(F)c(-c4cnc(C(=O)N5CCOCC5)nc4)c3s2)C1] | N-(7-bromo-6- fluorobenzo[d] thiazol-2-yl)- 3-fluoro-3- (methoxymethyl) azetidine-1- carboxamide and morpholino(5- (4,4,5,5- tetramethyl-1,3,2- dioxaborolan-2- yl)pyrimidin-2- yl)methanone | 505.2 |
| 162 | 5-(6-fluoro-2-(3-fluoro-3- (methoxymethyl)azetidine-1- carboxamido)benzo[d] thiazol-7-yl)-N-(2- methoxyethyl)picolinamide | 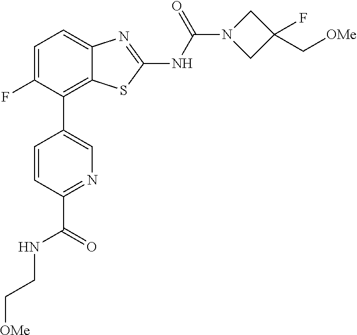 [SMILES: COCCNC(=O)c1ccc(-c2c(F)ccc3nc(NC(=O)N4CC(F)(COC)C4)sc23)cn1] | N-(7-bromo-6- fluorobenzo[d] thiazol-2-yl)- 3-fluoro-3- (methoxymethyl) azetidine-1- carboxamide and N-(2- methoxyethyl)-5- (4,4,5,5- tetramethyl-1,3,2- dioxaborolan-2- yl)picolinamide | 492.1 |
| 163 | 3-fluoro-N-(6-fluoro-7-(2- (((1-methyl-2- oxabicyclo[2.1.1]hexan-4- yl)methyl)amino)pyrimidin- 5-yl)benzo[d]thiazol-2-yl)-3- (methoxymethyl)azetidine-1- carboxamide | 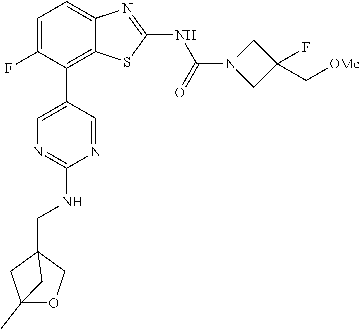 [SMILES: COCC1(F)CN(C(=O)Nc2nc3ccc(F)c(-c4cnc(NCC56COC(C)(C5)C6)nc4)c3s2)C1] | N-(7-bromo-6- fluorobenzo[d] thiazol-2-yl)- 3-fluoro-3- (methoxymethyl) azetidine-1- carboxamide and 2-((1-Methyl-2- oxabicyclo[2.1.1] hexan-4- yl)methoxy)-5- (4,4,5,5- tetramethyl-1,3- dioxolan-2- yl)pyrimidine | 517.2 |
| 164 | N-(7-(2- cyclobutoxypyrimidin-5-yl)- 6-fluorobenzo[d]thiazol-2- yl)-3-fluoro-3- (methoxymethyl)azetidine-1- carboxamide | 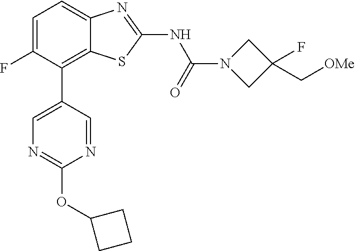 [SMILES: COCC1(F)CN(C(=O)Nc2nc3ccc(F)c(-c4cnc(OC5CCC5)nc4)c3s2)C1] | N-(7-bromo-6- fluorobenzo[d] thiazol-2-yl)- 3-fluoro-3- (methoxymethyl) azetidine-1- carboxamide | 462.1 |
| 165 | N-(6-fluoro-7-(6- ((tetrahydro-2H-pyran-4- yl)amino)pyridin-3- yl)benzo[d]thiazol-2-yl)-5,8- dioxa-2-azaspiro[3.5]nonane- 2-carboxamide | 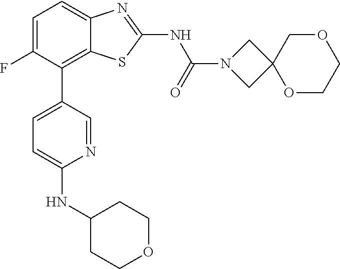 [SMILES: O=C(Nc1nc2ccc(F)c(-c3ccc(NC4CCOCC4)nc3)c2s1)N1CC2(COCCO2)C1] | N-(7-bromo-6- fluorobenzo[d] thiazol-2-yl)-5,8- dioxa-2- azaspiro[3.5]nonane- 2-carboxamide and N-(tetrahydro-2H- pyran-4-yl)-5- (4,4,5,5- tetramethyl-1,3,2- dioxaborolan-2- yl)pyridin-2-amine | 500.1 |
| 166 | 3-fluoro-N-(6-fluoro-7-(2- ((1r,3r)-3- hydroxycyclobutoxy) pyrimidin-5-yl)benzo [d]thiazol-2-yl)- 3-(methoxymethyl)azetidine- 1-carboxamide | 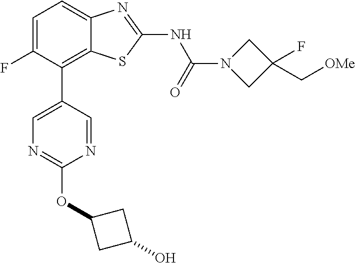 [SMILES: COCC1(F)CN(C(=O)Nc2nc3ccc(F)c(-c4cnc(O[C@H]5C[C@H](O)C5)nc4)c3s2)C1] | N-(7-bromo-6- fluorobenzo[d] thiazol-2-yl)- 3-fluoro-3- (methoxymethyl) azetidine-1- carboxamide and (1r,3r)-3-((5- (4,4,5,5- tetramethyl-1,3,2- dioxaborolan-2- yl)pyrimidin-2- yl)oxy)cyclobutan- 1-ol | 478.1 |
| 167 | N-(6-fluoro-7-(2-(oxetan-3- yloxy)pyrimidin-5- yl)benzo[d]thiazol-2-yl)-3- (methoxy-d3)-3- methylazetidine-1- carboxamide | 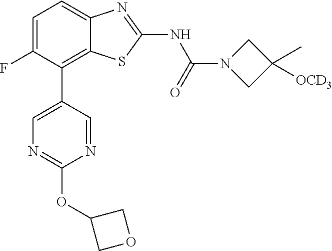 | N-(7-bromo-6- fluorobenzo[d] thiazol-2-yl)-3- (methoxy-d3)-3- methylazetidine-1- carboxamide | 449.1 |
| 168 | 3-fluoro-N-(6-fluoro-7-(2- (oxetan-3-yloxy)pyrimidin-5- yl)benzo[d]thiazol-2-yl)-3- ((methoxy- d3)methyl)azetidine-1- carboxamide | 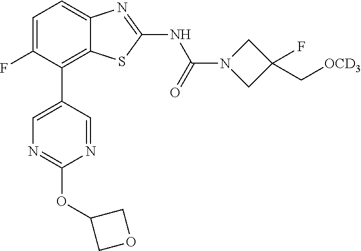 | N-(7-bromo-6- fluorobenzo[d] thiazol-2-yl)-3-fluoro- 3-((methoxy- d3)methyl)azetidine- 1-carboxamide | 467.1 |
| 169 | 3-fluoro-N-(6-fluoro-7- (pyridazin-4- yl)benzo[d]thiazol-2-yl)-3- (methoxymethyl)azetidine-1- carboxamide | 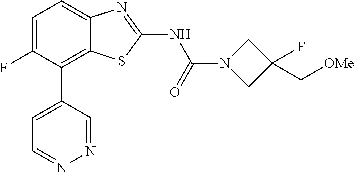 [SMILES: COCC1(F)CN(C(=O)Nc2nc3ccc(F)c(-c4ccnnc4)c3s2)C1] | N-(7-bromo-6- fluorobenzo[d] thiazol-2-yl)- 3-fluoro-3- (methoxymethyl) azetidine-1- carboxamide | 392.2 |
| 170 | N-(7-(2-(2- cyclopropoxyethoxy) pyrimidin-5-yl)-6- fluorobenzo[d]thiazol-2-yl)- 3-fluoro-3- (methoxymethyl)azetidine-1- carboxamide | 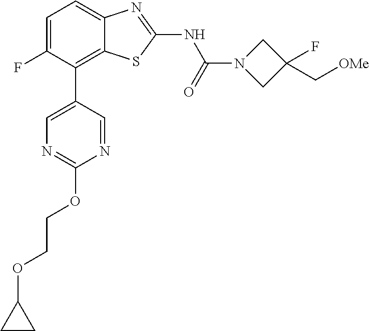 [SMILES: COCC1(F)CN(C(=O)Nc2nc3ccc(F)c(-c4cnc(OCCOC5CC5)nc4)c3s2)C1] | N-(7-bromo-6- fluorobenzo[d] thiazol-2-yl)- 3-fluoro-3- (methoxymethyl) azetidine-1- carboxamide 2-(2- cyclopropoxyethoxy)- 5-(4,4,5,5- tetramethyl-1,3,2- dioxaborolan-2- yl)pyrimidine | 492.2 |
| 171 | 3-fluoro-N-(6-fluoro-7-(2-(2- methoxyethoxy)pyrimidin-5- yl)benzo[d]thiazol-2-yl)-3- (methoxymethyl)azetidine-1- carboxamide | 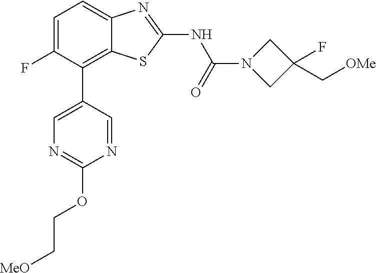 [SMILES: COCCOc1ncc(-c2c(F)ccc3nc(NC(=O)N4CC(F)(COC)C4)sc23)cn1] | N-(7-bromo-6- fluorobenzo[d] thiazol-2-yl)- 3-fluoro-3- (methoxymethyl) azetidine-1- carboxamide | 466.2 |
| 172 | N-(6-chloro-7-(2,3-dihydro- [1,4]dioxino[2,3-b]pyridin-7- yl)benzo[d]thiazol-2-yl)-3- fluoro-3-((methoxy- d3)methyl)azetidine-1- carboxamide | 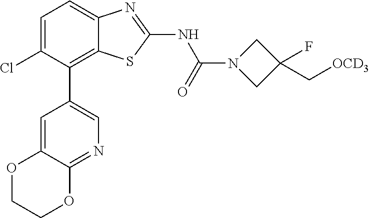 | N-(7-bromo-6- chlorobenzo[d] thiazol-2-yl)-3-fluoro- 3-((methoxy- d3)methyl)azetidine- 1-carboxamide | 468.2 |
| 173 | N-(6-chloro-7-(2,3-dihydro- [1,4]dioxino[2,3-b]pyridin-7- yl)benzo[d]thiazol-2-yl)-3- (methoxy-d3)-3- methylazetidine-1- carboxamide | 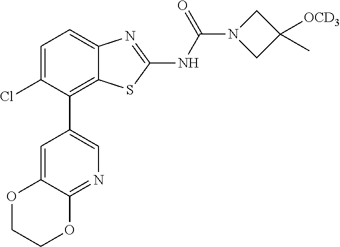 | N-(7-bromo-6- chlorobenzo[d] thiazol-2-yl)-3- (methoxy-d3)-3- methylazetidine-1- carboxamide | 450.1 |
| 174 | N-(6-chloro-7-(2-(2- hydroxypropan-2- yl)pyrimidin-5- yl)benzo[d]thiazol-2-yl)-3- fluoro-3-((methoxy- d3)methyl)azetidine-1- carboxamide | 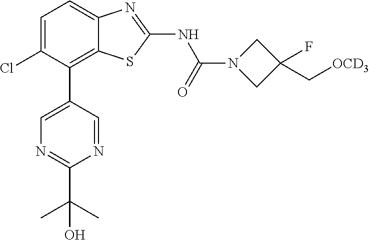 | N-(7-bromo-6- chlorobenzo[d] thiazol-2-yl)-3-fluoro- 3-((methoxy- d3)methyl)azetidine- 1-carboxamide and N-(7-bromo-6- fluorobenzo[d] thiazol-2-yl)-3-fluoro- 3-((methoxy- d3)methyl)azetidine- 1-carboxamide | 469.2 |
| 175 | N-(7-(6- (difluoromethoxy)pyridin-3- yl)-6-fluorobenzo[d]thiazol- 2-yl)-2-oxa-6- azaspiro[3.3]heptane-6- carboxamide | 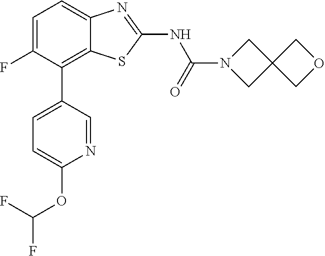 [SMILES: O=C(Nc1nc2ccc(F)c(-c3ccc(OC(F)F)nc3)c2s1)N1CC2(COC2)C1] | N-(7-bromo-6- fluorobenzo[d] thiazol-2-yl)-2-oxa-6- azaspiro[3.3]heptane- 6-carboxamide | 437.1 |
| 176 | N-(6-fluoro-7-(6- ((tetrahydro-2H-pyran-4- yl)amino)pyridin-3- yl)benzo[d]thiazol-2-yl)-2- oxa-6-azaspiro[3.3]heptane- 6-carboxamide | 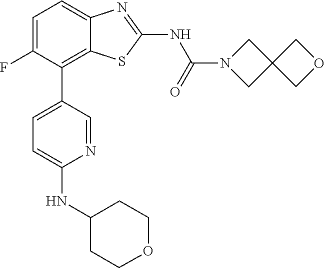 [SMILES: O=C(Nc1nc2ccc(F)c(-c3ccc(NC4CCOCC4)nc3)c2s1)N1CC2(COC2)C1] | N-(7-bromo-6- fluorobenzo[d] thiazol-2-yl)-2-oxa-6- azaspiro[3.3]heptane- 6-carboxamide | 470.1 |
| 177 | N-(6-fluoro-7-(5-fluoro-6- methoxypyridin-3- yl)benzo[d]thiazol-2-yl)-3- (methoxy-d3)-3- methylazetidine-1- carboxamide | 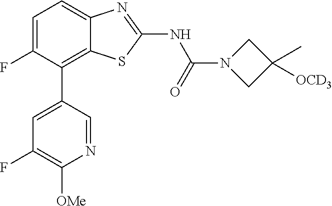 | N-(7-bromo-6- fluorobenzo[d] thiazol-2-yl)-3- (methoxy-d3)-3- methylazetidine-1- carboxamide | 424.1 |
| 178 | N-(6-fluoro-7-(6- methoxypyridin-3- yl)benzo[d]thiazol-2-yl)-3- (methoxy-d3)-3- methylazetidine-1- carboxamide | 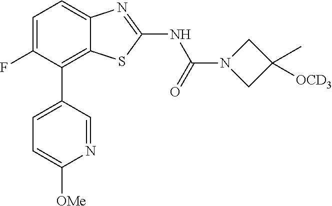 | N-(7-bromo-6- fluorobenzo[d] thiazol-2-yl)-3- (methoxy-d3)-3- methylazetidine-1- carboxamide | 406.1 |
| 179 | N-(6-chloro-7-(6- (difluoromethoxy)pyridin-3- yl)benzo[d]thiazol-2-yl)-2- oxa-6-azaspiro[3.3]heptane- 6-carboxamide | 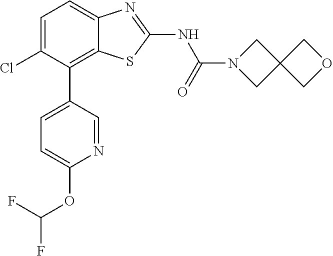 [SMILES: O=C(Nc1nc2ccc(Cl)c(-c3ccc(OC(F)F)nc3)c2s1)N1CC2(COC2)C1] | N-(7-bromo-6- chlorobenzo[d] thiazol-2-yl)-2-oxa-6- azaspiro[3.3]heptane- 6-carboxamide | 453.1 |
| 180 | N-(6-chloro-7-(2- isopropoxypyrimidin-5- yl)benzo[d]thiazol-2-yl)-3- fluoro-3-((methoxy- d3)methyl)azetidine-1- carboxamide | 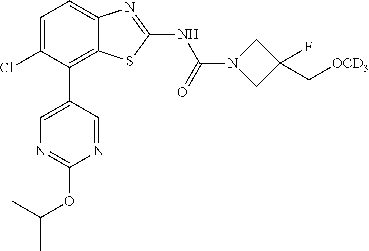 | N-(7-bromo-6- chlorobenzo[d] thiazol-2-yl)-3-fluoro- 3-((methoxy- d3)methyl)azetidine- 1-carboxamide | 469.1 |
| 181 | N-(6-chloro-7-(2- ethoxypyrimidin-5- yl)benzo[d]thiazol-2-yl)-3- fluoro-3-((methoxy- d3)methyl)azetidine-1- carboxamide | 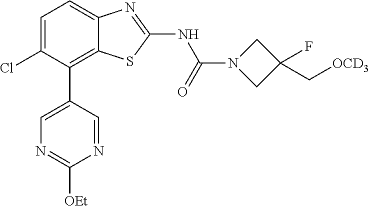 | N-(7-bromo-6- chlorobenzo[d] thiazol-2-yl)-3-fluoro- 3-((methoxy- d3)methyl)azetidine- 1-carboxamide | 455.1 |
| 182 | N-(6-chloro-7-(2- ((tetrahydro-2H-pyran-4- yl)amino)pyrimidin-5- yl)benzo[d]thiazol-2-yl)-3- (methoxy-d3)-3- methylazetidine-1- carboxamide | 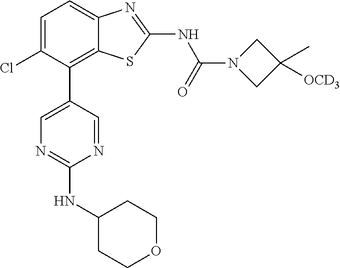 | N-(7-bromo-6- chlorobenzo[d] thiazol-2-yl)-3- (methoxy-d3)-3- methylazetidine-1- carboxamide | 492.1 |
| 183 | N-(6-chloro-7-(2- ((tetrahydro-2H-pyran-4- yl)amino)pyrimidin-5- yl)benzo[d]thiazol-2-yl)-3- fluoro-3-((methoxy- d3)methyl)azetidine-1- carboxamide | 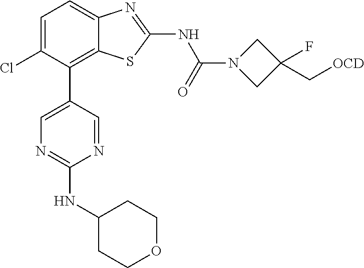 | N-(7-bromo-6- chlorobenzo[d] thiazol-2-yl)-3-fluoro- 3-((methoxy- d3)methyl)azetidine- 1-carboxamide N-(tetrahydro-2H- pyran-4-yl)-5- (4,4,5,5- tetramethyl-1,3,2- dioxaborolan-2- yl)pyrimidin-2- amine | 510.2 |
| 184 | N-(6-chloro-7-(6- (difluoromethoxy)pyridin-3- yl)benzo[d]thiazol-2-yl)-3- fluoro-3-((methoxy- d3)methyl)azetidine-1- carboxamide | 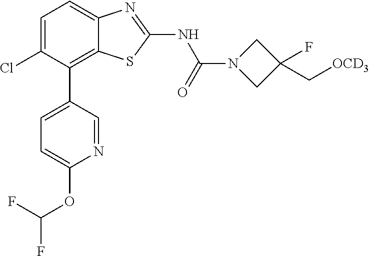 | N-(7-bromo-6- chlorobenzo[d] thiazol-2-yl)-3-fluoro- 3-((methoxy- d3)methyl)azetidine- 1-carboxamide | 476.1 |
| 185 | N-(6-chloro-7-(2- (difluoromethoxy)pyrimidin- 5-yl)benzo[d]thiazol-2-yl)-3- fluoro-3-((methoxy- d3)methyl)azetidine-1- carboxamide | 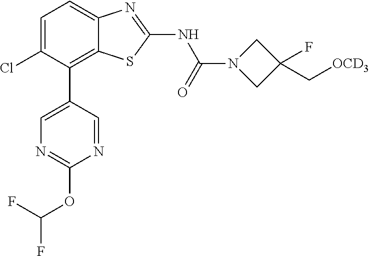 | N-(7-bromo-6- chlorobenzo[d] thiazol-2-yl)-3-fluoro- 3-((methoxy- d3)methyl)azetidine- 1-carboxamide | 477.0 |
| 186 | N-(6-chloro-7-(6-(2- morpholinoethoxy)pyridin-3- yl)benzo[d]thiazol-2-yl)-3- fluoro-3-((methoxy- d3)methyl)azetidine-1- carboxamide | 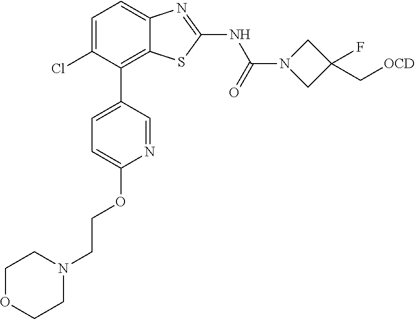 | N-(7-bromo-6- chlorobenzo[d] thiazol-2-yl)-3-fluoro- 3-((methoxy- d3)methyl)azetidine- 1-carboxamide | 539.2 |
| 187 | N-(6-chloro-7-(6-(1,1- difluoroethyl)pyridin-3- yl)benzo[d]thiazol-2-yl)-3- fluoro-3-((methoxy- d3)methyl)azetidine-1- carboxamide | 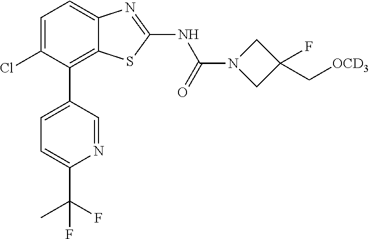 | N-(7-bromo-6- chlorobenzo[d] thiazol-2-yl)-3-fluoro- 3-((methoxy- d3)methyl)azetidine- 1-carboxamide | 474.1 |
| 188 | N-(6-chloro-7-(6- ((tetrahydro-2H-pyran-4- yl)amino)pyridin-3- yl)benzo[d]thiazol-2-yl)-3- fluoro-3-((methoxy- d3)methyl)azetidine-1- carboxamide | 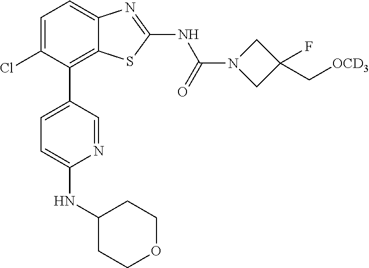 | N-(7-bromo-6- chlorobenzo[d] thiazol-2-yl)-3-fluoro- 3-((methoxy- d3)methyl)azetidine- 1-carboxamide | 509.2 |
| 189 | N-(6-chloro-7-(2- (difluoromethoxy)pyrimidin- 5-yl)benzo[d]thiazol-2-yl)-3- (methoxy-d3)-3- methylazetidine-1- carboxamide | 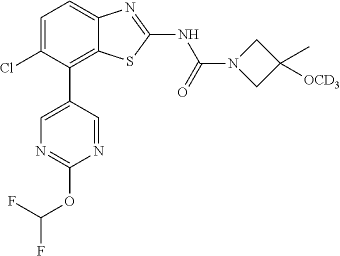 | N-(7-bromo-6- chlorobenzo[d] thiazol-2-yl)-3- (methoxy-d3)-3- methylazetidine-1- carboxamide | 459.2 |
| 190 | N-(6-chloro-7-(2- methoxypyrimidin-5- yl)benzo[d]thiazol-2-yl)-3- (methoxy-d3)-3- methylazetidine-1- carboxamide | 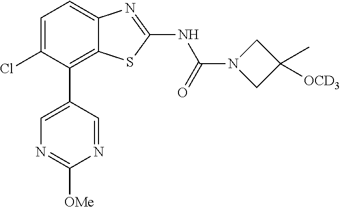 | N-(7-bromo-6- chlorobenzo[d] thiazol-2-yl)-3- (methoxy-d3)-3- methylazetidine-1- carboxamide | 423.1 |
| 191 | N-(6-chloro-7-(6- (difluoromethoxy)pyridin-3- yl)benzo[d]thiazol-2-yl)-3- (methoxy-d3)-3- methylazetidine-1- carboxamide | 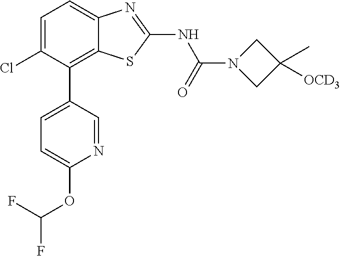 | N-(7-bromo-6- chlorobenzo[d] thiazol-2-yl)-3- (methoxy-d3)-3- methylazetidine-1- carboxamide | 458.1 |
| 192 | N-(6-chloro-7-(6- methoxypyridin-3- yl)benzo[d]thiazol-2-yl)-3- (methoxy-d3)-3- methylazetidine-1- carboxamide | 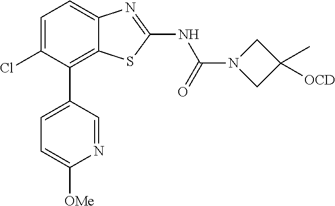 | N-(7-bromo-6- chlorobenzo[d] thiazol-2-yl)-3- (methoxy-d3)-3- methylazetidine-1- carboxamide | 422.1 |
| 193 | oxazol-2-ylmethyl (7-(1- methyl-6-oxo-1,6- dihydropyridin-3- yl)benzo[d]thiazol-2- yl)carbamate | 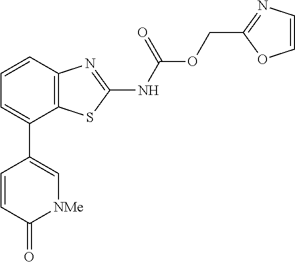 [SMILES: Cn1cc(-c2cccc3nc(NC(=O)OCc4ncco4)sc23)ccc1=O] | oxazol-2-ylmethyl (7-bromobenzo[d] thiazol-2-yl)carbamate | 383.1 |
| 194 | oxazol-2-ylmethyl (7-(6-oxo- 1,6-dihydropyridin-3- yl)benzo[d]thiazol-2- yl)carbamate | 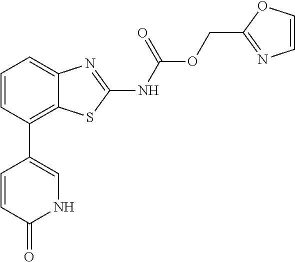 [SMILES: O=C(Nc1nc2cccc(-c3ccc(=O)[nH]c3)c2s1)OCc1ncco1] | oxazol-2-ylmethyl (7-bromobenzo[d] thiazol-2-yl)carbamate | 369.1 |
| 195 | oxazol-2-ylmethyl (7-(3- chloro-4- methoxyphenyl)benzo[d] thiazol-2-yl)carbamate | 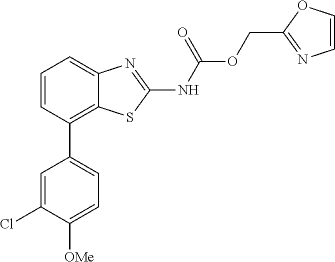 [SMILES: COc1ccc(-c2cccc3nc(NC(=O)OCc4ncco4)sc23)cc1Cl] | oxazol-2-ylmethyl (7-bromobenzo[d] thiazol-2-yl)carbamate | 416.0 |
| 196 | oxazol-2-ylmethyl (7-(3- fluoro-4- methoxyphenyl)benzo[d] thiazol-2-yl)carbamate | 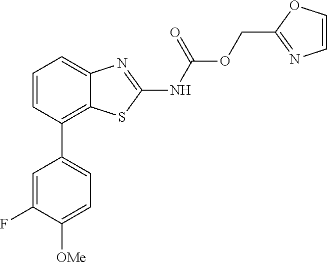 [SMILES: COc1ccc(-c2cccc3nc(NC(=O)OCc4ncco4)sc23)cc1F] | oxazol-2-ylmethyl (7- bromobenzo[d] thiazol-2-yl)carbamate | 400.1 |
| 197 | oxazol-2-ylmethyl (7-(2,4- dimethoxypyrimidin-5- yl)benzo[d]thiazol-2- yl)carbamate | 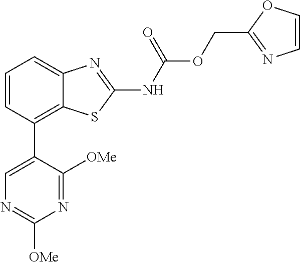 [SMILES: COc1ncc(-c2cccc3nc(NC(=O)OCc4ncco4)sc23)c(OC)n1] | oxazol-2-ylmethyl (7-bromobenzo[d] thiazol-2-yl)carbamate | 414.0 |
| 198 | oxazol-2-ylmethyl (7-(6- methylpyridin-3- yl)benzo[d]thiazol-2- yl)carbamate | 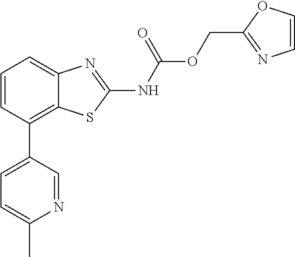 [SMILES: Cc1ccc(-c2cccc3nc(NC(=O)OCc4ncco4)sc23)cn1] | oxazol-2-ylmethyl (7-bromobenzo[d] thiazol-2-yl)carbamate | 367.1 |
| 199 | oxazol-2-ylmethyl (7-(2,6- dimethylpyridin-3- yl)benzo[d]thiazol-2- yl)carbamate | 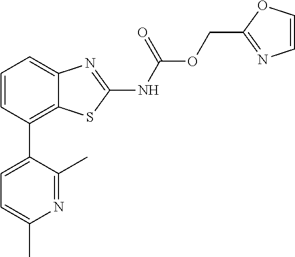 [SMILES: Cc1ccc(-c2cccc3nc(NC(=O)OCc4ncco4)sc23)c(C)n1] | oxazol-2-ylmethyl (7-bromobenzo[d] thiazol-2-yl)carbamate | 381.1 |
| 200 | oxazol-2-ylmethyl (7-(2- fluoro-6-methoxypyridin-3- yl)benzo[d]thiazol-2- yl)carbamate | 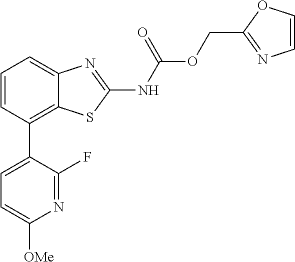 [SMILES: COc1ccc(-c2cccc3nc(NC(=O)OCc4ncco4)sc23)c(F)n1] | oxazol-2-ylmethyl (7-bromobenzo[d] thiazol-2-yl)carbamate | 401.0 |
| 201 | oxazol-2-ylmethyl (7-(6- methoxy-4-methylpyridin-3- yl)benzo[d]thiazol-2- yl)carbamate | 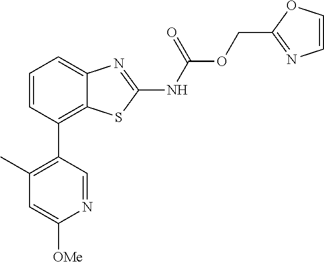 [SMILES: COc1cc(C)c(-c2cccc3nc(NC(=O)OCc4ncco4)sc23)cn1] | oxazol-2-ylmethyl (7-bromobenzo[d] thiazol-2-yl)carbamate | 397.1 |
| 202 | oxazol-2-ylmethyl (7-(4,6- dimethoxypyridin-3- yl)benzo[d]thiazol-2- yl)carbamate | 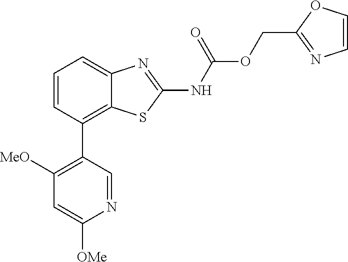 [SMILES: COc1cc(OC)c(-c2cccc3nc(NC(=O)OCc4ncco4)sc23)cn1] | oxazol-2-ylmethyl (7-bromobenzo[d] thiazol-2-yl)carbamate | 413.1 |
| 203 | oxazol-2-ylmethyl (7-(4- methyl-6- (trifluoromethyl)pyridin-3- yl)benzo[d]thiazol-2- yl)carbamate | 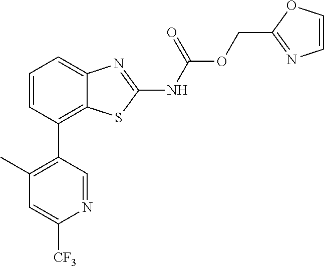 [SMILES: Cc1cc(C(F)(F)F)ncc1-c1cccc2nc(NC(=O)OCc3ncco3)sc12] | oxazol-2-ylmethyl (7-bromobenzo[d] thiazol-2-yl)carbamate | 435.0 |
| 204 | oxazol-2-ylmethyl (7-(5- fluoro-6-methoxypyridin-3- yl)benzo[d]thiazol-2- yl)carbamate | 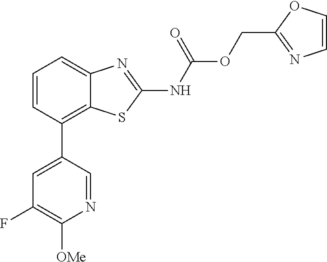 [SMILES: COc1ncc(-c2cccc3nc(NC(=O)OCc4ncco4)sc23)cc1F] | oxazol-2-ylmethyl (7-bromobenzo[d] thiazol-2-yl)carbamate | 401.0 |
| 205 | oxazol-2-ylmethyl (7-(5- chloro-6-methoxypyridin-3- yl)benzo[d]thiazol-2- yl)carbamate | 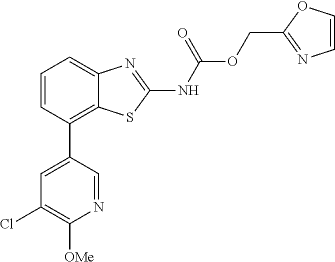 [SMILES: COc1ncc(-c2cccc3nc(NC(=O)OCc4ncco4)sc23)cc1Cl] | oxazol-2-ylmethyl (7-bromobenzo[d] thiazol-2-yl)carbamate | 417.0 |
| 206 | oxazol-2-ylmethyl (7-(5- amino-6-methoxypyridin-3- yl)benzo[d]thiazol-2- yl)carbamate | 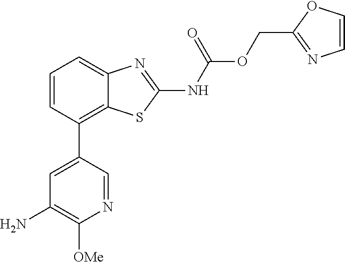 [SMILES: COc1ncc(-c2cccc3nc(NC(=O)OCc4ncco4)sc23)cc1N] | oxazol-2-ylmethyl (7-bromobenzo[d] thiazol-2-yl)carbamate | 398.0 |
| 207 | oxazol-2-ylmethyl (7-(6- isopropoxypyridin-3- yl)benzo[d]thiazol-2- yl)carbamate | 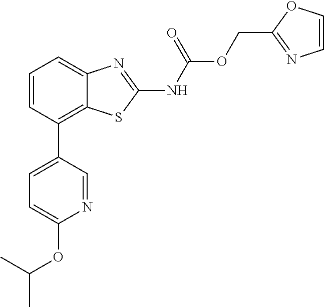 [SMILES: CC(C)Oc1ccc(-c2cccc3nc(NC(=O)OCc4ncco4)sc23)cn1] | oxazol-2-ylmethyl (7-bromobenzo[d] thiazol-2-yl)carbamate | 411.0 |
| 208 | oxazol-2-ylmethyl (7-(6- cyanopyridin-3- yl)benzo[d]thiazol-2- yl)carbamate | 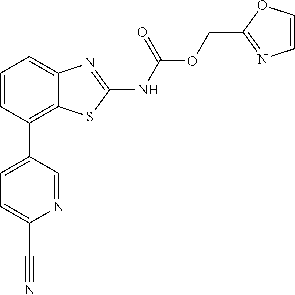 [SMILES: N#Cc1ccc(-c2cccc3nc(NC(=O)OCc4ncco4)sc23)cn1] | oxazol-2-ylmethyl (7-bromobenzo[d] thiazol-2-yl)carbamate | 378.0 |
| 209 | oxazol-2-ylmethyl (7-(6- carbamoylpyridin-3- yl)benzo[d]thiazol-2- yl)carbamate | 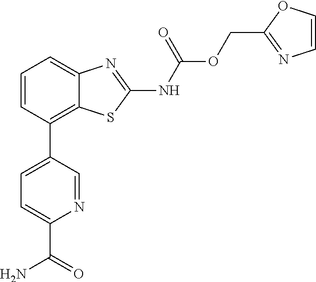 [SMILES: NC(=O)c1ccc(-c2cccc3nc(NC(=O)OCc4ncco4)sc23)cn1] | oxazol-2-ylmethyl (7-bromobenzo[d] thiazol-2-yl)carbamate | 396.0 |
| 210 | oxazol-2-ylmethyl (7-(6- (methylcarbamoyl)pyridin-3- yl)benzo[d]thiazol-2- yl)carbamate | 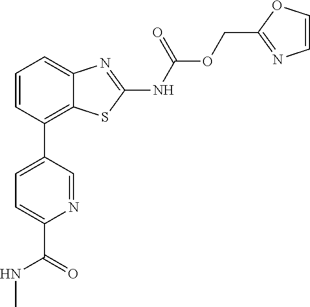 [SMILES: CNC(=O)c1ccc(-c2cccc3nc(NC(=O)OCc4ncco4)sc23)cn1] | oxazol-2-ylmethyl (7-bromobenzo[d] thiazol-2-yl)carbamate | 410.0 |
| 211 | oxazol-2-ylmethyl (7-(6- (dimethylcarbamoyl)pyridin- 3-yl)benzo[d]thiazol-2- yl)carbamate | 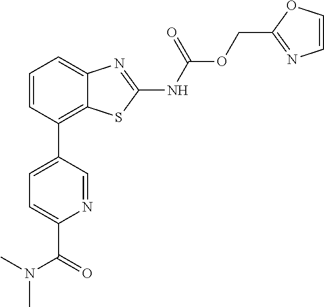 [SMILES: CN(C)C(=O)c1ccc(-c2cccc3nc(NC(=O)OCc4ncco4)sc23)cn1] | oxazol-2-ylmethyl (7-bromobenzo[d] thiazol-2-yl)carbamate | 424.1 |
| 212 | oxazol-2-ylmethyl (7-(6- aminopyridin-3- yl)benzo[d]thiazol-2- yl)carbamate | 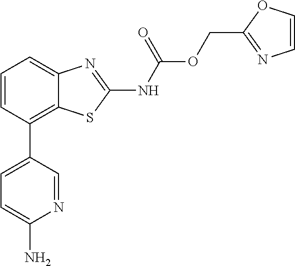 [SMILES: Nc1ccc(-c2cccc3nc(NC(=O)OCc4ncco4)sc23)cn1] | oxazol-2-ylmethyl (7-bromobenzo[d] thiazol-2-yl)carbamate | 368.0 |
| 213 | oxazol-2-ylmethyl (7-(6- (methylamino)pyridin-3- yl)benzo[d]thiazol-2- yl)carbamate | 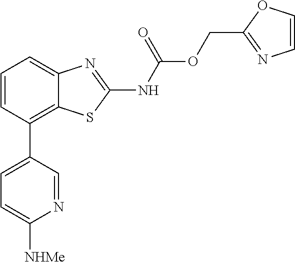 [SMILES: CNc1ccc(-c2cccc3nc(NC(=O)OCc4ncco4)sc23)cn1] | oxazol-2-ylmethyl (7-bromobenzo[d] thiazol-2-yl)carbamate | 382.0 |
| 214 | oxazol-2-ylmethyl (7-(6- (dimethylamino)pyridin-3- yl)benzo[d]thiazol-2- yl)carbamate | 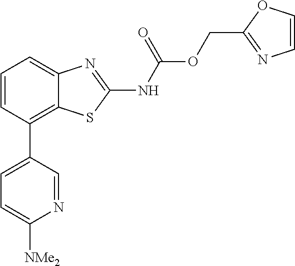 [SMILES: CN(C)c1ccc(-c2cccc3nc(NC(=O)OCc4ncco4)sc23)cn1] | oxazol-2-ylmethyl (7-bromobenzo[d] thiazol-2-yl)carbamate | 396.0 |
| 215 | oxazol-2-ylmethyl (7-(6- morpholinopyridin-3- yl)benzo[d]thiazol-2- yl)carbamate | 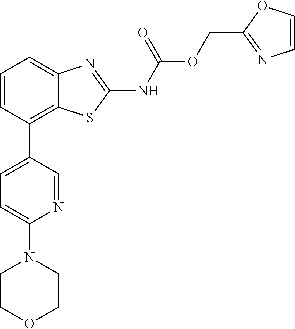 [SMILES: O=C(Nc1nc2cccc(-c3ccc(N4CCOCC4)nc3)c2s1)OCc1ncco1] | oxazol-2-ylmethyl (7-bromobenzo[d] thiazol-2-yl)carbamate | 438.2 |
| 216 | oxazol-2-ylmethyl (7-(6- (pyrrolidin-1-yl)pyridin-3- yl)benzo[d]thiazol-2- yl)carbamate | 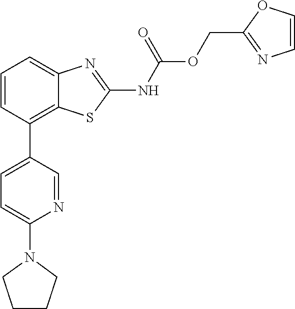 [SMILES: O=C(Nc1nc2cccc(-c3ccc(N4CCCC4)nc3)c2s1)OCc1ncco1] | oxazol-2-ylmethyl (7-bromobenzo[d] thiazol-2-yl)carbamate | 422.1 |
| 217 | oxazol-2-ylmethyl (7-(6- cyclopropylpyridin-3- yl)benzo[d]thiazol-2- yl)carbamate | 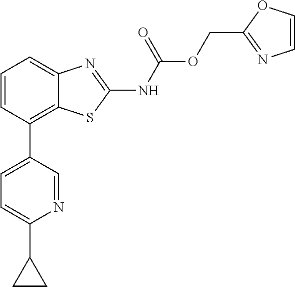 [SMILES: O=C(Nc1nc2cccc(-c3ccc(C4CC4)nc3)c2s1)OCc1ncco1] | oxazol-2-ylmethyl (7-bromobenzo[d] thiazol-2-yl)carbamate | 393.1 |
| 218 | oxazol-2-ylmethyl (7-(6- (methylsulfonyl)pyridin-3- yl)benzo[d]thiazol-2- yl)carbamate | 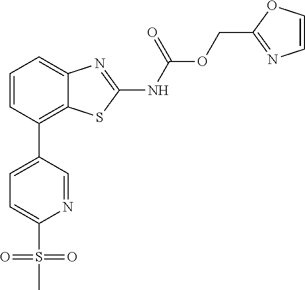 [SMILES: CS(=O)(=O)c1ccc(-c2cccc3nc(NC(=O)OCc4ncco4)sc23)cn1] | oxazol-2-ylmethyl (7-bromobenzo[d] thiazol-2-yl)carbamate | 431.1 |
| 219 | oxazol-2-ylmethyl (7-(6- acetylpyridin-3- yl)benzo[d]thiazol-2- yl)carbamate | 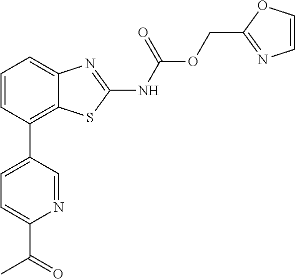 [SMILES: CC(=O)c1ccc(-c2cccc3nc(NC(=O)OCc4ncco4)sc23)cn1] | oxazol-2-ylmethyl (7-bromobenzo[d] thiazol-2-yl)carbamate | 395.1 |
| 220 | oxazol-2-ylmethyl (7-(6- (trifluoromethoxy)pyridin-3- yl)benzo[d]thiazol-2- yl)carbamate | 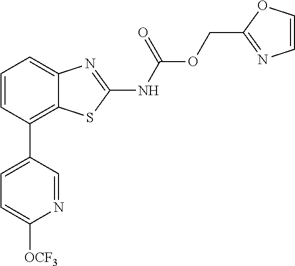 [SMILES: O=C(Nc1nc2cccc(-c3ccc(OC(F)(F)F)nc3)c2s1)OCc1ncco1] | oxazol-2-ylmethyl (7-bromobenzo[d] thiazol-2-yl)carbamate | 437.1 |
| 221 | oxazol-2-ylmethyl (7-(6- (cyclopropylmethoxy) pyridin-3-yl)benzo[d] thiazol-2- yl)carbamate | 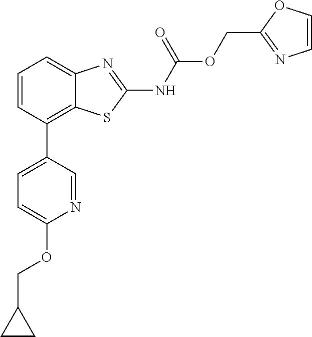 [SMILES: O=C(Nc1nc2cccc(-c3ccc(OCC4CC4)nc3)c2s1)OCc1ncco1] | oxazol-2-ylmethyl (7-bromobenzo[d] thiazol-2-yl)carbamate | 423.0 |
| 222 | oxazol-2-ylmethyl (7-(6- (morpholinomethyl)pyridin- 3-yl)benzo[d]thiazol-2- yl)carbamate | 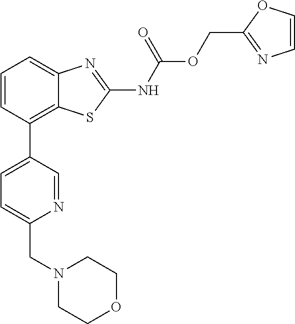 [SMILES: O=C(Nc1nc2cccc(-c3ccc(CN4CCOCC4)nc3)c2s1)OCc1ncco1] | oxazol-2-ylmethyl (7-bromobenzo[d] thiazol-2-yl)carbamate | 452.1 |
| 223 | oxazol-2-ylmethyl (7-(6- ((tetrahydro-2H-pyran-4- yl)oxy)pyridin-3- yl)benzo[d]thiazol-2- yl)carbamate | 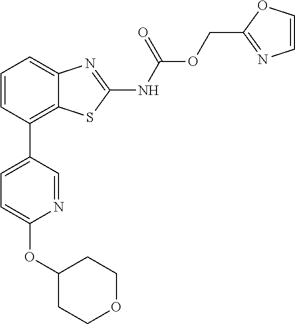 [SMILES: O=C(Nc1nc2cccc(-c3ccc(OC4CCOCC4)nc3)c2s1)OCc1ncco1] | oxazol-2-ylmethyl (7-bromobenzo[d] thiazol-2-yl)carbamate | 453.1 |
| 224 | oxazol-2-ylmethyl (7-(2,6- dimethoxypyridin-3- yl)benzo[d]thiazol-2- yl)carbamate | 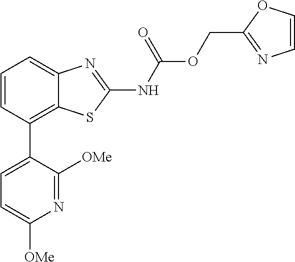 [SMILES: COc1ccc(-c2cccc3nc(NC(=O)OCc4ncco4)sc23)c(OC)n1] | oxazol-2-ylmethyl (7-bromobenzo[d] thiazol-2-yl)carbamate | 413.1 |
| 225 | oxazol-2-ylmethyl (7-(6- methoxy-2-methylpyridin-3- yl)benzo[d]thiazol-2- yl)carbamate | 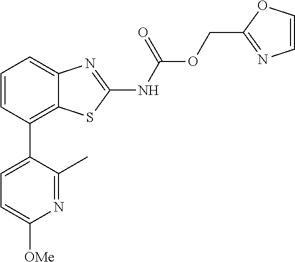 [SMILES: COc1ccc(-c2cccc3nc(NC(=O)OCc4ncco4)sc23)c(C)n1] | oxazol-2-ylmethyl (7-bromobenzo[d] thiazol-2-yl)carbamate | 397.1 |
| 226 | oxazol-2-ylmethyl (7-(2- methoxy-6-methylpyridin-3- yl)benzo[d]thiazol-2- yl)carbamate | 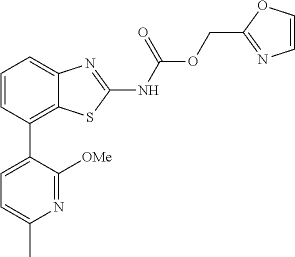 [SMILES: COc1nc(C)ccc1-c1cccc2nc(NC(=O)OCc3ncco3)sc12] | oxazol-2-ylmethyl (7-bromobenzo[d] thiazol-2-yl)carbamate | 397.1 |
| 227 | oxazol-2-ylmethyl (7-(6-(2- (dimethylamino)ethoxy) pyridin-3-yl)benzo[d] thiazol-2-yl)carbamate | 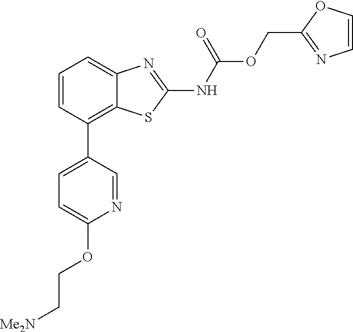 [SMILES: CN(C)CCOc1ccc(-c2cccc3nc(NC(=O)OCc4ncco4)sc23)cn1] | oxazol-2-ylmethyl (7- bromobenzo[d] thiazol-2-yl)carbamate | 440.1 |
| 228 | oxazol-2-ylmethyl (7-(6-(2- methoxyethoxy)pyridin-3- yl)benzo[d]thiazol-2- yl)carbamate | 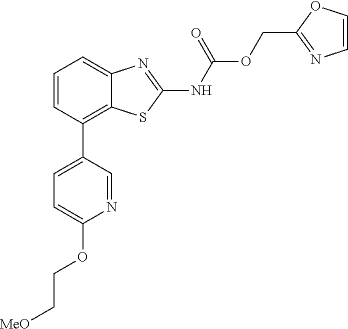 [SMILES: COCCOc1ccc(-c2cccc3nc(NC(=O)OCc4ncco4)sc23)cn1] | oxazol-2-ylmethyl (7-bromobenzo[d] thiazol-2-yl)carbamate | 427.1 |
| 229 | oxazol-2-ylmethyl (7-(2,3- dihydro-[1,4]dioxino[2,3- b]pyridin-7- yl)benzo[d]thiazol-2- yl)carbamate | 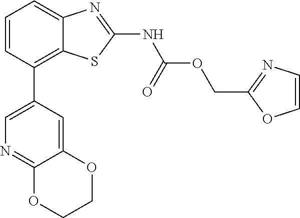 [SMILES: O=C(Nc1nc2cccc(-c3cnc4c(c3)OCCO4)c2s1)OCc1ncco1] | oxazol-2-ylmethyl (7-bromobenzo[d] thiazol-2-yl)carbamate | 411.0 |
| 230 | oxazol-2-ylmethyl (7-(6-(2- oxa-6-azaspiro[3.3]heptan-6- yl)pyridin-3- yl)benzo[d]thiazol-2- yl)carbamate | 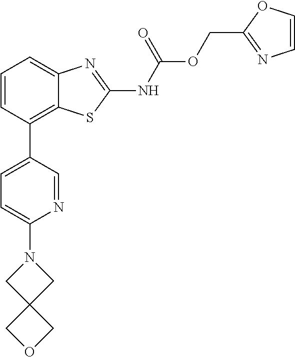 [SMILES: O=C(Nc1nc2cccc(-c3ccc(N4CC5(COC5)C4)nc3)c2s1)OCc1ncco1] | oxazol-2-ylmethyl (7-bromobenzo[d] thiazol-2-yl)carbamate and 6-(5-(4,4,5,5- tetramethyl-1,3,2- dioxaborolan-2- yl)pyridin-2-yl)-2- oxa-6- azaspiro[3.3]heptane | 450.1 |
| 231 | oxazol-2-ylmethyl (7-(1- methyl-1H-pyrazol-4- yl)benzo[d]thiazol-2- yl)carbamate | 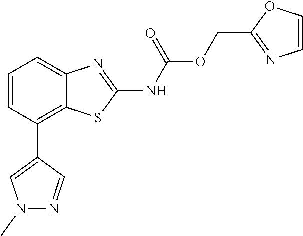 [SMILES: Cn1cc(-c2cccc3nc(NC(=O)OCc4ncco4)sc23)cn1] | oxazol-2-ylmethyl (7- bromobenzo[d] thiazol-2-yl)carbamate | 356.0 |
| 232 | oxazol-2-ylmethyl (7-(6-(1,1- difluoroethyl)pyridin-3- yl)benzo[d]thiazol-2- yl)carbamate | 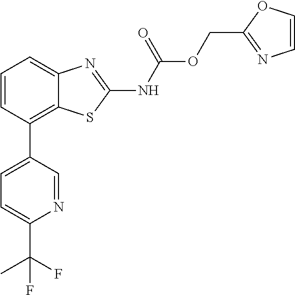 [SMILES: CC(F)(F)c1ccc(-c2cccc3nc(NC(=O)OCc4ncco4)sc23)cn1] | oxazol-2-ylmethyl (7-bromobenzo[d] thiazol-2-yl)carbamate | 417.0 |
| 233 | oxazol-2-ylmethyl (7-(2,4- dimethoxyphenyl)benzo [d]thiazol-2-yl)carbamate | 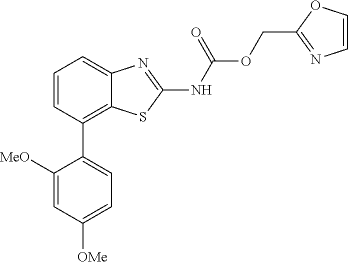 [SMILES: COc1ccc(-c2cccc3nc(NC(=O)OCc4ncco4)sc23)c(OC)c1] | oxazol-2-ylmethyl (7- bromobenzo[d] thiazol-2-yl)carbamate | 412.0 |
| 234 | oxazol-2-ylmethyl (7-(6- (morpholine-4- carbonyl)pyridin-3- yl)benzo[d]thiazol-2- yl)carbamate | 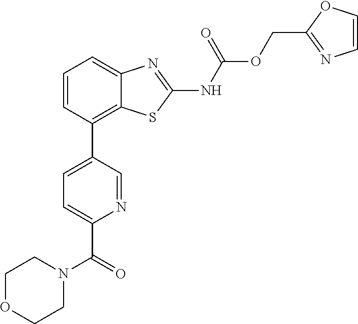 [SMILES: O=C(Nc1nc2cccc(-c3ccc(C(=O)N4CCOCC4)nc3)c2s1)OCc1ncco1] | oxazol-2-ylmethyl (7- bromobenzo[d] thiazol-2-yl)carbamate | 466.1 |
| 235 | oxazol-2-ylmethyl (7-(6-((2- methoxyethyl)carbamoyl) pyridin-3-yl)benzo[d] thiazol-2-yl)carbamate | 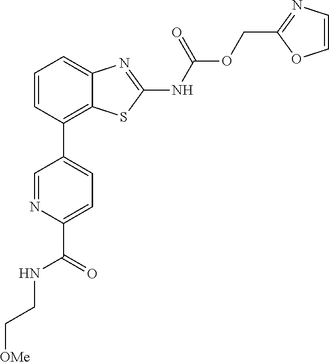 [SMILES: COCCNC(=O)c1ccc(-c2cccc3nc(NC(=O)OCc4ncco4)sc23)cn1] | oxazol-2-ylmethyl (7-bromobenzo[d] thiazol-2-yl)carbamate | 454.0 |
| 236 | oxazol-2-ylmethyl (7-(6-(3- (methylamino)oxetan-3- yl)pyridin-3- yl)benzo[d]thiazol-2- yl)carbamate | 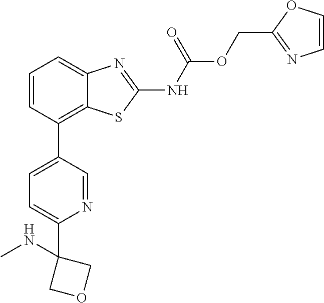 [SMILES: CNC1(c2ccc(-c3cccc4nc(NC(=O)OCc5ncco5)sc34)cn2)COC1] | oxazol-2-ylmethyl (7-bromobenzo[d] thiazol-2-yl)carbamate and N-methyl-3-(5- (4,4,5,5- tetramethyl-1,3,2- dioxaborolan-2- yl)pyridin-2- yl)oxetan-3-amine | 438.2 |
| 237 | oxazol-2-ylmethyl (7-(2- ((tetrahydro-2H-pyran-4- yl)oxy)pyrimidin-5- yl)benzo[d]thiazol-2- yl)carbamate | 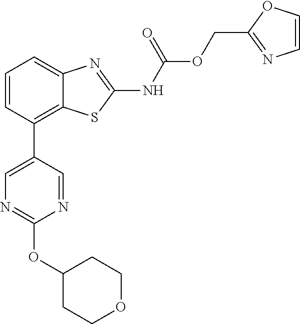 [SMILES: O=C(Nc1nc2cccc(-c3cnc(OC4CCOCC4)nc3)c2s1)OCc1ncco1] | oxazol-2-ylmethyl (7-bromobenzo[d] thiazol-2-yl)carbamate and 2-((tetrahydro-2H- pyran-4-yl)oxy)-5- (4,4,5,5- tetramethyl-1,3,2- dioxaborolan-2- yl)pyrimidine | 454.2 |
| 238 | oxazol-2-ylmethyl (7-(6- ((tetrahydro-2H-pyran-4- yl)amino)pyridin-3- yl)benzo[d]thiazol-2- yl)carbamate | 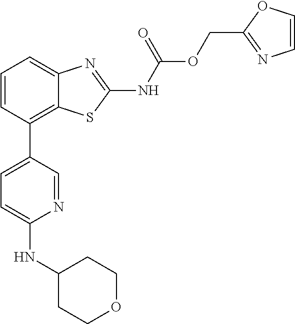 [SMILES: O=C(Nc1nc2cccc(-c3ccc(NC4CCOCC4)nc3)c2s1)OCc1ncco1] | oxazol-2-ylmethyl (7-bromobenzo[d] thiazol-2-yl)carbamate and N-(tetrahydro-2H- pyran-4-yl)-5- (4,4,5,5- tetramethyl-1,3,2- dioxaborolan-2- yl)pyridin-2-amine | 452.1 |
| 239 | oxazol-2-ylmethyl (7-(1-(2- hydroxy-2-methylpropyl)- 1H-pyrazol-4- yl)benzo[d]thiazol-2- yl)carbamate | 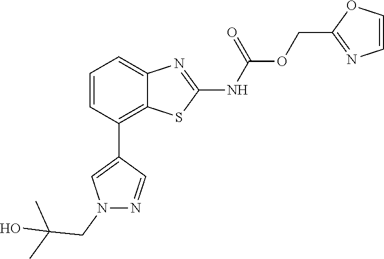 [SMILES: CC(C)(O)Cn1cc(-c2cccc3nc(NC(=O)OCc4ncco4)sc23)cn1] | oxazol-2-ylmethyl (7-bromobenzo[d] thiazol-2-yl)carbamate | 414.1 |
| 240 | oxazol-2-ylmethyl (7-(1-(2- methoxyethyl)-1H-pyrazol- 4-yl)benzo[d]thiazol-2- yl)carbamate | 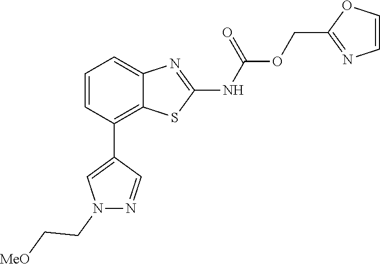 [SMILES: COCCn1cc(-c2cccc3nc(NC(=O)OCc4ncco4)sc23)cn1] | oxazol-2-ylmethyl (7-bromobenzo[d] thiazol-2-yl)carbamate | 400.1 |
| 241 | oxazol-2-ylmethyl (7-(1- (oxetan-3-yl)-1H-pyrazol- 4-yl)benzo[d]thiazol-2- yl)carbamate | 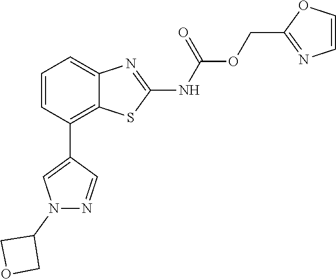 [SMILES: O=C(Nc1nc2cccc(-c3cnn(C4COC4)c3)c2s1)OCc1ncco1] | oxazol-2-ylmethyl (7-bromobenzo[d] thiazol-2-yl)carbamate | 398.1 |
| 242 | oxazol-2-ylmethyl (7-(1- (tetrahydrofuran-3-yl)-1H- pyrazol-4-yl)benzo[d] thiazol-2-yl)carbamate | 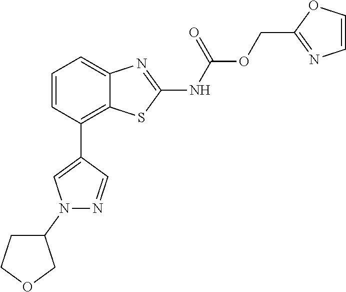 [SMILES: O=C(Nc1nc2cccc(-c3cnn(C4CCOC4)c3)c2s1)OCc1ncco1] | oxazol-2-ylmethyl (7-bromobenzo[d] thiazol-2-yl)carbamate | 412.1 |
| 243 | oxazol-2-ylmethyl (7-(6-((2- oxa-6-azaspiro[3.3]heptan- 6-yl)methyl)pyridin-3- yl)benzo[d]thiazol-2- yl)carbamate | 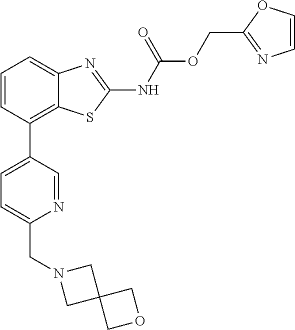 [SMILES: O=C(Nc1nc2cccc(-c3ccc(CN4CC5(COC5)C4)nc3)c2s1)OCc1ncco1] | oxazol-2-ylmethyl (7- bromobenzo[d] thiazol-2-yl)carbamate and 6-((5-(4,4,5,5- tetramethyl-1,3,2- dioxaborolan-2- yl)pyridin-2- yl)methyl)-2-oxa-6- azaspiro[3.3]heptane | 464.1 |
| 244 | oxazol-2-ylmethyl (7-(6- (oxetan-3- ylcarbamoyl)pyridin-3- yl)benzo[d]thiazol-2- yl)carbamate | 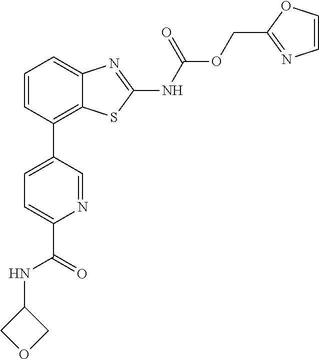 [SMILES: O=C(Nc1nc2cccc(-c3ccc(C(=O)NC4COC4)nc3)c2s1)OCc1ncco1] | oxazol-2-ylmethyl (7-bromobenzo[d] thiazol-2-yl)carbamate and N-(oxetan-3-yl)-5- (4,4,5,5- tetramethyl-1,3,2- dioxaborolan-2- yl)picolinamide | 452.1 |
| 245 | oxazol-2-ylmethyl (7-(1- ((tetrahydro-2H-pyran-4- yl)methyl)-1H-pyrazol-4- yl)benzo[d]thiazol-2- yl)carbamate | 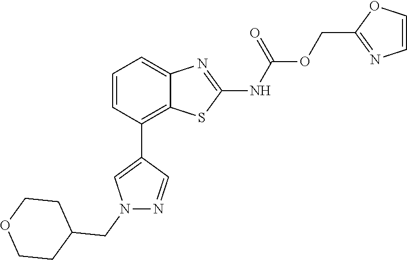 [SMILES: O=C(Nc1nc2cccc(-c3cnn(CC4CCOCC4)c3)c2s1)OCc1ncco1] | oxazol-2-ylmethyl (7-bromobenzo[d] thiazol-2-yl)carbamate | 440.2 |
| 246 | oxazol-2-ylmethyl (7-(4- methoxyphenyl)thiazolo [4,5-b]pyridin-2- yl)carbamate | 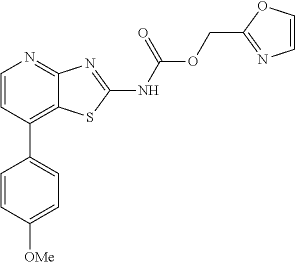 [SMILES: COc1ccc(-c2ccnc3nc(NC(=O)OCc4ncco4)sc23)cc1] | oxazol-2-ylmethyl (7-chlorothiazolo [4,5-b]pyridin-2- yl)carbamate | 383.1 |
| 247 | oxazol-2-ylmethyl (7-(4- chlorophenyl)thiazolo[4,5- b]pyridin-2-yl)carbamate | 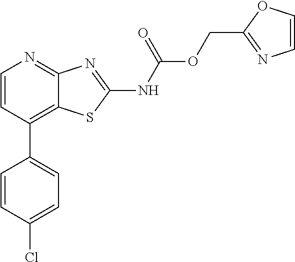 [SMILES: O=C(Nc1nc2nccc(-c3ccc(Cl)cc3)c2s1)OCc1ncco1] | oxazol-2-ylmethyl (7-chlorothiazolo [4,5-b]pyridin-2- yl)carbamate | 387.0 |
| 248 | oxazol-2-ylmethyl (7- phenylthiazolo[4,5-b] pyridin-2-yl)carbamate | 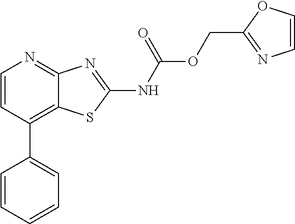 [SMILES: O=C(Nc1nc2nccc(-c3ccccc3)c2s1)OCc1ncco1] | oxazol-2-ylmethyl (7-chlorothiazolo [4,5-b]pyridin-2- yl)carbamate | 353.1 |
| 249 | oxazol-2-ylmethyl (7-(3,6- dihydro-2H-pyran-4- yl)benzo[d]thiazol-2- yl)carbamate | 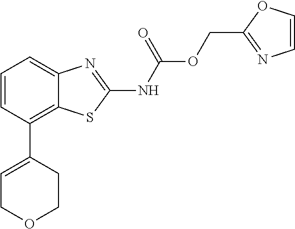 [SMILES: O=C(Nc1nc2cccc(C3=CCOCC3)c2s1)OCc1ncco1] | oxazol-2-ylmethyl (7-bromobenzo[d] thiazol-2-yl)carbamate | 358.1 |
| 250 | oxazol-2-ylmethyl (7-(1- acetyl-1,2,3,6- tetrahydropyridin-4- yl)benzo[d]thiazol-2- yl)carbamate | 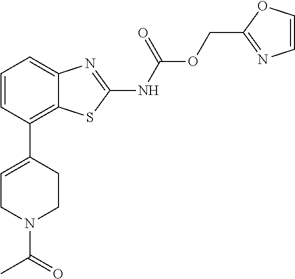 [SMILES: CC(=O)N1CC=C(c2cccc3nc(NC(=O)OCc4ncco4)sc23)CC1] | oxazol-2-ylmethyl (7-bromobenzo[d] thiazol-2-yl)carbamate | 399.1 |
| 251 | oxazol-2-ylmethyl (7-(1- methyl-1,2,3,6- tetrahydropyridin-4- yl)benzo[d]thiazol-2- yl)carbamate | 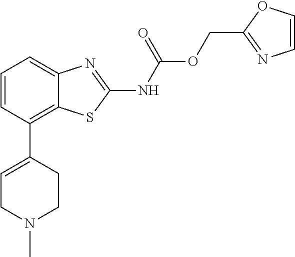 [SMILES: CN1CC=C(c2cccc3nc(NC(=O)OCc4ncco4)sc23)CC1] | oxazol-2-ylmethyl (7-bromobenzo[d] thiazol-2-yl)carbamate | 371.0 |
| 252 | oxazol-2-ylmethyl (7- ((1s,4s)-4- methoxycyclohexyl) benzo[d]thiazol-2- yl)carbamate | 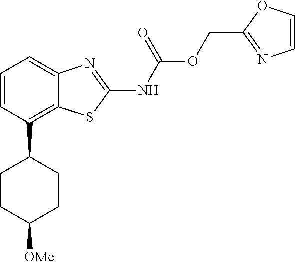 [SMILES: CO[C@H]1CC[C@@H](c2cccc3nc(NC(=O)OCc4ncco4)sc32)CC1] | 2-(4- methoxycyclohex- 1-en-1-yl)-4,4,5,5- tetramethyl-1,3,2- dioxaborolane | 388.0 |
| | | (Extra hydrogenation step | | |
| | | required: H₂, 15 PSI, THF/MeOH) | | |
| 253 | oxazol-2-ylmethyl (7- ((1r,4r)-4- methoxycyclohexyl) benzo[d]thiazol-2- yl)carbamate | 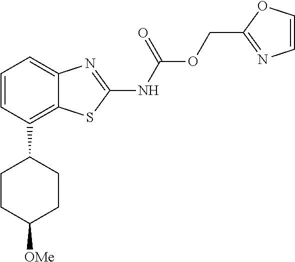 [SMILES: CO[C@H]1CC[C@H](c2cccc3nc(NC(=O)OCc4ncco4)sc32)CC1] | 2-(4- methoxycyclohex- 1-en-1-yl)-4,4,5,5- tetramethyl-1,3,2- dioxaborolane | 388.1 |
| | | (Extra hydrogenation step | | |
| | | required: H₂, 15 PSI, THF/MeOH) | | |
| 254 | oxazol-2-ylmethyl (7-(1- acetylpiperidin-4- yl)benzo[d]thiazol-2- yl)carbamate | 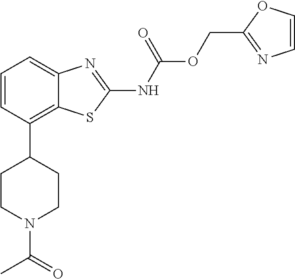 [SMILES: CC(=O)N1CCC(c2cccc3nc(NC(=O)OCc4ncco4)sc23)CC1] | tert-butyl 4- (4,4,5,5- tetramethyl-1,3,2- dioxaborolan-2- yl)piperidine-1- carboxylate | 401.1 |
| | | (Extra deBoc (HCI, EtOAc) and | | |
| | | acylation(acetic anhydride) steps | | |
| | | required | | |
| 255 | oxazol-2-ylmethyl (7-(1- carbamoylpiperidin-4- yl)benzo[d]thiazol-2- yl)carbamate | 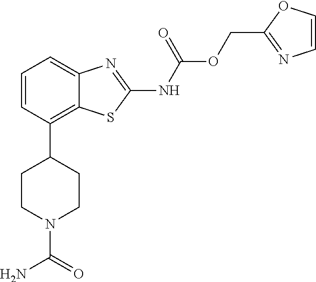 [SMILES: NC(=O)N1CCC(c2cccc3nc(NC(=O)OCc4ncco4)sc23)CC1] | tert-butyl 4- (4,4,5,5- tetramethyl-1,3,2- dioxaborolan-2- yl)piperidine-1- carboxylate | 402.1 |
| | | (Extra deBoc (HCI, EtOAc) and | | |
| | | urea formation | | |
| | | ((trimethylsilyl)isocyanate steps | | |
| | | required) | | |
| 256 | 3-fluoro-N-(6-fluoro-7-(6- methoxypyridazin-3- yl)benzo[d]thiazol-2-yl)-3- (methoxymethyl)azetidine- 1-carboxamide | 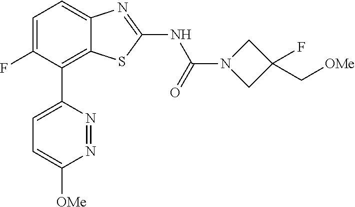 [SMILES: COCC1(F)CN(C(=O)Nc2nc3ccc(F)c(-c4ccc(OC)nn4)c3s2)C1] | N-(7-bromo-6- fluorobenzo[d] thiazol-2-yl)- 3-fluoro-3- (methoxymethyl) azetidine-1- carboxamide | 422.1 |
| | | (Stille coupling using 2-methoxy-5 | | |
| | | 3-methoxy-6- | | |
| | | (tributylstannyl)pyridazine: | | |
| | | Pd₂(dba)₃, PCy₃, LiCl, dioxane, | | |
| | | 90° C.) | | |
| 346 | 3-fluoro-N-(4-fluoro-7-(2- methoxypyrimidin-5- yl)benzo[d]thiazol-2- yl)-3-((methoxy- d3)methyl)azetidine-1- carboxamide | 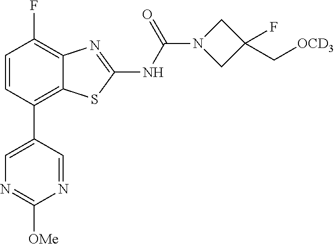 | N-(7-bromo-4- fluorobenzo[d] thiazol-2-yl)- 3-fluoro- 3-((methoxy- d3)methyl)azetidine- 1-carboxamide | 425.1 |
| 347 | N-(4,6-difluoro-7-(2- methoxypyrimidin-5- yl)benzo[d]thiazol-2-yl)- 3-fluoro-3-((methoxy- d3)methyl)azetidine-1- carboxamide | 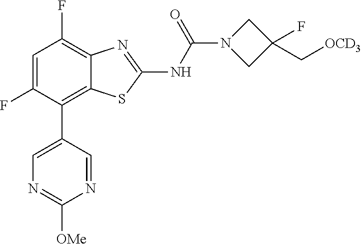 | N-(7-bromo-4,6- difluorobenzo[d] thiazol-2-yl)-3- fluoro-3-((methoxy- d3)methyl)azetidine- 1-carboxamide | 443.2 |
| 348 | N-(4,6-difluoro-7-(6- ((tetrahydro-2H-pyran-4- yl)amino)pyridin-3- yl)benzo[d]thiazol-2-yl)-3- fluoro-3-((methoxy- d3)methyl)azetidine-1- carboxamide | 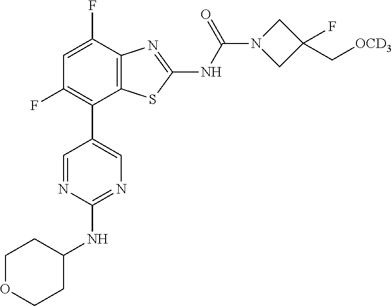 | N-(7-bromo-4,6- difluorobenzo[d] thiazol-2-yl)-3- fluoro-3-((methoxy- d3)methyl)azetidine- 1-carboxamide N-(tetrahydro-2H- pyran-4-yl)-5- (4,4,5,5- tetramethyl-1,3,2- dioxaborolan-2- yl)pyridin-2-amine | 511.2 |
| 349 | N-(6-chloro-7-(2- isopropoxypyrimidin-5- yl)benzo[d]thiazol-2-yl)-3- methoxy-3-methylazetidine- 1-carboxamide | 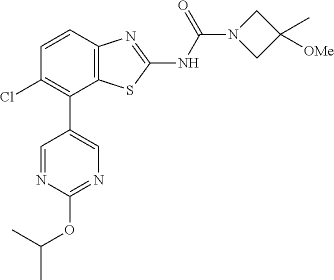 [SMILES: COC1(C)CN(C(=O)Nc2nc3ccc(Cl)c(-c4cnc(OC(C)C)nc4)c3s2)C1] | N-(7-bromo-6- chlorobenzo[d] thiazol-2-yl)-3- methoxy-3- methylazetidine-1- carboxamide | 448.2 |

### Synthetic Procedure E

### Example 257: Oxazol-2-ylmethyl (7-(6-methoxy-2,4-dimethylpyridin-3-yl)benzo[d]thiazol-2-yl)carbamate

### Step 1

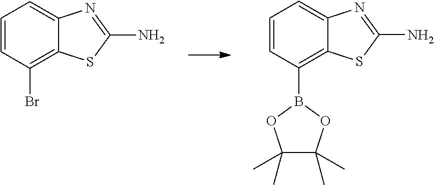 [SMILES: Nc1nc2cccc(Br)c2s1 ; CC1(C)OB(c2cccc3nc(N)sc23)OC1(C)C]

Pd(dppf)Cl₂ (32 mg, 0.04 mmol) was added to a solution of 7-bromobenzo[d]thiazol-2-amine (100 mg, 0.44 mmol), bis(pinacolato)diboron (166 mg, 0.65 mmol) and potassium acetate (129 mg, 1.31 mmol) in dioxane (2 mL). The resulting mixture was stirred at 100° C. for 16 h, filtered, concentrated in vacuo and purified by flash column chromatography on silica (EtOAc:PE, 0:100 to 50:50) to afford 7-(4,4,5,5-tetramethyl-1,3,2-dioxaborolan-2-yl)benzo[d]thiazol-2-amine (120 mg, 0.43, 100%) as a yellow gum; ES-MS [M+H]⁺: 277.1.

### Step 2

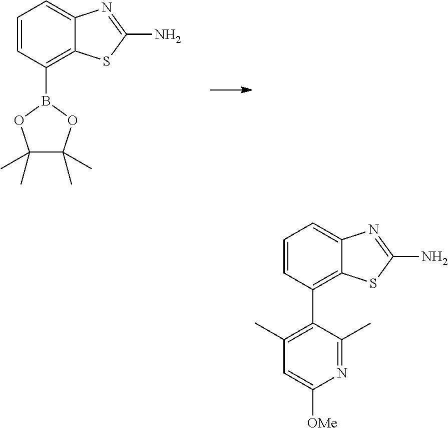 [SMILES: CC1(C)OB(c2cccc3nc(N)sc23)OC1(C)C ; COc1cc(C)c(-c2cccc3nc(N)sc23)c(C)n1]

Pd(dppf)Cl₂ (10 mg, 0.014 mmol) was added to a solution of 3-bromo-6-methoxy-2,4-dimethylpyridine (30 mg, 0.14 mmol), 7-(4,4,5,5-tetramethyl-1,3,2-dioxaborolan-2-yl)benzo[d]thiazol-2-amine (50 mg, 0.18 mmol) and potassium carbonate (58 mg, 0.42 mmol) in dioxane (1 mL) and water (0.1 mL). The resulting mixture was stirred at 80° C. for 16 h. Water (10 mL) was added at room temperature and the reaction mixture was extracted into ethyl acetate (3×10 mL). The combined organic extracts were dried over anhydrous sodium sulfate and then concentrated in vacuo. Preparative TLC (EtOAc:PE 1:1) afforded 7-(6-methoxy-2,4-dimethylpyridin-3-yl)benzo[d]thiazol-2-amine (15 mg, 0.052 mmol, 37%) as a yellow solid; ES-MS [M+H]⁺: 286.0.

### Step 3

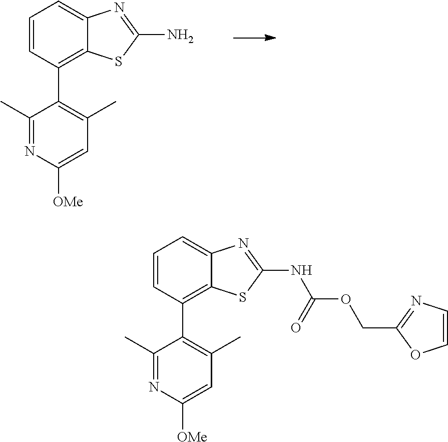 [SMILES: COc1cc(C)c(-c2cccc3nc(N)sc23)c(C)n1 ; COc1cc(C)c(-c2cccc3nc(NC(=O)OCc4ncco4)sc23)c(C)n1]

CDI (142 mg, 0.88 mmol) was added to a solution of oxazol-2-ylmethanol (87 mg, 0.88 mmol) in TIFF and the resulting solution was stirred at 60° C. for 2 h, then allowed to cool to 25° C. The mixture was then added to a solution of 7-(6-methoxy-2,4-dimethylpyridin-3-yl)benzo[d]thiazol-2-amine (50 mg, 0.18 mmol), triethylamine (122 μL, 0.88 μmol) and DMAP (2 mg, 0.18 mmol) in Tre (1 mL). The reaction mixture was stirred at 60° C. for 12 h, then concentrated in vacuo. Purification by preparative HPLC (column: Phenomenex Luna C18 150*25 mm 10 am; acetonitrile:water(formic acid); gradient: 35%-65% B over 10 min) afforded oxazol-2-ylmethyl (7-(6-methoxy-2,4-dimethylpyridin-3-yl)benzo[d]thiazol-2-yl)carbamate (6 mg, 0.15 mmol, 9) as a white solid; δ_{H} (400 MHz; DMSO-d6) 12.60-12.24 (1H, m), 8.19-8.14 (1H, m), 7.76-7.69 (1H, m), 7.57-7.48 (1H, m), 7.28-7.24 (1H, m), 7.14-7.08 (1H, m), 6.70-6.64 (1H, m), 5.31 (2H, s), 3.88 (3H, s), 2.07 (3H, s), 1.91 (3H, s); ES-MS [M+H]⁺: 411.0.

Other examples prepared using synthetic procedure E are shown in the following table. All intermediates are commercially available unless noted (see ‘Intermediate synthesis’ section).

| | | | Intermediates | |
|---|---|---|---|---|
| Ex. | | | requiring | ES-MS |
| No. | Name | Structure | synthesis | [M + H]⁺ |
| 258 | oxazol-2-ylmethyl (7-(5- (2-methoxyethoxy)-3- methylpyrazin-2- yl)benzo[d]thiazol-2- yl)carbamate | 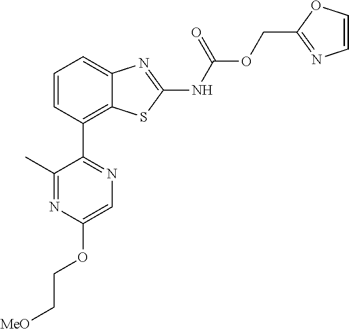 [SMILES: COCCOc1cnc(-c2cccc3nc(NC(=O)OCc4ncco4)sc23)c(C)n1] | 2-bromo-5-(2- methoxyethoxy)- 3- methylpyrazine | 442.2 |
| 259 | oxazol-2-ylmethyl (R)-(7- (6-((tetrahydrofuran-3- yl)oxy)pyridin-3- yl)benzo[d]thiazol-2- yl)carbamate | 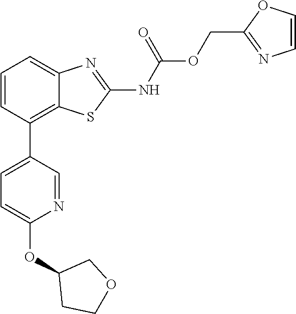 [SMILES: O=C(Nc1nc2cccc(-c3ccc(O[C@@H]4CCOC4)nc3)c2s1)OCc1ncco1] | n/a | 439.2 |
| 260 | oxazol-2-ylmethyl (S)-(7- (6-((tetrahydrofuran-3- yl)oxy)pyridin-3- yl)benzo[d]thiazol-2- yl)carbamate | 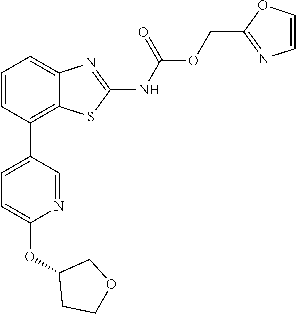 [SMILES: O=C(Nc1nc2cccc(-c3ccc(O[C@H]4CCOC4)nc3)c2s1)OCc1ncco1] | n/a | 439.1 |
| 261 | oxazol-2-ylmethyl (7-(5- methoxypyridin-2- yl)benzo[d]thiazol-2- yl)carbamate | 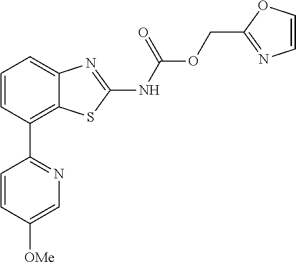 [SMILES: COc1ccc(-c2cccc3nc(NC(=O)OCc4ncco4)sc23)nc1] | n/a | 383.1 |
| 262 | oxazol-2-ylmethyl (7-(5- methoxy-3-methylpyrazin- 2-yl)benzo[d]thiazol-2- yl)carbamate | 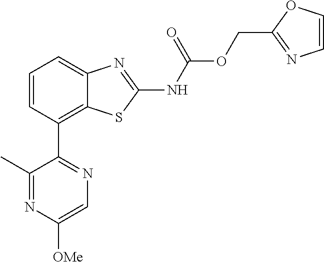 [SMILES: COc1cnc(-c2cccc3nc(NC(=O)OCc4ncco4)sc23)c(C)n1] | n/a | 398.0 |
| 263 | oxazol-2-ylmethyl (7-(3- methyl-5- (methylcarbamoyl)pyrazin- 2-yl)benzo[d]thiazol-2- yl)carbamate | 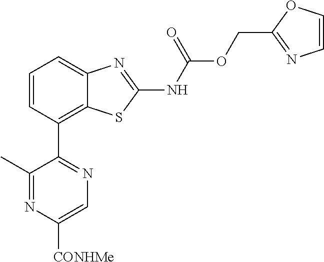 | n/a | 425.1 |
| 350 | oxazol-2-ylmethyl (7-(6- (oxetan-3-yl)pyridin-3- yl)benzo[d]thiazol-2- yl)carbamate | 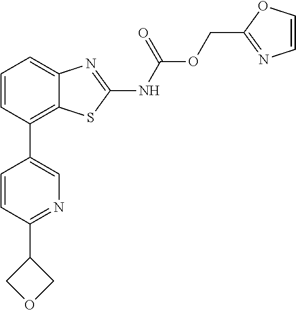 [SMILES: O=C(Nc1nc2cccc(-c3ccc(C4COC4)nc3)c2s1)OCc1ncco1] | n/a | 409.0 |

### Synthetic Procedure F

### Example 264: Oxazol-2-ylmethyl (7-(6-oxa-2-azaspiro[3.4]octan-2-yl)benzo[d]thiazol-2-yl)carbamate

### Step 1

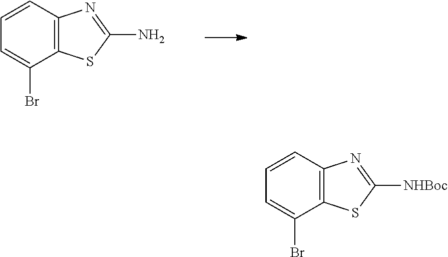 [SMILES: Nc1nc2cccc(Br)c2s1 ; CC(C)(C)OC(=O)Nc1nc2cccc(Br)c2s1]

Boc anhydride (1.26 mL, 5.48 mmol) and DMAP (60 mg, 0.49 mmol) were added to a mixture of 7-bromo-1, 3-benzothiazol-2-amine (900 mg, 3.93 mmol) in dichloromethane (10 mL) at 0° C. The mixture was stirred at 25° C. for 12 h, then water (50 mL) was added. The reaction mixture was extracted into ethyl acetate (3×50 mL) and the combined organic extracts were dried over anhydrous sodium sulfate and then concentrated in vacuo. Flash column chromatography on silica (EtOAc:PE 20:80) afforded tert-butyl (7-bromobenzo[d]thiazol-2-yl)carbamate (650 mg, 1.97 mmol, 72%) as a white solid: ES-MS [M+H]⁺: 329.0.

### Step 2

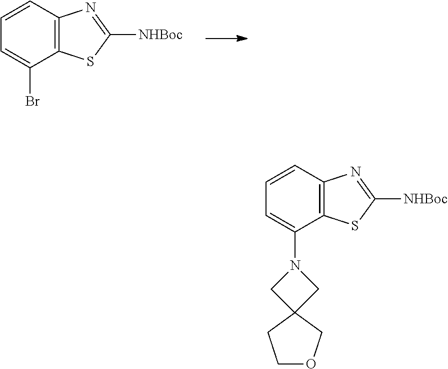 [SMILES: CC(C)(C)OC(=O)Nc1nc2cccc(Br)c2s1 ; CC(C)(C)OC(=O)Nc1nc2cccc(N3CC4(CCOC4)C3)c2s1]

6-Oxa-2-azaspiro[3.4]octane oxalate (216 mg, 683 μmol) and sodium tert-butoxide (131 mg, 1.37 mmol) were added to a solution of tert-butyl (7-bromobenzo[d]thiazol-2-yl)carbamate (150 mg, 0.46 mmol) in 2-methyl-2-butanol (3 mL). tBuXPhos Pd G3 (36 mg, 0.046 mmol) was then added and the resulting mixture was stirred at 100° C. for 16 h under nitrogen. Water (10 mL) was added and the mixture was extracted into ethyl acetate (3×10 mL). The combined extracts were washed with brine (10 mL), dried over anhydrous sodium sulfate and then concentrated in vacuo. Trituration with methanol afforded tert-butyl (7-(6-oxa-2-azaspiro[3.4]octan-2-yl)benzo[d]thiazol-2-yl)carbamate (140 mg, 0.39 mmol, 85%) as an off-white solid; ES-MS [M+H]⁺: 362.2.

### Step 3

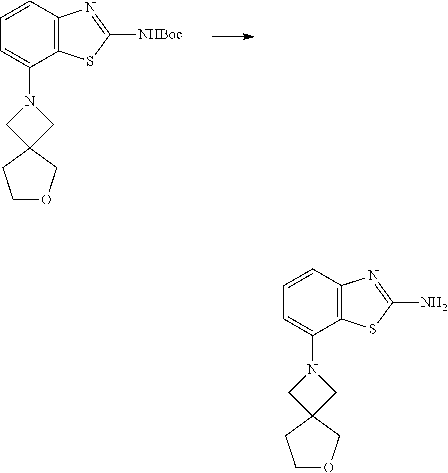 [SMILES: CC(C)(C)OC(=O)Nc1nc2cccc(N3CC4(CCOC4)C3)c2s1 ; Nc1nc2cccc(N3CC4(CCOC4)C3)c2s1]

TFA (0.3 mL, 4.04 mmol) was added to a solution of tert-butyl (7-(6-oxa-2-azaspiro[3.4]octan-2-yl)benzo[d]thiazol-2-yl)carbamate (140 mg, 0.39 mmol) in dichloromethane (1.5 mL) and the resulting mixture was stirred for 25° C. for 2 h. The reaction was adjusted to pH 7 with saturated sodium bicarbonate solution and extracted into EtOAc (3×10 mL). The combined organic extracts were washed with brine (10 mL), dried over anhydrous sodium sulfate and then concentrated in vacuo. Trituration with methanol afforded 7-(6-oxa-2-azaspiro[3.4]octan-2-yl)benzo[d]thiazol-2-amine (60 mg, 0.23 mmol, 59%) as a pink solid; ES-MS [M+H]⁺: 262.2.

### Step 4

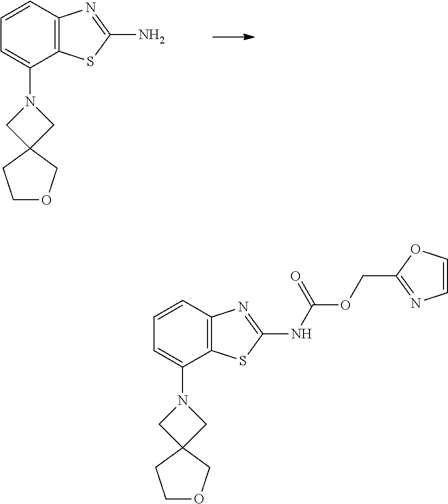 [SMILES: Nc1nc2cccc(N3CC4(CCOC4)C3)c2s1 ; O=C(Nc1nc2cccc(N3CC4(CCOC4)C3)c2s1)OCc1ncco1]

Oxazol-2-ylmethyl (7-(6-oxa-2-azaspiro[3.4]octan-2-yl)benzo[d]thiazol-2-yl)carbamate (41 mg, 0.098 mmol, 43%, white solid) was prepared using Step 3 from Synthetic procedure E; δ_{H} (400 MHz; DMSO-d6) 12.28 (1H, br d, J 1.2 Hz), 8.19 (1H, s), 7.29 (1H, s), 7.27-7.21 (1H, m), 7.11 (1H, d, J 8.0 Hz), 6.32 (1H, d, J 8.0 Hz), 5.36 (2H, s), 4.05-3.98 (4H, m), 3.86-3.81 (2H, m), 3.75 (2H, m), 2.21-2.14 (2H, m); ES-MS [M+H]⁺: 387.1.

Other examples prepared using synthetic procedure F are shown in the following table. All intermediates are commercially available.

| | | | ES-MS |
|---|---|---|---|
| Ex. No. | Name | Structure | [M + H]⁺ |
| 265 | oxazol-2-ylmethyl (7-(4- methoxypiperidin-1- yl)benzo[d]thiazol-2-yl)carbamate | 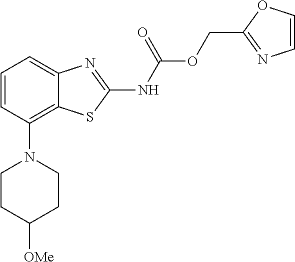 [SMILES: COC1CCN(c2cccc3nc(NC(=O)OCc4ncco4)sc23)CC1] | 389.2 |
| 266 | oxazol-2-ylmethyl (7-(3- (methoxymethyl)azetidin-1- yl)benzo[d]thiazol-2-yl)carbamate | 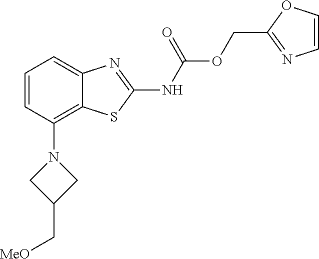 [SMILES: COCC1CN(c2cccc3nc(NC(=O)OCc4ncco4)sc23)C1] | 375.2 |
| 267 | oxazol-2-ylmethyl (7-(3- methoxyazetidin-1- yl)benzo[d]thiazol-2-yl)carbamate | 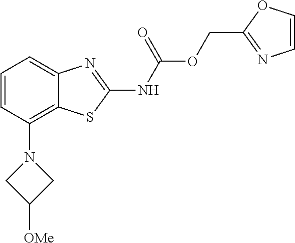 [SMILES: COC1CN(c2cccc3nc(NC(=O)OCc4ncco4)sc23)C1] | 361.1 |
| 268 | oxazol-2-ylmethyl (7-(2-oxa-6- azaspiro[3.3]heptan-6- yl)benzo[d]thiazol-2-yl)carbamate | 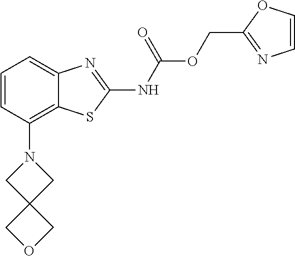 [SMILES: O=C(Nc1nc2cccc(N3CC4(COC4)C3)c2s1)OCc1ncco1] | 373.1 |
| 269 | oxazol-2-ylmethyl (7-(3- ((trifluoromethoxy)methyl)azetidin- 1-yl)benzo[d]thiazol-2- yl)carbamate | 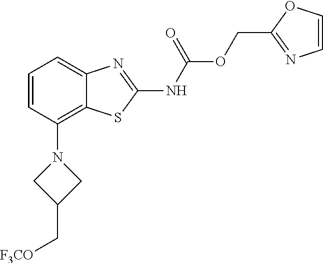 [SMILES: O=C(Nc1nc2cccc(N3CC(COC(F)(F)F)C3)c2s1)OCc1ncco1] | 429.1 |
| 270 | oxazol-2-ylmethyl (7-(3-(2,2,2- trifluoroethyl)azetidin-1- yl)benzo[d]thiazol-2-yl)carbamate | 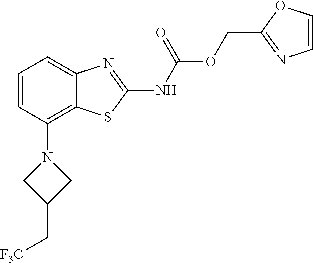 [SMILES: O=C(Nc1nc2cccc(N3CC(CC(F)(F)F)C3)c2s1)OCc1ncco1] | 413.1 |
| 271 | oxazol-2-ylmethyl (7-(1-oxa-6- azaspiro[3.3]heptan-6- yl)benzo[d]thiazol-2-yl)carbamate | 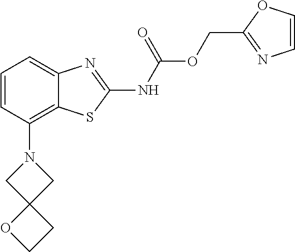 [SMILES: O=C(Nc1nc2cccc(N3CC4(CCO4)C3)c2s1)OCc1ncco1] | 373.1 |
| 272 | oxazol-2-ylmethyl (7-(3-(2- hydroxypropan-2-yl)azetidin-1- yl)benzo[d]thiazol-2-yl)carbamate | 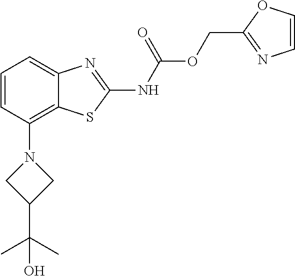 [SMILES: CC(C)(O)C1CN(c2cccc3nc(NC(=O)OCc4ncco4)sc23)C1] | 389.0 |
| 273 | oxazol-2-ylmethyl (7-(6-oxa-2- azaspiro[3.5]nonan-2- yl)benzo[d]thiazol-2-yl)carbamate | 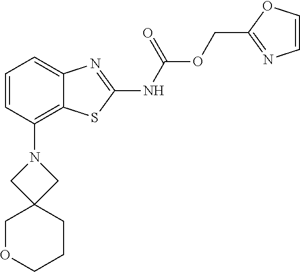 [SMILES: O=C(Nc1nc2cccc(N3CC4(CCCOC4)C3)c2s1)OCc1ncco1] | 401.1 |
| 274 | oxaozl-2-ylmethyl (7-(3- cyclopropyl-3-hydroxyazetidin-1- yl)benzo[d]thiaozl-2-yl)carbamate | 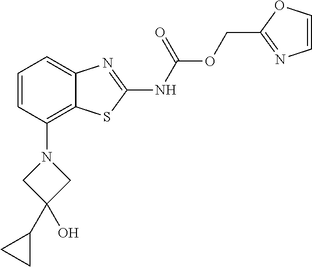 [SMILES: O=C(Nc1nc2cccc(N3CC(O)(C4CC4)C3)c2s1)OCc1ncco1] | 387.1 |
| 275 | oxazol-2-ylmethyl (7-(3-hydroxy- 3-(trifluoromethyl)azetidin-1- yl)benzo[d]thiazol-2-yl)carbamate | 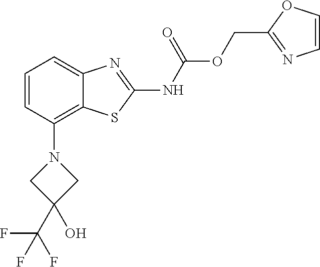 [SMILES: O=C(Nc1nc2cccc(N3CC(O)(C(F)(F)F)C3)c2s1)OCc1ncco1] | 415.1 |
| 276 | oxazol-2-ylmethyl (7-(5-oxa-2- azaspiro[3.5]nonan-2- yl)benzo[d]thiazol-2-yl)carbamate | 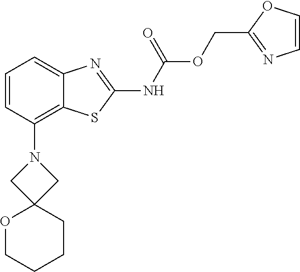 [SMILES: O=C(Nc1nc2cccc(N3CC4(CCCCO4)C3)c2s1)OCc1ncco1] | 401.1 |
| 277 | oxazol-2-ylmethyl (7-(3- (difluoromethoxy)azetidin-1- yl)benzo[d]thiazol-2-yl)carbamate | 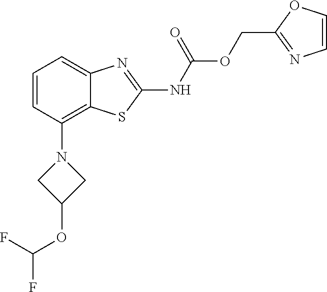 [SMILES: O=C(Nc1nc2cccc(N3CC(OC(F)F)C3)c2s1)OCc1ncco1] | 397.0 |
| 278 | oxazol-2-ylmethyl (7-(3- isopropoxyazetidin-1- yl)benzo[d]thiazol-2-yl)carbamate | 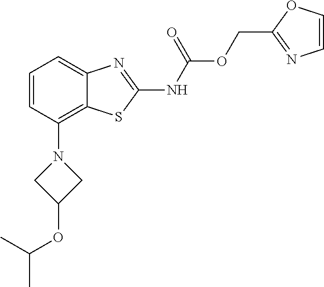 [SMILES: CC(C)OC1CN(c2cccc3nc(NC(=O)OCc4ncco4)sc23)C1] | 389.0 |
| 279 | oxaozl-2-ylmethyl (7-(3-hydroxy- 3-methylazetidin-1- yl)benzo[d]thiazol-2-yl)carbamate | 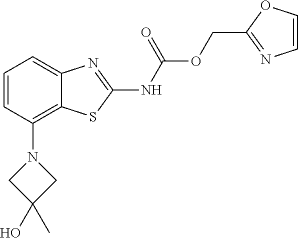 [SMILES: CC1(O)CN(c2cccc3nc(NC(=O)OCc4ncco4)sc23)C1] | 361.0 |
| 280 | oxazol-2-ylmethyl (7-(3-(2- oxopyrrolidin-1-yl)azetidin-1- yl)benzo[d]thiazol-2-yl)carbamate | 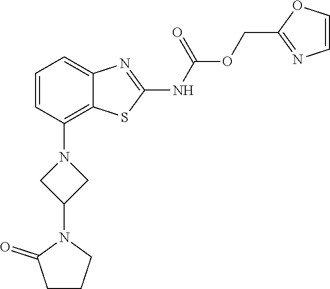 [SMILES: O=C(Nc1nc2cccc(N3CC(N4CCCC4=O)C3)c2s1)OCc1ncco1] | 414.1 |
| 281 | oxazol-2-ylmethyl (7-(6-hydroxy- 6-(trifluoromethyl)-2- azaspiro[3.3]heptan-2- yl)benzo[d]thiazol-2-yl)carbamate | 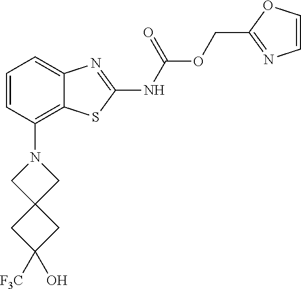 [SMILES: O=C(Nc1nc2cccc(N3CC4(C3)CC(O)(C(F)(F)F)C4)c2s1)OCc1ncco1] | 455.1 |
| 282 | oxazol-2-ylmethyl (7-(1,1-difluoro- 5-azaspiro[2.3]hexan-5- yl)benzo[d]thiazol-2-yl)carbamate | 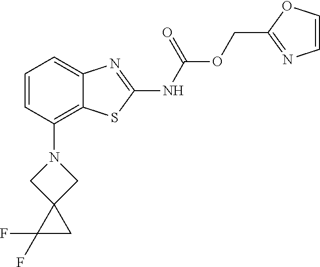 [SMILES: O=C(Nc1nc2cccc(N3CC4(C3)CC4(F)F)c2s1)OCc1ncco1] | 393.1 |
| 283 | oxazol-2-ylmethyl (7-(3- (fluoromethyl)azetidin-1- yl)benzo[d]thiazol-2-yl)carbamate | 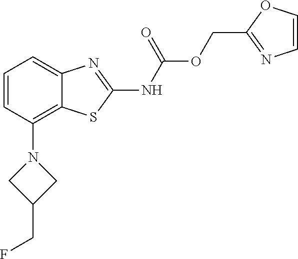 [SMILES: O=C(Nc1nc2cccc(N3CC(CF)C3)c2s1)OCc1ncco1] | 363.1 |

### Synthetic Procedure G

### Example 284: 3-Fluoro-3-(methoxymethyl)-N-(7-(5-methoxypyridin-2-yl)benzo[d]thiazol-2-yl)azetidine-1-carboxamide

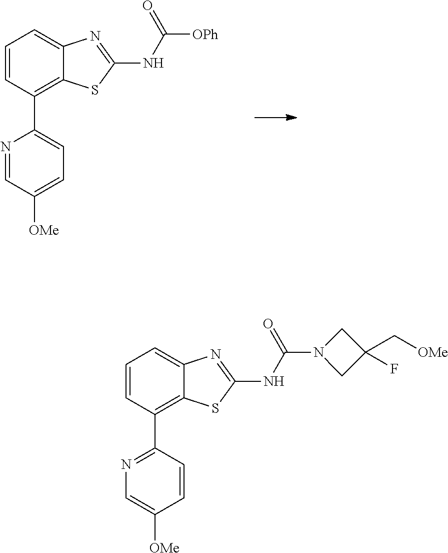 [SMILES: COc1ccc(-c2cccc3nc(NC(=O)Oc4ccccc4)sc23)nc1 ; COCC1(F)CN(C(=O)Nc2nc3cccc(-c4ccc(OC)cn4)c3s2)C1]

3-Fluoro-3-(methoxymethyl)azetidine (30 mg, 252 μmol) and triethylamine (55 μL, 395 μmol) were added to a solution of phenyl (7-(5-methoxypyridin-2-yl)benzo[d]thiazol-2-yl)carbamate* (50 mg, 132 μmol) in dichloromethane (1 mL) and the resulting mixture was stirred at 25° C. for 1 h. Water (20 mL) was added and the mixture was extracted into EtOAc (3×20 mL). The combined organic extracts were dried over anhydrous sodium sulfate and then concentrated in vacuo. Purification by preparative HPLC (column: Welch Xtimate C18 150*25 mm 5 am; acetonitrile:water(formic acid); gradient: 34%-64% B over 10 min)) afforded 3-fluoro-3-(methoxymethyl)-N-(7-(5-methoxypyridin-2-yl)benzo[d]thiazol-2-yl)azetidine-1-carboxamide (8 mg, 19.1 μmol) as a yellow solid; δ_{H} (400 MHz; CD₃OD) 8.45 (1H, d, J 2.8 Hz), 8.07-8.04 (1H, m), 7.81 (1H, d, J 7.1 Hz), 7.64 (1H, d, J 7.7 Hz), 7.53-7.46 (2H, m), 4.36-4.12 (4H, m), 3.95 (3H, s), 3.73 (2H, d, J 19.7 Hz), 3.46 (3H, s); δ_{F} (400 MHz; CD₃OD) −159.0; ES-MS [M+H]⁺: 403.1.
*prepared using Synthetic procedure E, steps 1 and 2, then Synthetic procedure A, step 2

Other examples prepared using synthetic procedure G are shown in the following table. All intermediates are commercially available unless noted (see ‘Intermediate synthesis’ section).

| Ex. | | | Intermediates | ES-MS |
|---|---|---|---|---|
| No. | Name | Structure | requiring synthesis | [M + H]⁺ |
| 285 | N-(7-(5- methoxypyrazin-2- yl)benzo[d]thiazol-2- yl)-2-oxa-6- azaspiro[3.3]heptane-6- carboxamide | 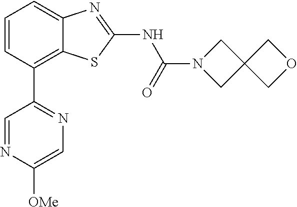 [SMILES: COc1cnc(-c2cccc3nc(NC(=O)N4CC5(COC5)C4)sc23)cn1] | n/a | 384.1 |
| 286 | N-(7-(3-chloro-5- methoxypyrazin-2- yl)benzo[d]thiazol-2- yl)-2-oxa-6- azaspiro[3.3]heptane-6- carboxamide | 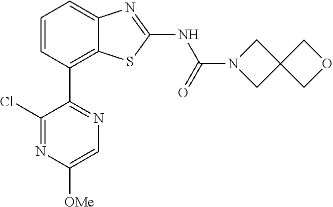 [SMILES: COc1cnc(-c2cccc3nc(NC(=O)N4CC5(COC5)C4)sc23)c(Cl)n1] | 2-bromo-3-chloro-5- methoxypyrazine | 418.1 |
| 287 | N-(7-(1-methyl-1H- 1,2,3-triazol-5- yl)benzo[d]thiazol-2- yl)-2-oxa-6- azaspiro[3.3]heptane-6- carboxamide | 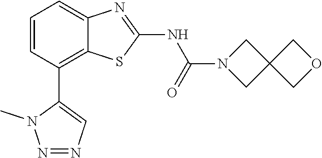 [SMILES: Cn1nncc1-c1cccc2nc(NC(=O)N3CC4(COC4)C3)sc12] | n/a | 357.1 |
| 288 | 3-(methoxy-d3)-3- methyl-N-(7-(6-(oxetan- 3-yloxy)pyridin-3- yl)benzo[d]thiazol-2- yl)azetidine-1- carboxamide | 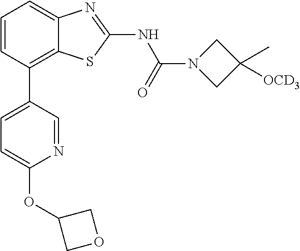 | 3-(methoxy-d3)-3- methylazetidine | 430.2 |
| 289 | 3-fluoro-3- (methoxymethyl)-N-(7- (5-((tetrahydro-2H- pyran-4- yl)amino)pyridin-2- yl)benzo[d]thiazol-2- yl)azetidine-1- carboxamide | 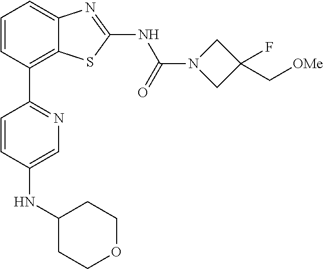 [SMILES: COCC1(F)CN(C(=O)Nc2nc3cccc(-c4ccc(NC5CCOCC5)cn4)c3s2)C1] | 6-bromo-N- (tetrahydro-2H-pyran- 4-yl)pyridin-3-amine | 472.2 |
| 290 | 3-fluoro-3-((methoxy- d3)methyl)-N-(7-(2- (oxetan-3- yloxy)pyrimidin-5- yl)benzo[d]thiazol-2- yl)azetidine-1- carboxamide | 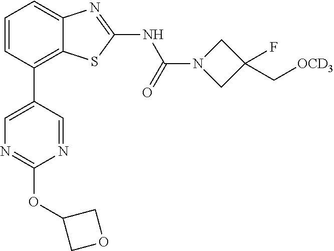 | 3-fluoro-3-((methoxy- d3)methyl)azetidine | 449.2 |
| 291 | 3-(methoxy-d3)-3- methyl-N-(7-(2-(oxetan- 3-yloxy)pyrimidin-5- yl)benzo[d]thiazol-2- yl)azetidine-1- carboxamide | 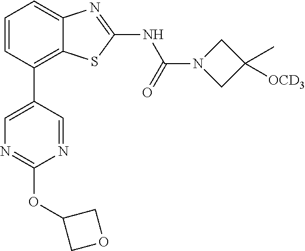 | 3-(methoxy-d3)-3- methylazetidine | 431.2 |
| 292 | N-(7-(5-methoxy-3- methylpyrazin-2- yl)benzo[d]thiazol-2- yl)-8-methyl-5-oxa-2,8- diazaspiro[3.5]nonane- 2-carboxamide | 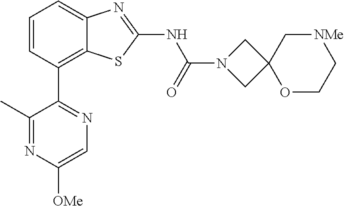 [SMILES: COc1cnc(-c2cccc3nc(NC(=O)N4CC5(CN(C)CCO5)C4)sc23)c(C)n1] | n/a | 441.2 |
| 293 | N-(7-(5-(1,1- difluoroethyl)-3- methylpyrazin-2- yl)benzo[d]thiazol-2- yl)-3-fluoro-3- (methoxymethyl)azetidine- 1-carboxamide | 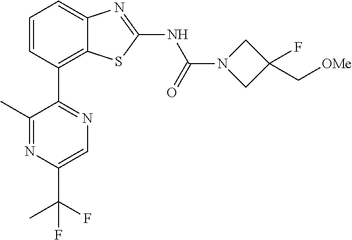 [SMILES: COCC1(F)CN(C(=O)Nc2nc3cccc(-c4ncc(C(C)(F)F)nc4C)c3s2)C1] | 2-chloro-5-(1,1- difluoroethyl)-3- methylpyrazine | 452.1 |
| 294 | N-(7-(5-methoxy-3- methylpyrazin-2- yl)benzo[d]thiazol-2- yl)-2-oxa-6- azaspiro[3.3]heptane-6- carboxamide | 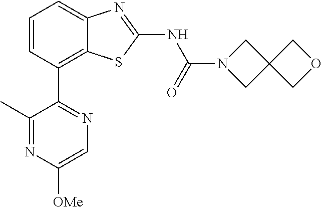 [SMILES: COc1cnc(-c2cccc3nc(NC(=O)N4CC5(COC5)C4)sc23)c(C)n1] | n/a | 398.1 |
| 295 | 3-fluoro-N-(7-(5- methoxy-3- methylpyrazin-2- yl)benzo[d]thiazol-2- yl)-3- (methoxymethyl)azetidine- 1-carboxamide | 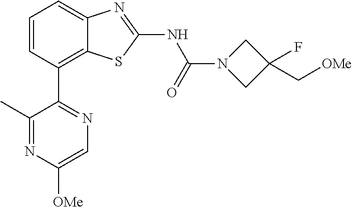 [SMILES: COCC1(F)CN(C(=O)Nc2nc3cccc(-c4ncc(OC)nc4C)c3s2)C1] | n/a | 418.0 |
| 296 | 3-fluoro-3- (methoxymethyl)-N-(7- (5-methyl-3,6-dihydro- 2H-pyran-4- yl)benzo[d]thiazol-2- yl)azetidine-1- carboxamide | 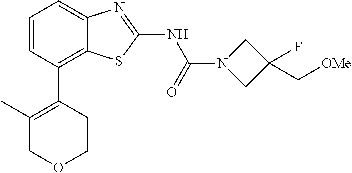 [SMILES: COCC1(F)CN(C(=O)Nc2nc3cccc(C4=C(C)COCC4)c3s2)C1] | 5-methyl-3,6-dihydro- 2H-pyran-4-yl trifluoromethane- sulfonate | 392.2 |
| 297 | 3-fluoro-N-(6-fluoro-7- (5-methoxy-3- methylpyrazin-2- yl)benzo[d]thiazol-2- yl)-3-((methoxy- d3)methyl)azetidine-1- carboxamide | 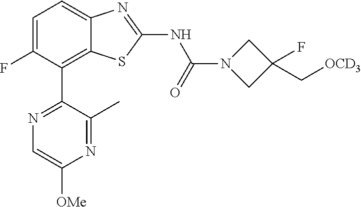 | 3-fluoro-3-((methoxy- d3)methyl)azetidine and phenyl (6-fluoro-7-(5- methoxy-3- methylpyrazin-2- yl)benzo[d]thiazol-2- yl)carbamate* | 439.2 |
| 298 | N-(6-fluoro-7-(5- methoxy-3- methylpyrazin-2- yl)benzo[d]thiazol-2- yl)-5,8-dioxa-2- azaspiro[3.5]nonane-2- carboxamide | 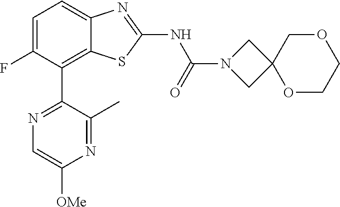 [SMILES: COc1cnc(-c2c(F)ccc3nc(NC(=O)N4CC5(COCCO5)C4)sc23)c(C)n1] | phenyl (6-fluoro-7-(5- methoxy-3- methylpyrazin-2- yl)benzo[d]thiazol-2- yl)carbamate* | 446.1 |

*Prepared using: (1) Procedure I, Step 1; (2) Procedure E, Step 1; (3) Procedure E, Step 2; (4) Procedure I, Step 3; (5) Procedure A, Step 2

### Synthetic Procedure H

### Example 299: Oxazol-2-ylmethyl (7-(2-methoxypyrimidin-5-yl)benzo[d]thiazol-2-yl)carbamate

### Step 1

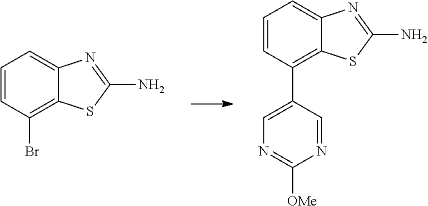 [SMILES: Nc1nc2cccc(Br)c2s1 ; COc1ncc(-c2cccc3nc(N)sc23)cn1]

Pd(dppf)Cl₂ (80 mg, 109 μmol) was added to a solution of 7-bromobenzo[d]thiazol-2-amine (250 mg, 1.09 mmol), (2-methoxypyrimidin-5-yl)boronic acid (202 mg, 1.31 mmol) and sodium carbonate (231 mg, 2.18 mmol) in dioxane (3 mL) and water (0.6 mL) at 25° C. The mixture was stirred at 80° C. for 16 h. Water (15 mL) was added and the mixture was extracted into EtOAc (3×15 mL). The combined organic extracts were washed with brine (15 mL), dried over anhydrous sodium sulfate and then concentrated in vacuo. Flash column chromatography on silica (EtOAc:PE 0:100 to 80:20) afforded 7-(2-methoxypyrimidin-5-yl)benzo[d]thiazol-2-amine (80 mg, 310 μmol, 28%); ES-MS [M+H]⁺: 259.1.

### Step 2

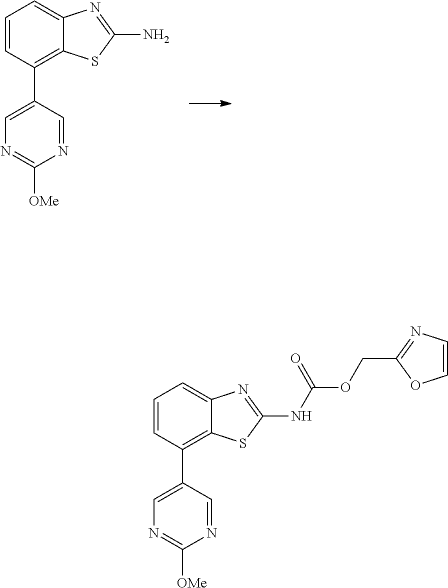 [SMILES: COc1ncc(-c2cccc3nc(N)sc23)cn1 ; COc1ncc(-c2cccc3nc(NC(=O)OCc4ncco4)sc23)cn1]

Oxazol-2-ylmethyl (7-(2-methoxypyrimidin-5-yl)benzo[d]thiazol-2-yl)carbamate was prepared from 7-(2-methoxypyrimidin-5-yl)benzo[d]thiazol-2-amine using Synthetic procedure E, Step 3; δ_{H} (400 MHz; DMSO-d6) 12.71-12.14 (1H, m), 8.94 (2H, s), 8.18 (1H, s), 7.77 (1H, m), 7.56 (1H, m), 7.43 (1H, m), 7.28 (1H, s), 5.37 (2H, s), 4.01 (3H, s, 3H); ES-MS [M+H]⁺: 384.1.

Other examples prepared using synthetic procedure H are shown in the following table. All intermediates are commercially available unless noted (see ‘Intermediate synthesis’ section).

| | | | Intermediates | |
|---|---|---|---|---|
| Ex. | | | requiring | ES-MS |
| No. | Name | Structure | synthesis | [M + H]⁺ |
| 300 | oxazol-2-ylmethyl (7-(6- (trifluoromethyl)pyridin- 3-yl)benzo[d]thiazol-2- yl)carbamate | 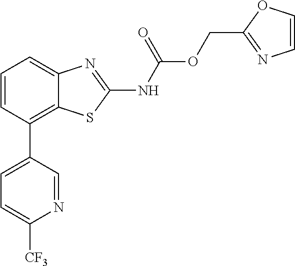 [SMILES: O=C(Nc1nc2cccc(-c3ccc(C(F)(F)F)nc3)c2s1)OCc1ncco1] | n/a | 421.0 |
| 301 | oxazol-2-ylmethyl (7- phenylbenzo[d]thiazol-2- yl)carbamate | 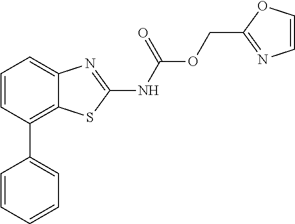 [SMILES: O=C(Nc1nc2cccc(-c3ccccc3)c2s1)OCc1ncco1] | n/a | 352.1 |
| 302 | pyridin-2-ylmethyl (7- phenylbenzo[d]thiazol-2- yl)carbamate | 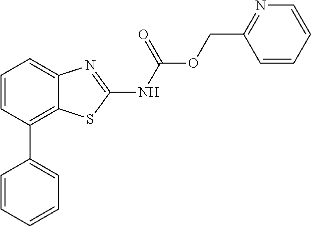 [SMILES: O=C(Nc1nc2cccc(-c3ccccc3)c2s1)OCc1ccccn1] | n/a | 362.0 |
| 303 | oxazol-2-ylmethyl (7- (pyridin-3- yl)benzo[d]thiazol-2- yl)carbamate | 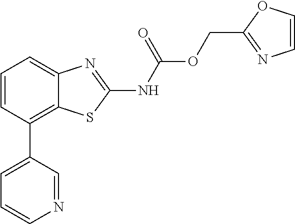 [SMILES: O=C(Nc1nc2cccc(-c3cccnc3)c2s1)OCc1ncco1] | n/a | 353.2 |
| 304 | oxazol-2-ylmethyl (7-(4- methoxyphenyl)benzo[d]thi- azol-2-yl)carbamate | 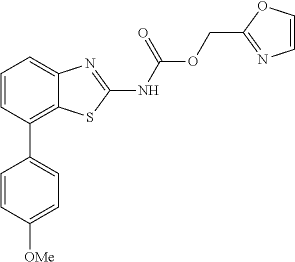 [SMILES: COc1ccc(-c2cccc3nc(NC(=O)OCc4ncco4)sc23)cc1] | n/a | 382.2 |
| 305 | oxazol-2-ylmethyl (7-(4- chlorophenyl)benzo[d]thi- azol-2-yl)carbamate | 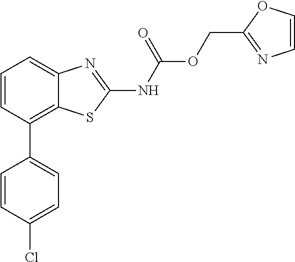 [SMILES: O=C(Nc1nc2cccc(-c3ccc(Cl)cc3)c2s1)OCc1ncco1] | n/a | 386.1 |
| 306 | oxazol-2-ylmethyl (7-(6- methoxypyridin-3- yl)benzo[d]thiazol-2- yl)carbamate | 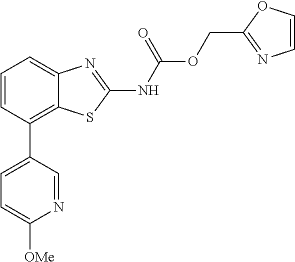 [SMILES: COc1ccc(-c2cccc3nc(NC(=O)OCc4ncco4)sc23)cn1] | n/a | 383.1 |
| 307 | oxazol-2-ylmethyl (7-(4- fluorophenyl)benzo[d]thia- zol-2-yl)carbamate | 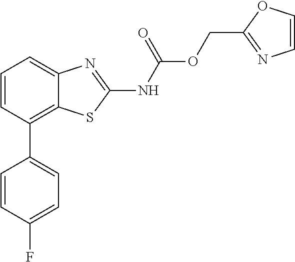 [SMILES: O=C(Nc1nc2cccc(-c3ccc(F)cc3)c2s1)OCc1ncco1] | n/a | 370.1 |
| 308 | oxazol-2-ylmethyl (7-(4- (trifluoromethyl)phenyl)ben- zo[d]thiazol-2- yl)carbamate | 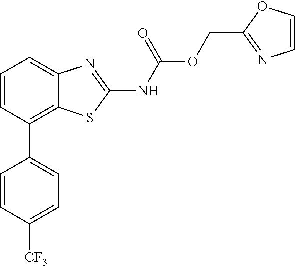 [SMILES: O=C(Nc1nc2cccc(-c3ccc(C(F)(F)F)cc3)c2s1)OCc1ncco1] | n/a | 420.1 |
| 309 | oxazol-2-ylmethyl (7-(4- (trifluoromethoxy)phenyl) benzo[d]thiazol-2- yl)carbamate | 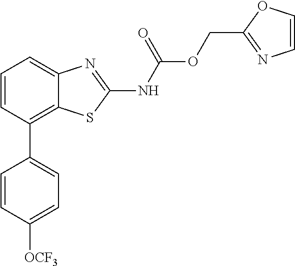 [SMILES: O=C(Nc1nc2cccc(-c3ccc(OC(F)(F)F)cc3)c2s1)OCc1ncco1] | n/a | 436.1 |
| 310 | pyridin-2-ylmethyl (7-(6- methoxypyridin-3- yl)benzo[d]thiazol-2- yl)carbamate | 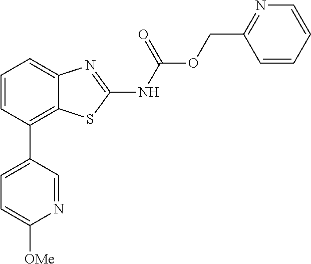 [SMILES: COc1ccc(-c2cccc3nc(NC(=O)OCc4ccccn4)sc23)cn1] | n/a | 393.1 |
| 311 | oxazol-2-ylmethyl (7-(6- methoxypyridin-3-yl)-6- (trifluoromethyl)benzo[d]thi- azol-2-yl)carbamate | 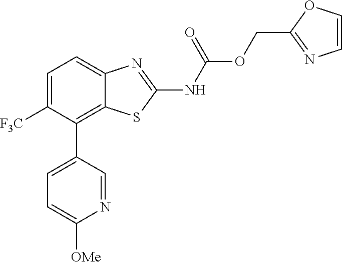 [SMILES: COc1ccc(-c2c(C(F)(F)F)ccc3nc(NC(=O)OCc4ncco4)sc23)cn1] | 7-bromo-6- (trifluoromethyl)ben- zo[d]thiazol-2- amine | 451.1 |
| 312 | oxazol-2-ylmethyl (6- methoxy-7-(6- methoxypyridin-3- yl)benzo[d]thiazol-2- yl)carbamate | 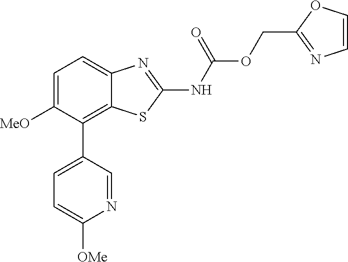 [SMILES: COc1ccc(-c2c(OC)ccc3nc(NC(=O)OCc4ncco4)sc23)cn1] | 7-bromo-6- methoxybenzo[d]thi- azol-2-amine | 413.0 |
| 313 | oxazol-2-ylmethyl (5- fluoro-7-(6- methoxypyridin-3- yl)benzo[d]thiazol-2- yl)carbamate | 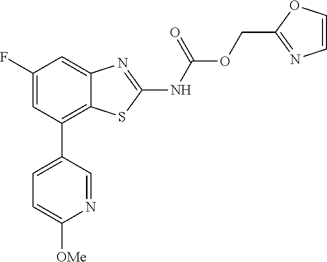 [SMILES: COc1ccc(-c2cc(F)cc3nc(NC(=O)OCc4ncco4)sc23)cn1] | 7-bromo-5- fluorobenzo[d]thia- zol-2-amine | 401.0 |
| 314 | oxazol-2-ylmethyl (4-(6- methoxypyridin-3- yl)thiazolo[5,4-c]pyridin- 2-yl)carbamate | 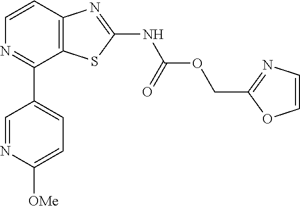 [SMILES: COc1ccc(-c2nccc3nc(NC(=O)OCc4ncco4)sc23)cn1] | 4- chlorothiazolo[5,4- c]pyridin-2-amine | 384.1 |
| 315 | oxazol-2-ylmethyl (4- phenylthiazolo[5,4- c]pyridin-2-yl)carbamate | 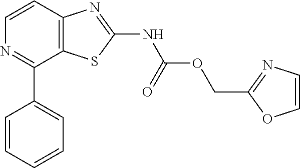 [SMILES: O=C(Nc1nc2ccnc(-c3ccccc3)c2s1)OCc1ncco1] | 4- chlorothiazolo[5,4- c]pyridin-2-amine | 353.1 |
| 316 | oxazol-2-ylmethyl (7-(6- methoxypyridin-3-yl)-4- methylbenzo[d]thiazol-2- yl)carbamate | 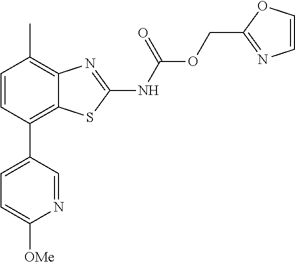 [SMILES: COc1ccc(-c2ccc(C)c3nc(NC(=O)OCc4ncco4)sc23)cn1] | n/a | 397.1 |
| 317 | oxazol-2-ylmethyl (7-(6- methoxypyridin-3-yl)-5- methylbenzo[d]thiazol-2- yl)carbamate | 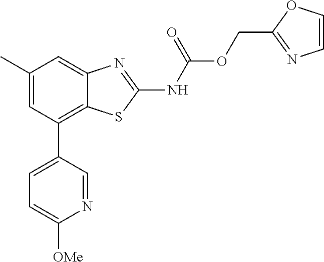 [SMILES: COc1ccc(-c2cc(C)cc3nc(NC(=O)OCc4ncco4)sc23)cn1] | 7-bromo-5- methylbenzo[d]thia- zol-2-amine | 397.1 |
| 318 | oxazol-2-ylmethyl (7-(6- methoxypyridin-3-yl)-6- methylbenzo[d]thiazol-2- yl)carbamate | 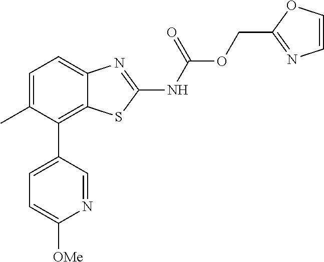 [SMILES: COc1ccc(-c2c(C)ccc3nc(NC(=O)OCc4ncco4)sc23)cn1] | n/a | 397.1 |
| 319 | oxazol-2-ylmethyl (7-(4- chlorophenyl)-4- fluorobenzo[d]thiazol-2- yl)carbamate | 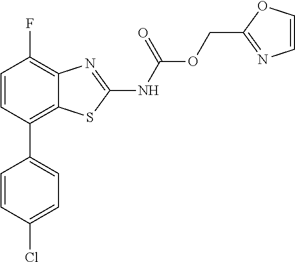 [SMILES: O=C(Nc1nc2c(F)ccc(-c3ccc(Cl)cc3)c2s1)OCc1ncco1] | n/a | 404.1 |
| 320 | oxazol-2-ylmethyl (6- chloro-7-(6- methoxypyridin-3- yl)benzo[d]thiazol-2- yl)carbamate | 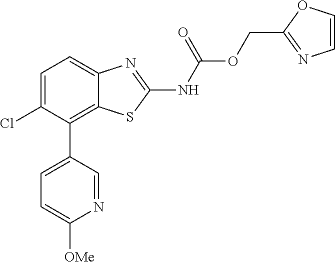 [SMILES: COc1ccc(-c2c(Cl)ccc3nc(NC(=O)OCc4ncco4)sc23)cn1] | 7-bromo-6- chlorobenzo[d]thia- zol-2-amine | 417.0 |
| 321 | oxazol-2-ylmethyl (6- cyano-7-(6- methoxypyridin-3- yl)benzo[d]thiazol-2- yl)carbamate | 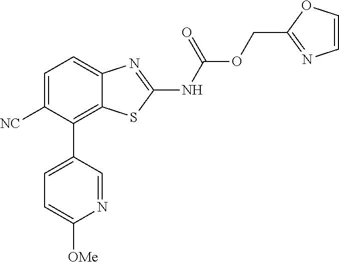 [SMILES: COc1ccc(-c2c(C#N)ccc3nc(NC(=O)OCc4ncco4)sc23)cn1] | 2-amino-7-(6- methoxypyridin-3- yl)benzo[d]thiazole- 6-carbonitrile | 408.1 |
| | | (Step 2 only) | | |
| 322 | oxazol-2-ylmethyl (6- fluoro-7-(6- methoxypyridin-3- yl)benzo[d]thiazol-2- yl)carbamate | 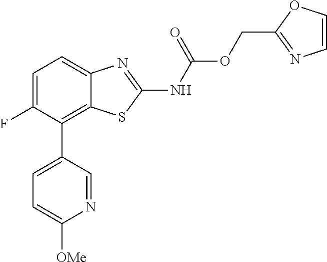 [SMILES: COc1ccc(-c2c(F)ccc3nc(NC(=O)OCc4ncco4)sc23)cn1] | 7-bromo-6- fluorobenzo[d]thia- zol-2-amine | 401.1 |
| 323 | oxazol-2-ylmethyl (7-(6- methoxypyridin-3-yl)-6- (methylsulfonyl)benzo[d]thi- azol-2-yl)carbamate | 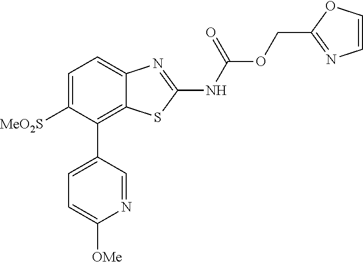 [SMILES: COc1ccc(-c2c(S(C)(=O)=O)ccc3nc(NC(=O)OCc4ncco4)sc23)cn1] | 7-(6- methoxypyridin-3- yl)-6- (methylsulfonyl)ben- zo[d]thiazol-2- amine | 461.1 |
| 324 | oxazol-2-ylmethyl (7- (pyridin-2- yl)benzo[d]thiazol-2- yl)carbamate | 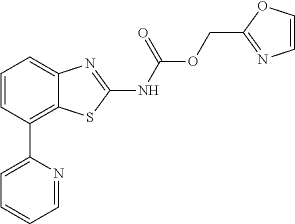 [SMILES: O=C(Nc1nc2cccc(-c3ccccn3)c2s1)OCc1ncco1] | n/a | 353.0 |
| | | (Stille coupling used in Step 1: 2- | | |
| | | (tributylstannyl)pyridine, Pd(Ph₃)₄, | | |
| | | DMF, 100° C.) | | |
| 351 | oxazol-2-ylmethyl (6- chloro-7-(5- methoxypyrazin-2- yl)benzo[d]thiazol-2- yl)carbamate | 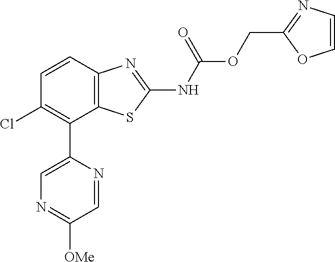 [SMILES: COc1cnc(-c2c(Cl)ccc3nc(NC(=O)OCc4ncco4)sc23)cn1] | n/a | 418.0 |
| | | (Stille coupling used in Step 1: 2- | | |
| | | methoxy-5- | | |
| | | (tributylstannyl)pyrazine, P(Cy₃)Pd | | |
| | | G3, EtOH, 90° C.) | | |

### Synthetic Procedure I

### Example 325: Oxazol-2-ylmethyl (7-(3-((difluoromethoxy)methyl)-3-fluoroazetidin-1-yl)benzo[d]thiazol-2-yl)carbamate

### Step 1

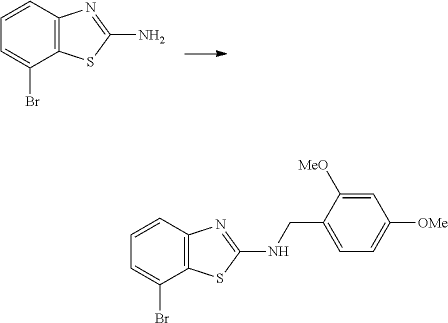 [SMILES: Nc1nc2cccc(Br)c2s1 ; COc1ccc(CNc2nc3cccc(Br)c3s2)c(OC)c1]

Titanium(IV) ethoxide (18.2 mL, 87.7 mmol) was added to a solution of 7-bromobenzo[d]thiazol-2-amine (10.0 g, 43.7 mmol) and 2,4-dimethoxybenzaldehyde (15.0 g, 90.3 mmol) in THF (100 mL) at 25° C. The mixture was stirred at 60° C. for 16 h. Sodium cyanoborohydride (5.60 g, 89.1 mmol) was then added at 25° C. and stirring was continued for 12 h at this temperature. Water (300 mL) was added and the mixture was extracted into EtOAc (3×200 mL). The combined organic extracts were dried over anhydrous sodium sulfate and then concentrated in vacuo. Purification by preparative HPLC (column: Welch Ultimate XB-NH2 250*50 10 μm; hexane:ethanol (0.11% NH₃·H₂O)]; gradient: 10%-50% B over 15 min) afforded 7-bromo-N-(2,4-dimethoxybenzyl)benzo[d]thiazol-2-amine (8.00 g, 20.7 mmol, 47%) as a yellow solid; ES-MS [M+H]⁺ 381.0.

### Step 2

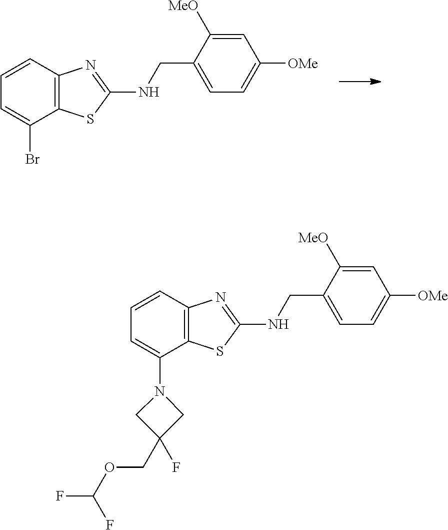 [SMILES: COc1ccc(CNc2nc3cccc(Br)c3s2)c(OC)c1 ; COc1ccc(CNc2nc3cccc(N4CC(F)(COC(F)F)C4)c3s2)c(OC)c1]

SPhos Pd G3 (210 mg, 269 μmol) and sodium tert-butoxide (700 mg, 8.01 mmol) were added to a solution of 3-((difluoromethoxy)methyl)-3-fluoroazetidine (400 mg, 2.58 mmol) and 7-bromo-N-(2,4-dimethoxybenzyl)benzo[d]thiazol-2-amine (1.00 g, 2.64 mmol) in 2-methylbutan-2-ol (10 mL). The mixture was stirred at 110° C. for 12 h. Water (50 mL) was added and the mixture was extracted into EtOAc (3×100 mL). The combined organic extracts were dried over anhydrous sodium sulfate and then concentrated in vacuo. Flash column chromatography on silica (EtOAc:PE, 30:70 to 40:60) afforded 7-(3-((difluoromethoxy)methyl)-3-fluoroazetidin-1-yl)-N-(2,4-dimethoxybenzyl)benzo[d]thiazol-2-amine (150 mg, 132 μmol, 5%) as a yellow oil; ES-MS [M+H]⁺ 454.2.

### Step 3

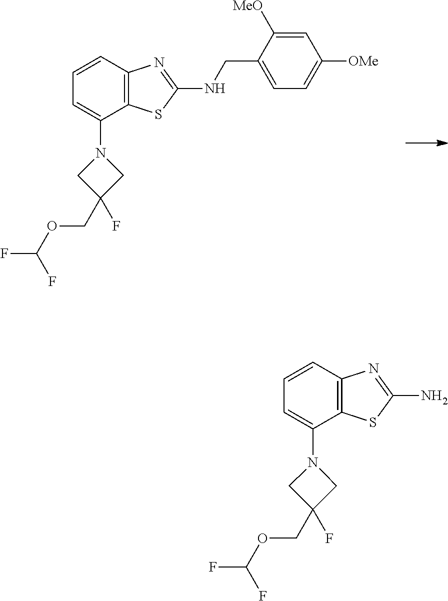 [SMILES: COc1ccc(CNc2nc3cccc(N4CC(F)(COC(F)F)C4)c3s2)c(OC)c1 ; Nc1nc2cccc(N3CC(F)(COC(F)F)C3)c2s1]

TFA (5 mL) was added to 7-(3-((difluoromethoxy)methyl)-3-fluoroazetidin-1-yl)-N-(2,4-dimethoxybenzyl)benzo[d]thiazol-2-amine (150 mg, 331 μmol) and the reaction mixture was stirred at 25° C. for 12 h. The mixture was adjusted to pH 7 with saturated aqueous bicarbonate solution and extracted into dichloromethane (3×20 mL). The combined organic extracts were dried over anhydrous sodium sulfate and then concentrated in vacuo. Flash column chromatography on silica (EtOAc:PE, 30:70 to 40:60) afforded 7-(3-((difluoromethoxy)methyl)-3-fluoroazetidin-1-yl)benzo[d]thiazol-2-amine (60 mg, 158 μmol, 48%) as a yellow oil; ES-MS [M+H]⁺ 304.0.

### Step 4

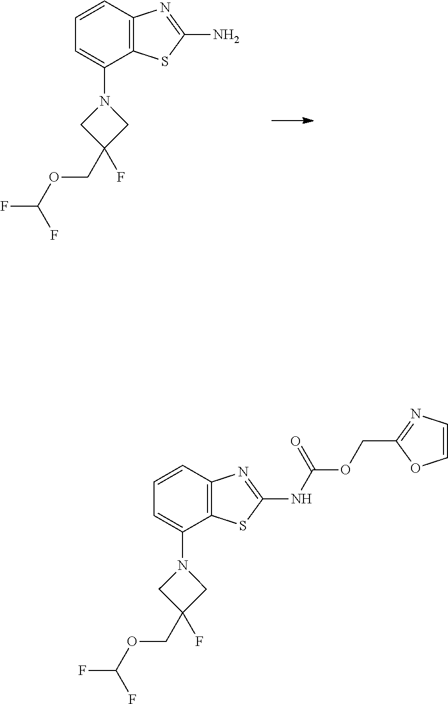 [SMILES: Nc1nc2cccc(N3CC(F)(COC(F)F)C3)c2s1 ; O=C(Nc1nc2cccc(N3CC(F)(COC(F)F)C3)c2s1)OCc1ncco1]

Oxazol-2-ylmethyl (7-(3-((difluoromethoxy)methyl)-3-fluoroazetidin-1-yl)benzo[d]thiazol-2-yl)carbamate was prepared from 7-(3-((difluoromethoxy)methyl)-3-fluoroazetidin-1-yl)benzo[d]thiazol-2-amine using Synthetic Procedure E, step 3; δ_{H} (400 MHz; DMSO-d6) 12.3 (1H, br s), 8.19 (1H, m), 7.28 (2H, m), 7.17 (1H, m), 6.81 (1H, m), 6.42 (1H, m), 5.35 (2H, m), 4.40-4.00 (6H, m); δ_{F} (400 MHz; CD₃OD) −85.9, −157.6; ES-MS [M+H]⁺: 429.1.

Other examples prepared using synthetic procedure I are shown in the following table.

| | | | ES-MS |
|---|---|---|---|
| Ex. No. | Name | Structure | [M + H]⁺ |
| 326 | oxazol-2-ylmethyl (7-(3-fluoro-3- (methoxymethyl)azetidin-1- yl)benzo[d]thiazol-2-yl)carbamate | 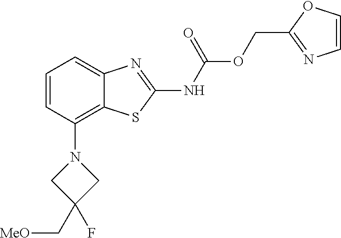 [SMILES: COCC1(F)CN(c2cccc3nc(NC(=O)OCc4ncco4)sc23)C1] | 393.1 |

### Synthetic Procedure J

### Example 327: Oxazol-2-ylmethyl (7-morpholinobenzo[d]thiazol-2-yl)carbamate

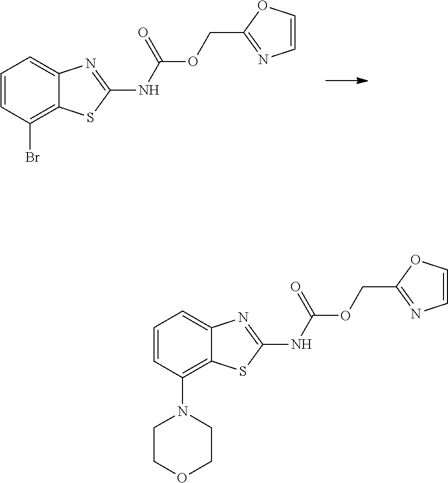 [SMILES: O=C(Nc1nc2cccc(Br)c2s1)OCc1ncco1 ; O=C(Nc1nc2cccc(N3CCOCC3)c2s1)OCc1ncco1]

SPhos Pd G3 (20 mg, 26 μmol) and potassium phosphate (180 mg, 0.85 mmol) were added to a solution of oxazol-2-ylmethyl (7-bromobenzo[d]thiazol-2-yl)carbamate (400 mg, 2.58 mmol) and morpholine (30 μL, 344 μmol) in 2-methylbutan-2-ol (2 mL). The mixture was stirred at 110° C. for 12 h. The reaction mixture was cooled, filtered and concentrated in vacuo. Purification by preparative HPLC ((column: Welch Xtimate C₁₈ 150*25 mm 5 μm; acetonitrile:water (formic acid); gradient: 28%-48% B over 10 min) afforded oxazol-2-ylmethyl (7-morpholinobenzo[d]thiazol-2-yl)carbamate (5 mg, 13.6 μmol, 5%) as a white solid; δ_{H} (400 MHz; DMSO-d6) 8.18 (1H, s), 7.39-7.31 (2H, m), 7.28 (1H, s), 6.93-6.84 (1H, m), 5.33 (2H, s), 3.82-3.75 (4H, m), 3.14-3.06 (4H, m); ES-MS [M+H]+ 361.1.

Other examples prepared using synthetic procedure J are shown in the following table. All intermediates are commercially available unless noted (see ‘Intermediate synthesis’ section).

| | | | Intermediates | |
|---|---|---|---|---|
| Ex. | | | requiring | ES-MS |
| No. | Name | Structure | synthesis | [M + H]⁺ |
| 328 | oxazol-2-ylmethyl (7-(3- ((difluoromethoxy)methyl)a- zetidin-1- yl)benzo[d]thiazol-2- yl)carbamate | 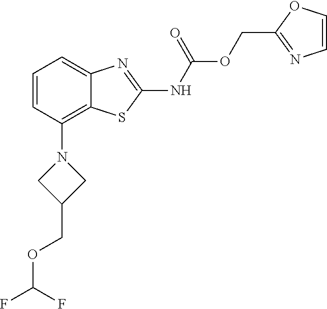 [SMILES: O=C(Nc1nc2cccc(N3CC(COC(F)F)C3)c2s1)OCc1ncco1] | n/a | 411.2 |
| 329 | oxazol-2-ylmethyl (7-(3- fluoro-3-((methoxy- d3)methyl)azetidin-1- yl)benzo[d]thiazol-2- yl)carbamate | 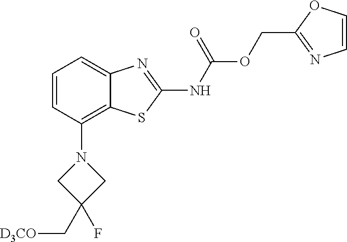 | 3-fluoro-3- ((methoxy- d3)methyl)azeti- dine | 396.1 |

### Synthetic Procedure K

### Example 330: Sodium (N-(7-(2-ethoxypyrimidin-5-yl)benzo[d]thiazol-2-yl)-3-fluoro-3-((methoxy-d3)methyl)azetidine-1-carboxamido)methyl phosphate

### Step 1

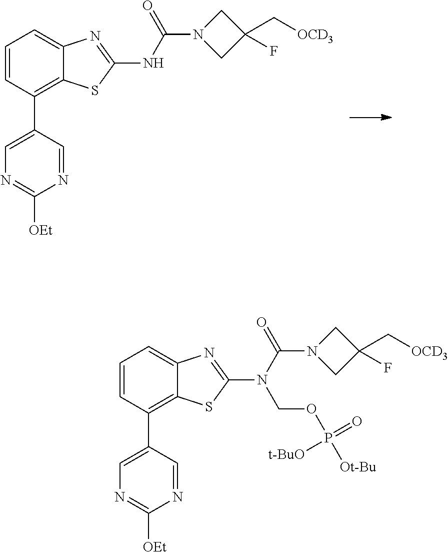

Di-tert-butyl chloromethyl phosphate (292 mg, 1.13 mmol) was added to a mixture of Example 30 (350 mg, 0.83 mmol), potassium iodide (292 mg, 1.76 mmol) and potassium carbonate (292 mg, 2.11 mmol) in DMF (8 mL). The resulting mixture was stirred at 60° C. for 12 h. Water (100 mL) was added and the mixture was extracted into EtOAc (3×100 mL). The combined organic extracts were washed with brine (2×100 mL), dried over anhydrous sodium sulfate and then concentrated in vacuo. Purification by lash column chromatography on silica (EtOAc:PE, 0:100 to 80:20) then preparative HPLC (column: Welch Ultimate XB-CN 250*50 10 μm; hexane:ethanol; gradient: 1%-40% B over 15 min) afforded di-tert-butyl ((N-(7-(2-ethoxypyrimidin-5-yl)benzo[d]thiazol-2-yl)-3-fluoro-3-((methoxy-d3)methyl)azetidine-1-carboxamido)methyl) phosphate (250 mg, 0.35 mmol, 42%) as a yellow oil; ES-MS [M+H]⁺: 643.4.

### Step 2

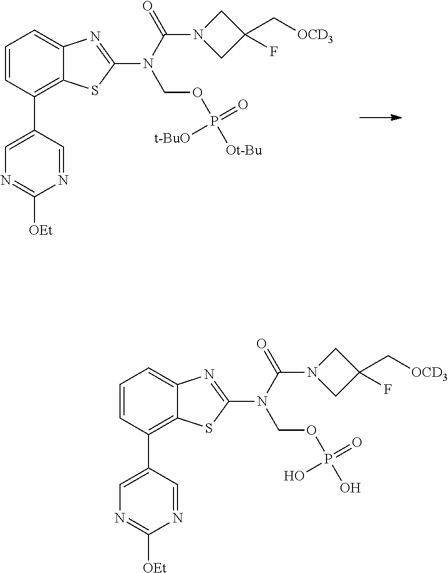

TFA (0.7 mL, 8.97 mmol) was added to a solution of di-tert-butyl ((N-(7-(2-ethoxypyrimidin-5-yl)benzo[d]thiazol-2-yl)-3-fluoro-3-((methoxy-d3)methyl)azetidine-1-carboxamido)methyl) phosphate (200 mg, 0.31 mmol) in dichloromethane (2 mL). The mixture was stirred at 25° C. for 2 h. The reaction mixture was concentrated and purified by preparative HPLC (column: Phenomenex Luna C18 150*25 mm 10 μm; acetonitrile:water (formic acid); gradient: 35%-65% B over 10 min) to afford (N-(7-(2-ethoxypyrimidin-5-yl)benzo[d]thiazol-2-yl)-3-fluoro-3-((methoxy-d3)methyl)azetidine-1-carboxamido)methyl dihydrogen phosphate as a white solid ES-MS [M+H]⁺: 531.2.

### Step 3

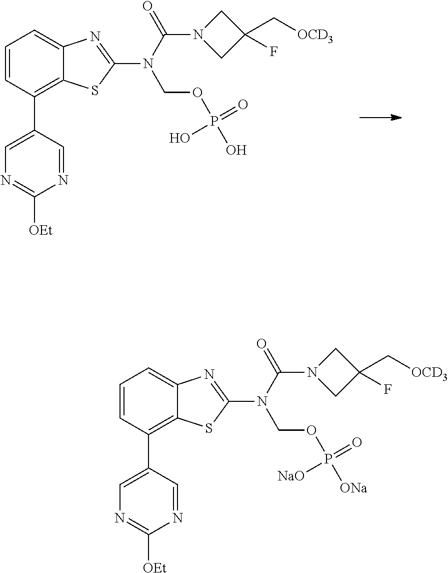

A solution of (N-(7-(2-ethoxypyrimidin-5-yl)benzo[d]thiazol-2-yl)-3-fluoro-3-((methoxy-d3)methyl)azetidine-1-carboxamido)methyl dihydrogen phosphate (50 mg, 0.094 mmol) in water (10 mL) and acetonitrile (2 mL) was filtered through Dowex 50W X8 Ion Exchange Resin (sodium form, 100-200 mesh, strongly acidic, 500 mg). The filtrate was lyophilized to afford sodium (N-(7-(2-ethoxypyrimidin-5-yl)benzo[d]thiazol-2-yl)-3-fluoro-3-((methoxy-d3)methyl)azetidine-1-carboxamido)methyl phosphate (40 mg, 0.069 mmol, 99%) as a white solid; δ_{H} (400 MHz; CD₃OD) 8.85-8.73 (2H, m), 7.84-7.73 (1H, m), 7.60-7.48 (1H, m), 7.37-7.27 (1H, m), 6.16-5.98 (2H, m), 4.58-4.48 (2H, m), 4.39-3.97 (4H, m), 3.76-3.63 (2H, m), 1.52-1.37 (3H, m); δ_{F} (400 MHz; CD₃OD) −1.18; ES-MS [M+H]⁺: 531.0.

Other examples prepared using synthetic procedure K are shown in the following table.

| Ex. | | | Starting | ES-MS |
|---|---|---|---|---|
| No. | Name | Structure | material | [M + H]⁺ |
| 331 | sodium (N-(6-chloro-7-(6- methoxypyridin-3- yl)benzo[d]thiazol-2-yl)-3- (methoxy-d3)-3- methylazetidine-1- carboxamido)methyl phosphate | 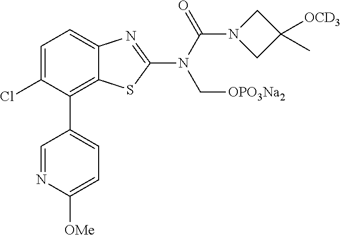 | Example 192 | 532.1 |
| 332 | sodium (3-fluoro-3- (methoxymethyl)-N-(7-(2- methoxypyrimidin-5- yl)benzo[d]thiazol-2- yl)azetidine-1- carboxamido)methyl phosphate | 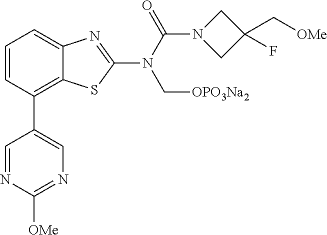 | Example 1 | 514.2 |

### Synthetic Procedure L

### Example 333: N-(7-(3-((Difluoromethoxy)methyl)-3-fluoroazetidin-1-yl)benzo[d]thiazol-2-yl)-5-oxa-2-azaspiro[3.4]octane-2-carboxamide

### Step 1

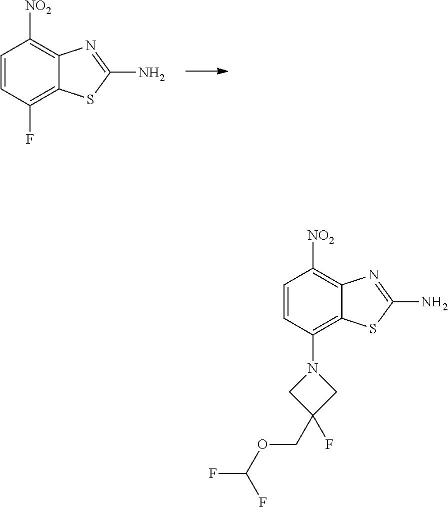 [SMILES: Nc1nc2c([N+](=O)[O-])ccc(F)c2s1 ; Nc1nc2c([N+](=O)[O-])ccc(N3CC(F)(COC(F)F)C3)c2s1]

3-((Difluoromethoxy)methyl)-3-fluoroazetidine TFA salt (3.50 g, 13.0 mmol) was added to a mixture of 7-floro-4-nitrobenzo[d]thiazol-2-amine* (2.50 g, 11.7 mmol) and potassium carbonate (5.39 g, 39.0 mmol) in DMF and the reaction mixture was stirred at 100° C. for 12 h. Water (80 mL) was added and the mixture was extracted into EtOAc (3×40 mL). The combined organic extracts were dried over anhydrous sodium sulfate and then concentrated in vacuo. Trituration with acetonitrile afforded 7-(3-((difluoromethoxy)methyl)-3-fluoroazetidin-1-yl)-4-nitrobenzo[d]thiazol-2-amine (2.50 g, 3.01 mmol, 23%) as a yellow solid; ES-MS [M+H]⁺: 349.1. *see ‘Intermediate synthesis’ section

### Step 2

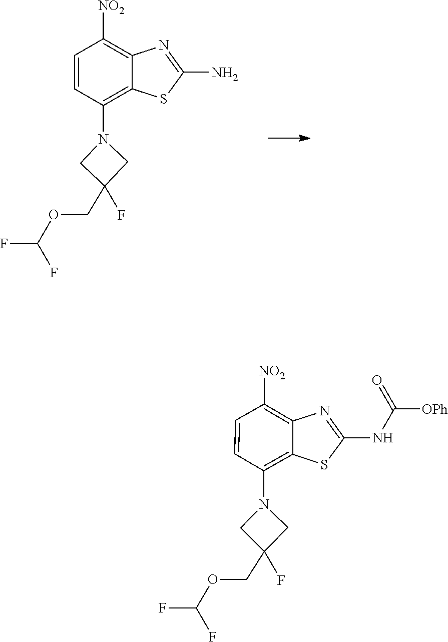 [SMILES: Nc1nc2c([N+](=O)[O-])ccc(N3CC(F)(COC(F)F)C3)c2s1 ; O=C(Nc1nc2c([N+](=O)[O-])ccc(N3CC(F)(COC(F)F)C3)c2s1)Oc1ccccc1]

Phenyl (7-(3-((difluoromethoxy)methyl)-3-fluoroazetidin-1-yl)-4-nitrobenzo[d]thiazol-2-yl)carbamate was prepared from 7-(3-((difluoromethoxy)methyl)-3-fluoroazetidin-1-yl)-4-nitrobenzo[d]thiazol-2-amine using Synthetic Procedure E, Step 3 (600 mg, 1.28 mmol, 56%); ES-MS [M+H]⁺: 469.1.

### Step 3

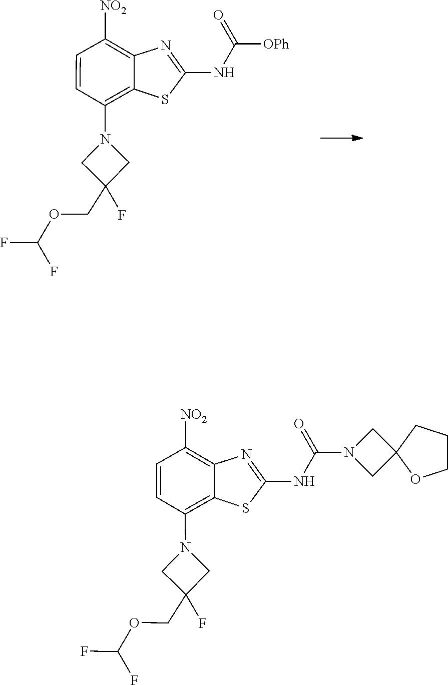 [SMILES: O=C(Nc1nc2c([N+](=O)[O-])ccc(N3CC(F)(COC(F)F)C3)c2s1)Oc1ccccc1 ; O=C(Nc1nc2c([N+](=O)[O-])ccc(N3CC(F)(COC(F)F)C3)c2s1)N1CC2(CCCO2)C1]

N-(7-(3-((Difluoromethoxy)methyl)-3-fluoroazetidin-1-yl)-4-nitrobenzo[d]thiazol-2-yl)-5-oxa-2-azaspiro[3.4]octane-2-carboxamide was prepared from phenyl (7-(3-((difluoromethoxy)methyl)-3-fluoroazetidin-1-yl)-4-nitrobenzo[d]thiazol-2-yl)carbamate using Synthetic Procedure A, Step 3 (250 mg, 0.51 mmol, 48%); δ_{H} (400 MHz; DMSO-d6) 11.87 (1H, br s), 8.12 (1H, d, J 8.9 Hz), 7.05-6.62 (1H, m), 7.04-6.59 (1H, m), 6.38 (1H, d, J 9.0 Hz), 4.57-4.33 (6H, m), 4.12 (2H, br s), 3.78 (2H, t, J 6.8 Hz), 3.37 (2H, br s), 2.12-2.05 (2H, m), 1.87 (2H, m).

### Step 4

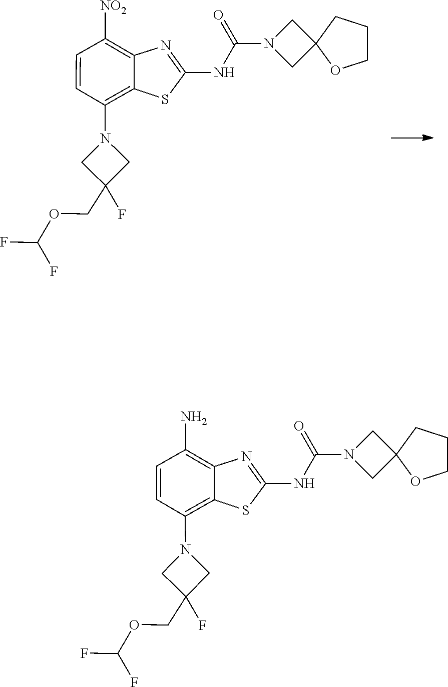 [SMILES: O=C(Nc1nc2c([N+](=O)[O-])ccc(N3CC(F)(COC(F)F)C3)c2s1)N1CC2(CCCO2)C1 ; Nc1ccc(N2CC(F)(COC(F)F)C2)c2sc(NC(=O)N3CC4(CCCO4)C3)nc12]

A mixture of N-(7-(3-((difluoromethoxy)methyl)-3-fluoroazetidin-1-yl)-4-nitrobenzo[d]thiazol-2-yl)-5-oxa-2-azaspiro[3.4]octane-2-carboxamide (250 mg, 0.51 mmol) and 10% Pd/C (100 mg, 0.94 mmol) in THF (5 mL) was degassed and purged with hydrogen three times, and then stirred at 25° C. for 12 h under a hydrogen (15 psi). The mixture was filtered and concentrated in vacuo to afford N-(4-amino-7-(3-((difluoromethoxy)methyl)-3-fluoroazetidin-1-yl)benzo[d]thiazol-2-yl)-5-oxa-2-azaspiro[3.4]octane-2-carboxamide (200 mg, 0.44 mmol, 85%) as a white solid; δ_{H} (400 MHz; DMSO-d6) 11.52-10.71 (1H, m), 7.03-6.62 (1H, m), 6.58 (1H, d, J 8.1 Hz), 6.30 (1H, d, J 8.2 Hz), 4.70 (2H, br s), 4.38-4.30 (2H, m), 4.11-3.96 (6H, m), 3.91-3.80 (2H, m), 3.77 (2H, m), 2.11-2.04 (2H, m), 1.86 (2H, m).

### Step 5

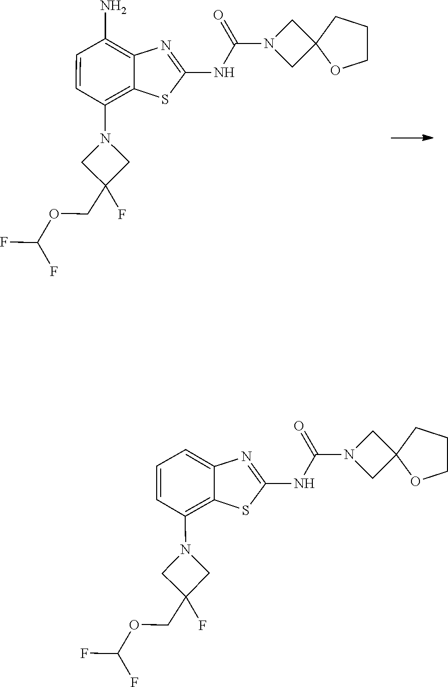 [SMILES: Nc1ccc(N2CC(F)(COC(F)F)C2)c2sc(NC(=O)N3CC4(CCCO4)C3)nc12 ; O=C(Nc1nc2cccc(N3CC(F)(COC(F)F)C3)c2s1)N1CC2(CCCO2)C1]

A solution of tert-butyl nitrite (68 mg, 0.66 mmol) in THF (1 mL) was added to a mixture of N-(4-amino-7-(3-((difluoromethoxy)methyl)-3-fluoroazetidin-1-yl)benzo[d]thiazol-2-yl)-5-oxa-2-azaspiro[3.4]octane-2-carboxamide (100 mg, 0.22 mmol) in THF (1 mL) at 0° C. The resulting mixture was stirred at 25° C. for 5 h then water (20 mL) was added. The mixture was extracted into EtOAc (3×10 mL) and the combined organic extracts were dried over anhydrous sodium sulfate, then concentrated in vacuo. Purification by preparative HPLC (column: Waters Xbridge 150*25 mm 5 μm; acetonitrile:water (NH₄CO₃); gradient: 30%-60% B over 10 min) afforded N-(7-(3-((difluoromethoxy)methyl)-3-fluoroazetidin-1-yl)benzo[d]thiazol-2-yl)-5-oxa-2-azaspiro[3.4]octane-2-carboxamide (4 mg, 9 mol, 4%) as a yellow solid; δ_{H} (400 MHz; DMSO-d6) 11.67-10.97 (1H, m), 7.28-7.21 (1H, m), 7.17-7.06 (1H, m), 6.82 (1H, m), 6.39 (1H, d, J 7.9 Hz), 4.36 (2H, d, J 23.8 Hz), 4.23 (2H, m), 4.13-3.94 (6H, m), 3.76 (2H, m), 2.13-2.02 (2H, m), 1.86 (2H, m); δ_{F} (400 MHz; DMSO-d6) −82.7, −155.1; ES-MS [M+H]⁺: 443.2.

Other examples prepared using synthetic procedure L are shown in the following table.

| Ex. | | | ES-MS |
|---|---|---|---|
| No. | Name | Structure | [M + H]⁺ |
| 334 | N-(7-(3-((difluoromethoxy)methyl)-3- fluoroazetidin-1-yl)benzo[d]thiazol-2-yl)- 6-oxa-2-azaspiro[3.4]octan2- carboxamide | 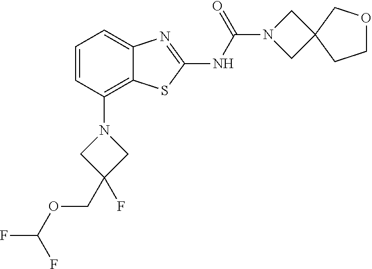 [SMILES: O=C(Nc1nc2cccc(N3CC(F)(COC(F)F)C3)c2s1)N1CC2(CCOC2)C1] | 443.2 |
| 335 | N-(7-(3-((difluoromethoxy)methyl)-3- fluoroazetidin-1-yl)benzo[d]thiazol-2-yl)- 3-fluoro-3-(methoxymethyl)azetidine-1- carboxamide | 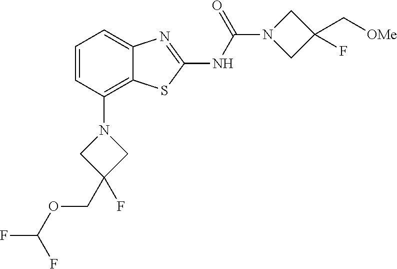 [SMILES: COCC1(F)CN(C(=O)Nc2nc3cccc(N4CC(F)(COC(F)F)C4)c3s2)C1] | 449.2 |
| 336 | 3-fluoro-N-(7-(3-fluoro-3- (morpholinomethyl)azetidin-1- yl)benzo[d]thiazol-2-yl)-3- (methoxymethyl)azetidine-1-carboxamide | 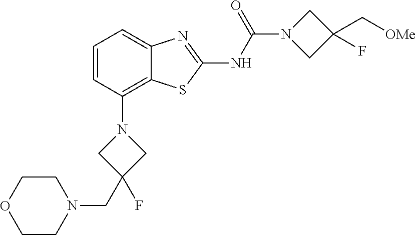 [SMILES: COCC1(F)CN(C(=O)Nc2nc3cccc(N4CC(F)(CN5CCOCC5)C4)c3s2)C1] | 468.1 |
| 337 | oxazol-2-ylmethyl (7-(3-fluoro-3- (morpholinomethyl)azetidin-1- yl)benzo[d]thiazol-2-yl)carbamate | 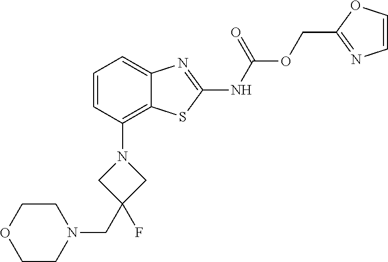 [SMILES: O=C(Nc1nc2cccc(N3CC(F)(CN4CCOCC4)C3)c2s1)OCc1ncco1] | 448.1 |
| | | (Steps 2 and 3 replaced with | |
| | | Procedure E, step 3) | |

### Synthetic Procedure M

### Example 338: Oxazol-2-ylmethyl (6-((3-fluoroazetidin-1-yl)methyl)-7-(6-methoxypyridin-3-yl)benzo[d]thiazol-2-yl)carbamate

### Step 1

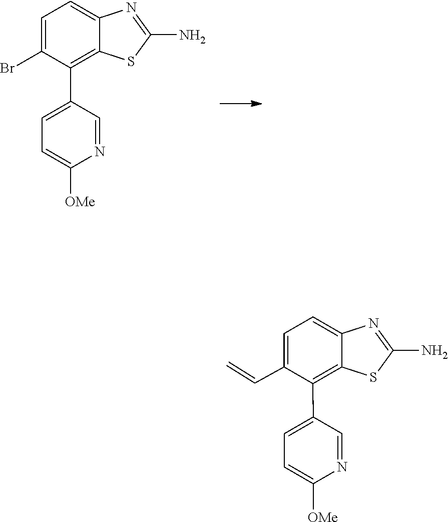 [SMILES: COc1ccc(-c2c(Br)ccc3nc(N)sc23)cn1 ; C=Cc1ccc2nc(N)sc2c1-c1ccc(OC)nc1]

Pd(PPh₃)₂Cl₂ (2.09 g, 2.97 mmol) was added to a mixture of 6-bromo-7-(6-methoxypyridin-3-yl)benzo[d]thiazol-2-amine* (10.0 g, 29.7 mmol), potassium vinyltrifluoroborate (5.98 g, 44.6 mmol) and potassium carbonate (12.3 g, 89.2 mmol) in dioxane (200 mL) and water (20 mL). The reaction mixture was stirred at 80° C. for 16 h. Water (50 mL) was added and the mixture was extracted into EtOAc (3×50 mL) and the combined organic extracts were dried over anhydrous sodium sulfate, then concentrated in vacuo. Flash column chromatography on silica (EtOAc:PE, 0:100 to 50:50) afforded 7-(6-methoxypyridin-3-yl)-6-vinylbenzo[d]thiazol-2-amine (3.50 g, 11.2 mmol, 38% yield) as a yellow solid; ES-MS [M+H]⁺: 284.0. *see ‘Intermediate synthesis’ section

### Step 2

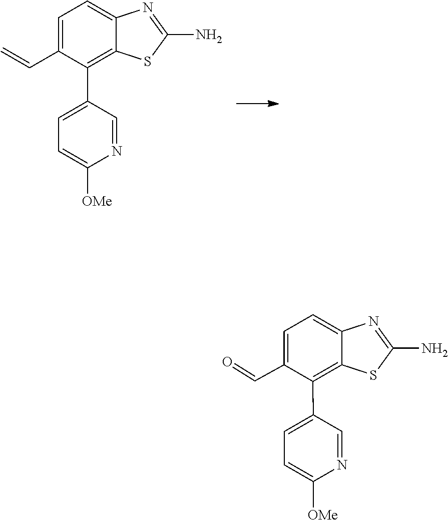 [SMILES: C=Cc1ccc2nc(N)sc2c1-c1ccc(OC)nc1 ; COc1ccc(-c2c(C=O)ccc3nc(N)sc23)cn1]

Potassium osmate(VI) dihydrate (195 mg, 0.53 mmol) and sodium periodate (5.66 g, 26.5 mmol) were added to a solution of 7-(6-methoxypyridin-3-yl)-6-vinylbenzo[d]thiazol-2-amine (1.50 g, 5.29 mmol) in THF (60 mL) and water (15 mL) at 0° C. The reaction mixture was stirred at 25° C. for 16 h. The reaction mixture was quenched with saturated aqueous sodium bisulfite solution (30 mL) and extracted into EtOAc (3×30 mL). The combined organic extracts were dried over anhydrous sodium sulfate, then concentrated in vacuo. Flash column chromatography on silica (EtOAc:PE, 0:100 to 60:40) afforded 2-amino-7-(6-methoxypyridin-3-yl)benzo[d]thiazole-6-carbaldehyde (930 mg, 2.57 mmol, 49%) as a white solid; ES-MS [M+H]⁺: 286.0.

### Step 3

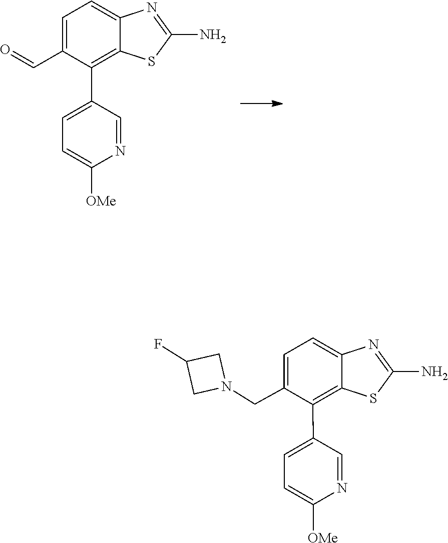 [SMILES: COc1ccc(-c2c(C=O)ccc3nc(N)sc23)cn1 ; COc1ccc(-c2c(CN3CC(F)C3)ccc3nc(N)sc23)cn1]

Sodium cyanoborohydride (165 mg, 2.63 mmol), 3-fluoroazetidine HCl salt (146 mg, 1.31 mmol) and acetic acid (50 μL, 876 μmol) were added to a solution of 2-amino-7-(6-methoxypyridin-3-yl)benzo[d]thiazole-6-carbaldehyde (250 mg, 876 μmol) in methanol (5 mL). The reaction mixture was stirred at 40° C. for 16 h then concentrated in vacuo. Water (10 mL) was added and the mixture was extracted into ethyl acetate (3×10 mL). The combined organic extracts were dried over anhydrous sodium sulfate, then concentrated in vacuo. Flash column chromatography on silica (EtOAc:PE, 0:100 to 80:20) afforded 6-((3-fluoroazetidin-1-yl)methyl)-7-(6-methoxypyridin-3-yl)benzo[d]thiazol-2-amine (200 mg, 523 mol, 60%) as a white solid; ES-MS [M+H]⁺: 345.1.

### Step 4

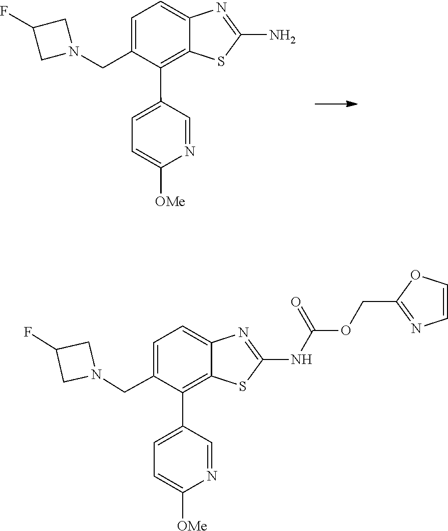 [SMILES: COc1ccc(-c2c(CN3CC(F)C3)ccc3nc(N)sc23)cn1 ; COc1ccc(-c2c(CN3CC(F)C3)ccc3nc(NC(=O)OCc4ncco4)sc23)cn1]

Oxazol-2-ylmethyl (6-((3-fluoroazetidin-1-yl)methyl)-7-(6-methoxypyridin-3-yl)benzo[d]thiazol-2-yl)carbamate was prepared from 6-((3-fluoroazetidin-1-yl)methyl)-7-(6-methoxypyridin-3-yl)benzo[d]thiazol-2-amine using Synthetic Procedure E, step 3; δ_{H} (400 MHz; DMSO-d6) 12.40-12.24 (1H, m), 8.31-8.23 (1H, m), 8.19-8.14 (1H, m), 7.87-7.79 (1H, m), 7.71-7.63 (1H, m), 7.57-7.49 (1H, m), 7.29-7.22 (1H, m), 7.03-6.95 (1H, m), 5.36-5.26 (2H, m), 5.23-5.01 (1H, m), 3.99-3.90 (3H, m), 3.58-3.41 (4H, m), 3.13-2.98 (2H, m); δ_{F} (400 MHz, DMSO-d6), −178.0; CDCl₃ES-MS [M+H]⁺: 470.1.

Other examples prepared using synthetic procedure M are shown in the following table.

| Ex. | | | ES-MS |
|---|---|---|---|
| No. | Name | Structure | [M + H]⁺ |
| 339 | oxazol-2-ylmethyl (7-(6-methoxypyridin-3- yl)-6-(pyrrolidin-1-ylmethyl)benzo[d]thiazol- 2-yl)carbamate | 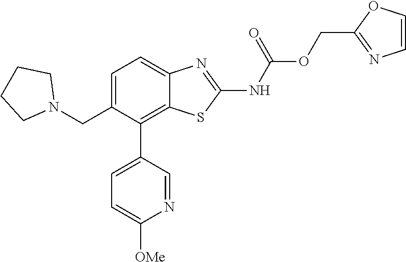 [SMILES: COc1ccc(-c2c(CN3CCCC3)ccc3nc(NC(=O)OCc4ncco4)sc23)cn1] | 466.2 |
| 340 | oxazol-2-ylmethyl (7-(6-methoxypyridin-3- yl)-6-(morpholinomethyl)benzo[d]thiazol-2- yl)carbamate | 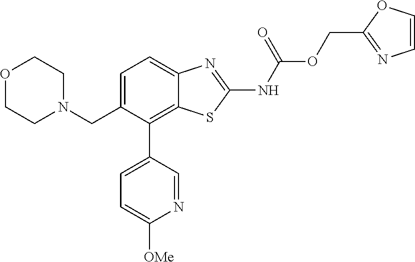 [SMILES: COc1ccc(-c2c(CN3CCOCC3)ccc3nc(NC(=O)OCc4ncco4)sc23)cn1] | 482.2 |
| 341 | oxazol-2-ylmethyl (7-(6-methoxypyridin-3- yl)-6-vinylbenzo[d]thiazol-2-yl)carbamate | 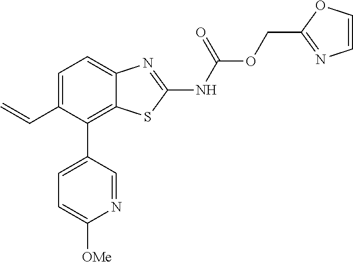 [SMILES: C=Cc1ccc2nc(NC(=O)OCc3ncco3)sc2c1-c1ccc(OC)nc1] | 409.2 |
| | | Steps 1 and 4 only | |

### Synthetic Procedure N

### Example 342: 3-Fluoro-N-(6-fluoro-7-(4-(morpholinomethyl)-1H-pyrazol-1-yl)benzo[d]thiazol-2-yl)-3-(methoxymethyl)azetidine-1-carboxamide

### Step 1

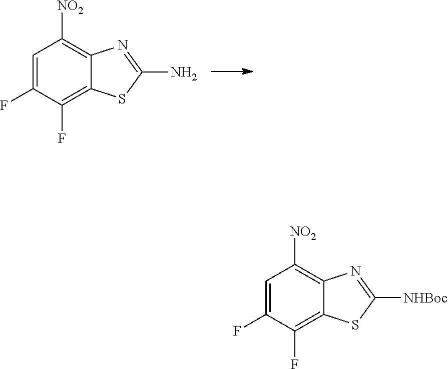 [SMILES: Nc1nc2c([N+](=O)[O-])cc(F)c(F)c2s1 ; CC(C)(C)OC(=O)Nc1nc2c([N+](=O)[O-])cc(F)c(F)c2s1]

tert-Butyl (6,7-difluoro-4-nitrobenzo[d]thiazol-2-yl)carbamate was prepared from 6,7-difluoro-4-nitrobenzo[d]thiazol-2-amine using Procedure F, Step 1; δ_{H} (400 MHz; DMSO-d6) 13.20-12.23 (1H, m), 8.39 (1H, m), 1.51 (9H, s).

### Step 2

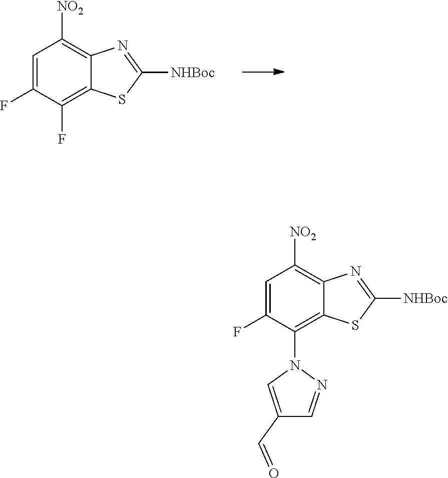 [SMILES: CC(C)(C)OC(=O)Nc1nc2c([N+](=O)[O-])cc(F)c(F)c2s1 ; CC(C)(C)OC(=O)Nc1nc2c([N+](=O)[O-])cc(F)c(-n3cc(C=O)cn3)c2s1]

Cesium carbonate (1.73 g, 5.31 mmol) was added to a solution of tert-butyl (6,7-difluoro-4-nitrobenzo[d]thiazol-2-yl)carbamate (880 mg, 2.66 mmol) and 1H-pyrazole-4-carbaldehyde (383 mg, 3.98 mmol) in DMF (9 mL). The resulting mixture was heated at 60° C. for 16 h. Water (20 mL) was added and the mixture was extracted into ethyl acetate (3×20 mL). The combined organic extracts were washed with brine (20 mL), dried over anhydrous sodium sulfate, then concentrated in vacuo. Trituration with acetonitrile afforded tert-butyl (6-fluoro-7-(4-formyl-1H-pyrazol-1-yl)-4-nitrobenzo[d]thiazol-2-yl)carbamate (900 mg, 2.21 mmol, 83%) as a light yellow solid; δ_{H} (400 MHz; DMSO-d6) 12.44 (1H, br s), 10.01 (1H, s), 9.19 (1H, d, J 2.4 Hz), 8.52 (1H, s), 8.39 (1H, d, J 12.0 Hz), 1.51 (9H, s).

### Step 3

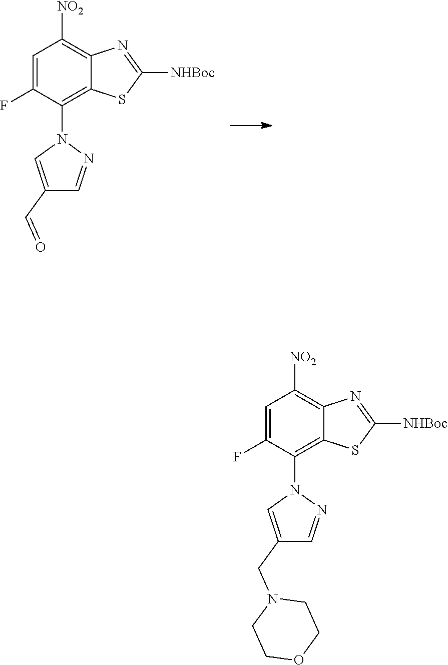 [SMILES: CC(C)(C)OC(=O)Nc1nc2c([N+](=O)[O-])cc(F)c(-n3cc(C=O)cn3)c2s1 ; CC(C)(C)OC(=O)Nc1nc2c([N+](=O)[O-])cc(F)c(-n3cc(CN4CCOCC4)cn3)c2s1]

Acetic acid (126 μL, 2.21 mmol) was added to a solution of tert-butyl (6-fluoro-7-(4-formyl-1H-pyrazol-1-yl)-4-nitrobenzo[d]thiazol-2-yl)carbamate (900 mg, 2.21 mmol) and morpholine (0.39 mL, 4.42 mmol) in DCE (18 mL). The mixture was stirred at 25° C. for 2 h, then sodium triacetoxyborohydride (0.94 g, 4.42 mmol) was added at 0° C. Stirring was continued at 60° C. for 16 h. Water (20 mL) was added and the mixture extracted into ethyl acetate (3×20 mL). The combined organic extracts were washed with brine (20 mL), dried over anhydrous sodium sulfate and concentrated in vacuo. Trituration with acetonitrile afforded tert-butyl (6-fluoro-7-(4-(morpholinomethyl)-1H-pyrazol-1-yl)-4-nitrobenzo[d]thiazol-2-yl)carbamate (1.00 g, 2.09 mmol, 95%) as a light yellow solid; ES-MS [M+H]⁺: 479.2.

### Step 4

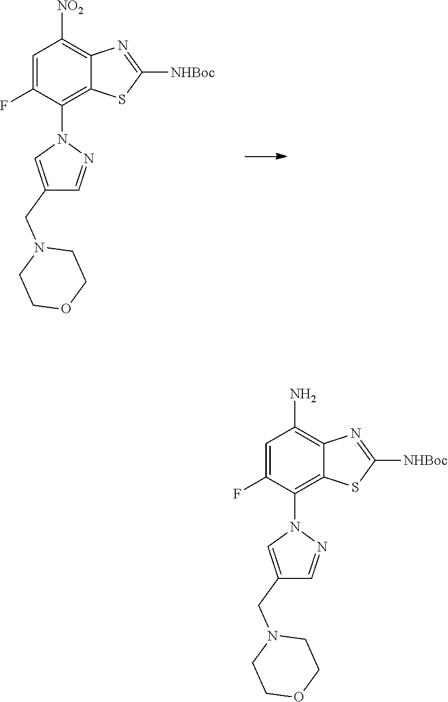 [SMILES: CC(C)(C)OC(=O)Nc1nc2c([N+](=O)[O-])cc(F)c(-n3cc(CN4CCOCC4)cn3)c2s1 ; CC(C)(C)OC(=O)Nc1nc2c(N)cc(F)c(-n3cc(CN4CCOCC4)cn3)c2s1]

tert-Butyl (4-amino-6-fluoro-7-(4-(morpholinomethyl)-1H-pyrazol-1-yl)benzo[d]thiazol-2-yl)carbamate was prepared from tert-butyl (6-fluoro-7-(4-(morpholinomethyl)-1H-pyrazol-1-yl)-4-nitrobenzo[d]thiazol-2-yl)carbamate using Procedure L, Step 4; ES-MS [M+H]⁺: 449.2.

### Step 5

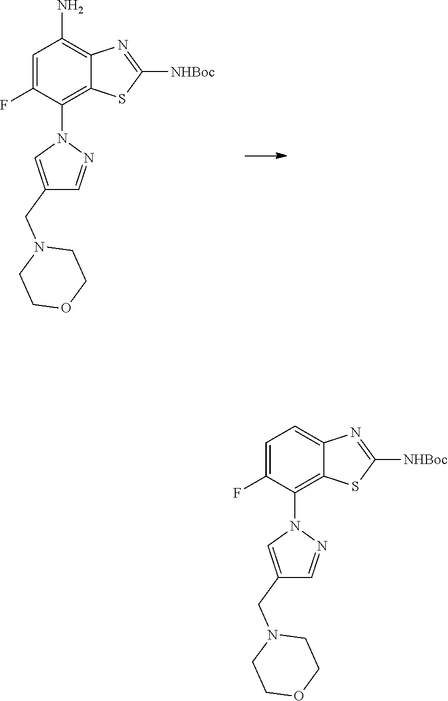 [SMILES: CC(C)(C)OC(=O)Nc1nc2c(N)cc(F)c(-n3cc(CN4CCOCC4)cn3)c2s1 ; CC(C)(C)OC(=O)Nc1nc2ccc(F)c(-n3cc(CN4CCOCC4)cn3)c2s1]

tert-Butyl (6-fluoro-7-(4-(morpholinomethyl)-1H-pyrazol-1-yl)benzo[d]thiazol-2-yl)carbamate was prepared from tert-butyl (4-amino-6-fluoro-7-(4-(morpholinomethyl)-1H-pyrazol-1-yl)benzo[d]thiazol-2-yl)carbamate using Procedure L, step 5; ES-MS [M+H]⁺: 434.3.

### Step 6

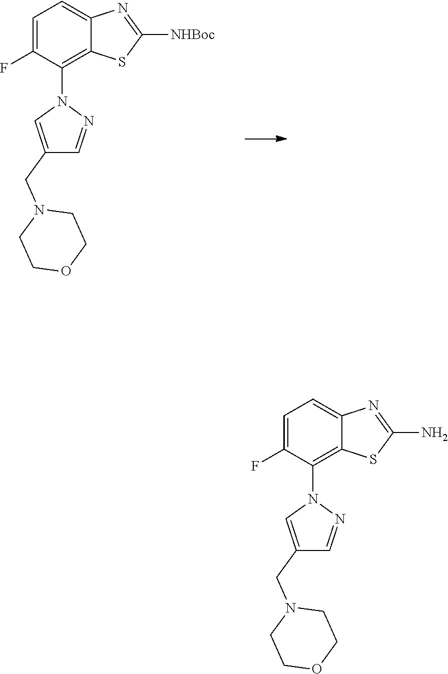 [SMILES: CC(C)(C)OC(=O)Nc1nc2ccc(F)c(-n3cc(CN4CCOCC4)cn3)c2s1 ; Nc1nc2ccc(F)c(-n3cc(CN4CCOCC4)cn3)c2s1]

HCl (2.0 M in dioxane, 10 mL, 20.0 mmol) was added to a solution of tert-butyl (6-fluoro-7-(4-(morpholinomethyl)-1H-pyrazol-1-yl)benzo[d]thiazol-2-yl)carbamate (550 mg, 1.27 mmol) in dioxane (2 mL). The mixture was stirred at 60° C. for 2 h, then concentrated in vacuo. Trituration with ethyl acetate afforded 6-fluoro-7-(4-(morpholinomethyl)-1H-pyrazol-1-yl)benzo[d]thiazol-2-amine (460 mg, 1.24 mmol, 98%) as an orange solid; ES-MS [M+H]⁺: 334.2.

### Steps 7 and 8

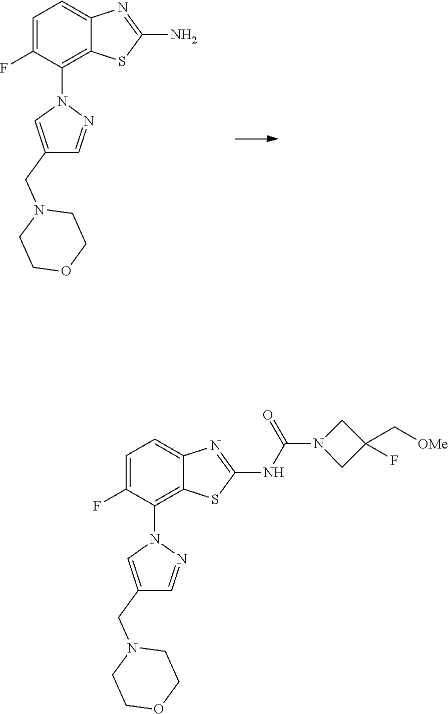 [SMILES: Nc1nc2ccc(F)c(-n3cc(CN4CCOCC4)cn3)c2s1 ; COCC1(F)CN(C(=O)Nc2nc3ccc(F)c(-n4cc(CN5CCOCC5)cn4)c3s2)C1]

3-Fluoro-N-(6-fluoro-7-(4-(morpholinomethyl)-1H-pyrazol-1-yl)benzo[d]thiazol-2-yl)-3-(methoxymethyl)azetidine-1-carboxamide was prepared from 6-fluoro-7-(4-(morpholinomethyl)-1H-pyrazol-1-yl)benzo[d]thiazol-2-amine using Procedure A, Steps 2 and 3; δ_{H} (400 MHz; CDCl₃) 8.23 (1H, br s), 8.18 (1H, d, J 2.4 Hz), 7.76 (1H, s), 7.46 (1H, m), 7.34-7.28 (1H, m), 4.30 (4H, d, J 19.2 Hz), 3.78-3.74 (4H, m), 3.69 (2H, d, J 18.0 Hz), 3.58 (2H, s), 3.47 (3H, s), 2.56 (4H, br s); δ_{F} (400 MHz; CDCl₃) −130.1, −157.5; ES-MS [M+H]⁺: 479.2.

Other examples prepared using synthetic procedure N are shown in the following table. All intermediates are commercially available unless noted (see ‘Intermediate synthesis’ section).

| Ex. | | | Intermediates | ES-MS |
|---|---|---|---|---|
| No. | Name | Structure | requiring synthesis | [M + H]⁺ |
| 343 | 3-fluoro-3- (methoxymethyl)-N- (7-(4- (morpholinomethyl)- 1H-pyrazol-1- yl)benzo[d]thiazol- 2-yl)azetidine-1- carboxamide | 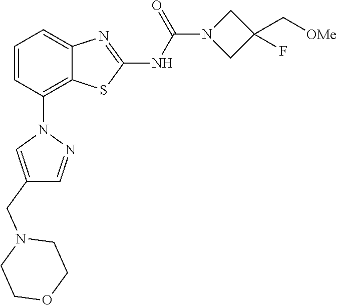 [SMILES: COCC1(F)CN(C(=O)Nc2nc3cccc(-n4cc(CN5CCOCC5)cn4)c3s2)C1] | 7-fluoro-4- nitrobenzo[d]thiazol- 2-amine | 461.1 |
| 344 | 3-fluoro-N-(7-(4- methoxy-1H- pyrazol-1- yl)benzo[d]thiazol- 2-yl)-3-((methoxy- d3)methyl)azetidine- 1-carboxamide | 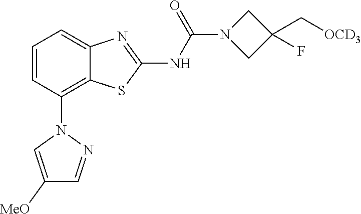 | 3-fluoro-3- ((methoxy- d3)methyl)azetidine and 7-fluoro-4- nitrobenzo[d]thiazol- 2-amine | 395.1 |
| | | Prepared using these Steps in order | | |
| | | shown: 1, 6, 7, 4, 5 | | |

### Intermediate Synthesis

### 3-(Methoxy-d3)-3-methylazetidine

### Step 1

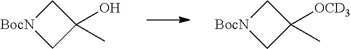

A mixture of tert-butyl 3-hydroxy-3-methyl azetidine-1-carboxylate (0.50 g, 2.67 mmol) and iodomethane-d3 (0.33 mL, 5.34 mmol) in TIFF (10 mL) was degassed and purged with nitrogen three times. Sodium hydride (60% dispersion in oil, 160 mg, 4.01 mmol) was added at 0° C. under nitrogen and the resulting mixture was stirred at 25° C. for 2 h. The reaction mixture was quenched with saturated aqueous ammonium chloride solution (50 mL) and extracted into ethyl acetate (3×30 mL). The combined organic extracts were dried over anhydrous sodium sulfate and then concentrated in vacuo to afford tert-butyl 3-(methoxy-d3)-3-methyl azetidine-1l-carboxylate (0.60 g, 2.26 mmol, 8500 yield, 7700 purity) as a white solid; δ_{H} (400 MHz; DMSO-d6) 3.78-3.55 (4H, m), 1.39-1.34 (12H, m); ES-MS [M+H]⁺: 205.2.

### Step 2

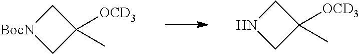

Trifluoroacetic acid (0.55 mL, 7.34 mmol) was added to a solution of tert-butyl 3-(methoxy-d3)-3-methyl azetidine-1l-carboxylate (0.30, 1.47 mmol) in dichloromethane (5 mL) and the resulting mixture was stirred at 20° C. for 2 h. The reaction mixture was diluted with water (50 mL) and then solid sodium bicarbonate was added in portions until the pH was ˜8. The mixture was extracted into ethyl acetate (3×30 mL). The combined organic extracts were dried over anhydrous sodium sulfate and then concentrated in vacuo to afford 3-(methoxy-d3)-3-methylazetidine (0.10 g, 0.77 mmol, 52% yield, 80% purity) as a white solid; ES-MS [M+H]⁺: 105.1.

### 8-Cyclopropyl-5-oxa-2,8-diazaspiro[3.5]nonane

### Step 1

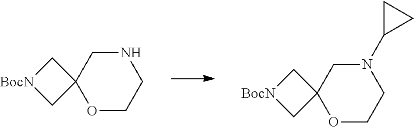 [SMILES: CC(C)(C)OC(=O)N1CC2(CNCCO2)C1 ; CC(C)(C)OC(=O)N1CC2(C1)CN(C1CC1)CCO2]

Cyclopropylboronic acid (100 mg, 1.16 mmol), copper(II) acetate (60 mg, 0.33 mmol) and sodium carbonate (50 mg, 0.47 mmol) were added to a solution of tert-butyl 5-oxa-2,8-diazaspiro[3.5]nonane-2-carboxylate (50 mg, 0.22 mmol) in dichloromethane (3 mL). The resulting mixture was stirred at 35° C. for 12 h then filtered. The filtrate was concentrated in vacuo to afford tert-butyl 8-cyclopropyl-5-oxa-2,8-diazaspiro[3.5]nonane-2-carboxylate (58 mg) as a yellow oil which was used without further purification.

### Step 2

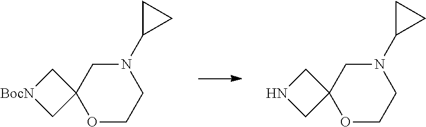 [SMILES: CC(C)(C)OC(=O)N1CC2(C1)CN(C1CC1)CCO2 ; C1CN(C2CC2)CC2(CNC2)O1]

Trifluoroacetic acid (0.50 mL, 6.73 mmol) was added to a solution of tert-butyl 8-cyclopropyl-5-oxa-2,8-diazaspiro[3.5]nonane-2-carboxylate (58 mg, 0.22 mmol) in dichloromethane (0.5 mL) and the resulting mixture was stirred at 25° C. for 1 h. The reaction mixture was concentrated in vacuo to afford 8-cyclopropyl-5-oxa-2,8-diazaspiro[3.5]nonane (36 mg) as a yellow oil which used without further purification.

### 3-((difluoromethoxy)methyl)-3-fluoroazetidine

### Step 1

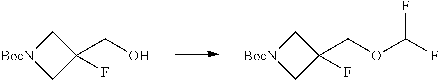 [SMILES: CC(C)(C)OC(=O)N1CC(F)(CO)C1 ; CC(C)(C)OC(=O)N1CC(F)(COC(F)F)C1]

(Bromodifluoromethyl)trimethylsilane (495 mg, 2.44 mmol) was added to a mixture of tert-butyl 3-fluoro-3-(hydroxymethyl)azetidine-1-carboxylate (200 mg, 0.97 mmol), potassium acetate (383 mg, 3.9 mmol), water (2 mL) and dichloromethane (2 mL). The resulting mixture was stirred at 20° C. for 12 h, then washed with brine, dried over anhydrous sodium sulfate and concentrated in vacuo. Flash column chromatography on silica (EtOAc:PE, 0:100 to 10:90) afforded tert-butyl 3-((difluoromethoxy)methyl)-3-fluoroazetidine-1-carboxylate (50 mg, 0.20 mmol, 20%) as a colorless oil; δ_{H} (400 MHz; DMSO-d6) 6.75 (1H, m), 4.22 (2H, d, J 22.5 Hz), 4.02-3.89 (4H, m), 1.39 (9H, s).

### Step 2

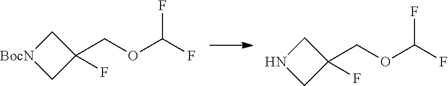 [SMILES: CC(C)(C)OC(=O)N1CC(F)(COC(F)F)C1 ; FC(F)OCC1(F)CNC1]

Trifluoroacetic acid (0.30 mL, 4.04 mmol) was added to a solution of tert-butyl 3-((difluoromethoxy)methyl)-3-fluoroazetidine-1-carboxylate (50 mg, 0.20 mmol) in dichloromethane (1 mL) and the resulting mixture was stirred at 20° C. for 1.5 h. The reaction mixture was concentrated in vacuo to afford 3-((difluoromethoxy)methyl)-3-fluoroazetidine (30 mg, TFA salt) as a colorless oil which used without further purification.

### 3-Fluoro-3-((methoxy-d3)methyl)azetidine

### Step 1

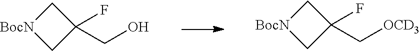

tert-Butyl 3-fluoro-3-(hydroxymethyl)azetidine-1-carboxylate (200 mg, 0.97 mmol) was added to a mixture of sodium hydride (60% dispersion in oil, 80 mg, 2.00 mmol) in THF (3 mL) at 0° C. The resulting mixture was stirred at 25° C. for 30 min. Iodomethane-d3 (64 μL, 1.03 mmol) was added at 0° C. and the mixture was stirred at 25° C. for another 2 h. The reaction mixture was quenched with saturated aqueous ammonium chloride solution (20 mL) and extracted into EtOAc (3×50 mL). The combined organic extracts were dried over anhydrous sodium sulfate and then concentrated in vacuo to afford tert-butyl 3-fluoro-3-((methoxy-d3)methyl)azetidine-1-carboxylate (200 mg) as a yellow oil which was used without further purification.

### Step 2

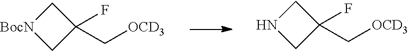

Trifluoroacetic acid (1.00 mL, 13.5 mmol) was added to a solution of tert-butyl 3-fluoro-3-((methoxy-d3)methyl)azetidine-1-carboxylate (200 mg, 0.90 mmol) in dichloromethane (2 mL) and the resulting mixture was stirred at 25° C. for 1 h. The reaction mixture was concentrated in vacuo to afford 3-fluoro-3-((methoxy-d3)methyl)azetidine (200 mg) as a yellow oil which used without further purification.

### 9,9-Difluoro-6-methyl-2,6-diazaspiro[3.5]nonane

### Step 1

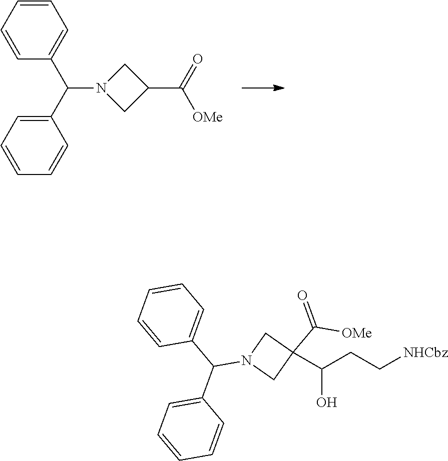 [SMILES: COC(=O)C1CN(C(c2ccccc2)c2ccccc2)C1 ; COC(=O)C1(C(O)CCNC(=O)OCc2ccccc2)CN(C(c2ccccc2)c2ccccc2)C1]

Using a flow reactor, LDA (1 M in THF, 120 mL) was added to a solution of methyl 1-benzhydrylazetidine-3-carboxylate (30.0 g, 107 mmol) in THF (300 mL) at −40° C. A solution of benzyl (3-oxopropyl)carbamate (10.0 g, 48.2 mmol) in THF (100 mL) was then added. The reaction mixture was quenched with saturated aqueous ammonium chloride solution (200 mL) and extracted into EtOAc (3×500 mL). The combined organic extracts were washed with brine (3×300 mL), dried over anhydrous sodium sulfate and then concentrated in vacuo. Flash column chromatography on silica (EtOAc:PE, 0:100 to 50:50) afforded methyl 1-benzhydryl-3-(3-(((benzyloxy)carbonyl)amino)-1-hydroxypropyl)azetidine-3-carboxylate (19.0 g, 33.1 mmol, 69%); δ_{H} (400 MHz; CDCl₃) 9.79 (1H, s), 7.40-7.33 (15H, m), 5.14-5.11 (1H, m), 5.09 (2H, s), 4.14-4.05 (1H, m), 3.79-3.63 (3H, m), 3.41-3.35 (4H, m), 2.76-2.68 (2H, m), 1.67-0.49 (2H, m); ES-MS [M+H]⁺: 489.3.

### Step 2

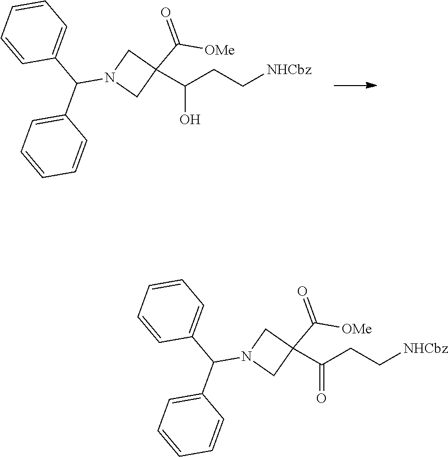 [SMILES: COC(=O)C1(C(O)CCNC(=O)OCc2ccccc2)CN(C(c2ccccc2)c2ccccc2)C1 ; COC(=O)C1(C(=O)CCNC(=O)OCc2ccccc2)CN(C(c2ccccc2)c2ccccc2)C1]

Dess-Martin periodinane (20.0 g, 47.2 mmol) was added to a solution of methyl 1-benzhydryl-3-(3-(((benzyloxy)carbonyl)amino)-1-hydroxypropyl)azetidine-3-carboxylate (10.0 g, 20.5 mmol) in dichloromethane (100 mL). The resulting mixture was stirred at 20° C. for 12 h, then concentrated in vacuo. Flash column chromatography on silica (EtOAc:PE, 0:100 to 40:60) afforded methyl 1-benzhydryl-3-(3-(((benzyloxy)carbonyl)amino)propanoyl)azetidine-3-carboxylate (6.0 g, 9.25 mmol, 45%) as a yellow oil; 6_{H} (400 MHz; CDCl₃) 7.47-7.27 (12H, m), 7.26-7.16 (3H, m), 5.09 (2H, s), 4.38 (1H, s), 3.74 (3H, s), 3.57-3.45 (6H, m), 2.86-2.77 (2H, m).

### Step 3

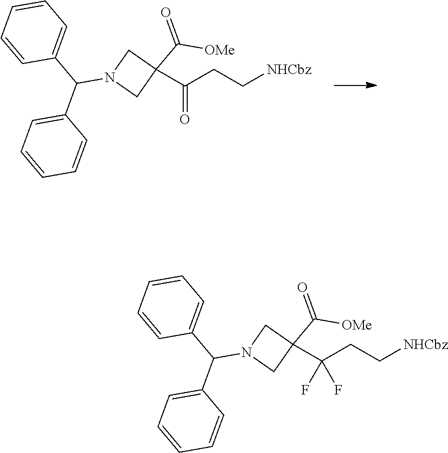 [SMILES: COC(=O)C1(C(=O)CCNC(=O)OCc2ccccc2)CN(C(c2ccccc2)c2ccccc2)C1 ; COC(=O)C1(C(F)(F)CCNC(=O)OCc2ccccc2)CN(C(c2ccccc2)c2ccccc2)C1]

(Diethylamino)sulfur trifluoride (4 mL, 30.3 mmol) was added to a solution of methyl 1-benzhydryl-3-(3-(((benzyloxy)carbonyl)amino)propanoyl)azetidine-3-carboxylate (5.80 g, 11.9 mmol) in dichloromethane (50 mL) at 0° C. and the resulting mixture was stirred at 20° C. for 12 h. The reaction was quenched with saturated sodium bicarbonate solution (100 mL) and extracted into EtOAc (3×500 mL). The combined organic extracts were washed with brine (100 mL), dried over anhydrous sodium sulfate and then concentrated in vacuo. Flash column chromatography on silica (EtOAc:PE, 0:100 to 40:60) afforded methyl 1-benzhydryl-3-(3-(((benzyloxy)carbonyl)amino)-1,1-difluoropropyl)azetidine-3-carboxylate (2.30 g, 1.99 mmol, 17%) as a yellow oil; δ_{H} (400 MHz; CDCl₃) 7.47-7.28 (12H, m), 7.25-7.16 (3H, m), 5.18-5.05 (2H, m), 5.05-4.91 (1H, m), 4.46-4.34 (1H, m), 3.89-3.76 (3H, m), 3.65-3.54 (2H, m), 3.52-3.42 (2H, m), 3.35 (2H, m), 2.23-2.02 (2H, m); ES-MS [M+H]⁺: 509.2.

### Step 4

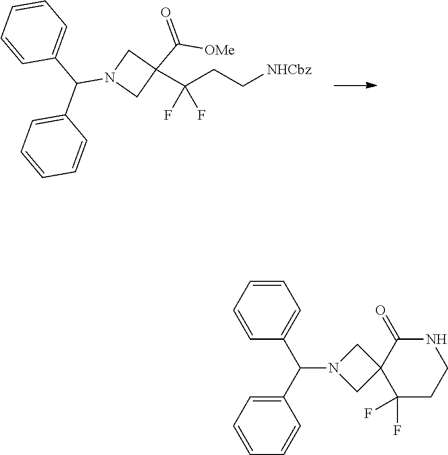 [SMILES: COC(=O)C1(C(F)(F)CCNC(=O)OCc2ccccc2)CN(C(c2ccccc2)c2ccccc2)C1 ; O=C1NCCC(F)(F)C12CN(C(c1ccccc1)c1ccccc1)C2]

Trifluoroacetic acid (4.40 mL, 59.2 mmol) was added to a solution of methyl 1-benzhydryl-3-(3-(((benzyloxy)carbonyl)amino)-1,1-difluoropropyl)azetidine-3-carboxylate (2.20 g, 4.33 mmol) in dichloromethane (30 mL) and the resulting mixture was stirred at 60° C. for 12 h. A solution of triethylamine (13.2 mL, 94.8 mmol) in dichloromethane (30 mL) was then added at 40° C. and stirring was continued at this temperature for 4 h. The reaction mixture was concentrated in vacuo and flash column chromatography on silica (EtOAc:PE, 0:100 to 100:0) afforded 2-benzhydryl-9,9-difluoro-2,6-diazaspiro[3.5]nonan-5-one (0.70 g, 1.94 mmol), 45%) as a yellow oil; ES-MS [M+H]⁺: 343.2.

### Step 5

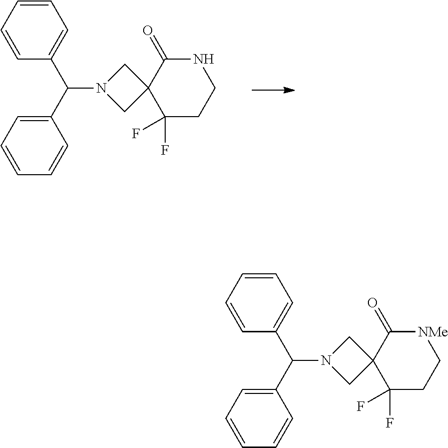 [SMILES: O=C1NCCC(F)(F)C12CN(C(c1ccccc1)c1ccccc1)C2 ; CN1CCC(F)(F)C2(CN(C(c3ccccc3)c3ccccc3)C2)C1=O]

Sodium hydride (60% dispersion in oil, 70 mg, 1.75 mmol) was added at 0° C. under nitrogen to a solution of 2-benzhydryl-9,9-difluoro-2,6-diazaspiro[3.5]nonan-5-one (350 mg, 1.02 mmol) in DMF (5 mL). The resulting mixture was stirred at 25° C. for 30 min. Iodomethane (123 μL, 1.97 mmol) was added at 0° C. and stirring was continued for another 2 h. The reaction was quenched with water (20 mL) and extracted into EtOAc (2×40 mL). The combined organic extracts were washed with brine (200 mL), dried over anhydrous sodium sulfate and then concentrated in vacuo. Flash column chromatography on silica (EtOAc:PE, 0:100 to 60:40) afforded 2-benzhydryl-9,9-difluoro-6-methyl-2,6-diazaspiro[3.5]nonan-5-one (300 mg, 0.92 mmol, 90%) as a yellow oil; δ_{H} (400 MHz; CDCl₃) 7.51-7.44 (4H, m), 7.31-7.26 (4H, m), 7.21-7.14 (2H, m), 4.72-4.62 (1H, m), 3.59-3.50 (2H, m), 3.49-3.42 (2H, m), 3.36-3.24 (2H, m), 2.97 (3H, s), 2.29-2.16 (2H, m). ES-MS [M+H]⁺: 357.1.

### Step 6

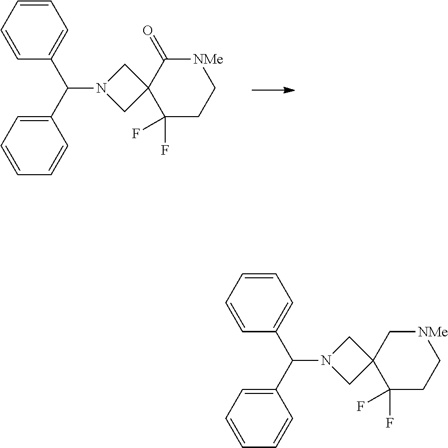 [SMILES: CN1CCC(F)(F)C2(CN(C(c3ccccc3)c3ccccc3)C2)C1=O ; CN1CCC(F)(F)C2(C1)CN(C(c1ccccc1)c1ccccc1)C2]

BH₃·THF (1.0 M, 3.0 mL, 3 mmol) was added to a solution of 2-benzhydryl-9,9-difluoro-6-methyl-2,6-diazaspiro[3.5]nonan-5-one (300 mg, 0.84 mmol) in THF (10 mL) and the resulting mixture was stirred at 60° C. for 2 h. The reaction was quenched carefully with methanol (10 mL) and extracted concentrated in vacuo. Flash column chromatography on silica (EtOAc:PE, 0:100 to 100:0) afforded 2-benzhydryl-9,9-difluoro-6-methyl-2,6-diazaspiro[3.5]nonane (250 mg, 0.37 mmol, 43%) as a yellow oil. ES-MS [M+H]⁺: 343.2.

### Step 7

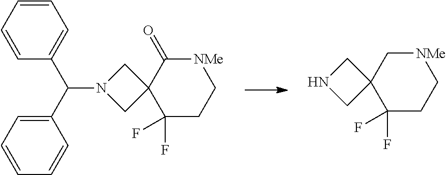 [SMILES: CN1CCC(F)(F)C2(CN(C(c3ccccc3)c3ccccc3)C2)C1=O ; CN1CCC(F)(F)C2(CNC2)C1]

A mixture of 2-benzhydryl-9,9-difluoro-6-methyl-2,6-diazaspiro[3.5]nonane (220 mg, 0.64 mmol), 15% Pd/C (1.0 g, 1.41 mmol) and TFA (53 μL, 0.71 mmol) in ethanol (5 mL) was degassed and purged with hydrogen three times, and then stirred at 35° C. for 12 h under a hydrogen atmosphere. Ethanol (20 mL) was added and the mixture was filtered. The filtrate was concentrated in vacuo to afford 9,9-difluoro-6-methyl-2,6-diazaspiro[3.5]nonane as a yellow gum that was used without further purification; ES-MS [M+H]⁺: 177.2.

### 3-(Ethoxymethyl)-3-fluoroazetidine

### Step 1

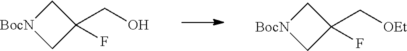 [SMILES: CC(C)(C)OC(=O)N1CC(F)(CO)C1 ; CCOCC1(F)CN(C(=O)OC(C)(C)C)C1]

A mixture of tert-butyl 3-hydroxy-3-methylazetidine-1-carboxylate (2.00 g, 9.75 mmol) and iodoethane (1.56 mL, 19.5 mmol) in THF (20 mL) was degassed and purged with nitrogen three times. Sodium hydride (60% dispersion in oil, 585 mg, 14.6 mmol) was added at 0° C. under nitrogen and the resulting mixture was stirred at 20° C. for 12 h. The reaction mixture was quenched with saturated aqueous ammonium chloride solution (20 mL) and extracted into ethyl acetate (3×30 mL). The combined organic extracts were dried over anhydrous sodium sulfate and then concentrated in vacuo to afford tert-butyl 3-(ethoxymethyl)-3-fluoroazetidine-1-carboxylate (2.40 g, 9.26 mmol, 95% yield) as a white solid; δ_{H} (400 MHz; DMSO-d6) 4.04-3.80 (4H, m), 3.75-3.62 (2H, m), 3.52 (2H, m), 1.38 (9H, s), 1.12 (3H, m); ES-MS [M-Boc+H]⁺: 134.3.

### Step 2

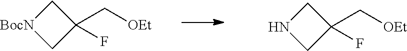 [SMILES: CCOCC1(F)CN(C(=O)OC(C)(C)C)C1 ; CCOCC1(F)CNC1]

HCl (4.0 M in dioxane, 1 mL, 13.5 mmol) was added to a solution of tert-butyl tert-butyl 3-(ethoxymethyl)-3-fluoroazetidine-1-carboxylate (100 mg, 0.43 mmol) in dichloromethane (2 mL) and the resulting mixture was stirred at 20° C. for 2 h. The reaction mixture was concentrated in vacuo to afford 3-(ethoxymethyl)-3-fluoroazetidine (60 mg) as a yellow oil which used without further purification; ES-MS [M+H]⁺: 134.2.

### 3-(Methoxy-d3)azetidine

### Step 1

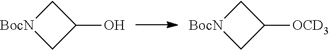

tert-Butyl 3-hydroxyazetidine-1-carboxylate (500 mg, 2.89 mmol) was added to a mixture of sodium hydride (60% dispersion in oil, 254 mg, 6.35 mmol) in THF (10 mL) at 0° C. The resulting mixture was stirred at 0° C. for 30 min. Iodomethane-d3 (395 μL, 6.35 mmol) was added at 0° C. and the mixture was stirred at 25° C. for another 1.5 h. The reaction mixture was quenched with saturated aqueous ammonium chloride solution (10 mL) and extracted into EtOAc (2×10 mL). The combined organic extracts were washed with brine (10 mL), dried over anhydrous sodium sulfate and then concentrated in vacuo to afford tert-butyl 3-(methoxy-d3)azetidine-1-carboxylate (550 mg) as a yellow oil which was used without further purification; δ_{H} (400 MHz; DMSO-d6) 4.16-4.08 (1H, m), 4.01-3.93 (2H, m), 3.63 (2H, m), 1.37 (9H, s).

### Step 2

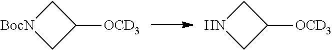

Trifluoroacetic acid (2.00 mL, 27.0 mmol) was added to a solution of tert-butyl 3-(methoxy-d3)azetidine-1-carboxylate (550 mg, 2.90 mmol) in dichloromethane (6 mL) and the resulting mixture was stirred at 25° C. for 2 h. The reaction mixture was concentrated in vacuo to afford 3-(methoxy-d3)azetidine TFA salt (260 mg) as a yellow oil which used without further purification.

### 7-Bromo-6-fluorobenzo[d]thiazol-2-amine

### Step 1

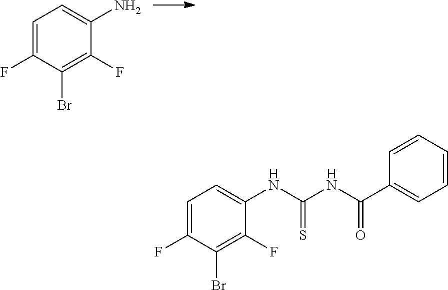 [SMILES: Nc1ccc(F)c(Br)c1F ; O=C(NC(=S)Nc1ccc(F)c(Br)c1F)c1ccccc1]

A solution of benzoyl isothiocyanate (43.2 g, 264 mmol) in acetonitrile (500 mL) was slowly added to a solution of 3-bromo-2,4-difluoroaniline (50.0 g, 240 mmol) in acetonitrile (1 L) and the resulting mixture was stirred at 20° C. for 2 h. The solid was filtered and dried in vacuo to afford N-((3-bromo-2,4-difluorophenyl)carbamothioyl)benzamide (80.0 g. 216 mmol. 90%) as a light yellow solid which used without further purification; δ_{H} (400 MHz; DMSO-d6) 12.32 (1H, s), 11.92 (1H, s), 8.04 (2H, d, J 7.4 Hz), 7.86 (1H, m), 7.76-7.67 (1H, m), 7.65-7.55 (2H, m), 7.39 (1H, m).

### Step 2

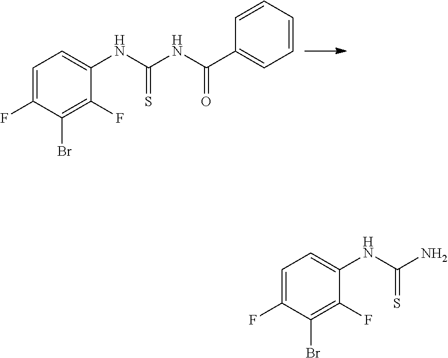 [SMILES: O=C(NC(=S)Nc1ccc(F)c(Br)c1F)c1ccccc1 ; NC(=S)Nc1ccc(F)c(Br)c1F]

Aqueous sodium hydroxide (2.0 M, 161 mL, 3.23 mmol) was added to a solution of N-((3-bromo-2,4-difluorophenyl)carbamothioyl)benzamide (80.0 g, 216 mmol) in methanol (800 mL) and the resulting mixture was stirred at 20° C. for 12 h. Aqueous hydrochloric acid (1.0 M) was added until the pH was ˜5-6. The resulting solid was filtered, washed with water (100 mL) and dried in vacuo to afford 1-(3-bromo-2,4-difluorophenyl)thiourea (50.0 g, 162 mmol, 75%) as a light yellow solid which used without further purification; δ_{H} (400 MHz; DMSO-d6) 9.41 (1H, s), 8.19-7.71 (1H, m), 7.56 (1H, m), 7.26 (1H, m).

### Step 3

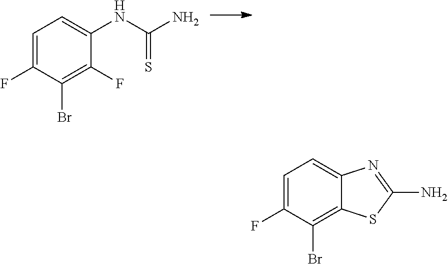 [SMILES: NC(=S)Nc1ccc(F)c(Br)c1F ; Nc1nc2ccc(F)c(Br)c2s1]

Sodium hydride (60% dispersion in oil, 11.2 g, 281 mmol) was added portion wise to a solution of 1-(3-bromo-2,4-difluorophenyl)thiourea (50.0 g, 187 mmol) in NMP (500 mL) at 0° C. The resulting mixture was stirred at 20° C. for 30 min and then 80° C. for 12 h. The reaction mixture was cooled to room temperature and poured slowly into saturated aqueous ammonium chloride solution (1 L). Water was added (1 L) and the mixture was extracted into ethyl acetate (3×500 mL). The combined organic extracts were washed with brine (3×500 mL), dried over anhydrous sodium sulfate and concentrated in vacuo. Trituration with dichloromethane afforded 7-bromo-6-fluorobenzo[d]thiazol-2-amine (40.0 g, 155 mmol, 83%) as a brown solid; δ_{H} (400 MHz; DMSO-d6) 7.74 (2H, s), 7.35 (1H, m), 7.30-7.21 (1H, m); ES-MS [M+H]⁺: 248.9.

The following intermediates were prepared in an analogous manner:

| Name | Structure | Starting material | Analytical data |
|---|---|---|---|
| 7-Bromo-6- chlorobenzo[d]thiazol-2- amine | 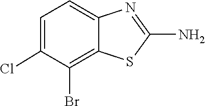 [SMILES: Nc1nc2ccc(Cl)c(Br)c2s1] | 3-bromo-4-chloro-2- fluoroaniline | ES-MS [M + H]⁺: 264.9 |
| 7-bromo-5- fluorobenzo[d]thiazol-2- amine | 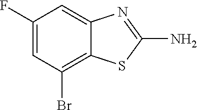 [SMILES: Nc1nc2cc(F)cc(Br)c2s1] | 3-bromo-2,5- difluoroaniline | ES-MS [M + H]⁺: 248.9 |
| 4-chlorothiazolo[5,4- c]pyridin-2-amine | 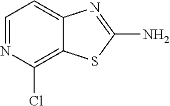 [SMILES: Nc1nc2ccnc(Cl)c2s1] | 3-bromo-2- chloropyridin-4-amine | ES-MS [M + H]⁺: 186.2 |
| 6-bromo-7-(6- methoxypyridin-3- yl)benzo[d]thiazol-2-amine | 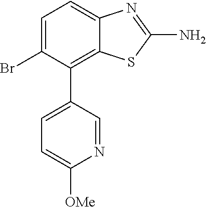 [SMILES: COc1ccc(-c2c(Br)ccc3nc(N)sc23)cn1] | 4-bromo-2-fluoro-3-(6- methoxypyridin-3- yl)aniline | ES-MS [M + H]⁺: 336.0 |

### 4-Bromo-2-fluoro-3-(6-methoxypyridin-3-yl)aniline

### Step 1

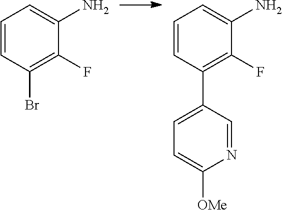 [SMILES: Nc1cccc(Br)c1F ; COc1ccc(-c2cccc(N)c2F)cn1]

Pd(dppf)Cl₂ (2.89 g, 3.95 mmol) was added to a solution of 3-bromo-2-fluoroaniline (15.0 g, 78.9 mmol), (6-methoxypyridin-3-yl)boronic acid (18.1 g, 118 mmol) and potassium carbonate (21.8 g, 158 mmol) in dioxane (150 mL) and water (30 mL). The resulting mixture was heated at 80° C. for 16 h. Water (150 mL) was added and the mixture was extracted into ethyl acetate (3×150 mL). The combined organic extracts were washed with brine (3×150 mL), dried over anhydrous sodium sulfate and concentrated in vacuo. Flash column chromatography on silica (EtOAc:PE, 0:100 to 25:75) afforded 2-fluoro-3-(6-methoxypyridin-3-yl)aniline (16.8 g, 77.0 mmol, 98%) as a yellow solid; ES-MS [M+H]⁺: 219.4.

### Step 2

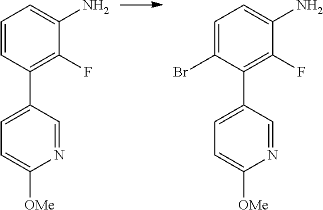 [SMILES: COc1ccc(-c2cccc(N)c2F)cn1 ; COc1ccc(-c2c(Br)ccc(N)c2F)cn1]

NBS (13.7 g, 77.0 mmol) was added to a solution of 2-fluoro-3-(6-methoxypyridin-3-yl)aniline (16.8 g, 77.0 mmol) in DMF (168 mL). The resulting mixture was stirred at 25° C. for 2 h. Water (150 mL) was added and the mixture was extracted into ethyl acetate (3×150 mL). The combined organic extracts were washed with brine (150 mL), dried over anhydrous sodium sulfate and concentrated in vacuo. Flash column chromatography on silica (EtOAc:PE, 0:100 to 15:85) afforded 4-bromo-2-fluoro-3-(6-methoxypyridin-3-yl)aniline (21.5 g, 72.4 mmol, 94%) as a yellow solid; ES-MS [M+H]⁺: 297.2.

### 2-Amino-7-(6-methoxypyridin-3-yl)benzo[d]thiazole-6-carbonitrile

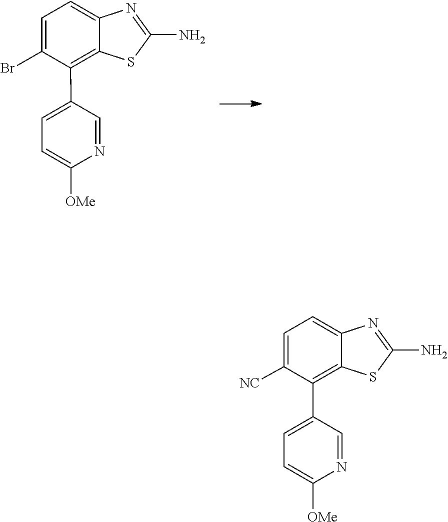 [SMILES: COc1ccc(-c2c(Br)ccc3nc(N)sc23)cn1 ; COc1ccc(-c2c(C#N)ccc3nc(N)sc23)cn1]

Tetrakis(triphenylphosphine)palladium(0) (172 mg, 0.15 mmol) was added to a mixture of 6-bromo-7-(6-methoxypyridin-3-yl)benzo[d]thiazol-2-amine (500 mg, 1.49 mmol), zinc cyanide (349 mg, 2.97 mmol) and zinc (19 mg, 0.30 mmol) in DMF (5 mL). The resulting mixture was stirred at 120° C. for 16 h. Water (5 mL) was added and the mixture was extracted into ethyl acetate (3×5 mL). The combined organic extracts were washed with brine (5 mL), dried over anhydrous sodium sulfate and concentrated in vacuo. Trituration (1:1 EtOAc:PE) afforded 2-amino-7-(6-methoxypyridin-3-yl)benzo[d]thiazole-6-carbonitrile (210 mg, 0.74 mmol, 50%) as a yellow solid; ES-MS [M+H]⁺: 283.1.

### 7-(6-Methoxypyridin-3-yl)-6-(methylsulfonyl)benzo[d]thiazol-2-amine

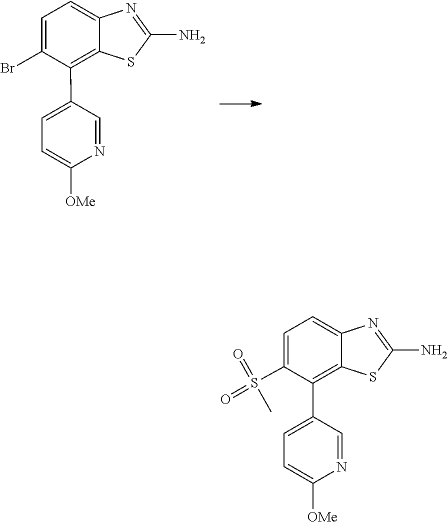 [SMILES: COc1ccc(-c2c(Br)ccc3nc(N)sc23)cn1 ; COc1ccc(-c2c(S(C)(=O)=O)ccc3nc(N)sc23)cn1]

DMEDA (262 mg, 2.97 mmol) and copper(II) triflate (538 mg, 1.49 mmol) were added to a solution of 6-bromo-7-(6-methoxypyridin-3-yl)benzo[d]thiazol-2-amine (500 mg, 1.49 mmol) and sodium methylsulfinate (304 mg, 2.97 mmol) in DMSO (10 mL). The resulting mixture was heated at 120° C. for 16 h. Water (10 mL) was added and the mixture was extracted into ethyl acetate (3×10 mL). The combined organic extracts were washed with brine (10 mL), dried over anhydrous sodium sulfate and concentrated in vacuo. Purification by flash column chromatography on silica (EtOAc:PE, 0:100 to 95:5) afforded 7-(6-methoxypyridin-3-yl)-6-(methylsulfonyl)benzo[d]thiazol-2-amine (100 mg, 298 μmol, 20%) as a yellow solid; ES-MS [M+H]⁺: 336.1.

### 7-bromo-6-methoxybenzo[d]thiazol-2-amine

### Step 1

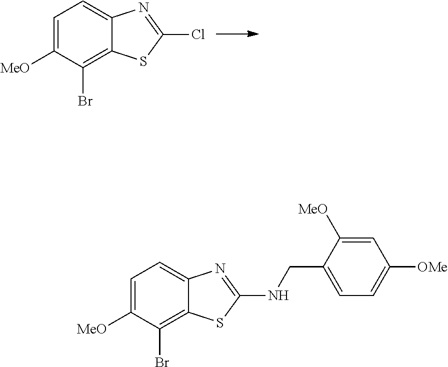 [SMILES: COc1ccc2nc(Cl)sc2c1Br ; COc1ccc(CNc2nc3ccc(OC)c(Br)c3s2)c(OC)c1]

DIPEA (3.38 mL. 19.4 mmol) was added to a solution of 2,4-dimethoxybenzylamine (1.94 mL, 12.9 mmol) and 7-bromo-2-chloro-6-methoxybenzo[d]thiazole (1.8 g, 6.46 mmol) in dioxane (18 mL) at 25° C. and the resulting mixture was stirred at 120° C. for 16 h. The reaction mixture was concentrated in vacuo and triturated with ethyl acetate (15 mL) to afford 7-bromo-N-(2,4-dimethoxybenzyl)-6-methoxybenzo[d]thiazol-2-amine (2.60 g, 5.89 mmol, 91%) as a white solid; ES-MS [M+H]⁺: 409.0.

### Step 2

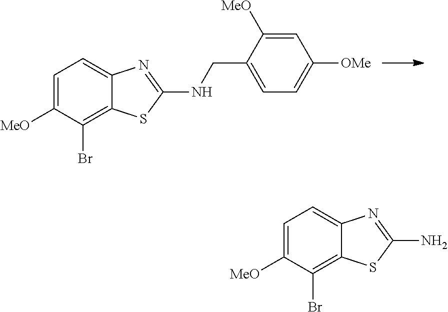 [SMILES: COc1ccc(CNc2nc3ccc(OC)c(Br)c3s2)c(OC)c1 ; COc1ccc2nc(N)sc2c1Br]

A mixture of 7-bromo-N-(2,4-dimethoxybenzyl)-6-methoxybenzo[d]thiazol-2-amine (250 mg, 0.61 mmol) in TFA (10 mL) was stirred at 25° C. for 16 h, then saturated sodium carbonate aqueous solution was slowly added until the pH was ˜8. The mixture was extracted into ethyl acetate (3×500 mL) and the combined organic extracts were dried over anhydrous sodium sulfate then concentrated in vacuo. Trituration with ethyl acetate (3 mL) afforded 7-bromo-6-methoxybenzo[d]thiazol-2-amine (100 mg, 374 mmol, 61%) as a yellow solid; δ_{H} (400 MHz; DMSO-d6) 7.50-7.40 (2H, m), 7.32-7.22 (1H, m), 7.04-6.94 (1H, m), 3.82 (3H, s); ES-MS [M+H]⁺: 258.9.

### 7-bromo-6-methylbenzo[d]thiazol-2-amine

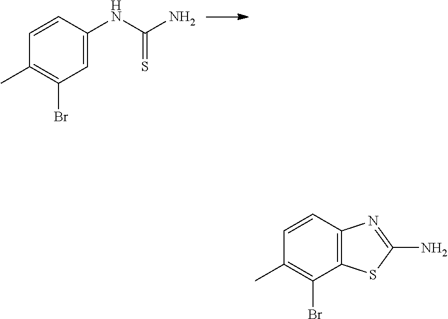

Bromine (462 μL, 8.97 mmol) was added to a solution of 1-(3-bromo-4-methylphenyl)thiourea* (2.0 g, 8.16 mmol) in acetic acid (15 mL) at 0° C. The resulting mixture was warmed to 20° C. then stirred at 80° C. for 16 h. The reaction mixture was concentrated in vacuo, and triturated with ethyl acetate (10 mL). The solid was purified by preparative HPLC (column: Phenomenex Luna C18 150*40 15 μm; acetonitrile:[water (0.1% TFA)]; gradient: 10%-40% water (0.1% TFA) over 12 min; flow rate 60 mL/min) to afford 7-bromo-6-methylbenzo[d]thiazol-2-amine (260 mg, 1.03 mmol) as a white solid; δ_{H} (400 MHz; DMSO-d6) 8.07-7.82 (2H, m), 7.28-7.19 (2H, m), 2.36 (3H, s); ES-MS [M+H]⁺: 244.9. *prepared in an analogous manner to 1-(3-bromo-2,4-difluorophenyl)thiourea from 3-bromo-4-methylaniline

The following intermediates were prepared in an analogous manner:

| Name | Structure | Starting material | Analytical data |
|---|---|---|---|
| 7-bromo-6-(trifluoro- methyl)benzo[d] thiazol-2-amine | 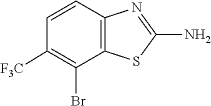 [SMILES: Nc1nc2ccc(C(F)(F)F)c(Br)c2s1] | 1-(3-bromo-4-(trifluoro- methyl)phenyl)thiourea (prepared from 3-bromo- 4-trifluoromethyl)aniline) | ES-MS [M + H]⁺: 297.2 |
| 7-bromo-5-methyl- benzo[d]thiazol-2- amine | 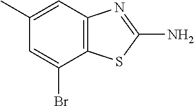 | 1-(3-bromo-5-methyl- phenyl)thiourea (pre- pared from 3-bromo-5- methylaniline) | δ_{H}(400 MHz; CDCl₃) 9.74-8.68 (2H, m), 7.32 (1H, m), 7.26 (1H, s), 2.43 (3H, s) |
| 7-fluoro-4-nitro- benzo[d]thiazol-2- amine | 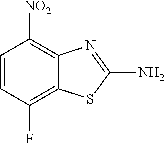 [SMILES: Nc1nc2c([N+](=O)[O-])ccc(F)c2s1] | 1-(5-fluoro-2-nitrophen- yl)thiourea (prepared from 5-fluoro-2-nitro- aniline) | ES-MS [M + H]⁺: 214.2 |
| 7-bromo-4,6- difluorobenzo [d]thiazol-2- amine | 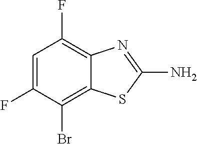 [SMILES: Nc1nc2c(F)cc(F)c(Br)c2s1] | 5-bromo-2,4-difluoro- aniline | ES-MS [M + H]⁺: 371.0 |
| 6,7-difluoro-4-nitro- benzo[d]thiazol-2- amine | 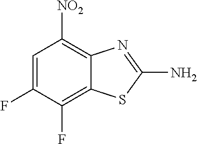 [SMILES: Nc1nc2c([N+](=O)[O-])cc(F)c(F)c2s1] | 4,5-difluoro-2-nitro- aniline | ES-MS [M + H]⁺: 232.1 |

### N-(tetrahydro-2H-pyran-4-yl)-5-(4,4,5,5-tetramethyl-1,3,2-dioxaborolan-2-yl)pyridin-2-amine

### Step 1

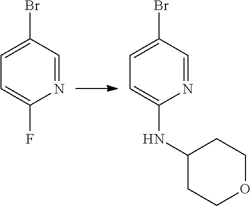 [SMILES: Fc1ccc(Br)cn1 ; Brc1ccc(NC2CCOCC2)nc1]

4-Aminotetrahydropyran (4.31 g, 42.6 mmol) was added to a solution of DIPEA (14.9 mL, 85.2 mmol) and 5-bromo-2-fluoropyridine (2.92 mL, 28.4 mmol) in DMSO (50 mL). The resulting mixture was stirred at 100° C. for 12 h. Water was added (30 mL) and the mixture was extracted into ethyl acetate (3×30 mL). The combined organic extracts were washed with brine (2×20 mL), dried over anhydrous sodium sulfate and concentrated in vacuo to afford 5-bromo-N-(tetrahydro-2H-pyran-4-yl)pyridin-2-amine (4.00 g, 15.6 mmol, 55%) as a white solid; δ_{H} (400 MHz; DMSO-d6) 8.00 (1H, d, J 2.40 Hz), 7.48 (1H, m), 6.77 (1H, m), 6.47 (1H, d, J 8.80 Hz), 3.90-3.79 (3H, m), 3.42-3.35 (2H, m), 1.84 (2H, m), 1.47-1.32 (2H, m). ES-MS [M+H]⁺: 259.1.

### Step 2

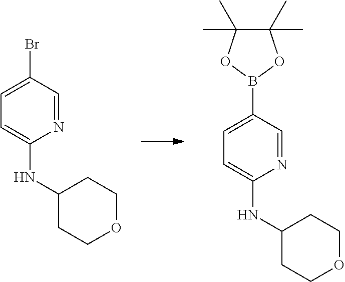 [SMILES: Brc1ccc(NC2CCOCC2)nc1 ; CC1(C)OB(c2ccc(NC3CCOCC3)nc2)OC1(C)C]

Pd(dppf)Cl₂ (285 mg, 0.39 mmol) was added to a solution of bromo-N (tetrahydro-2H-pyran-4-yl)pyridin-2-amine (1.00 g, 3.89 mmol), bis(pinacolato)diboron (1.48 g, 5.83 mmol) and potassium acetate (1.15 g, 11.7 mmol) in dioxane (10 mL). The resulting mixture was stirred at 80° C. for 4 h, then concentrated in vacuo. The mixture was diluted with ethyl acetate (50 mL) and filtered. The filter cake was washed with ethyl acetate (2×30 mL) and the filtrate was concentrated in vacuo to afford N-(tetrahydro-2H-pyran-4-yl)-5-(4,4,5,5-tetramethyl-1,3,2-dioxaborolan-2-yl)pyridin-2-amine (1.00 g. 2.63 mmol, 68%) as a white solid; δ_{H} (400 MHz; DMSO-d6) 8.20 (1H, m), 7.50 (1H, m), 6.93 (1H, m), 6.48-6.40 (1H, m), 3.89-3.80 (2H, m), 3.44-3.34 (2H, m), 1.89-1.78 (3H, m), 1.46-1.37 (2H, m), 1.24 (12H, s). ES-MS [M+H]⁺: 305.2.

The following intermediates were prepared in an analogous manner:

| Name | Structure | Starting material | Analytical data |
|---|---|---|---|
| N-methyl-N-(tetrahydro-2H- pyran-4-yl)-5-(4,4,5,5-tetra- methyl-1,3,2-dioxaborolan- 2-yl)pyridin-2-amine | 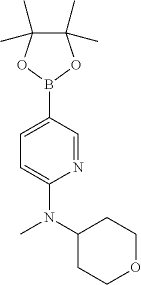 [SMILES: CN(c1ccc(B2OC(C)(C)C(C)(C)O2)cn1)C1CCOCC1] | N-methyltetrahydro- pyran-4-amine | δ_{H}(400 MHz; DMSO-d6) 8.30 (1H, d, J 1.38 Hz), 7.67 (1H, m), 6.63 (1H, d, J 8.75 Hz), 4.80-4.67 (1H, m), 3.92 (2H, m), 3.49- 3.46 (2H, m), 2.85 (3H, s), 1.85-1.69 (2H, m), 1.48 (2H, m); 1.26 (12H, s) |
| N-(4-methyltetrahydro-2H- pyran-4-yl)-5-(4,4,5,5-tetra- methyl-1,3,2-dioxaborolan- 2-yl)pyridin-2-amine | 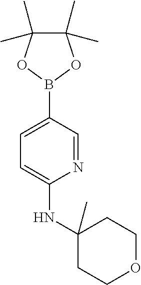 [SMILES: CC1(Nc2ccc(B3OC(C)(C)C(C)(C)O3)cn2)CCOCC1] | 4-methyloxan-4- amine | δ_{H}(400 MHz; DMSO-d6) 8.17 (1H, m), 7.48 (1H, m), 6.56-6.51 (2H, m), 3.57-3.54 (4H, m), 2.20 (2H, m), 1.61-1.54 (2H, m), 1.41 (3H, s), 1.24 (12H, s) |

### 2,2-difluoro-2-(5-(4,4,5,5-tetramethyl-1,3,2-dioxaborolan-2-yl)pyridin-2-yl)ethan-1-ol

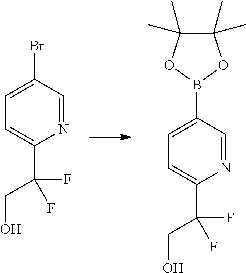

Pd(dppf)Cl₂ (31 mg, 0.04 mol) was added to a solution of 2-(5-bromopyridin-2-yl)-2,2-difluoroethan-1-ol (100 mg, 0.42 mmol), bis(pinacolato)diboron (213 mg, 0.84 mmol) and potassium acetate (124 mg, 1.26 mmol) in dioxane (2 mL). The resulting mixture was stirred at 80° C. for 4 h, concentrated in vacuo and purified by flash column chromatography on silica (EtOAc:PE, 0:100 to 50:50) to afford 2,2-difluoro-2-(5-(4,4,5,5-tetramethyl-1,3,2-dioxaborolan-2-yl)pyridin-2-yl)ethan-1-ol (80 mg, 224 μmol, 53%) as a yellow oil; ES-MS [M+H]⁺: 204.0 (boronic acid).

The following intermediates were prepared in an analogous manner:

| Name | Structure | Starting material | Analytical data |
|---|---|---|---|
| N-(oxetan-3-y1)-5- (4,4,5,5-tetramethyl- 1,3,2-dioxaborolan- 2-yl)picolinamide | 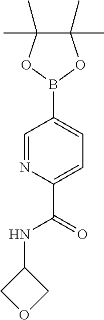 [SMILES: CC1(C)OB(c2ccc(C(=O)NC3COC3)nc2)OC1(C)C] | 5-bromo-N-(oxetan-3-yl)- pyridine-2-carboxamide | δ_{H}(400 MHz; DMSO-d6) 9.57 (1H, d, J 6.75 Hz), 8.82 (1H, s), 8.20 (1H, m), 8.03 (1H, d, J 7.63 Hz), 5.11-4.98 (1H, m), 4.76-4.64 (4H, m), 1.33 (12H, s) |
| 2-cyclopropoxy-5- (4,4,5,5-tetramethyl- 1,3,2-dioxaborolan- 2-yl)pyrimidine | 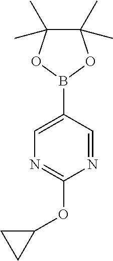 [SMILES: CC1(C)OB(c2cnc(OC3CC3)nc2)OC1(C)C] | 5-bromo-2-(cyclopropyl- oxy)pyrimidine | δ_{H}(400 MHz; CDCl₃) 8.83 (2H, m), 4.40 (1H, m), 1.36 (12H, s), 0.84 (1H, s) |
| 2-(2-methoxyethoxy)- 5-(4,4,5,5-tetramethyl- 1,3,2-dioxaborolan- 2-yl)pyrimidine | 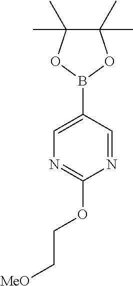 [SMILES: COCCOc1ncc(B2OC(C)(C)C(C)(C)O2)cn1] | 5-bromo-2-(2-methoxy- ethoxy)pyrimidine | ES-MS [M + H]⁺: 281.2 |
| 4-(2-((5-(4,4,5,5- tetramethyl-1,3,2- dioxaborolan-2-yl)- pyrimidin-2-yl)oxy)- ethyl)morpholine | 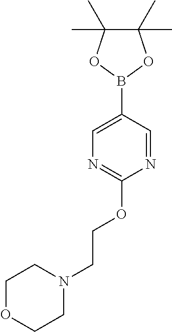 | 4-(2-((5-bromopyrimidin- 2-yl)oxy)-ethyl)morpholine | ES-MS [M + H]⁺: 254.1 (boronic acid) |
| morpholino(5- (4,4,5,5-tetrameth- yl-1,3,2-dioxaboro- lan-2-yl)pyrimidin- 2-yl)methanone | 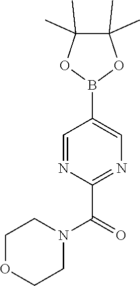 | (5-bromopyrimidin-2-yl)- (morpholino)methanone | ES-MS [M + H]⁺: 238.2 (boronic acid) |
| 2-((tetrahydro-2H- pyran-4-yl)oxy)-5- (4,4,5,5-tetramethyl- 1,3,2-dioxaborolan- 2-yl)pyrimidine | 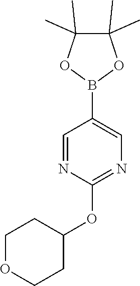 | 5-bromo-2-(tetrahydro- 2h-pyran-4-yloxy)pyrim- idine | ES-MS [M + H]⁺: 225.2 (boronic acid) |

### 2-Methyl-N-(oxetan-3-yl)-4-(4,4,5,5-tetramethyl-1,3,2-dioxaborolan-2-yl)benzamide

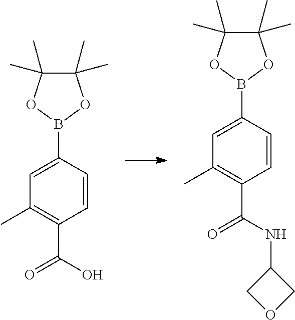

3-Aminooxetane (84 mg, 1.14 mmol) was added to a solution of 2-methyl-4-(4,4,5,5-tetramethyl-1,3,2-dioxaborolan-2-yl)benzoic acid (200 mg, 0.76 mmol), DIPEA (399 μL, 2.29 mmol) and HATU (348 mg, 0.97 mmol) in DMF (3 mL) and the resulting mixture was stirred at 25° C. for 1 h. Water was added (10 mL) and the mixture was extracted into ethyl acetate (3×10 mL). The combined organic extracts were washed with brine (2×20 mL), dried over anhydrous sodium sulfate and concentrated in vacuo. Flash column chromatography on silica (EtOAc:PE, 0:100 to 60:40) afforded 2-methyl-N-(oxetan-3-yl)-4-(4,4,5,5-tetramethyl-1,3,2-dioxaborolan-2-yl)benzamide (160 mg, 0.50 mmol, 65%) as a yellow oil; δ_{H} (400 MHz; CDCl₃) 7.71-7.62 (2H, m), 7.40-7.34 (1H, m), 6.41-6.30 (1H, m), 5.29-5.18 (1H, m), 5.07-4.97 (2H, m), 4.63-4.55 (2H, m), 2.44 (3H, s), 1.36 (12H, s); ES-MS [M+H]⁺: 318.0.

The following intermediate was prepared in an analogous manner:

| Name | Structure | Starting material | Analytical data |
|---|---|---|---|
| N-(oxetan-3-yl)-4- (4,4,5,5-tetramethyl- 1,3,2-dioxaborolan- 2-yl)benzamide | 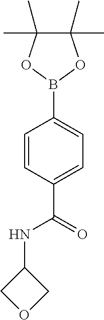 [SMILES: CC1(C)OB(c2ccc(C(=O)NC3COC3)cc2)OC1(C)C] | 4-(4,4,5,5-tetramethyl- 1,3,2-dioxaborolan-2- yl)benzoic acid | δ_{H}(400 MHz; DMSO-d6) 9.22 (1H, d, J 6.4 Hz), 7.94 (2H, d, J 8.1 Hz), 7.80 (2H, d, J 8.1 Hz), 5.12-4.99 (1H, m), 4.81 (2H, m), 4.64 (2H, m), 1.35 (12H, s) |

### (R)—N-(Tetrahydrofuran-3-yl)-5-(4,4,5,5-tetramethyl-1,3,2-dioxaborolan-2-yl)pyridin-2-amine

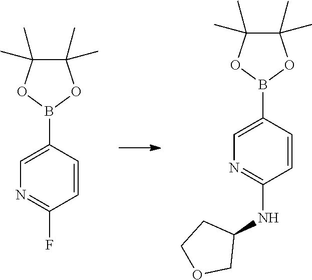

(R)-3-Aminotetrahydrofuran (0.59 g, 6.72 mmol) was added to a solution of DIPEA (2.34 mL, 13.5 mmol) and 2-fluoro-5-(4,4,5,5-tetramethyl-1,3,2-dioxaborolan-2-yl)pyridine (1.00 g, 4.48 mmol) in DMSO (10 mL). The resulting mixture was stirred at 100° C. for 12 h. Water was added (30 mL) and the mixture was extracted into ethyl acetate (3×30 mL). The combined organic extracts were washed with brine (2×20 mL), dried over anhydrous sodium sulfate and concentrated in vacuo to afford (R)—N-(tetrahydrofuran-3-yl)-5-(4,4,5,5-tetramethyl-1,3,2-dioxaborolan-2-yl)pyridin-2-amine (1.00 g, 3.10 mmol, 69%) as a white solid; δ_{H} (400 MHz; DMSO-d6) 8.23 (s, 1H), 7.54 (1H, m), 7.11 (1H, d, J 6.4 Hz), 6.45 (1H, d, J 8.4 Hz), 4.46-4.34 (1H, m), 3.89-3.78 (2H, m), 3.70 (1H, m), 3.50 (1H, m), 2.15 (1H, m), 1.82-1.71 (m, 1H), 1.24 (12H, s); ES-MS [M+H]⁺: 291.2.

The following intermediates were prepared in an analogous manner:

| Name | Structure | Starting material | Analytical data |
|---|---|---|---|
| (S)-N-(tetrahydrofuran- 3-yl)-5-(4,4,5,5-tetra- methyl-1,3,2-dioxaboro- lan-2-yl)pyridin-2-amine | 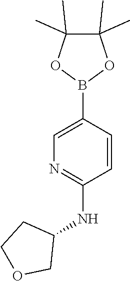 [SMILES: CC1(C)OB(c2ccc(N[C@H]3CCOC3)nc2)OC1(C)C] | (S)-3-aminotetrahydro- furan | ES-MS [M + H]⁺: 291.1 |
| 6-(5-(4,4,5,5-tetramethyl- 1,3,2-dioxaborolan-2-yl)- pyridin-2-yl)-2-oxa-6- azaspiro [3.3]heptane | 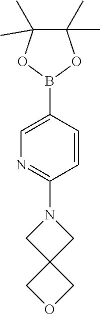 | 2-oxa-6-azaspiro[3.3] heptane oxalate | ES-MS [M + H]⁺: 303.3 |

### 2-(Oxetan-3-yl)-5-(4,4,5,5-tetramethyl-1,3,2-dioxaborolan-2-yl)isoindolin-1-one

### Step 1

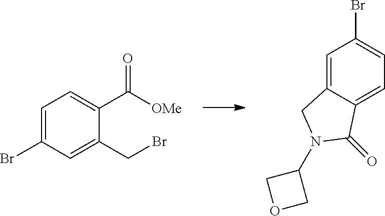 [SMILES: COC(=O)c1ccc(Br)cc1CBr ; O=C1c2ccc(Br)cc2CN1C1COC1]

3-Aminooxetane (522 mg, 7.14 mmol) was added to a solution of methyl 4-bromo-2-(bromomethyl)benzoate (2.00 g, 6.49 mmol) and DIPEA (3.39 mL, 19.48 mmol) in THF (20 mL) and the resulting mixture was stirred at 60° C. for 12 h. Water was added (20 mL) and the mixture was extracted into ethyl acetate (2×20 mL). The combined organic extracts were washed with brine (2×20 mL), dried over anhydrous sodium sulfate and concentrated in vacuo. Flash column chromatography on silica (EtOAc:PE, 0:100 to 100:0) afforded 5-bromo-2-(oxetan-3-yl)isoindolin-1-one (1.20 g, 4.48 mmol, 69%) as a yellow solid; δ_{H} (400 MHz; DMSO-d6) 7.97 (1H, m), 7.76-7.71 (1H, m), 7.69-7.63 (1H, m), 5.59-5.37 (1H, m), 4.91-4.82 (6H, m); ES-MS [M+H]⁺: 268.1.

### Step 2

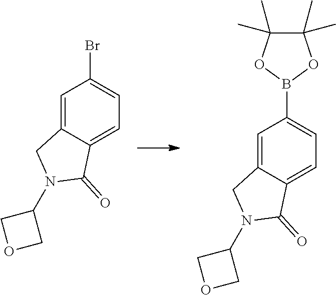 [SMILES: O=C1c2ccc(Br)cc2CN1C1COC1 ; CC1(C)OB(c2ccc3c(c2)CN(C2COC2)C3=O)OC1(C)C]

2-(Oxetan-3-yl)-5-(4,4,5,5-tetramethyl-1,3,2-dioxaborolan-2-yl)isoindolin-1-one was prepared in an analogous manner to 2,2-difluoro-2-(5-(4,4,5,5-tetramethyl-1,3,2-dioxaborolan-2-yl)pyridin-2-yl)ethan-1-ol from 5-bromo-2-(oxetan-3-yl)isoindolin-1-one; δ_{H} (400 MHz; DMSO-d6) 7.93 (1H, s), 7.78 (1H, d, J 7.70 Hz), 7.71-7.67 (1H, m), 5.43 (1H, m), 4.91-4.76 (6H, m), 1.33 (12H, s); ES-MS [M+H]⁺: 316.2.

### Tert-Butyl 3-(4,4,5,5-tetramethyl-1,3,2-dioxaborolan-2-yl)-6a, 7,9,10-tetrahydropyrazino[1,2-d]pyrido[3,2-b][1,4]oxazine-8(6H)-carboxylate

### Step 1

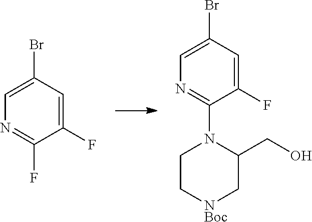 [SMILES: Fc1cc(Br)cnc1F ; CC(C)(C)OC(=O)N1CCN(c2ncc(Br)cc2F)C(CO)C1]

DIPEA (2.42 mL, 13.9 mmol) was added to a solution of 5-bromo-2,3-difluoropyridine (2.69 g, 13.9 mmol) and tert-butyl 3-(hydroxymethyl)piperazine-1-carboxylate (2.00 g, 9.25 mmol) in DMSO (20 mL) and the resulting mixture was stirred at 100° C. for 16 h. Water was added (30 mL) and the mixture was extracted into ethyl acetate (3×30 mL). The combined organic extracts were washed with brine (2×30 mL), dried over anhydrous sodium sulfate and concentrated in vacuo. Flash column chromatography on silica (EtOAc:PE, 0:100 to 30:70) afforded tert-butyl 4-(5-bromo-3-fluoropyridin-2-yl)-3-(hydroxymethyl)piperazine-1-carboxylate (1.50 g, 3.73 mmol, 40%) as a colorless oil; ES-MS [M+H]⁺: 392.1.

### Step 2

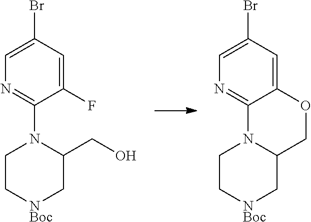 [SMILES: CC(C)(C)OC(=O)N1CCN(c2ncc(Br)cc2F)C(CO)C1 ; CC(C)(C)OC(=O)N1CCN2c3ncc(Br)cc3OCC2C1]

Sodium hydride (60% dispersion in oil, 37 mg, 0.92 mmol) was added to a solution of tert-butyl 4-(5-bromo-3-fluoropyridin-2-yl)-3-(hydroxymethyl)piperazine-1-carboxylate (300 mg, 0.77 mmol) in DMF (4 mL) at 0° C. The resulting mixture was stirred at 20° C. for 30 min and then 50° C. for 1 h. The reaction mixture was cooled to room temperature and quenched with saturated aqueous ammonium chloride solution (30 mL). The mixture was extracted into ethyl acetate (3×30 mL) and the combined organic extracts were dried over anhydrous sodium sulfate and concentrated in vacuo to afford tert-butyl 3-bromo-6a,7,9,10-tetrahydropyrazino[1,2-d]pyrido[3,2-b][1,4]oxazine-8(6H)-carboxylate (280 mg, 0.75 mmol, 97%) as a yellow oil; δ_{H} (400 MHz; CDCl3) 7.85-7.80 (1H, m), 7.12-7.08 (1H, m), 4.52-4.41 (1H, m), 4.30-4.21 (1H, m), 4.21-4.03 (2H, m), 4.00-3.89 (1H, m), 3.40-3.27 (1H, m), 2.96-2.90 (1H, m), 2.81-2.71 (1H, m), 2.68-2.53 (1H, m), 1.52-1.47 (9H, m); ES-MS [M+H]⁺: 372.1.

### Step 3

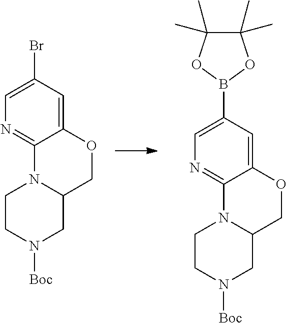 [SMILES: CC(C)(C)OC(=O)N1CCN2c3ncc(Br)cc3OCC2C1 ; CC(C)(C)OC(=O)N1CCN2c3ncc(B4OC(C)(C)C(C)(C)O4)cc3OCC2C1]

tert-Butyl 3-(4,4,5,5-tetramethyl-1,3,2-dioxaborolan-2-yl)-6a,7,9,10-tetrahydropyrazino[1,2-d]pyrido[3,2-b][1,4]oxazine-8(6H)-carboxylate was prepared in an analogous manner to 2,2-difluoro-2-(5-(4,4,5,5-tetramethyl-1,3,2-dioxaborolan-2-yl)pyridin-2-yl)ethan-1-ol from tert-butyl 3-bromo-6a,7,9,10-tetrahydropyrazino[1,2-d]pyrido[3,2-b][1,4]oxazine-8(6H)-carboxylate; ES-MS [M+H]⁺: 336.2 (boronic acid).

### (R)-1-((1,4-Dioxan-2-yl)methyl)-4-(4,4,5,5-tetramethyl-1,3,2-dioxaborolan-2-yl)-1H-pyrazole

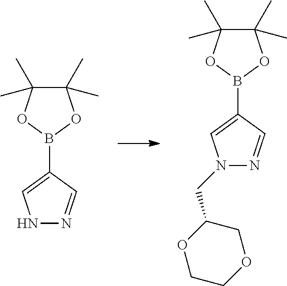

DIAD (1.04 g, 5.16 mmol) was added to a solution of 4-(4,4,5,5-tetramethyl-1,3,2-dioxaborolan-2-yl)-1H-pyrazole (500 mg, 2.58 mmol), (R)-(1,4-dioxan-2-yl)methanol (370 mg, 3.13 mmol) and triphenylphosphine (1.35 g, 5.15 mmol) in THF (10 mL). The resulting mixture stirred at 20° C. for 12 h, then concentrated in vacuo. Preparative HPLC purification (column: Waters Xbridge Prep OBD C₁₈ 150*40 mm 10 μm; acetonitrile:water(TFA); gradient: 15%-45% B over 20 min) afforded (R)-1-((1,4-dioxan-2-yl)methyl)-4-(4,4,5,5-tetramethyl-1,3,2-dioxaborolan-2-yl)-1H-pyrazole (800 mg, 2.45 mmol. 95%) as a white solid; δ_{H} (400 MHz; DMSO-d6) 8.87 (2H, s), 3.49 (3H, m), 3.32-3.21 (6H, m), 1.20-1.14 (12H, m); ES-MS [M+H]⁺: 213.1 (boronic acid).

The following intermediate was prepared in an analogous manner:

| Name | Structure | Starting material | Analytical data |
|---|---|---|---|
| (S)-1-((1,4-Dioxan-2-yl)- methyl)-4-(4,4,5,5-tetra- methyl-1,3,2-dioxaborolan- 2-yl)-1H-pyrazole | 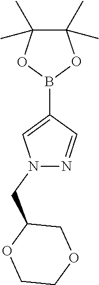 | (S)-(1,4-dioxan-2-yl)- methanol | ES-MS[M + H]⁺: 294.9 |

### 2-Methyl-1-(3-methyl-4-(4,4,5,5-tetramethyl-1,3,2-dioxaborolan-2-yl)-1H-pyrazol-1-yl)propan-2-ol

### Step 1

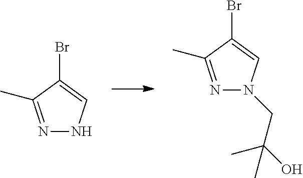

Cesium carbonate (15.2 g, 46.6 mmol) was added to a solution 4-bromo-3-methyl-1H-pyrazole (5.00 g, 31.1 mmol) in DMF (20 mL) and the resulting mixture was stirred at 25° C. for 10 min. 2,2-Dimethyloxirane (8.27 mL, 93.2 mmol) was added and stirring was continued at 25° C. for 16 h. Water was added (100 mL) and the mixture was extracted into ethyl acetate (3×100 mL). The combined organic extracts were dried over anhydrous sodium sulfate and concentrated in vacuo. Preparative HPLC purification (Welch Ultimate XB-SiOH 250*70 10 μm; hexane:ethanol; B %: 10%, isocratic elution mode) afforded 1-(4-bromo-3-methyl-1H-pyrazol-1-yl)-2-methylpropan-2-ol (4.50 g, 18.5 mmol, 60%) as a yellow oil; δ_{H} (400 MHz; CDCl3) 7.48-7.44 (1H, m), 3.96 (2H, s), 2.24 (3H, s), 1.16 (6H, s); ES-MS [M+H]⁺: 232.9.

### Step 2

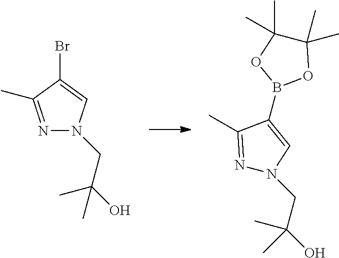

2-Methyl-1-(3-methyl-4-(4,4,5,5-tetramethyl-1,3,2-dioxaborolan-2-yl)-1H-pyrazol-1-yl)propan-2-ol was prepared in an analogous manner to 2,2-difluoro-2-(5-(4,4,5,5-tetramethyl-1,3,2-dioxaborolan-2-yl)pyridin-2-yl)ethan-1-ol from 1-(4-bromo-3-methyl-1H-pyrazol-1-yl)-2-methylpropan-2-ol; δ_{H} (400 MHz; CDCl3) 7.72-7.70 (1H, m), 4.66-4.64 (1H, m), 3.93-3.91 (2H, m), 2.22 (3H, s), 1.24 (12H, s), 1.04-1.02 (6H, m); ES-MS [M+H]⁺: 281.1.

### 4-(2-(Methoxy-d3)ethoxy)-1H-pyrazole

### Step 1

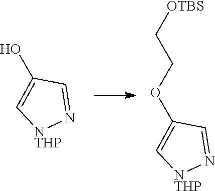

Cesium carbonate (7.43 g, 17.9 mmol) was added to a solution of 1-(tetrahydro-2H-pyran-2-yl)-1H-pyrazol-4-ol (3.00 g, 17.9 mmol) and (2-bromoethoxy)-tert-butyldimethylsilane (5.13 g, 21.6 mmol) in DMF (10 mL). The resulting mixture was stirred at 80° C. for 2 h. Water was added (100 mL) and the mixture was extracted into ethyl acetate (3×50 mL). The combined organic extracts were dried over anhydrous sodium sulfate and concentrated in vacuo to afford 4-(2-((tert-butyldimethylsilyl)oxy)ethoxy)-1-(tetrahydro-2H-pyran-2-yl)-1H-pyrazole (5.00 g, 15.3 mmol, 85%) as a white solid.

### Step 2

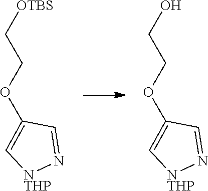

TBAF (5.21 g, 19.9 mmol) was added to a solution of 4-(2-((tert-Butyldimethylsilyl)oxy)ethoxy)-1-(tetrahydro-2H-pyran-2-yl)-1H-pyrazole (5.00 g, 15.3 mmol) in THF (30 mL) and the resulting mixture was stirred at 25° C. for 12 h. Water was added (20 mL) and the mixture was extracted into ethyl acetate (3×10 mL). The combined organic extracts were dried over anhydrous sodium sulfate and concentrated in vacuo. Flash column chromatography on silica (EtOAc:PE, 0:100 to 80:20) afforded 2-((1-(tetrahydro-2H-pyran-2-yl)-1H-pyrazol-4-yl)oxy)ethan-1-ol (2.00 g, 9.42 mmol, 62%) as a white solid; δ_{H} (400 MHz; DMSO-d6) 7.59 (1H, s), 7.25 (1H, s), 5.24 (1H, m), 4.82 (1H, m), 3.93-3.84 (3H, m), 3.69-3.55 (3H, m), 2.10-1.97 (1H, m), 1.97-1.79 (2H, m), 1.73-1.57 (1H, m), 1.56-1.46 (2H, m).

### Step 3

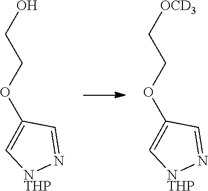

4-(2-(Methoxy-d3)ethoxy)-1-(tetrahydro-2H-pyran-2-yl)-1H-pyrazole was prepared in an analogous manner to 3-(methoxy-d3)azetidine; δ_{H} (400 MHz; DMSO-d6) 7.60 (1H, s), 7.26 (1H, s), 5.23 (1H, m), 3.95 (2H, m), 3.92-3.84 (1H, m), 3.65-3.55 (3H, m), 2.10-1.98 (1H, m), 1.96-1.79 (2H, m), 1.72-1.58 (1H, m), 1.55-1.44 (2H, m).

### Step 4

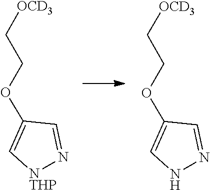

HCl (1.0 M in dioxane, 8.72 mL, 8.72 mmol) was added to a solution of 4-(2-(methoxy-d3)ethoxy)-1-(tetrahydro-2H-pyran-2-yl)-1H-pyrazole (400 mg, 1.74 mmol) in dichloromethane (5 mL). The mixture was stirred at 25° C. for 12 h, then adjusted to pH˜9 by addition of saturated aqueous sodium bicarbonate solution. Water was added (10 mL) and the mixture was extracted into ethyl acetate (3×10 mL). The combined organic extracts were dried over anhydrous sodium sulfate and concentrated in vacuo. Flash column chromatography on silica (EtOAc:PE, 0:100 to 75:25) afforded 4-(2-(methoxy-d3)ethoxy)-1H-pyrazole (100 mg, 0.69 mmol, 39%) as a white solid; δ_{H} (400 MHz; DMSO-d6) 12.35 (1H, br s), 7.57-7.05 (2H, m), 3.98-3.87 (2H, m), 3.63-3.55 (2H, m).

### 4-(2-(Difluoromethoxy)ethoxy)-1H-pyrazole

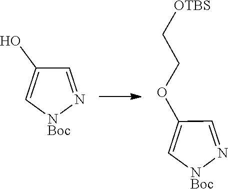 [SMILES: CC(C)(C)OC(=O)n1cc(O)cn1 ; CC(C)(C)OC(=O)n1cc(OCCO[Si](C)(C)C(C)(C)C)cn1]

Cesium carbonate (10.6 g, 32.6 mmol) was added to a solution of tert-butyl 4-hydroxy-1H-pyrazole-1-carboxylate (3.00 g, 16.3 mmol) and (2-bromoethoxy)(tert-butyl)dimethylsilane (4.68 g, 19.5 mmol) in DMF (30 mL). The resulting mixture was stirred at 80° C. for 1 h. Water was added (60 mL) and the mixture was extracted into ethyl acetate (3×50 mL). The combined organic extracts were dried over anhydrous sodium sulfate and concentrated in vacuo to afford tert-butyl 4-(2-((tert-butyldimethylsilyl)oxy)ethoxy)-1H-pyrazole-1-carboxylate (3.00 g, 8.76 mmol, 54%) which was used without further purification.

### Step 2

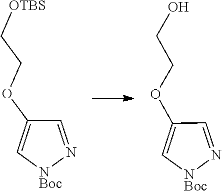 [SMILES: CC(C)(C)OC(=O)n1cc(OCCO[Si](C)(C)C(C)(C)C)cn1 ; CC(C)(C)OC(=O)n1cc(OCCO)cn1]

p-Toluenesulfonic acid (302 mg, 1.75 mmol) was added to a solution of tert-butyl 4-(2-((tert-butyldimethylsilyl)oxy)ethoxy)-1H-pyrazole-1-carboxylate (3.00 g, 8.76 mmol) in DMF (20 mL) and the resulting mixture was stirred at 20° C. for 12 h. Water was added (40 mL) and the mixture was extracted into ethyl acetate (2×20 mL). The combined organic extracts were dried over anhydrous sodium sulfate and concentrated in vacuo. Flash column chromatography on silica (EtOAc:PE, 0:100 to 100:0) afforded tert-butyl 4-(2-hydroxyethoxy)-1H-pyrazole-1-carboxylate (1.50 g, 6.24 mmol, 71%); ES-MS [M+H]⁺: 229.2.

### Step 3

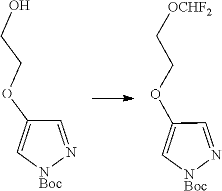 [SMILES: CC(C)(C)OC(=O)n1cc(OCCO)cn1 ; CC(C)(C)OC(=O)n1cc(OCCOC(F)F)cn1]

(Bromodifluoromethyl)trimethylsilane (2.94 g, 14.5 mmol) was added to a mixture of tert-butyl 4-(2-hydroxyethoxy)-1H-pyrazole-1-carboxylate (1.50 g, 6.57 mmol), potassium acetate (1.93 g, 19.7 mmol), dichloromethane (10 mL) and water (10 mL). The resulting mixture was stirred at 20° C. for 12 h, then extracted into dichloromethane (3×5 mL). The combined organic extracts were dried over anhydrous sodium sulfate and concentrated in vacuo. Flash column chromatography on silica (EtOAc:PE, 0:100 to 100:0) afforded tert-butyl 4-(2-(difluoromethoxy)ethoxy)-1H-pyrazole-1-carboxylate (300 mg, 099 mmol, 15%) as a colorless oil; δ_{H} (400 MHz; DMSO-d6) 8.00 (1H, d, J 0.63 Hz), 7.74 (1H, d, J 0.75 Hz), 6.56-6.98 (1H, m), 4.13-4.22 (4H, m), 1.60 (9H, s).

### Step 4

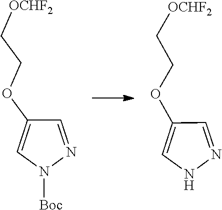 [SMILES: CC(C)(C)OC(=O)n1cc(OCCOC(F)F)cn1 ; FC(F)OCCOc1cn[nH]c1]

HCl (1.0 M in dioxane, 2 mL, 2 mmol) was added to a solution of tert-butyl 4-(2-(difluoromethoxy)ethoxy)-1H-pyrazole-1-carboxylate (300 mg, 1.08 mmol). The resulting mixture was stirred at 20° C. for 1 h, then concentrated in vacuo to afford 4-(2-(difluoromethoxy)ethoxy)-1H-pyrazole (180 mg) which was used without further purification.

### 4-(2-((1H-Pyrazol-4-yl)oxy)ethyl)morpholine

Step 1

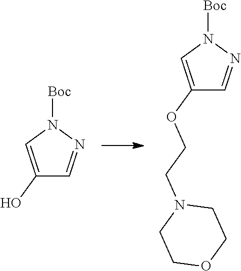 [SMILES: CC(C)(C)OC(=O)n1cc(O)cn1 ; CC(C)(C)OC(=O)n1cc(OCCN2CCOCC2)cn1]

Cesium carbonate (2.30 g, 7.06 mmol) was added to a solution of tert-butyl 4-hydroxy-1H-pyrazole-1-carboxylate (0.65 g, 3.53 mmol) and 4-(2-bromoethyl)morpholine (821 mg, 4.23 mmol) in DMF (7 mL). The resulting mixture was stirred at 80° C. for 2 h. Water was added (20 mL) and the mixture was extracted into ethyl acetate (3×10 mL). The combined organic extracts were washed with brine (20 mL), dried over anhydrous sodium sulfate and concentrated in vacuo. Flash column chromatography on silica (EtOAc:PE, 0:100 to 60:40) afforded tert-butyl 4-(2-morpholinoethoxy)-1H-pyrazole-1-carboxylate (800 mg, 2.69 mmol, 76%) as a yellow oil; δ_{H} (400 MHz; DMSO-d6) 7.91 (1H, d, J 0.75 Hz), 7.66 (1H, d, J=0.75), 4.03 (1H, m), 4.02 (1H, m), 3.60-3.52 (4H, m), 2.63 (2H, m), 2.48-2.39 (4H, m), 1.55 (s, 9H).

### Step 2

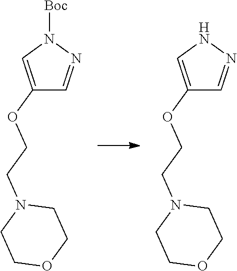 [SMILES: CC(C)(C)OC(=O)n1cc(OCCN2CCOCC2)cn1 ; c1n[nH]cc1OCCN1CCOCC1]

TFA (3 mL, 40.4 mmol) was added to a solution of tert-butyl 4-(2-morpholinoethoxy)-1H-pyrazole-1-carboxylate (800 mg, 2.69 mmol) in dichloromethane (9 mL) and the resulting mixture was stirred at 20° C. for 2 h. The reaction mixture was then adjusted to pH˜8 by the addition of sodium bicarbonate. Water was added (10 mL) and the mixture was extracted into ethyl acetate (3×10 mL). The combined organic extracts were washed with brine (30 mL), dried over anhydrous sodium sulfate and concentrated in vacuo to afford 4-(2-((1H-pyrazol-4-yl)oxy)ethyl)morpholine (200 mg, 1.01 mmol, 38%) as a yellow solid; δ_{H} (400 MHz; DMSO-d6) 12.32 (1H, br s), 7.48-7.23 (2H, m), 3.93 (2H, m), 3.59-3.50 (4H, m), 2.61 (2H, m), 2.46-2.36 (4H, m).

The following intermediate was prepared in an analogous manner:

| Name | Structure | Starting material | Analytical data |
|---|---|---|---|
| 4-(1-(((1H-pyrazol- 4-yl)oxy)methyl)- cyclopropyl)mor- pholine | 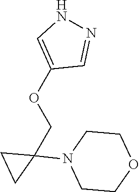 [SMILES: c1n[nH]cc1OCC1(N2CCOCC2)CC1] | (1-morpholinocyclo- propyl)methanol | δ_{H}(400 MHz; DMSO-d6) 11.67-10.64 (1H, m), 7.41 (2H, s), 4.12 (2H, br s), 3.89 (4H, br s), 3.60-3.20 (2H, m), 1.88-1.19 (4H, m), 0.99 (2H, br s) |

### (R)-4-(1-((1H-Pyrazol-4-yl)oxy)propan-2-yl)morpholine

### Step 1

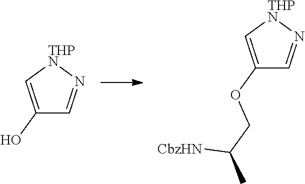

(Tributylphosphoranylidene)acetonitrile (6.89 g, 28.5 mmol) was added to a solution of 1-(oxan-2-yl)-1H-pyrazol-4-ol (3.20 g, 19.0 mmol) and benzyl N-[(2R)-1-hydroxypropan-2-yl]carbamate (5.97 g, 28.5 mmol) in toluene (50 mL). The resulting mixture was stirred at 100° C. for 16 h then concentrated in vacuo. Flash column chromatography on silica (EtOAc:PE, 0:100 to 22:78) afforded benzyl ((2R)-1-((1-(tetrahydro-2H-pyran-2-yl)-1H-pyrazol-4-yl)oxy)propan-2-yl)carbamate (1.00 g, 2.78 mmol, 15%) as a yellow oil; δ_{H} (400 MHz; DMSO-d6) 7.60 (1H, s), 7.38-7.33 (5H, m), 7.25 (1H, s), 5.22 (1H, m), 5.02 (2H, s), 3.92-3.74 (3H, m), 3.72-3.66 (1H, m), 3.63-3.51 (1H, m), 2.01 (1H, br s), 1.94-1.79 (2H, m), 1.70-1.58 (1H, m), 1.55-1.46 (2H, m), 1.11 (3H, d, J 6.60 Hz).

### Step 2

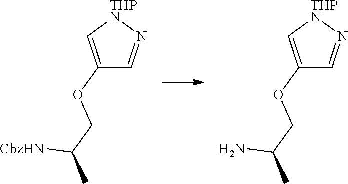

Benzyl ((2R)-1-((1-(tetrahydro-2H-pyran-2-yl)-1H-pyrazol-4-yl)oxy)propan-2-yl)carbamate (1.00 g, 2.78 mmol) was added to a suspension of 10% Pd/C (100 mg, 94 μmol) in THF (10 mL) under nitrogen. The resulting mixture was degassed and purged with hydrogen three times, then stirred under hydrogen (50 PSI) at 25° C. for 4.5 h. The mixture was then filtered through celite and concentrated in vacuo to afford (2R)-1-((1-(tetrahydro-2H-pyran-2-yl)-1H-pyrazol-4-yl)oxy)propan-2-amine (500 mg) as a yellow oil.

### Step 3

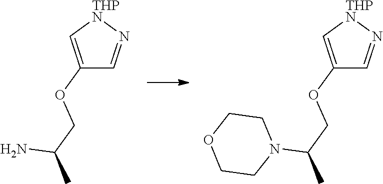

Potassium carbonate (736 mg, 5.33 mmol) and 1-bromo-2-(2-bromoethoxy)ethane (453 mg, 1.95 mmol) were added to a solution of (2R)-1-((1-(tetrahydro-2H-pyran-2-yl)-1H-pyrazol-4-yl)oxy)propan-2-amine (400 mg, 1.78 mmol) in ethanol (4 mL). The resulting mixture was stirred at 60° C. for 16 h, then filtered and concentrated in vacuo. Preparative HPLC purification (column: Welch Ultimate XB-CN 250*70 mm 10 μm; hexane:ethanol; gradient: 1%-30% B over 15 min) afforded 4-((2R)-1-((1-(tetrahydro-2H-pyran-2-yl)-1H-pyrazol-4-yl)oxy)propan-2-yl)morpholine (535 mg, 1.47 mmol, 83%) as a yellow oil; ES-MS [M+H]⁺: 296.2.

### Step 4

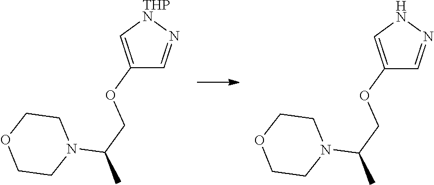

HCl (2.0 M dioxane, 1 mL, 1 mmol) was added to a solution of 4-((2R)-1-((1-(tetrahydro-2H-pyran-2-yl)-1H-pyrazol-4-yl)oxy)propan-2-yl)morpholine (100 mg, 339 μmol) in dichloromethane (0.5 mL). The resulting mixture was stirred at 25° C. for 4 h. Water was added (2 mL) and the mixture was extracted into dichloromethane (3×2 mL). The combined organic extracts were washed with brine (6 mL), dried over anhydrous sodium sulfate and concentrated in vacuo to afford (R)-4-(1-((1H-pyrazol-4-yl)oxy)propan-2-yl)morpholine (100 mg) as a white solid that was used without further purification; δ_{H} (400 MHz; DMSO-d6) 12.32 (1H, m), 7.44 (1H, br s), 7.25 (1H, m), 3.91 (1H, m), 3.72 (1H, in), 3.55 (4H, m), 2.86-2.77 (1H, m), 2.52 (4H, m), 1.03 (3H, d, J 6.75 Hz).

The following intermediates were prepared in an analogous manner:

| Name | Structure | Starting material | Analytical data |
|---|---|---|---|
| (S)-4-(1-((1H-pyrazol- 4-yl)oxy)propan-2-yl)- morpholine | 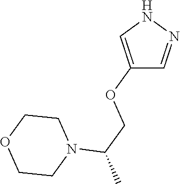 | benzyl N-[(2S)-1-hydroxy- propan-2-yl]carbamate | δ_{H}(400 MHz; DMSO-d6) 13.46-12.26(1H, m), 7.98- 7.18 (2H, m), 3.92 (1H, m), 3.75-3.71 (1H, m), 3.58-3.53 (4H, m), 2.86- 2.77 (1H, m), 2.73 (5H, m), 1.04 (3H, d, J 6.75 Hz); ES-MS [M + H]⁺: 212.2 |
| 4-(2-((1H-pyrazol-4- yl)oxy)ethyl)morph- olin-3-one* | 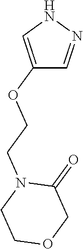 [SMILES: O=C1COCCN1CCOc1cn[nH]c1] | 4-(2-hydroxyethyl)morpho- lin-3-one | δ_{H}(400 MHz; DMSO-d6) 12.36 (1H, br s), 7.60-7.08 (2H, m), 4.03 (2H, s), 3.98 (2H, m), 3.84-3.76 (2H, m), 3.62 (2H, m), 3.47- 3.42 (2H, m) |
| 4-((1-methyl-2-oxa- bicyclo[2.1.1]hexan- 4-yl)methoxy)-1H- pyrazole* | 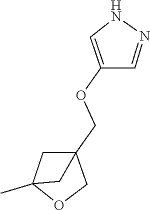 | (1-methyl-2-oxabicyclo [2.1.1]hexan-4-yl)methanol | δ_{H}(400 MHz; DMSO-d6) 7.35 (2H, s) 4.10 (2H, s) 3.61 (2H, s) 1.67 (2H, m) 1.47 (2H, m) 1.35 (3H, s) |

*Prepared using steps 1 and 4 only

### N-(7-bromo-6-fluorobenzo[d]thiazol-2-yl)-3-fluoro-3-(methoxymethyl)azetidine-1-carboxamide

### Step 1

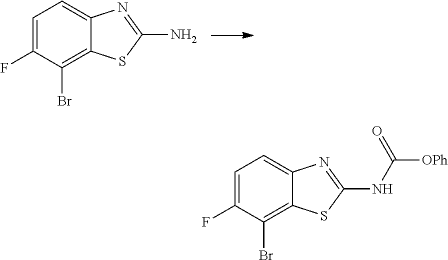 [SMILES: Nc1nc2ccc(F)c(Br)c2s1 ; O=C(Nc1nc2ccc(F)c(Br)c2s1)Oc1ccccc1]

Phenyl chloroformate (15.2 mL, 121 mmol) was added to a solution of 7-bromo-6-fluorobenzo[d]thiazol-2-amine (see D, steps 2-4, 10.0 g, 40.5 mmol) and 2,6-lutidine (23.6 mL, 202 mmol) in dichloromethane (100 mL). The resulting mixture was stirred at 20° C. for 12 h. Water was added (30 mL) and the mixture was extracted into ethyl acetate (3×30 mL). The combined organic extracts were washed with brine (6 mL), dried over anhydrous sodium sulfate and concentrated in vacuo. Trituration with methanol afforded phenyl (7-bromo-6-fluorobenzo[d]thiazol-2-yl)carbamate (10.0 g, 24.8 mmol, 61%) as a white solid; δ_{H} (400 MHz; DMSO-d6) 12.86 (1H, br s), 7.78 (1H, m), 7.51-7.44 (3H, m), 7.36-7.29 (3H, m); ES-MS [M+H]⁺: 369.0.

### Step 2

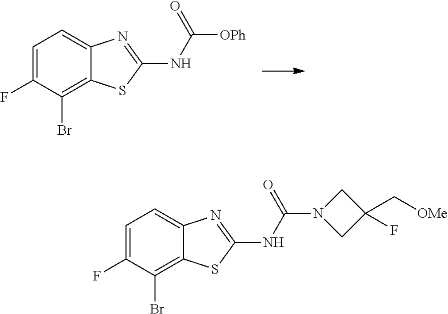 [SMILES: O=C(Nc1nc2ccc(F)c(Br)c2s1)Oc1ccccc1 ; COCC1(F)CN(C(=O)Nc2nc3ccc(F)c(Br)c3s2)C1]

3-Fluoro-3-(methoxymethyl)azetidine (1.17 g, 9.80 mmol) was added to a solution of phenyl (7-bromo-6-fluorobenzo[d]thiazol-2-yl)carbamate (2.40 g, 6.54 mmol) and triethylamine (0.91 mL, 6.54 mmol) in dichloromethane (30 mL). The resulting solution was stirred for at 40° C. for 12 h. Water was added (30 mL) and the mixture was extracted into ethyl acetate (3×30 mL). The combined organic extracts were washed with brine (2×20 mL), dried over anhydrous sodium sulfate and concentrated in vacuo. Flash column chromatography on silica (EtOAc:PE, 0:100 to 40:60) afforded N-(7-bromo-6-fluorobenzo[d]thiazol-2-yl)-3-fluoro-3-(methoxymethyl)azetidine-1-carboxamide (2.50 g, 6.25 mmol, 96%) as a white solid; δ_{H} (400 MHz; DMSO-d6); 11.68 (1H, br s), 7.65 (1H, m), 7.40 (1H, m), 4.30-4.03 (4H, m), 3.77-3.65 (2H, m), 3.34 (3H, s); ES-MS [M+H]⁺: 392.0.

The following intermediates were prepared in an analogous manner:

| Name | Structure | Starting material(s) | Analytical data |
|---|---|---|---|
| N-(7-bromo-6- fluorobenzo[d]thi- azol-2-yl)-5-oxa- 2-azaspiro[3.4] octane-2-carbox- amide | 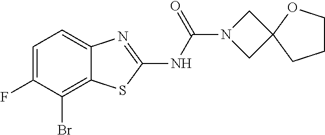 [SMILES: O=C(Nc1nc2ccc(F)c(Br)c2s1)N1CC2(CCCO2)C1] | 5-oxa-2-azaspiro[3.4] octane | δ_{H}(400 MHz; DMSO-d6) 12.47-12.22 (1H, m), 7.60- 7.15 (2H, m), 3.98-3.85 (4H, m) 2.57-2.55 (2H, m), 2.48 (2H, m) 2.15 (2H, m) 1.12 (6H, d, J 6.36 Hz); ES-MS [M + H]⁺: 388.0 |
| N-(7-bromo-6- fluorobenzo[d]thi- azol-2-yl)-6-oxa- 2-azaspiro[3.4] octane-2-carbox- amide | 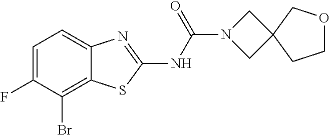 [SMILES: O=C(Nc1nc2ccc(F)c(Br)c2s1)N1CC2(CCOC2)C1] | 6-oxa-2-azaspiro[3.4] octane | [M + H]⁺: 388.0 |
| N-(7-bromo-6- fluorobenzo[d]thi- azol-2-yl)-3- fluoro-3-((meth- oxy-d3)methyl)- azetidine-1- carboxamide | 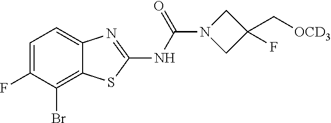 | 3-fluoro-3-((methoxy- d3)methyl)azetidine | [M + H]⁺: 397.1 |
| N-(7-bromo-6- fluorobenzo[d]thi- azol-2-yl)-3- (methoxy-d3)-3- methylazetidine- 1-carboxamide | 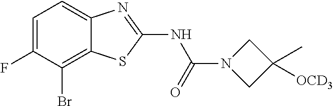 | 3-(methoxy-d3)-3- methylazetidine | δ_{H}(400 MHz; DMSO-d6) 11.53 (1H, br s), 7.71-7.56 (1H, m), 7.40 (1H, m), 4.07-3.93 (2H, m), 3.86 (2H, m), 1.42 (3H, s) |
| N-(7-bromo-6- fluorobenzo[d]thi- azol-2-yl)-3-meth- oxy-3-methylazet- idine-1-carbox- amide | 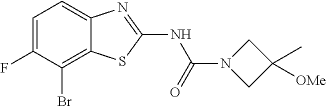 [SMILES: COC1(C)CN(C(=O)Nc2nc3ccc(F)c(Br)c3s2)C1] | 3-methoxy-3-methyl- azetidine | δ_{H}(400 MHz; DMSO-d6) 11.52 (1H, br s), 7.64 (1H, m), 7.38 (1H, m), 4.06- 3.78 (4H, m), 3.18 (3H, s), 1.42 (3H, s); [M + H]⁺: 374.1 |
| N-(7-bromo-6- chlorobenzo[d] thiazol-2-yl)-3- fluoro-3-((meth- oxy-d3)methyl)- azetidine-1- | 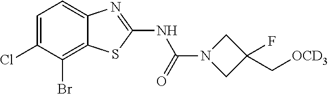 | 7-bromo-6-chloro- benzo[d]thiazol-2- amine 3-fluoro-3- ((methoxy-d3)meth- yl)azetidine | δ_{H}(400 MHz; DMSO-d6) 11.97-11.51 (1H, m), 7.69-7.54 (2H, m), 4.29- 4.11 (4H, m), 3.77-3.65 (2H, m); [M + H]⁺: 413.0 |
| carboxamide | | | |
| N-(7-bromo-6- chlorobenzo[d] thiazol-2-yl)-3- (methoxy-d3)-3- methylazetidine- 1-carboxamide | 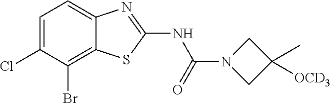 | 7-bromo-6-chloro- benzo[d]thiazol-2- amine 3-(methoxy- d3)-3-methylazetidine | δ_{H}(400 MHz; DMSO-d6) 11.61 (1H, br s), 7.68-7.54 (2H, m), 4.02-3.93 (2H, m), 3.87 (2H, br s), 1.42 (3H, s) |
| N-(7-bromo-6- fluorobenzo[d]thi- azol-2-yl)-2-oxa- 6-azaspiro[3.3] heptane-6-carbox- amide | 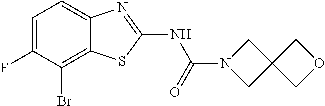 [SMILES: O=C(Nc1nc2ccc(F)c(Br)c2s1)N1CC2(COC2)C1] | 2-oxa-6-azaspiro[3.3] heptane | [M + H]⁺: 374.0 |
| N-(7-bromo-6- chlorobenzo[d]thi- azol-2-yl)-2-oxa- 6-azaspiro[3.3] heptane-6-carbox- amide | 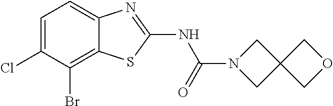 [SMILES: O=C(Nc1nc2ccc(Cl)c(Br)c2s1)N1CC2(COC2)C1] | 2-oxa-6-azaspiro[3.3] heptane7-bromo-6- chlorobenzo[d]thiazol- 2-amine | δ_{H}(400 MHz; DMSO-d6) 11.18-11.95 (1H, m), 7.54-7.68 (2H, m), 4.67 (4H, s), 4.22 (4H, br s); [M + H]⁺: 390.0 |
| N-(7-bromo-4,6- difluorobenzo[d] thiazol-2-yl)-3- fluoro-3-(meth- oxymethyl)azet- idine-1-carbox- amide | 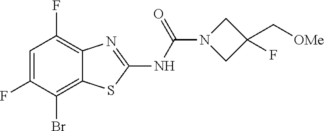 [SMILES: COCC1(F)CN(C(=O)Nc2nc3c(F)cc(F)c(Br)c3s2)C1] | 7-bromo-4,6-difluoro- benzo[d]thiazol-2- amine | δ_{H}(400 MHz; DMSO-d6) 11.91 (1H, br d, J 1.38 Hz), 7.55 (1H, m), 4.51- 3.99 (4H, m), 3.84-3.59 (2H, m), 3.34 (3H, s) |

### N-(Tetrahydro-2H-pyran-4-yl)-1H-pyrazol-4-amine

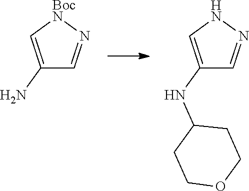 [SMILES: CC(C)(C)OC(=O)n1cc(N)cn1 ; c1n[nH]cc1NC1CCOCC1]

Acetic acid (312 μL, 5.46 mmol) was added to a solution of tert-butyl 4-amino-1H-pyrazole-1-carboxylate (1.00 g, 5.46 mmol) and tetrahydropyran-4-one (1.09 g, 10.9 mmol) in DCE (10 mL). The mixture was stirred at 25° C. for 2 h, then sodium triacetoxyborohydride (2.31 g, 10.9 mmol) was added at 0° C. Stirring was continued at 25° C. for 16 h. The mixture was adjusted to pH 7 with saturated aqueous bicarbonate solution and extracted into ethyl acetate (3×20 mL). The combined organic extracts were washed with brine (20 mL), dried over anhydrous sodium sulfate and concentrated in vacuo. Flash column chromatography on silica (EtOAc:PE, 0:100 to 50:50) followed by preparative HPLC (column: Waters Xbridge 150*25 mm 5 μm; acetonitrile:water (NH₄CO₃); gradient: 22%-42% B over 42 min) afforded N-(tetrahydro-2H-pyran-4-yl)-1H-pyrazol-4-amine (250 mg, 1.50 mmol, 27%) as a yellow solid; δ_{H} (400 MHz; DMSO-d6); 12.06 (1H, br s), 7.07 (2H, s), 4.14 (1H, br d, J 7.6 Hz), 3.83 (2H, m), 3.35-3.28 (2H, m), 3.05-2.91 (1H, m), 1.84 (2H, m), 1.33-1.24 (2H, m).

### 3-(2-((1H-Pyrazol-4-yl)oxy)ethyl)-6-oxa-3-azabicyclo[3.1.1]heptane

### Step 1

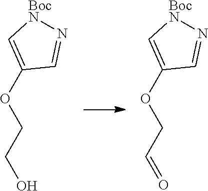 [SMILES: CC(C)(C)OC(=O)n1cc(OCCO)cn1 ; CC(C)(C)OC(=O)n1cc(OCC=O)cn1]

IBX (1.60 g, 5.71 mmol) was added to a solution of tert-butyl 4-(2-hydroxyethoxy)-1H-pyrazole-1-carboxylate* (410 mg, 1.80 mmol) in acetonitrile (5 mL) and the mixture was stirred at 80° C. for 2 h. The reaction mixture was filtered and washed with acetonitrile (5 mL). The resulting solution of tert-butyl 4-(2-oxoethoxy)-1H-pyrazole-1-carboxylate in acetonitrile (5 mL) was used directly in Step 2. *see Step 2 of 4-(2-(difluoromethoxy)ethoxy)-1H-pyrazole synthesis

### Step 2

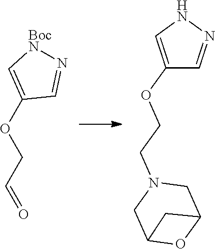 [SMILES: CC(C)(C)OC(=O)n1cc(OCC=O)cn1 ; c1n[nH]cc1OCCN1CC2CC(C1)O2]

Sodium cyanoborohydride (240 mg, 3.82 mmol), 6-oxa-3-azabicyclo[3.1.1]heptane (480 mg, 3.54 mmol) and acetic acid (20 mg, 333 μmol) were added to a solution of tert-butyl 4-(2-oxoethoxy)-1H-pyrazole-1-carboxylate (600 mg, 2.65 mmol) in acetonitrile (10 mL). The reaction mixture was stirred at 25° C. for 16 h then quenched with water (50 mL). The mixture was extracted into ethyl acetate (3×20 mL) and 3:1 ethyl acetate:MeOH (3×50 mL). The combined organic extracts were washed with brine (20 mL), dried over anhydrous sodium sulfate and concentrated in vacuo. Purification by preparative HPLC (column: Phenomenex Luna C₁₈ 150*40 mm 15 μm; acetonitrile:water (NH₄CO₃); gradient: 5%-35% B over 15 min) afforded 3-(2-((1H-pyrazol-4-yl)oxy)ethyl)-6-oxa-3-azabicyclo[3.1.1]heptane (44 mg, 210 μmol, 8%) as a yellow oil; δ_{H} (400 MHz; CDCl₃) 7.32 (2H, s), 4.52 (2H, d, J 6.3 Hz), 4.11 (2H, m), 3.25 (2H, br d, J 11.3 Hz), 3.05 (3H, m), 2.95 (2H, br d, J 11.3 Hz), 2.39 (1H, br d, J 8.1 Hz); ES-MS [M+H]⁺: 210.1.

The following intermediate was prepared in an analogous manner:

| Name | Structure | Starting material | Analytical data |
|---|---|---|---|
| 3-(2-((1H-pyrazol-4- yl)oxy)ethyl)-8-oxa-3- azabicyclo[3.2.1]octane | 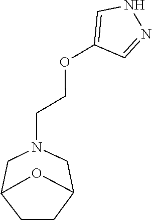 [SMILES: c1n[nH]cc1OCCN1CC2CCC(C1)O2] | 8-oxa-3-azabicyclo [3.2.1]octane | δ_{H}(400 MHz; DMSO-d6) 10.64 (1H,br s), 7.43 (2H, s), 4.46 (2H, br d, J 2.5 Hz), 4.32 (2H, t, J 5.1 Hz), 3.39 (2H, br s), 3.31 (2H, br d, J 12.4 Hz), 3.21-3.15 (2H, m,), 2.28-2.19 (2H, m), 1.98-1.86 (2H, m) |

### Cis-4-(2-((1H-Pyrazol-4-yl)oxy)ethyl)-2,6-dimethylmorpholine

### Step 1

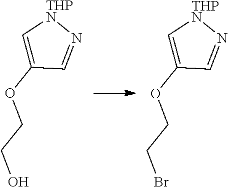

Carbon tetrabromide (12.0 g, 36.2 mmol) was added to a mixture of 2-((1-(tetrahydro-2H-pyran-2-yl)-1H-pyrazol-4-yl)oxy)ethan-1-ol* (5.50 g, 25.9 mmol) and triphenylphosphine (10.0 g, 38.1 mmol) in dichloromethane (30 mL) at 0° C. The resulting mixture was stirred at 25° C. for 12 h, concentrated in vacuo and purified by flash column chromatography on silica (EtOAc:PE, 0:100 to 30:70) to afford 4-(2-bromoethoxy)-1-(tetrahydro-2H-pyran-2-yl)-1H-pyrazole (5.0 g, 18.0 mmol, 70%) as a yellow oil; δ_{H} (400 MHz; CDCl₃) 7.36-7.28 (2H, m), 5.29 (1H, m), 4.21 (2H, m), 4.05 (1H, m), 3.71 (1H, m), 3.60 (2H, m), 2.06 (3H, m), 1.70 (3H, m); ES-MS [M-THP+H]⁺: 210.1. *see Step 2 of 4-(2-(methoxy-d3)ethoxy)-1H-pyrazole synthesis

### Step 2

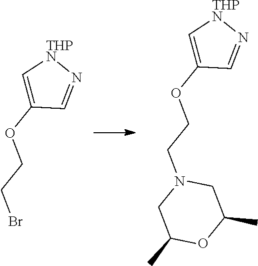

A mixture of 4-(2-bromoethoxy)-1-(tetrahydro-2H-pyran-2-yl)-1H-pyrazole (1.00 g, 3.63 mmol), cis-2,6-dimethylmorpholine (600 mg, 5.21 mmol) and potassium carbonate (1.20 g, 8.68 mmol) in DMF (10 mL) was stirred at 60° C. for 12 h, then concentrated in vacuo. Water was added (50 mL) and the mixture was extracted into ethyl acetate (2×50 mL). The combined organic extracts were washed with water (50 mL), brine (50 mL), dried over anhydrous sodium sulfate and concentrated in vacuo to afford cis-2,6-dimethyl-4-(2-((1-(tetrahydro-2H-pyran-2-yl)-1H-pyrazol-4-yl)oxy)ethyl)morpholine (1.10 g) which was used without further purification; ES-MS [M+H]⁺: 310.1.

### Step 3

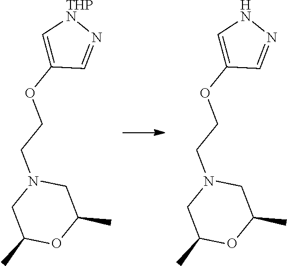

HCl (2.0 M dioxane, 2 mL, 4 mmol) was added to a solution of cis-2,6-dimethyl-4-(2-((1-(tetrahydro-2H-pyran-2-yl)-1H-pyrazol-4-yl)oxy)ethyl)morpholine (1.10 g, 3.56 mmol) in dioxane (5 mL) and the resulting mixture was stirred at 25° C. for 12 h. Saturated sodium bicarbonate solution (20 mL) was added and the mixture was extracted into ethyl acetate (3×30 mL). The combined organic extracts were washed with brine (20 mL), dried over anhydrous sodium sulfate and concentrated in vacuo to afford cis-4-(2-((1H-pyrazol-4-yl)oxy)ethyl)-2,6-dimethylmorpholine (0.60 g, 2.53 mmol, 71%) as a yellow oil; ES-MS [M+H]⁺: 226.1.

The following intermediates were prepared in an analogous manner:

| Name | Structure | Starting material(s) | Analytical data |
|---|---|---|---|
| trans-4-(2-((1H-pyrazol- 4-yl)oxy)ethyl)-2,6- dimethylmorpholine | 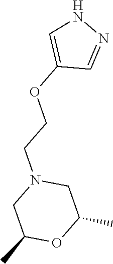 | trans-2,6-dimehyl- morpholine | δ_{H}(400 MHz; DMSO-d6) 12.47-12.22 (1H, m), 7.60-7.15 (2H, m), 3.98- 3.85 (4H, m) 2.57-2.55 (2H, m), 2.48 (2H, m) 2.15 (2H, m) 1.12 (6H, d, J 6.36 Hz) |
| 4-(2-(1H-pyrazol-4- yl)ethyl)morpholine | 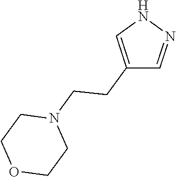 [SMILES: c1n[nH]cc1CCN1CCOCC1] | 2-(1-(tetrahydro- 2H-pyran-2-yl)-1H- pyrazol-4-yl)ethan- 1-ol morpholine | δ_{H}(400 MHz; DMSO-d6) 12.72-12.24 (1H, m), 7.64-7.23 (2H, m), 3.61- 3.54 (4H, m), 2.59-2.54 (2H, m), 2.49-2.31 (6H, m); ES-MS [M + H]⁺: 182.1 |

### 6-(2-((1H-Pyrazol-4-yl)oxy)ethyl)-2-oxa-6-azaspiro[3.3]heptane

### Step 1

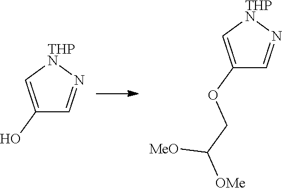

Cesium carbonate (3.87 g, 11.9 mmol) was added to a solution of 1-(tetrahydro-2H-pyran-2-yl)-1H-pyrazol-4-ol (1.00 g, 5.95 mmol) and 2-bromo-1,1-dimethoxy-ethane (1.51 g, 8.92 mmol) in DMF (10 mL). The mixture was stirred at 60° C. for 2 h. Water was added (30 mL) and the mixture was extracted into ethyl acetate (3×10 mL). The combined organic extracts were washed with brine (20 mL), dried over anhydrous sodium sulfate and concentrated in vacuo. Flash column chromatography on silica (EtOAc:PE, 0:100 to 40:60) afforded 4-(2,2-dimethoxyethoxy)-1-(tetrahydro-2H-pyran-2-yl)-1H-pyrazole (800 mg, 3.12 mmol, 53%) as a yellow oil.

### Step 2

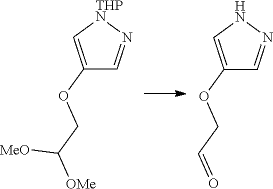

HCl (4.0 M in dioxane, mL, 16 mmol) was added to a solution of 4-(2,2-dimethoxyethoxy)-1-(tetrahydro-2H-pyran-2-yl)-1H-pyrazole (800 mg, 3.12 mmol) in THF (4 mL) and the mixture was stirred at 25° C. for 2 h. Sodium bicarbonate was added until the pH was ˜7 and the mixture was extracted into EtOAc (3×20 mL). The combined organic extracts were dried over anhydrous sodium sulfate and concentrated in vacuo to afford 2-((1H-pyrazol-4-yl)oxy)acetaldehyde (300 mg, 2.38 mmol, 76%) as a yellow oil which was used without further purification.

### Step 3

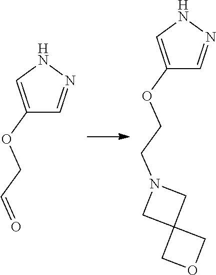 [SMILES: O=CCOc1cn[nH]c1 ; c1n[nH]cc1OCCN1CC2(COC2)C1]

Sodium cyanoborohydride (224 mg, 3.57 mmol), 2-oxa-6-azaspiro[3.3]heptane (283 mg, 2.85 mmol) and acetic acid (14 μL, 238 μmol) were added to a solution of 2-((1H-pyrazol-4-yl)oxy)acetaldehyde (300 mg, 2.38 mmol) in acetonitrile (3 mL). The reaction mixture was stirred at 25° C. for 16 h then quenched with water (3 mL). The mixture was extracted into ethyl acetate (3×2 mL). The aqueous phase was purified by preparative HPLC (column: Waters Xbridge 150*25 mm 10 μm; acetonitrile:water (NH₄OH); gradient: 1%-29% B over 1 min) to 6-(2-((1H-pyrazol-4-yl)oxy)ethyl)-2-oxa-6-azaspiro[3.3]heptane (110 mg, 526 μmol, 22%) as a white solid; δ_{H} (400 MHz; DMSO-d6) 12.30 (1H, br d, J 1.22 Hz), 7.30 (2H, s), 4.58 (4H, s), 3.75 (2H, m), 3.30 (4H, s), 2.59 (2H, m).

### 4-(1-((1H-pyrazol-4-yl)oxy)-2-methylpropan-2-yl)morpholine

### Step 1

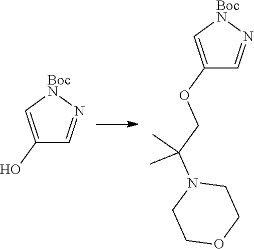 [SMILES: CC(C)(C)OC(=O)n1cc(O)cn1 ; CC(C)(C)OC(=O)n1cc(OCC(C)(C)N2CCOCC2)cn1]

DIAD (1.48 g, 7.33 mmol) was added to a solution of tert-butyl 4-hydroxy-1H-pyrazole-1-carboxylate (900 mg, 4.89 mmol), 2-methyl-2-morpholinopropan-1-ol (856 mg, 5.37 mmol) and triphenylphosphine (1.92 g, 7.33 mmol) in THF (20 mL) at 0° C. The reaction mixture was stirred at 25° C. for 16 h. Water was added (30 mL) and the mixture was extracted into ethyl acetate (3×30 mL). The combined organic extracts were dried over anhydrous sodium sulfate and concentrated in vacuo. Flash column chromatography on silica (EtOAc:PE, 0:100 to 40:60) afforded tert-butyl 4-(2-methyl-2-morpholinopropoxy)-1H-pyrazole-1-carboxylate (610 mg, 1.82 mmol, 37%) as a yellow oil; ES-MS [M+H]⁺: 326.1.

### Step 2

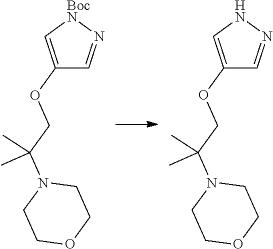 [SMILES: CC(C)(C)OC(=O)n1cc(OCC(C)(C)N2CCOCC2)cn1 ; CC(C)(COc1cn[nH]c1)N1CCOCC1]

HCl (2.0 M in dioxane, 6 mL, 12 mmol) was added to tert-butyl 4-(2-methyl-2-morpholinopropoxy)-1H-pyrazole-1-carboxylate (400 mg, 1.23 mmol) and the mixture was stirred at 25° C. for 1 h. Saturated aqueous sodium bicarbonate solution (30 mL) was added and the mixture was extracted into EtOAc (3×30 mL). The combined organic extracts were dried over anhydrous sodium sulfate and concentrated in vacuo. Preparative HPLC (Welch Ultimate XB-SiOH 250*50 mm 10 μm; hexane:ethanol; gradient: 1%-15% B over 15 min) afforded 4-(1-((1H-pyrazol-4-yl)oxy)-2-methylpropan-2-yl)morpholine (180 mg, 0.80 mmol, 65%) as a white solid; δ_{H} (400 MHz; CD₃OD) 7.49-7.14 (2H, m), 3.74-3.65 (4H, m), 2.68-2.58 (4H, m), 2.48 (2H, s), 1.24 (6H, s).

### N-(tetrahydro-2H-pyran-4-yl)-5-(4,4,5,5-tetramethyl-1,3,2-dioxaborolan-2-yl)pyrimidin-2-amine

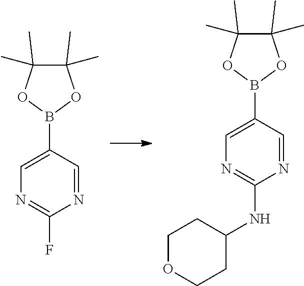

DIPEA (9 mL, 51.6 mmol) was added to a solution of 2-fluoro-5-(4,4,5,5-tetramethyl-1,3,2-dioxaborolan-2-yl)pyrimidine (3.60 g, 35.5 mmol) and tetrahydropyran-4-amine (3.60 g, 35.5 mmol) in DMSO (30 mL). The resulting mixture was heated at 100° C. for 16 h. Water was added (50 mL) and the mixture was extracted into EtOAc (3×50 mL). The combined organic extracts were dried over anhydrous sodium sulfate and concentrated in vacuo to afford N-(tetrahydro-2H-pyran-4-yl)-5-(4,4,5,5-tetramethyl-1,3,2-dioxaborolan-2-yl)pyrimidin-2-amine (3.3 g, 10.8 mmol, 67%) as a white solid; δ_{H} (400 MHz; DMSO-d6) 8.40 (2H, s), 7.58 (1H, br d, J 7.8 Hz), 4.05-3.92 (1H, m), 3.85 (2H, br d, J 10.9 Hz), 3.41-3.35 (2H, m), 1.78 (2H, m), 1.58-1.43 (2H, m), 1.26 (12H, s).

The following intermediates were prepared in an analogous manner:

| Name | Structure | Starting material(s) | Analytical data |
|---|---|---|---|
| (1-((5-(4,4,5,5-tetramethyl- 1,3,2-dioxaborolan-2-yl)- pyrimidin-2-yl)amino)cyclo- butyl)methanol | 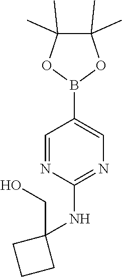 [SMILES: CC1(C)OB(c2cnc(NC3(CO)CCC3)nc2)OC1(C)C] | 1-Aminocyclobutane methanol | δ_{H} (400 MHz; DMSO-d6) 8.36 (2H, s), 7.48 (1H, s), 4.84 (1H, br s), 3.62 (2H, br s), 2.21-2.09 (4H, m), 1.77-1.60 (2H, m), 1.25 (12H, s); ES-MS [M + H]⁺: 306.2 |
| (3S,4R)-4-((5-(4,4,5,5-tetra- methyl-1,3,2-dioxaborolan- 2-yl)pyrimidin-2-yl)amino)- tetrahydro-2H-pyran-3-ol | 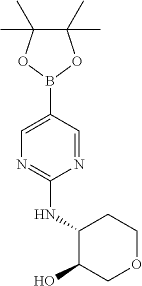 [SMILES: CC1(C)OB(c2cnc(N[C@@H]3CCOC[C@H]3O)nc2)OC1(C)C] | (3S,4R)-4-amino- tetrahydro-2H-pyran- 3-ol | δ_{H}(400 MHz; DMSO-d6) 8.39 (2H, s), 7.47 (1H, d, J 8.2 Hz), 4.92 (1H, d, J 5.5 Hz), 3.45 (1H, m), 3.17 (1H, d, J 5.1 Hz), 3.11- 2.92 (2H, m), 1.91-1.85 (1H, m), 1.35 (2H, br d, J 6.6 Hz), 1.26 (12H, s); ES-MS [M + H]⁺: 322.2 |

### 2-((1-Methyl-2-oxabicyclo[2.1.1]hexan-4-yl)methoxy)-5-(4,4,5,5-tetramethyl-1,3-dioxolan-2-yl)pyrimidine

### Step 1

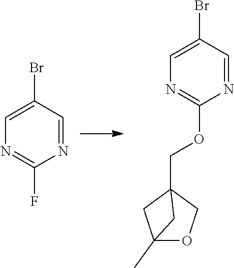 [SMILES: Fc1ncc(Br)cn1 ; CC12CC(COc3ncc(Br)cn3)(CO1)C2]

Sodium hydride (60% dispersion in oil, 125 mg, 3.12 mmol) was added to a solution of (1-methyl-2-oxabicyclo[2.1.1]hexan-4-yl)methanol (200 mg, 1.56 mmol) in THF (4 mL) at 0° C. and the resulting mixture was stirred at 0° C. for 30 min. 5-Bromo-2-fluoropyrimidine (304 mg, 1.72 mmol) was added and the mixture was stirred at 25° C. for 90 min. The reaction mixture was quenched with saturated aqueous ammonium chloride solution (2 mL) and extracted into ethyl acetate (2×2 mL). The combined organic extracts were dried over anhydrous sodium sulfate and then concentrated in vacuo. Flash column chromatography on silica (EtOAc:PE, 100:0 to 50:50) afforded 5-bromo-2-((1-methyl-2-oxabicyclo[2.1.1]hexan-4-yl)methoxy)pyrimidine (200 mg, 0.69 mmol, 45%) as a white solid; ES-MS [M+H]⁺: 258.1.

### Step 2

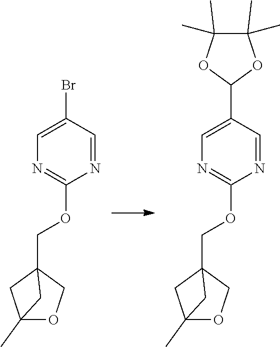

2-((1-Methyl-2-oxabicyclo[2.1.1]hexan-4-yl)methoxy)-5-(4,4,5,5-tetramethyl-1,3-dioxolan-2-yl)pyrimidine was prepared in an analogous manner to 2,2-difluoro-2-(5-(4,4,5,5-tetramethyl-1,3,2-dioxaborolan-2-yl)pyridin-2-yl)ethan-1-ol.

The following intermediates were prepared in an analogous manner:

| Name | Structure | Starting material(s) | Analytical data |
|---|---|---|---|
| (lr,3r)-3-((5-(4,4,5,5- tetramethyl-1,3,2- dioxaborolan-2-yl)- pyrimidin-2-yl)oxy)- cyclobutan-1-ol | 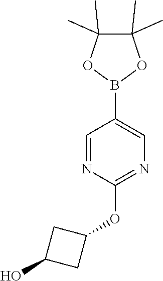 [SMILES: CC1(C)OB(c2cnc(O[C@H]3C[C@H](O)C3)nc2)OC1(C)C] | (1r,3r)-cyclobutane- 1,3-diol | ES-MS [M + H]⁺: 293.1 |
| 2-(2-cyclopropoxy- ethoxy)-5-(4,4,5,5- tetramethyl-1,3,2- dioxaborolan-2-yl)- pyrimidine | 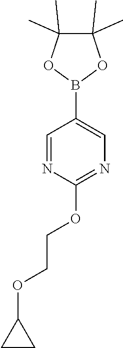 [SMILES: CC1(C)OB(c2cnc(OCCOC3CC3)nc2)OC1(C)C] | 2-cyclopropoxyethan- 1-ol | δ_{H}(400 MHz; DMSO-d6) 8.72 (2H, s), 4.44 (2H, m), 3.77 (2H, m), 3.30 (1H, m), 1.30 (12H, s), 0.52-0.40 (4H, m) |

### 2-Bromo-5-(2-methoxyethoxy)-3-methylpyrazine

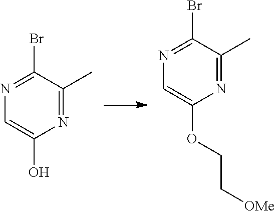

1-Iodo-2-methoxyethane (236 mg, 1.27 mmol) and silver carbonate were added to a solution of 5-bromo-6-methylpyrazin-2-ol (200 mg, 1.06 mmol) in DMF (5 mL). The resulting mixture was stirred at 60° C. for 12 h, then filtered and concentrated in vacuo. Purification by preparative HPLC (column: Phenomenex Luna C18 150*25 mm 10 μm; acetonitrile:water (formic acid); gradient: 35%-65% B over 10 min) afforded 2-bromo-5-(2-methoxyethoxy)-3-methylpyrazine (60 mg, 0.24 mmol, 23%) as a yellow oil; ES-MS [M+H]⁺: 249.1.

The following intermediate was prepared in an analogous manner:

| Name | Structure | Starting material(s) | Analytical data |
|---|---|---|---|
| 2-bromo-3-chloro- 5-methoxypyrazine | 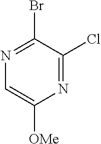 | 5-bromo-6-chloro- pyrazin-2-ol | δ_{H}(400 MHz; DMSO-d6) 8.23 (1H, s), 3.93 (3H, s) |

### 6-Bromo-N-(tetrahydro-2H-pyran-4-yl)pyridin-3-amine

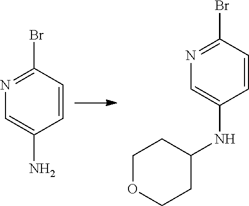 [SMILES: Nc1ccc(Br)nc1 ; Brc1ccc(NC2CCOCC2)cn1]

Sodium cyanoborohydride (1.13 g, 18.0 mmol), 6-bromopyridin-3-amine (1.04 g, 5.99 mmol) and acetic acid (343 μL, 5.99 mmol) were added to a solution of tetrahydropyran-4-one (600 mg, 5.99 mmol) in methanol (15 mL). The reaction mixture was stirred at 40° C. for 12 h then quenched with water (20 mL). The mixture was extracted into ethyl acetate (3×10 mL). The combined organic extracts were dried over anhydrous sodium sulfate and then concentrated in vacuo. Preparative HPLC purification (column: Phenomenex Luna C18 150*25 mm 10 μm; acetonitrile:water (formic acid); gradient: 21%-51% B over 10 min) afforded 6-bromo-N-(tetrahydro-2H-pyran-4-yl)pyridin-3-amine (650 mg, 2.53 mmol, 42%) as a white solid; ES-MS [M+H]⁺: 258.9.

### 2-Chloro-5-(1,1-difluoroethyl)-3-methylpyrazine

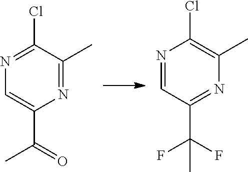

DAST (2.14 g, 9.67 mmol) was added to a solution of 1-(5-chloro-6-methylpyrazin-2-yl)ethan-1-one (330 mg, 1.93 mmol) in dichloromethane (3.3 mL) at 0° C. and the resulting mixture was stirred at 25° C. for 16 h. The reaction quenched with saturated sodium bicarbonate solution (10 mL) and extracted into dichloromethane (3×10 mL). The combined organic extracts were washed with brine (10 mL), dried over anhydrous sodium sulfate and then concentrated in vacuo. Flash column chromatography on silica (EtOAc:PE, 0:100 to 8:92) afforded 2-chloro-5-(1,1-difluoroethyl)-3-methylpyrazine (120 mg, 0.62 mmol, 32%) as a colorless oil; δ_{H} (400 MHz; CDCl₃) 8.54 (1H, s), 2.71 (3H, s), 2.07-1.98 (3H, m). ES-MS [M+H]⁺: 193.2.

### Oxazol-2-ylmethyl (7-bromobenzo[d]thiazol-2-yl)carbamate

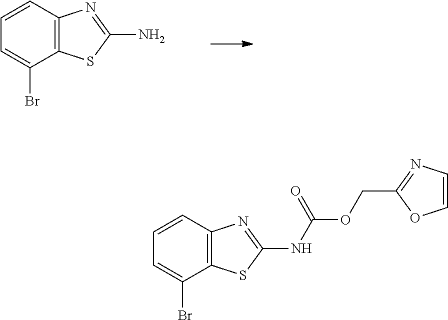 [SMILES: Nc1nc2cccc(Br)c2s1 ; O=C(Nc1nc2cccc(Br)c2s1)OCc1ncco1]

Oxazol-2-ylmethyl (7-bromobenzo[d]thiazol-2-yl)carbamate was prepared from 7-bromobenzo[d]thiazol-2-amine using Synthetic Procedure E, step 3; ES-MS [M+H]⁺: 356.0.

The following intermediate was prepared in an analogous manner:

| Name | Structure | Starting material | Analytical data |
|---|---|---|---|
| oxazol-2-ylmethyl (7-chloro- thiazolo[4,5-b]pyridin-2-yl)- carbamate | 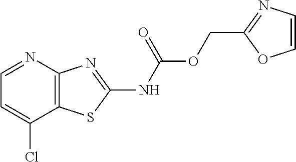 | 7-chlorothiazolo[4,5- b]pyridin-2-amine* | ES-MS[M + H]⁺: 310.9 |

*7-Chlorothiazolo[4,5-b]pyridin-2-amine was prepared from 3-bromo-4-chloropyridin-2-amine in an analogous manner to 7-bromo-6-fluorobenzo[d]thiazol-2-amine

### N-Methyl-3-(5-(4,4,5,5-tetramethyl-1,3,2-dioxaborolan-2-yl)pyridin-2-yl)oxetan-3-amine

### Step 1

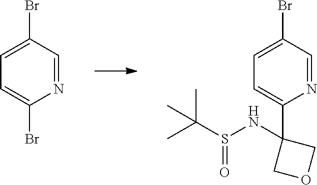 [SMILES: Brc1ccc(Br)nc1 ; CC(C)(C)S(=O)NC1(c2ccc(Br)cn2)COC1]

n-BuLi (2.5 M, 9.29 mL, 3.70 mmol) was added to a solution of 2,5-dibromopyridine (5 g, 21.1 mmol) at −60° C. and stirred for 15 min at this temperature. A solution of 2-Methyl-N-(oxetan-3-ylidene)propane-2-sulfinamide (4.07 g, 23.2 mmol) in toluene (5 mL) was added and the mixture was stirred at −60° C. for a further 30 min. The reaction mixture was quenched with saturated aqueous ammonium chloride solution (30 mL) and extracted into ethyl acetate (3×30 mL). The combined organic extracts were washed with brine (30 mL), dried over anhydrous sodium sulfate and then concentrated in vacuo. Flash column chromatography on silica (EtOAc:PE, 0:100 to 50:50) afforded N-(3-(5-bromopyridin-2-yl)oxetan-3-yl)-2-methylpropane-2-sulfinamide (4.10 g, 12.0 mmol, 57%) as a yellow solid; ES-MS [M+H]⁺: 334.9.

### Step 2

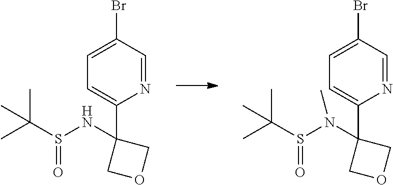 [SMILES: CC(C)(C)S(=O)NC1(c2ccc(Br)cn2)COC1 ; CN(S(=O)C(C)(C)C)C1(c2ccc(Br)cn2)COC1]

Sodium hydride (60% dispersion in oil, 180 mg, 4.50 mmol) was added at 0° C. under nitrogen to a solution of N-(3-(5-bromopyridin-2-yl)oxetan-3-yl)-2-methylpropane-2-sulfinamide (1.0 g, 3.00 mmol) in THF (10 mL). The resulting mixture was stirred at 0° C. for 30 min. A solution of iodomethane (373 μL, 6.00 mmol) in THF (2 mL) was added at 0° C. and stirring was continued at 25° C. for another 12 h. The reaction was quenched with saturated aqueous ammonium chloride solution (15 mL) and extracted into EtOAc (2×15 mL). The combined organic extracts were washed with brine (20 mL), dried over anhydrous sodium sulfate and then concentrated in vacuo to afford N-(3-(5-bromopyridin-2-yl)oxetan-3-yl)-N,2-dimethylpropane-2-sulfinamide (300 mg, 0.92 mmol, 90%) as a yellow oil; ES-MS [M+H]⁺: 348.9.

### Step 3

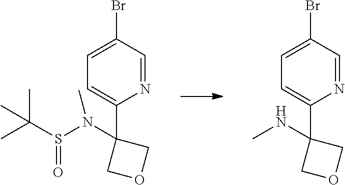 [SMILES: CN(S(=O)C(C)(C)C)C1(c2ccc(Br)cn2)COC1 ; CNC1(c2ccc(Br)cn2)COC1]

HCl (4.0 M in dioxane, 8 mL, 2 mmol) was added to a solution N-(3-(5-bromopyridin-2-yl)oxetan-3-yl)-N,2-dimethylpropane-2-sulfinamide (800 mg, 2.30 mmol) in THF (4 mL) at 0° C. and stirred for 1 h at this temperature. The mixture adjusted to pH 8 with saturated sodium bicarbonate at 0° C. and extracted into ethyl acetate (3×50 mL). The combined organic extracts were washed with brine (30 mL), dried over anhydrous sodium sulfate and then concentrated in vacuo to afford N-(3-(5-bromopyridin-2-yl)oxetan-3-yl)-N,2-dimethylpropane-2-sulfinamide (500 mg, 2.13 mmol, 92% as a red oil; ES-MS [M+H]⁺: 242.9.

### Step 4

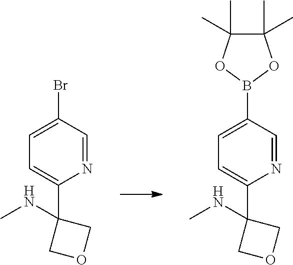

N-Methyl-3-(5-(4,4,5,5-tetramethyl-1,3,2-dioxaborolan-2-yl)pyridin-2-yl)oxetan-3-amine was prepared from 3-(5-bromopyridin-2-yl)-N-methyloxetan-3-amine in an analogous manner to 2,2-difluoro-2-(5-(4,4,5,5-tetramethyl-1,3,2-dioxaborolan-2-yl)pyridin-2-yl)ethan-1-ol; ES-MS [M+H]⁺: 209.1 (boronic acid).

### 6-((5-(4,4,5,5-Tetramethyl-1,3,2-dioxaborolan-2-yl)pyridin-2-yl)methyl)-2-oxa-6-azaspiro[3.3]heptane

### Step 1

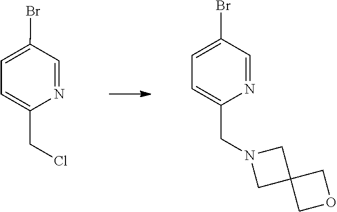 [SMILES: ClCc1ccc(Br)cn1 ; Brc1ccc(CN2CC3(COC3)C2)nc1]

Potassium carbonate was added to a solution of 5-bromo-2-(chloromethyl)pyridine (200 mg, 0.97 mmol) and 2-oxa-6-azaspiro[3.3]heptane (144 mg, 1.45 mmol) in acetonitrile (5 mL) and the resulting mixture was heated at 80° C. for 12 h. Water (5 mL) was added and the mixture was extracted into ethyl acetate (3×10 mL). The combined organic extracts were dried over anhydrous sodium sulfate and then concentrated in vacuo to afford 6-((5-bromopyridin-2-yl)methyl)-2-oxa-6-azaspiro[3.3]heptane (200 mg, 0.74 mmol, 77%) as a white solid; ES-MS [M+H]⁺: 269.9.

### Step 2

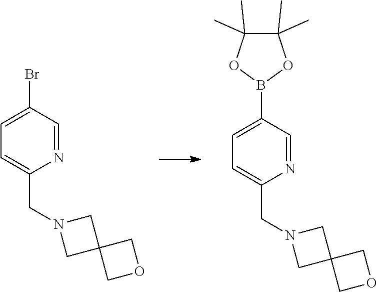 [SMILES: Brc1ccc(CN2CC3(COC3)C2)nc1 ; CC1(C)OB(c2ccc(CN3CC4(COC4)C3)nc2)OC1(C)C]

6-((5-(4,4,5,5-tetramethyl-1,3,2-dioxaborolan-2-yl)pyridin-2-yl)methyl)-2-oxa-6-azaspiro[3.3]heptane was prepared from 6-((5-bromopyridin-2-yl)methyl)-2-oxa-6-azaspiro[3.3]heptane in an analogous manner to 2,2-difluoro-2-(5-(4,4,5,5-tetramethyl-1,3,2-dioxaborolan-2-yl)pyridin-2-yl)ethan-1-ol; ES-MS [M+H]⁺: 235.1 (boronic acid).

### 5-Methyl-3,6-dihydro-2H-pyran-4-yl trifluoromethanesulfonate

### Step 1

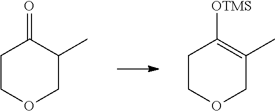

Trimethylsilyl trifluoromethanesulfonate (2.4 mL, 13.3 mmol) was slowly added to a solution of 3-methyltetrahydro-4H-pyran-4-one (2.00 g, 17.5 mmol) and triethylamine (2.40 mL, 17.24 mmol) in dichloromethane (20 mL) at 0° C. and stirred for 30 min at this temperature. Water (20 mL) was added and the reaction mixture was extracted into dichloromethane (3×20 mL). The combined organic extracts were dried over anhydrous sodium sulfate and then concentrated in vacuo to afford trimethyl((5-methyl-3,6-dihydro-2H-pyran-4-yl)oxy)silane (3.70 g) as a yellow oil which was used without further purification.

### Step 2

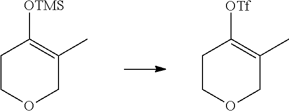

Methyl lithium (1.6 M in THF, 13 mL, 20.8 mmol) was slowly added to a solution of trimethyl((5-methyl-3,6-dihydro-2H-pyran-4-yl)oxy)silane (3.70 g, 19.9 mmol) in THF (30 mL) at 0° C. and stirred for 30 min at this temperature. Phenyl triflimide (14.0 g, 39.2 mmol) was then added and stirring was continued at 30° C. for 30 min. The reaction was quenched with saturated aqueous ammonium chloride solution (10 mL) and acetic acid was added until the pH was ˜6. Water (20 mL) was added and the reaction mixture was extracted into EtOAc (3×20 mL). The combined organic extracts were dried over anhydrous sodium sulfate and then concentrated in vacuo. Flash column chromatography on silica (EtOAc:PE, 0:100 to 5:95) afforded 5-methyl-3,6-dihydro-2H-pyran-4-yl trifluoromethanesulfonate (1.6 g, 6.50 mmol, 33%) as a yellow oil; δ_{H} (400 MHz; CDCl₃) 4.13-4.10 (2H, m), 3.89 (2H, m), 2.46 (2H, m), 1.72 (3H, s).

### tert-butyl 3-((1H-pyrazol-4-yl)oxy)azetidine-1-carboxylate

### Step 1

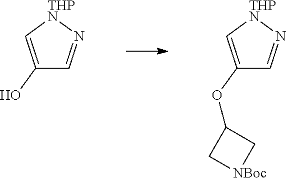

Cesium carbonate (11.6 g, 35.7 mmol) was added to a solution of 1-(tetrahydro-2H-pyran-2-yl)-1H-pyrazol-4-ol (3.00 g, 17.8 mmol) and tert-butyl 3-iodoazetidine-1-carboxylate (7.57 g, 26.8 mmol) in DMF (30 mL). The mixture was stirred at 40° C. for 1 h. Water (50 mL) was added and the reaction mixture was extracted into EtOAc (3×20 mL). The combined organic extracts were washed with brine (20 mL), dried over anhydrous sodium sulfate and then concentrated in vacuo. Flash column chromatography on silica (EtOAc:PE, 0:100 to 40:50) afforded tert-butyl 3-((1-(tetrahydro-2H-pyran-2-yl)-1H-pyrazol-4-yl)oxy)azetidine-1-carboxylate (3.60 g, 11.1 mmol, 62%) as a yellow oil.

### Step 2

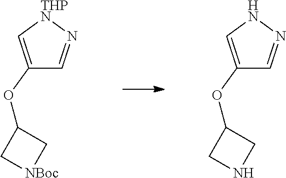

TFA (15 mL, 202 mmol) was added to a solution of tert-butyl 3-((1-(tetrahydro-2H-pyran-2-yl)-1H-pyrazol-4-yl)oxy)azetidine-1-carboxylate (3.40 g, 10.5 mmol) in dichloromethane (30 mL) and the mixture was stirred at 25° C. for 1 h. The reaction mixture was concentrated in vacuo to afford 4-(azetidin-3-yloxy)-1H-pyrazole (1.40 g, 10.1 mmol, 96%) as a yellow oil.

### Step 3

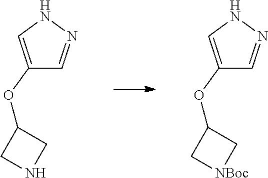 [SMILES: c1n[nH]cc1OC1CNC1 ; CC(C)(C)OC(=O)N1CC(Oc2cn[nH]c2)C1]

Di-tert-butyl decarbonate (2.20 g, 10.1 mmol) was added to a solution of 4-(azetidin-3-yloxy)-1H-pyrazole (1.40 g, 10.1 mmol) and triethylamine (7.00 mL, 50.3 mmol) in methanol (15 mL). The mixture was stirred at 25° C. for 1 h, then concentrated in vacuo. Purification by preparative HPLC (column: Kromasil Eternity XT 250*80 mm 10 μm; acetonitrile:water (NH₄HCO₃); gradient: 20%-50% B over 20 min) afforded tert-butyl 3-((1H-pyrazol-4-yl)oxy)azetidine-1-carboxylate as a yellow solid; δ_{H} (400 MHz; DMSO-d6) 12.47 (1H, br s), 7.44-7.24 (2H, m), 4.76-4.65 (1H, m), 4.26-4.10 (2H, m), 3.82-3.68 (2H, m), 1.41-1.35 (9H, m).

### Biological Assays

### DLD-1 Cell Viability Assay

Compound cytotoxicity in DLD-1 human colorectal adenocarcinoma cells (ATCC CCL-221) was gauged using the CellTiter Glo 2.0 Luminescent Cell Viability Assay (Promega). DLD-1 cells maintained in growth medium (RPMI, 10% FBS, 1% P/S) were seeded into 1536-well black clear bottom plates (Corning 3836) at a density of 400 cells/5 μL/well (0.08×10\hat{ }⁶ cells/mL) and incubated overnight in a 5% CO₂ incubator at 37° C. Compounds in 10 point 1:3 serial dilution in DMSO (provided by compound management in 384-well Low Dead Volume Echo certified plates, Labcyte LP-0200) were stamped (15 nL) onto 1536-well plates containing DLD-1 cells (at about 30-40% cell density) for a final compound concentration ranging from 30 μM to 1.5 nM. 30 μM (final concentration) of Toxoflavin served as a low control while DMSO only served as a high control in these experiments. The final DMSO concentration in the assay plate is 0.3%. Each sample was tested in triplicate.

72 hours post compound introduction, 5 μL of CellTiter Glo 2.0 at room temperature is added to the cells in media using a multidrop combi and incubated for 30 minutes at room temperature. Luminescence from the samples is recorded with a Clariostar Plus plate reader and all data is normalized to the average of DMSO treated group and expressed as the percentage of control. IC₅₀ values based on curve classification for each compound were calculated from the concentration response curves (percentage inhibition) using Dotmatics data management software.

### Immunofluorescence Nuclear Condensate Assay

An immunofluorescence (IF) assay was used to measure the sequestration of beta catenin into nuclear condensates. DLD-1 cells (ATCC CCL-221) were maintained in growth medium RPMI 1640 (ATCC modification) (Thermo, A1049101) supplemented with 10% fetal bovine serum (VWR, 89510-196). Twenty-four hours prior to compound treatment, cells were seeded into 1536-well black clear bottom plates (Greiner, GR 789866) at a density of 600 cells/5 μL/well and incubated overnight in a 5% CO₂ incubator at 37° C. for 24 hours. Compounds in 10 point 1:3 serial dilution in DMSO in 384-well Low Dead Volume Echo certified plates (Labcyte, LP-0200) were stamped (15 nL) onto 1536-well plates containing DLD-1 cells for a final compound concentration ranging from 30 μM to 1.5 nM. NCB-0846 (30 μM) (Selleck, 58392) served as a high control while DMSO served as a low control in these experiments. The final DMSO concentration in the assay plate is 0.3%. Each sample was tested in triplicate.

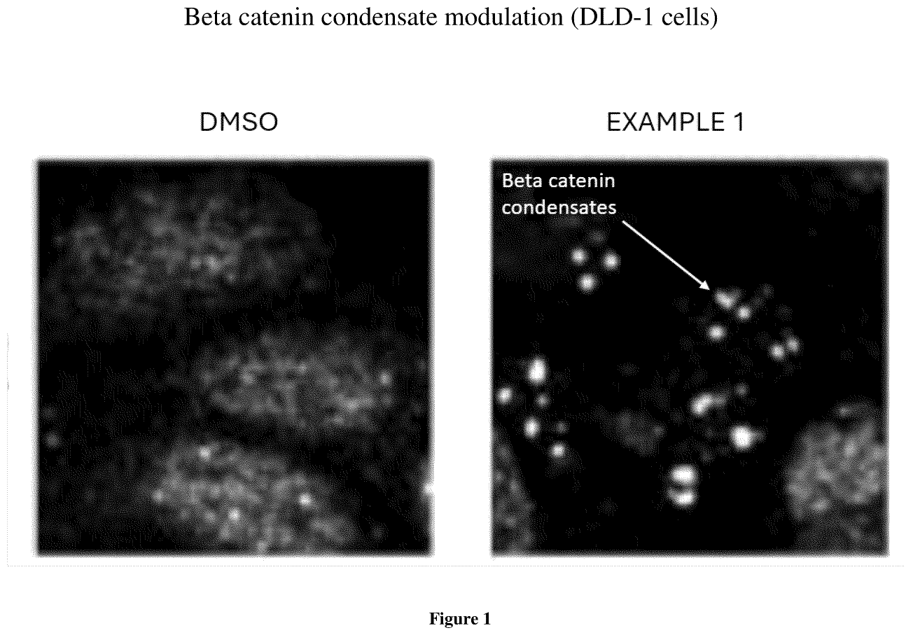

Twenty-four hours after compound treatment, cells were fixed with the addition of 2 μL of 12% formaldehyde (Sigma, 1.04003) at room temperature for 15 minutes, permeabilized in 0.1% Triton X-100 (Sigma, 93443) in PBS for 15 minutes, blocked in 0.2% gelatin from cold water fish (Sigma, G7765) in PBS for 1 hour, and then stained with a 1:1000 dilution of the phospho beta catenin antibody (Invitrogen, 703638) in 0.2% gelatin overnight at 4° C. followed by incubation with a 1:10000 dilution of Hoechst (Thermo, 113570) and with a 1:1000 dilution of secondary antibody (Thermo, A11070) for 1 hour at room temperature. Plates were then imaged (Opera Phenix, Perkin Elmer), and analyzed with the Harmony software quantitating the fraction of cells with bright phospho beta catenin depots (see FIG. 1). The data was normalized to the high (NCB-0846, 30 M) and low (DMSO) controls. EC₅₀ values based on curve classification for each compound were calculated from the concentration response curves (percentage response) using Dotmatics data management software.

The data from the viability IC₅₀ and immunofluorescence (IF) EC₅₀ values are shown in the table below:

| Ex. No. | DLD-1 Cell Viability IC₅₀ | DLD-1 IF EC₅₀ |
|---|---|---|
| 1 | A | A |
| 2 | A | A |
| 3 | A | A |
| 4 | A | A |
| 5 | B | B |
| 6 | C | C |
| 7 | A | A |
| 8 | A | A |
| 9 | C | C |
| 10 | A | B |
| 11 | A | A |
| 12 | A | A |
| 13 | B | B |
| 14 | C | C |
| 15 | B | B |
| 16 | C | C |
| 17 | B | B |
| 18 | B | B |
| 19 | A | A |
| 20 | C | C |
| 21 | A | A |
| 22 | A | A |
| 23 | A | A |
| 24 | B | B |
| 25 | B | B |
| 26 | B | B |
| 27 | C | C |
| 28 | A | B |
| 29 | C | C |
| 30 | A | A |
| 31 | C | nd |
| 32 | B | A |
| 33 | B | B |
| 34 | C | B |
| 35 | B | A |
| 36 | A | A |
| 37 | A | A |
| 38 | A | A |
| 39 | A | A |
| 40 | A | A |
| 41 | A | A |
| 42 | A | A |
| 43 | A | A |
| 44 | B | B |
| 45 | A | A |
| 46 | C | C |
| 47 | A | A |
| 48 | B | B |
| 49 | C | C |
| 50 | B | B |
| 51 | B | B |
| 52 | A | A |
| 53 | A | A |
| 54 | B | B |
| 55 | A | A |
| 56 | A | A |
| 57 | A | B |
| 58 | A | A |
| 59 | B | B |
| 60 | B | B |
| 61 | A | A |
| 62 | A | A |
| 63 | C | C |
| 64 | A | A |
| 65 | B | B |
| 66 | A | A |
| 67 | A | A |
| 68 | B | B |
| 69 | B | B |
| 70 | B | B |
| 71 | C | C |
| 72 | B | nd |
| 73 | B | A |
| 74 | B | C |
| 75 | A | A |
| 76 | B | B |
| 77 | C | C |
| 78 | B | C |
| 79 | A | B |
| 80 | C | C |
| 81 | A | A |
| 82 | B | B |
| 83 | A | A |
| 84 | B | A |
| 85 | A | A |
| 86 | C | C |
| 87 | A | A |
| 88 | B | B |
| 89 | B | B |
| 90 | B | A |
| 91 | A | A |
| 92 | C | B |
| 93 | B | B |
| 94 | C | C |
| 95 | B | A |
| 96 | C | C |
| 97 | A | A |
| 98 | B | B |
| 99 | A | A |
| 100 | A | A |
| 101 | B | C |
| 102 | C | C |
| 103 | C | C |
| 104 | B | C |
| 105 | B | A |
| 106 | A | A |
| 107 | B | nd |
| 108 | A | A |
| 109 | A | A |
| 110 | A | A |
| 111 | A | A |
| 112 | A | A |
| 113 | A | A |
| 114 | A | A |
| 115 | A | A |
| 116 | A | A |
| 117 | A | A |
| 118 | A | A |
| 119 | A | A |
| 120 | A | A |
| 121 | A | A |
| 122 | A | A |
| 123 | A | A |
| 124 | B | B |
| 125 | A | A |
| 126 | A | A |
| 127 | A | A |
| 128 | A | A |
| 129 | A | A |
| 130 | A | C |
| 131 | A | A |
| 132 | B | B |
| 133 | A | A |
| 134 | A | A |
| 135 | A | A |
| 136 | A | C |
| 137 | A | nd |
| 138 | A | A |
| 139 | A | A |
| 140 | A | A |
| 141 | A | A |
| 142 | B | B |
| 143 | A | A |
| 144 | A | A |
| 145 | A | A |
| 146 | A | A |
| 147 | B | B |
| 148 | B | B |
| 149 | A | A |
| 150 | A | A |
| 151 | B | B |
| 152 | B | B |
| 153 | A | B |
| 154 | B | A |
| 155 | B | B |
| 156 | B | B |
| 157 | B | B |
| 158 | A | A |
| 159 | A | A |
| 160 | A | A |
| 161 | C | C |
| 162 | A | A |
| 163 | B | B |
| 164 | A | A |
| 165 | B | nd |
| 166 | A | A |
| 167 | B | B |
| 168 | A | A |
| 169 | A | A |
| 170 | B | B |
| 171 | A | A |
| 172 | A | A |
| 173 | A | B |
| 174 | B | B |
| 175 | B | B |
| 176 | B | nd |
| 177 | C | nd |
| 178 | A | nd |
| 179 | B | A |
| 180 | A | A |
| 181 | A | A |
| 182 | B | B |
| 183 | B | B |
| 184 | A | A |
| 185 | A | A |
| 186 | A | A |
| 187 | A | A |
| 188 | A | A |
| 189 | B | B |
| 190 | B | B |
| 191 | B | B |
| 192 | A | A |
| 193 | B | B |
| 194 | C | C |
| 195 | B | B |
| 196 | A | A |
| 197 | B | B |
| 198 | B | B |
| 199 | B | B |
| 200 | B | A |
| 201 | A | B |
| 202 | B | C |
| 203 | B | B |
| 204 | A | A |
| 205 | B | C |
| 206 | B | C |
| 207 | A | A |
| 208 | C | C |
| 209 | B | B |
| 210 | A | B |
| 211 | B | B |
| 212 | C | B |
| 213 | C | B |
| 214 | C | C |
| 215 | C | C |
| 216 | C | C |
| 217 | A | A |
| 218 | B | B |
| 219 | B | B |
| 220 | A | B |
| 221 | A | B |
| 222 | A | A |
| 223 | A | A |
| 224 | A | A |
| 225 | A | A |
| 226 | B | B |
| 227 | B | C |
| 228 | A | A |
| 229 | A | A |
| 230 | C | C |
| 231 | A | A |
| 232 | A | A |
| 233 | A | A |
| 234 | A | B |
| 235 | A | A |
| 236 | C | C |
| 237 | A | A |
| 238 | A | A |
| 239 | A | A |
| 240 | A | A |
| 241 | B | A |
| 242 | A | A |
| 243 | B | C |
| 244 | A | B |
| 245 | A | A |
| 246 | B | A |
| 247 | C | B |
| 248 | B | B |
| 249 | A | A |
| 250 | B | B |
| 251 | C | C |
| 252 | B | B |
| 253 | B | B |
| 254 | B | B |
| 255 | B | B |
| 256 | C | B |
| 257 | B | B |
| 258 | A | A |
| 259 | B | B |
| 260 | B | nd |
| 261 | A | A |
| 262 | A | A |
| 263 | C | C |
| 264 | B | B |
| 265 | B | C |
| 266 | A | A |
| 267 | B | B |
| 268 | C | C |
| 269 | B | A |
| 270 | B | B |
| 271 | A | B |
| 272 | C | C |
| 273 | B | B |
| 274 | A | A |
| 275 | B | B |
| 276 | A | A |
| 277 | B | B |
| 278 | B | B |
| 279 | B | B |
| 280 | B | A |
| 281 | C | C |
| 282 | B | B |
| 283 | B | B |
| 284 | A | nd |
| 285 | C | C |
| 286 | B | B |
| 287 | C | nd |
| 288 | B | B |
| 289 | B | B |
| 290 | A | A |
| 291 | B | B |
| 292 | C | C |
| 293 | B | B |
| 294 | B | B |
| 295 | A | A |
| 296 | B | A |
| 297 | A | A |
| 298 | B | B |
| 299 | A | A |
| 300 | A | A |
| 301 | A | A |
| 302 | B | B |
| 303 | B | B |
| 304 | A | A |
| 305 | A | B |
| 306 | A | A |
| 307 | B | A |
| 308 | A | A |
| 309 | A | B |
| 310 | A | A |
| 311 | A | A |
| 312 | A | A |
| 313 | A | B |
| 314 | B | B |
| 315 | C | C |
| 316 | B | B |
| 317 | B | B |
| 318 | A | A |
| 319 | B | B |
| 320 | A | A |
| 321 | A | nd |
| 322 | A | A |
| 323 | C | B |
| 324 | B | B |
| 325 | A | A |
| 326 | A | A |
| 327 | B | B |
| 328 | A | A |
| 329 | A | A |
| 330 | A | A |
| 331 | A | B |
| 332 | A | A |
| 333 | B | B |
| 334 | C | C |
| 335 | A | A |
| 336 | C | C |
| 337 | B | C |
| 338 | C | C |
| 339 | C | C |
| 340 | C | nd |
| 341 | A | A |
| 342 | A | A |
| 343 | B | A |
| 344 | A | A |
| 345 | C | C |
| 346 | B | A |
| 347 | B | A |
| 348 | A | A |
| 349 | B | B |
| 350 | A | A |
| 351 | A | A |

A: IC₅₀ or EC₅₀ <0.1 μM
B: IC₅₀ or EC₅₀ ≥0.1 and <1 μM
C: IC₅₀ or EC₅₀ ≥1 and <10 μM
nd: not determined

### Mouse Pharmacokinetic Study

Example 1 was formulated in DMSO:PEG400:water (1:4:5) for IV dosing and Cremophor® RH 40: 20% HP-β-CD (1:19) for PO dosing. Three female BALB/c mice were dosed IV (1 mg/kg) and serial bleed time-points were taken at 0 min, 5 min, 15 min, 30 min, 1 h, 2 h, 4 h, 8 h and 24 h. Ten female BALB/c mice were dosed PO (10 mg/kg) and bleeds were taken at 0 min, 30 min, 1 h, 2 h, 4 h, 8 h and 24 h across the group (maximum two samples per mouse). Samples were prepared by protein precipitation with acetonitrile and analyzed by quantitative LC/MS using a AB API 5500 LC/MS/MS. PK parameters were estimated by non-compartmental model using WinNonlin 8.3. The PK data for Example 1 is shown in the table below:

| Route of | | | | |
|---|---|---|---|---|
| administration | Dose | Parameter | Value | |
| PO | 10 mg/kg | T_{1/2} | 7.7 | h |
| | | Cmax | 2.19 | μM |
| | | AUCₗₐₛₜ | 3.18 h · μM | |
| | | F % | 52% | |
| IV | 1 mg/kg | Cl | 64 | mL/min/kg |
| | | Vₛₛ | 16.9 | L/kg |

### Colorectal Cancer Cell-Line Derived Xenograft Study

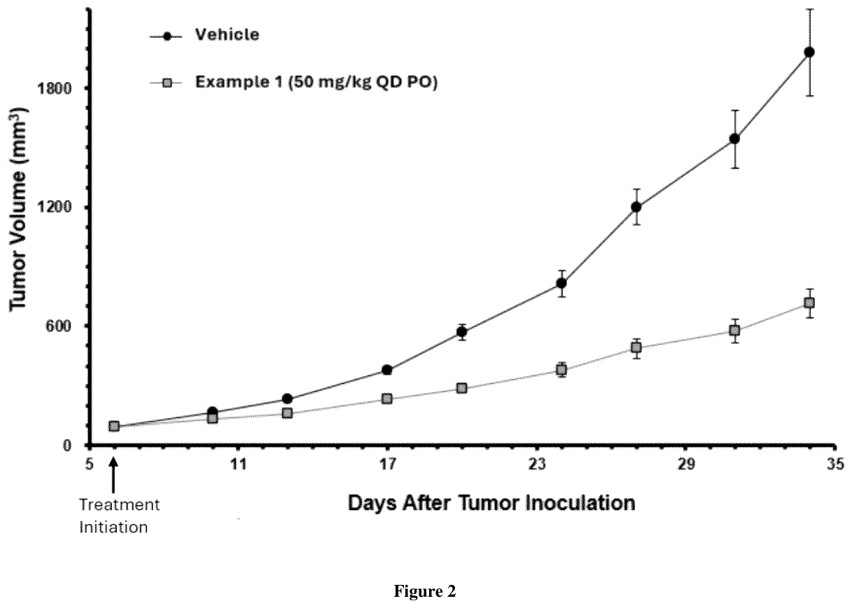

HCT-116 cells (ATCC CCL-247) were cultured in McCoy's % A media until sufficient cell numbers for implantation into mice were obtained. Cells were harvested and inoculated (5×10⁶ in 100 μL in McCoy's 5a medium) subcutaneously into the right flank of nude mice and tumor growth was monitored by caliper measurement. When tumors reached ˜70-100 mm³, mice were randomized into treatment groups (5 groups, n=10/group) and treatment was initiated. Example 1, formulated in Cremophor® RH 40: 20% HP-β-CD (1:19) for PO dosing, was administered as follows: Vehicle; Example 1-50 mg/kg QD. Tumor volume measurements were captured twice weekly using digital calipers and the data is represented as the mean group tumor volume+/−S.E.M. FIG. 2 shows the tumor volumes for mice treated with Example 1 and mice provided with vehicle.

qPCR Assay

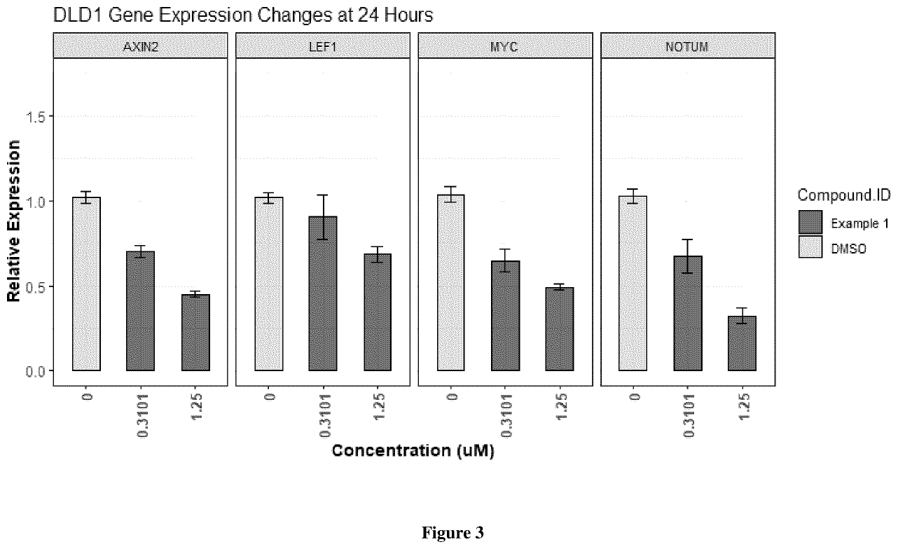

Gene expression of beta catenin target genes was measured by duplexed Real-Time quantitative Polymerase Chain Reaction (RT-qPCR) in DLD-1 human colorectal adenocarcinoma cells (ATCC CCL-221). DLD-1 cells maintained in growth medium (RPMI ATCC formulation, 10% FBS) were seeded in 384-well black clear bottom plates (Revvity, Phenoplate) at a density of 2000 cells/40 μL/well (5.0×10⁴ cells/mL) and incubated overnight at 37° C. with 5% CO₂. The example compound diluted in DMSO at the indicated concentrations was added to the cells. The final DMSO concentration in each well was 0.1%. 24 hours after compound addition, cells were washed once with 1× Phosphate Buffered Saline (PBS) and lysed using 30 μL of cell lysis buffer (ThermoFisher #4391851C). Cell lysis occurs over an 8-minute period at room temperature while shaking the plate on a platform shaker. After cell lysis, 3 μL of stop solution (ThermoFisher #4391851C) was added. 4 μL of cell lysate was then transferred to 384-well qPCR-compatible plates (ThermoFisher #4483285). qPCR Master Mix (ThermoFisher #A15299) containing target and housekeeping TaqMan probes is then dispensed, and each reaction was assembled according to the manufacturer's instructions. Transcript abundance was measured by qPCR using a Quantstudio 7 Flex thermocycler. Each sample was tested in duplicate across 4 beta catenin target genes: Axin2 (ThermoFisher Hs00610344_m1), Lef1 (ThermoFisher Hs01547250_m1), Myc (ThermoFisher Hs00153408_m1), and Notum (ThermoFisher Hs00394510_m1). The housekeeping control gene, RNase P, was duplexed with one target gene per well. Quantification was performed by calculating the ΔΔCt and relative quantification (2^{-ΔΔCt}) values. FIG. 3 shows the gene expression changes in DLD-1 cells treated with Example 1 or DMSO.

1. A compound having the structural Formula I: 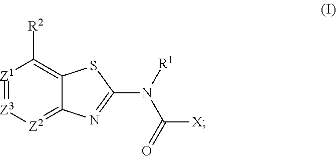
or a pharmaceutically acceptable salt thereof, wherein:
z¹ is N or —CR³;
z² is N or —CR⁵;
z³ is N or —CR⁴;
R¹ is hydrogen, (C₁-C₄)alkyl, hydroxy(C₁-C₄)alkyl, (C₁-C₄)alkylene(C₁-C₄)alkoxy, or (C₁-C₄)alkylene[OP(O)(OH)₂];
R² is aryl, heteroaryl, heterocyclyl, or cycloalkyl, each of which are optionally substituted with 1 to 3 groups selected from R^{1A};
R³ and R⁴ are each independently hydrogen, halo, cyano, (C₁-C₄)alkyl, halo(C₁-C₄)alkyl, (C₁-C₄)alkoxy, halo(C₁-C₄)alkoxy, (C₂-C₄)alkenyl, (C₁-C₄)alkylence[heterocyclyl], (C₁-C₄)alkylene[aryl], (C₁-C₄)alkylene[heteroaryl], (C₁-C₄)alkylene[cycloalkyl], —S(O)₂(C₁-C₄)alkyl, or —S(O)(C₁-C₄)alkyl, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano;
R⁵ is hydrogen, halo, or (C₁-C₄)alkyl;
X is —OR⁶ or —NR⁷R⁸;
R⁶ is cycloalkyl, heterocyclyl, (C₁-C₄)alkylene(C₁-C₄alkoxy), (C₁-C₄)alkylene[aryl], (C₁-C₄)alkylene[heteroaryl], (C₁-C₄)alkylene[heterocyclyl], or (C₁-C₄)alkylene[cycloalkyl], wherein the aryl, heteroaryl, and cycloalkyl are each optionally substituted with 1 to 3 groups selected from R^{2A};
R⁷ and R⁸ are taken together to form a heterocyclyl optionally substituted with 1 to 3 groups selected from R^{2A};
each R^{1A} is independently selected from halo, cyano, (C₁-C₄)alkyl, hydroxy(C₁-C₄)alkyl, hydroxy[halo(C₁-C₄)alkyl], cyano(C₁-C₄)alkyl, halo(C₁-C₄)alkyl, (C₁-C₄)alkoxy, deuterated(C₁-C₄)alkoxy, (C₁-C₄)alkoxyO(C₁-C₄)alkyl, (C₁-C₄)alkoxyO[deuterated(C₁-C₄)alkyl], halo(C₁-C₄)alkoxy, (C₁-C₄)alkoxyO[halo(C₁-C₄)alkyl], oxo, hydroxy, —O[hydroxy(C₁-C₄)alkyl], —O[cycloalkyl], —O[heterocyclyl], —O[aryl], —O[heteroaryl], (C₁-C₄)alkylene[cycloalkyl], (C₁-C₄)alkylene[aryl], (C₁-C₄)alkylene[heterocyclyl], (C₁-C₄)alkylene[heteroaryl], —O(C₁-C₄)alkylene[NR^{a}R^{b}], —O(C₁-C₄)alkylene[NR^{a}C(O)R^{b}], —O(C₁-C₄)alkylene[cycloalkyl], —O(C₁-C₄)alkylene[aryl], —O(C₁-C₄)alkylene[heterocyclyl], —O(C₁-C₄)alkylene[heteroaryl], cycloalkyl, heterocyclyl, aryl, heteroaryl, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], (C₁-C₄)alkoxyO[cycloalkyl], (C₁-C₄)alkoxyO[heterocyclyl], (C₁-C₄)alkoxyO[heteroaryl], (C₁-C₄)alkoxyO[phenyl], —C(O)[heterocyclyl], —SR^{a}, —S(O)R^{a}, —SO₂R^{a}, —S(O)(NR^{a})R^{b}, —NR^{b}SO₂R^{b}, —SO₂NR^{a}, —C(O)R^{a}, —C(O)OR^{a}, —C(O)NR^{a}R^{b}, —NR^{a}C(O)R^{b}, —NR^{a}C(O)NR^{b}, —NR^{a}R^{b}, —NH(C₁-C₄)alkylene[heterocyclyl], and (C₁-C₄)alkyleneNR^{a}R^{b}, where each occurrence of cycloalkyl, heterocyclyl, aryl, and heteroaryl alone, or as part of another group, is optionally substituted with 1 to 3 groups selected from halo, oxo, (C₁-C₄)alkyl, halo(C₁-C₄)alkyl, hydroxy(C₁-C₄)alkyl, (C₁-C₄)alkoxy, halo(C₁-C₄)alkoxy, hydroxy, —NH₂, —NH(C₁-C₄)alkyl, —N((C₁-C₄)alkyl)₂, cycloalkyl, heterocyclyl, aryl, heteroaryl, and cyano;
each R^{2A} is independently selected from halo, (C₁-C₄)alkyl, halo(C₁-C₄)alkyl, hydroxy(C₁-C₄)alkyl, deuterated(C₁-C₄)alkyl, (C₁-C₄)alkoxy, halo(C₁-C₄)alkoxy, deuterated(C₁-C₄)alkoxy, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], oxo, —O[cycloalkyl], cyano, hydroxy, S(O)(C₁-C₄)alkyl, —S(O)₂(C₁-C₄)alkyl, hydroxy, cycloalkyl, heterocyclyl, aryl, heteroaryl, (C₁-C₄)alkylene[cycloalkyl], (C₁-C₄)alkylene[aryl], (C₁-C₄)alkylene[heterocyclyl], (C₁-C₄)alkylene[heteroaryl], —C(O)R^{a1}, —C(O)OR^{a1}, —C(O)NR^{a1}R^{b1}, —NR^{a1}C(O)R^{b1}, —NR^{a1}C(O)NR^{b1}, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano; and
each R^{a}, R^{a1}, R^{b}, and R^{b1} is each independently selected from hydrogen, (C₁-C₄)alkyl, halo(C₁-C₄)alkyl, (C₁-C₄)alkylene(C₁-C₄)alkoxy, (C₁-C₄)alkyleneNH₂, (C₁-C₄)alkyleneNH((C₁-C₄alkyl), (C₁-C₄)alkyleneN((C₁-C₄alkyl)₂, cycloalkyl, and heterocyclyl, wherein the cycloalkyl and heterocyclyl are each optionally substituted with (C₁-C₄)alkyl, hydroxy, or hydroxy(C₁-C₄)alkyl;
provided that:
(i) if R⁷ and R⁸, taken together with the N to which they are attached, form pyrrolidinyl, then R^{2A} is not —C(O)NH₂;
(ii) if R⁷ and R⁸, taken together with the N to which they are attached, form piperidinyl, then R^{2A} is not benzyl; and
(iii) if R⁷ and R⁸, taken together with the N to which they are attached, form 6- to 12-membered heterocyclyl, then R² is substituted by 1 to 3 groups selected from R^{1A}.

2. The compound of claim 1, or a pharmaceutically acceptable salt thereof, wherein:
z³ is —CR⁴; and
each R^{2A} is independently selected from halo, (C₁-C₄)alkyl, halo(C₁-C₄)alkyl, hydroxy(C₁-C₄)alkyl, deuterated(C₁-C₄)alkyl, (C₁-C₄)alkoxy, halo(C₁-C₄)alkoxy, deuterated(C₁-C₄)alkoxy, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxy], oxo, cyano, hydroxy, S(O)(C₁-C₄)alkyl, —S(O)₂(C₁-C₄)alkyl, hydroxy, cycloalkyl, heterocyclyl, aryl, heteroaryl, (C₁-C₄)alkylene[cycloalkyl], (C₁-C₄)alkylene[heterocyclyl], (C₁-C₄)alkylene[heteroaryl], —C(O)Rai, —C(O)OR^{a1}, —NR^{a1}C(O)R^{b1}, —NR^{a1}C(O)NR^{b1}, wherein said heterocyclyl, aryl, heteroaryl, and cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, and cyano.

3. The compound of claim 1, or a pharmaceutically acceptable salt thereof, wherein:
(i) z¹ is N, z² is —CR⁵, and z³ is —CR⁴;
(ii) z¹ is —CR³, z³ is —CR⁴, and z² is N; or
(iii) z¹ is —CR³, z² is —CR⁵, and z³ is —CR⁴.

4. (canceled)

5. The compound of claim 1, or a pharmaceutically acceptable salt thereof, wherein R³ is:
(i) hydrogen, halo, (C₁-C₄)alkyl, halo(C₁-C₄)alkyl, (C₂-C₄)alkenyl, (C₁-C₄)alkoxy, cyano, —S(O)₂(C₁-C₄)alkyl, or (C₁-C₄)alkylene[heterocyclyl], wherein said heterocyclyl is optionally substituted by 1 halo; or
(ii) hydrogen, —F, —Cl, —CH₃, —CF₃, —CH═CH₂, —OCH₃, cyano, —S(O)₂CH₃,

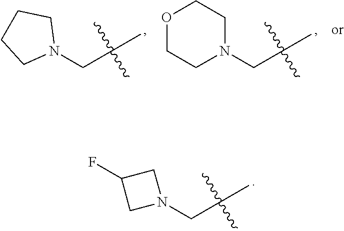 [SMILES: *CN1CCCC1 ; *CN1CCOCC1 ; *CN1CC(F)C1]

6. (canceled)

7. The compound of claim 1, or a pharmaceutically acceptable salt thereof, wherein R⁴ is:
(i) hydrogen, halo, or (C₁-C₄)alkyl; or
(ii) hydrogen, —F, or —CH₃.

8. (canceled)

9. The compound of claim 1, or a pharmaceutically acceptable salt thereof, wherein R⁵ is:
(i) hydrogen, halo, or (C₁-C₄)alkyl; or
(ii) hydrogen, —F, or —CH.

10. (canceled)

11. The compound of claim 1, or a pharmaceutically acceptable salt thereof, wherein R¹ is:
(i) hydrogen or (C₁-C₄)alkylene[OP(O)(OH)₂]; or
(ii) hydrogen.

12. (canceled)

13. The compound of claim 1, having the structural Formula II: 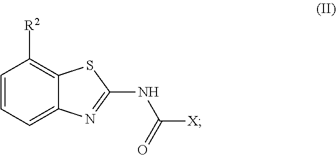
or a pharmaceutically acceptable salt thereof.

14. The compound of claim 1, or a pharmaceutically acceptable salt thereof, wherein R² is:
(i) phenyl, cyclohexyl, azetidinyl, morpholinyl, tetrahydropyridinyl, tetrahydropyranyl, dihydropyranyl, piperdinyl, azaspirohexanyl, azaspiroheptanyl, oxaazaspiroheptanyl, oxaazaspirooctanyl, oxaazaspirononanyl, pyrazolyl, triazolyl, pyridinyl, pyrimidinyl, pyrazinyl, pyridazinyl, isoindolinyl, dihydropyrazolooxazinyl, dihydrodioxinopyridinyl, or hexahydropyrazinopyridooxazinyl, each of which is optionally substituted with 1 to 3 groups selected from R^{1A};
(ii) 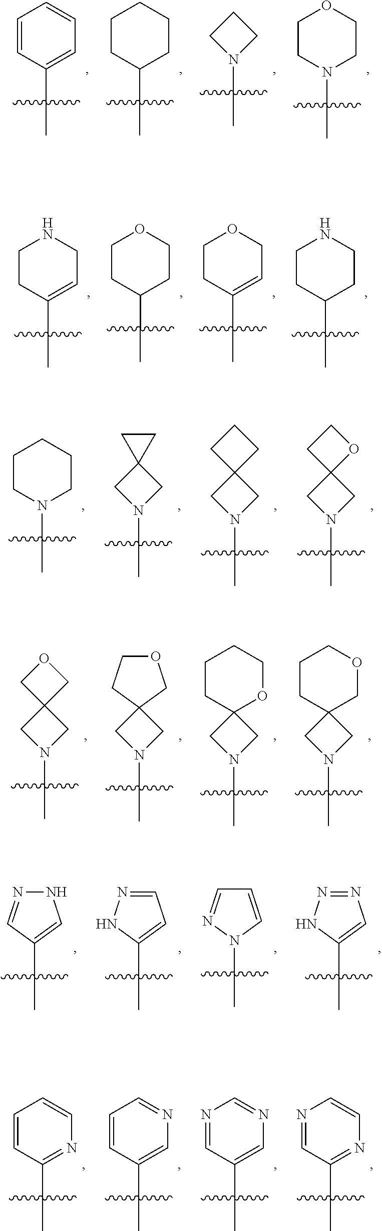 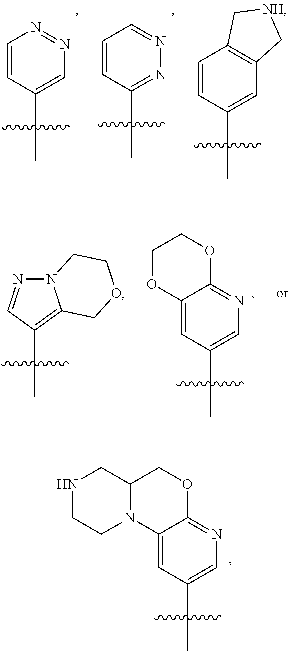
each of which is optionally substituted with 1 or 2 groups selected from R^{1A}; or
(iii) 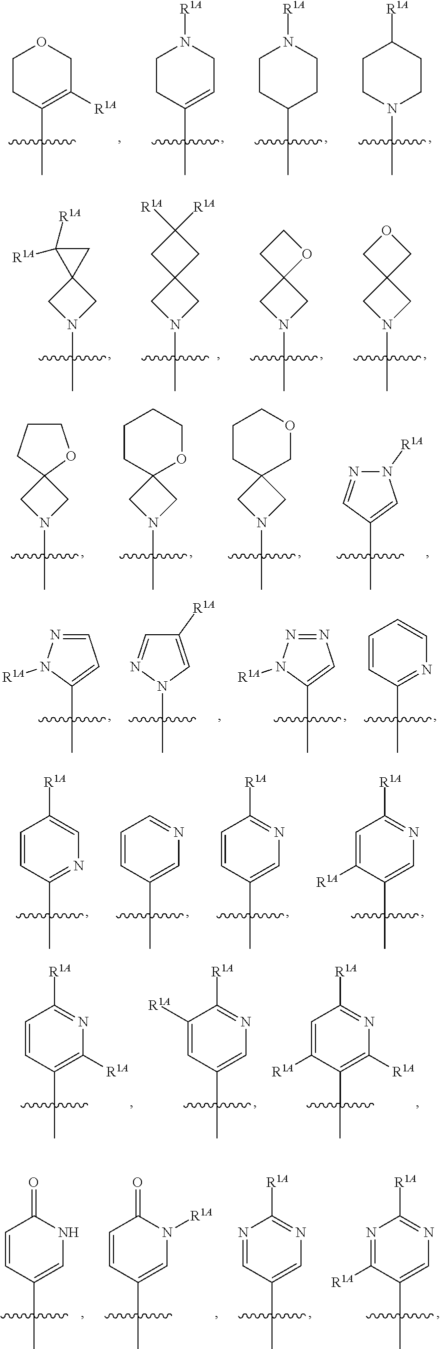 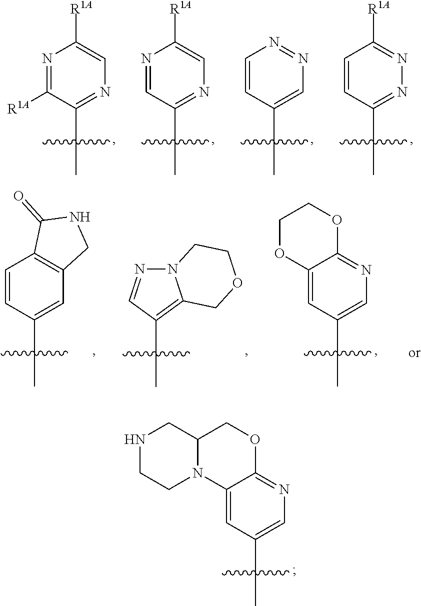
and wherein
(a) each R^{1A} is independently halo, (C₁-C₄)alkyl, halo(C₁-C₄)alkyl, hydroxy(C₁-C₄)alkyl, hydroxy[halo(C₁-C₄)alkyl], (1-C₄)alkylene(C₁-C₄)alkoxy, (C₁-C₄)alkylene[deuterated(C₁-C₄)alkoxy], (C₁-C₄)alkylene[halo(C₁-C₄)alkoxy], hydroxy, (C₁-C₄)alkoxy, halo(C₁-C₄)alkoxy, (C₁-C₄)alkoxyO(C₁-C₄)alkyl, (C₁-C₄)alkoxyO[deuterated(C₁-C₄)alkyl], (C₁-C₄)alkoxyO[halo(C₁-C₄)alkyl], —O(C₁-C₄)alkylene[NR^{a}R^{b}], —NR^{a}R^{b}, —NH(C₁-C₄)alkylene[heterocyclyl], cyano, —C(O)R^{a}, —C(O)NR^{a}R^{b}, —SO₂R^{a}, cycloalkyl, heterocyclyl, (C₁-C₄)alkylene[heterocyclyl], —O(C₁-C₄)alkylene[cycloalkyl], —O(C₁-C₄)alkylene[heterocyclyl], (C₁-C₄)alkoxyO[cycloalkyl], —O[cycloalkyl], or —O[heterocyclyl], where each occurrence of cycloalkyl and heterocyclyl, alone, or as part of another group, is optionally substituted with 1 to 3 groups selected from oxo, (C₁-C₄)alkyl, hydroxy, —NH(C₁-C₄alkyl), and heterocyclyl; and wherein R^{a} and R^{b} are each independently hydrogen, (C₁-C₄)alkyl, (C₁-C₄)alkylene(C₁-C₄)alkoxy, cycloalkyl, or heterocyclyl, wherein the cycloalkyl and heterocyclyl are each optionally substituted with (C₁-C₄)alkyl, hydroxy, or hydroxy(C₁-C₄)alkyl; or
(b) each R^{1A} is independently —F, —Cl, —CH₃, —CH(CH₃)₂, —CH₂F, —CHF₂, —CF₃, —CF₂CH₃, —CH₂CF₃, —CF₂CH₂OH, —CH₂OCH₃, —CH₂OCD₃, —CH₂OCHF₂, —CH₂OCF₃, —CH₂CH₂OCH₃, —C(OH)(CH₃)₂, —CH₂C(OH)(CH₃)₂, hydroxy, —OCH₃, —OCH₂CH₃, —OCH(CH₃)₂, —OCHF₂, —OCF₃, —OCH₂CH₂OCH₃, —OCH₂CH₂OCD₃, —OCH₂CH₂OCHF₂, —OCH₂CH₂N(CH₃)₂, —NH₂, —NHCH₃, —N(CH₃)₂, cyano, —C(O)CH₃, —C(O)NH₂, —C(O)NHCH₃, —C(O)N(CH₃)₂, —C(O)NHCH₂CH₂OCH₃, —S(O)₂CH₃, cyclopropyl,

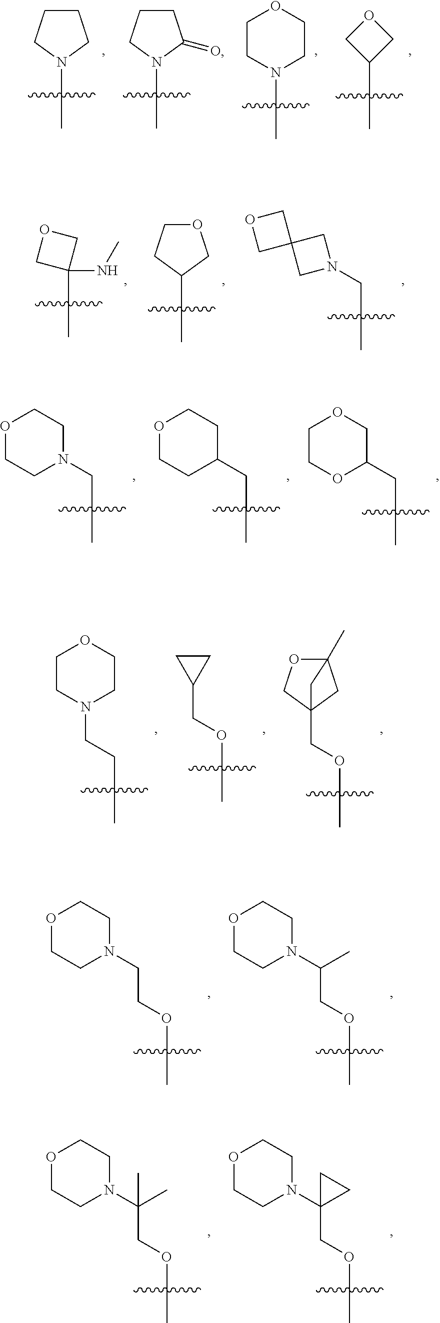

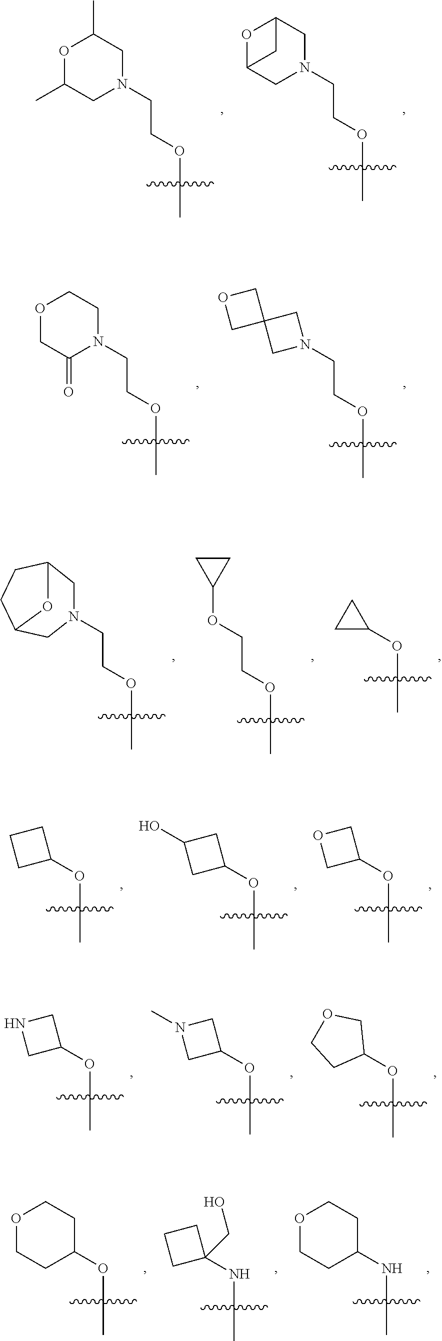

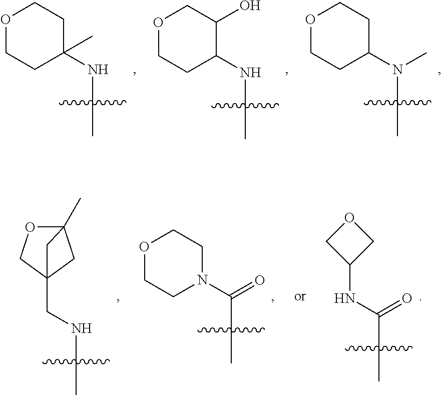

15.-19. (canceled)

20. The compound of claim 1, or a pharmaceutically acceptable salt thereof, wherein:
(i) X is —OR⁶; and
(ii) R⁶ is:
(a) (C₁-C₄)alkylene[heteroaryl]; or
(b)

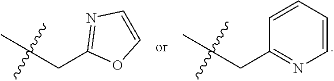 [SMILES: *Cc1ncco1 ; *Cc1ccccn1]

21.-23. (canceled)

24. The compound of claim 1, or a pharmaceutically acceptable salt thereof, wherein:
(i) X is —NR⁷R⁸; and
(ii) R⁷ and R⁸, together with the N to which they are attached, form:
(a) azetidinyl, azaspirohexanyl, azaspiroheptanyl, oxaazaspiroheptanyl, thiaazaspiroheptanyl, diazaspirooctanyl, oxaazaspirooctanyl, oxaazaspirononanyl, diazaspirononanyl, oxadiazaspirononanyl, or dioxaazaspirononanyl, each of which are optionally substituted with 1 to 3 groups selected from R^{2A};
(b) 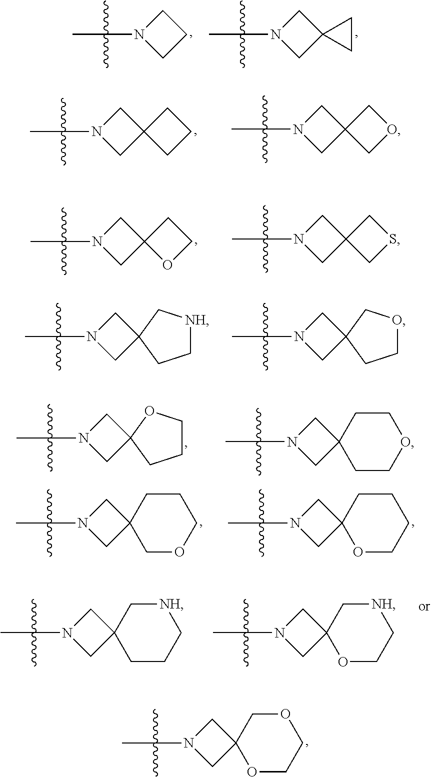
each of which are optionally substituted with 1 to 3 groups selected from R^{2A}; or
(c) 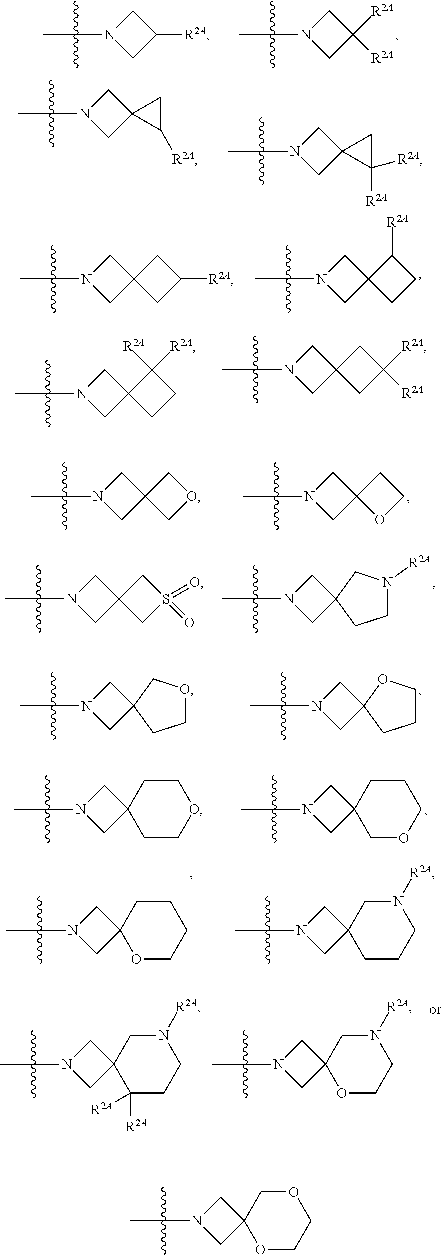
and wherein:
each R^{2A} is independently:
(I) halo, (C₁-C₄)alkyl, deuterated(C₁-C₄)alkyl, halo(C₁-C₄)alkyl, (C₁-C₄)alkylene(C₁-C₄)alkoxy, (C₁-C₄)alkylene[deuterated(C₁-C₄)alkoxy], (C₁-C₄)alkylene[halo(C₁-C₄)alkoxyl, hydroxy(C₁-C₄)alkyl, hydroxy, (C₁-C₄)alkoxy, deuterated(C₁-C₄)alkoxy, —S(O)₂(C₁-C₄)alkyl, cyano, or cycloalkyl; or
(II) —F, —CH₃, —CD₃, —CH₂F, —CF₃, —CH₂CF₃, —CH₂OCH₃, —CH₂OCH₂CH₃, —CH₂OCD₃, —CH₂OCHF₂, —CH₂OCF₃, —C(OH)(CH₃)₂, —C(CH₃)₂OCH₃, hydroxy, —OCH₃, —OCD₃, —OCH₂CH₃, —OCH(CH₃)₂, —S(O)₂CH₃, cyano, or cyclopropyl.

25.-28. (canceled)

29. The compound of claim 1, having the structural Formula III: 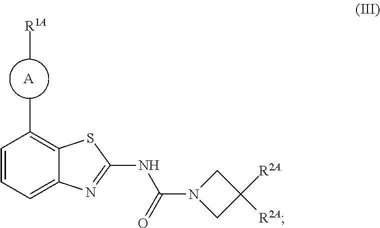
or a pharmaceutically acceptable salt thereof, wherein Ring A is a 5- to 6-membered nitrogen containing heteroaryl.

30. The compound of claim 29, or a pharmaceutically acceptable salt thereof, wherein Ring A is;
(i) pyrazolyl, pyridinyl, or pyrimidinyl, each of which is substituted by R^{1A};
(ii) 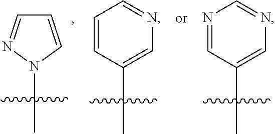
each of which is substituted by R^{1A}; or
(iii)

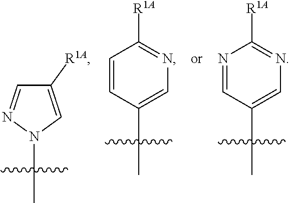

31.-32. (canceled)

33. The compound of claim 29, or a pharmaceutically acceptable salt thereof, wherein R^{1A} is;
(i) (C₁-C₄)alkoxy, halo(C₁-C₄)alkoxy, (C₁-C₄)alkoxyO(C₁-C₄)alkyl, —O[cycloalkyl], or —O[heterocyclyl]; or
(ii) —OCH₃, —OCHF₂, —OCH₂CH₃, —OCH(CH₃)₂, —O-cyclopropyl, —OCH₂CH₂OCH₃, or

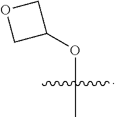 [SMILES: *OC1COC1]

34. (canceled)

35. The compound of claim 29, or a pharmaceutically acceptable salt thereof, wherein each R^{2A} is independently:
(i) halo, (C₁-C₄)alkyl, (C₁-C₄)alkylene(C₁-C₄)alkoxy, (C₁-C₄)alkylene[deuterated(C₁-C₄)alkoxy], (C₁-C₄)alkoxy, or deuterated(C₁-C₄)alkoxy; or
(ii) —F, —CH₃, —CH₂OCH₃, —CH₂OCH₂CH₃, —CH₂OCD₃, or —OCD₃.

36. (canceled)

37. The compound of claim 29, or a pharmaceutically acceptable salt thereof, wherein:
(i) R^{A} is (C₁-C₄)alkoxy; and
each R^{2A} is independently halo, (C₁-C₄)alkylene(C₁-C₄)alkoxy, or (C₁-C₄)alkylene[deuterated(C₁-C₄)alkoxy]; or
(ii) R^{1A} is —OCH₃ or —OCH₂CH₃; and
each R^{2A} is independently —F, —CH₂OCH₃, or —CH₂OCD₃.

38. (canceled)

39. The compound of claim 1, wherein the compound is: 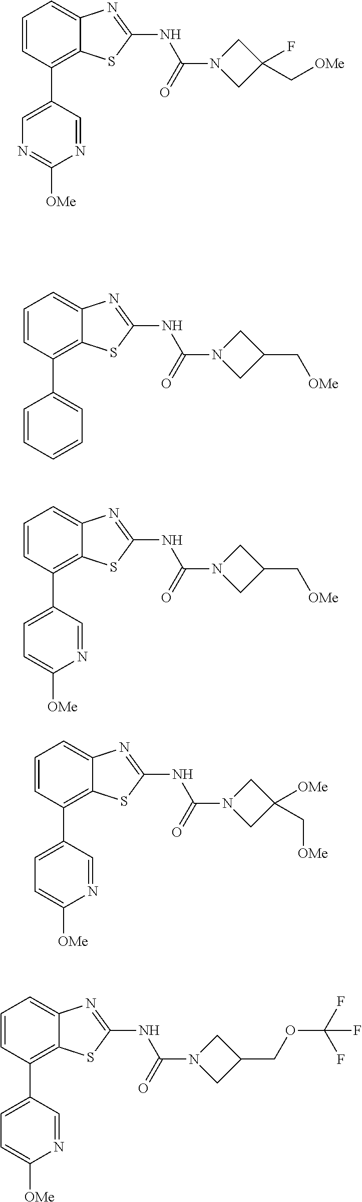 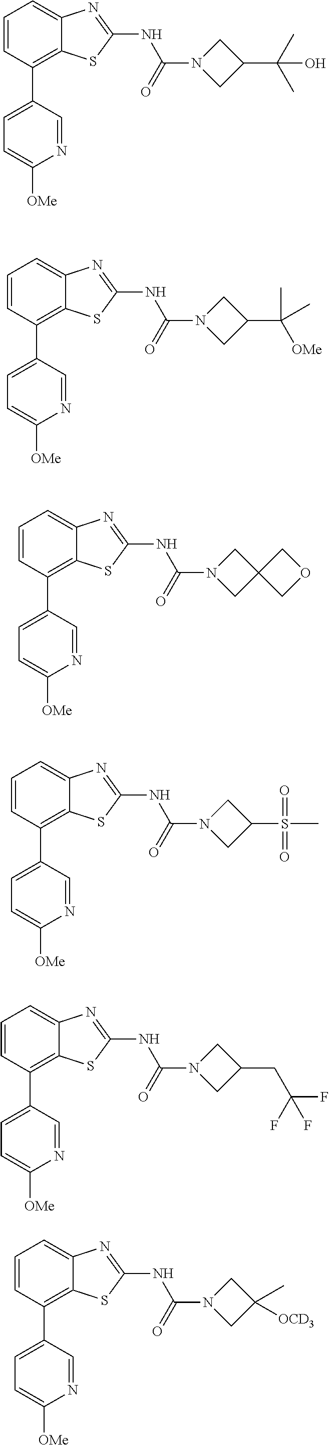 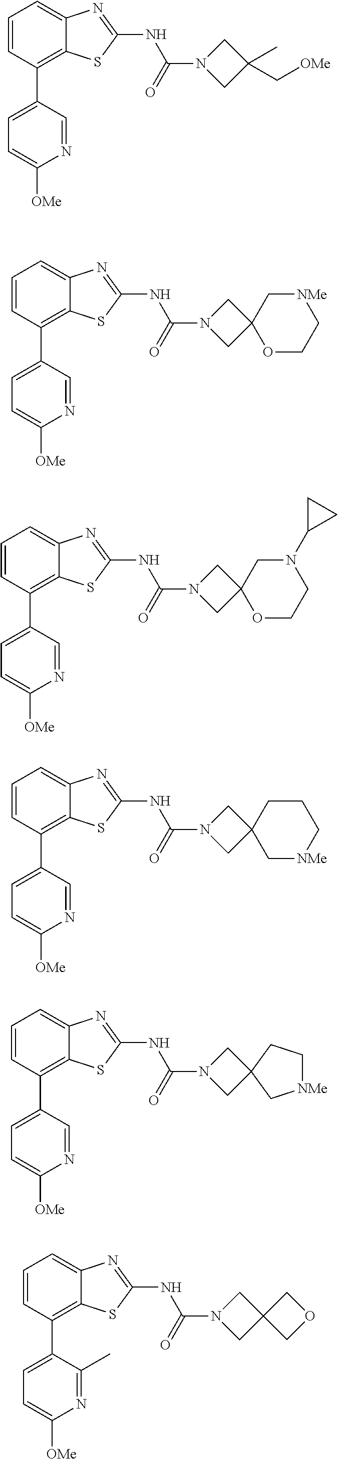 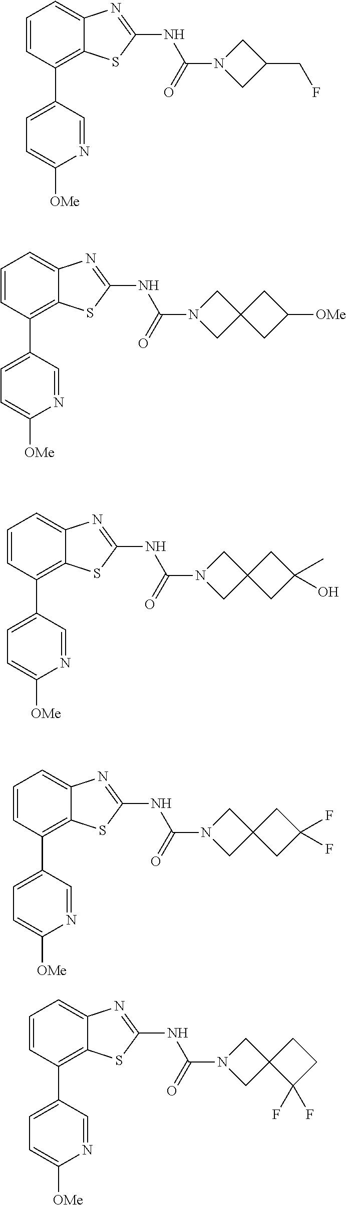 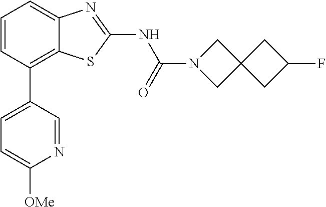 [SMILES: COc1ccc(-c2cccc3nc(NC(=O)N4CC5(CC(F)C5)C4)sc23)cn1] 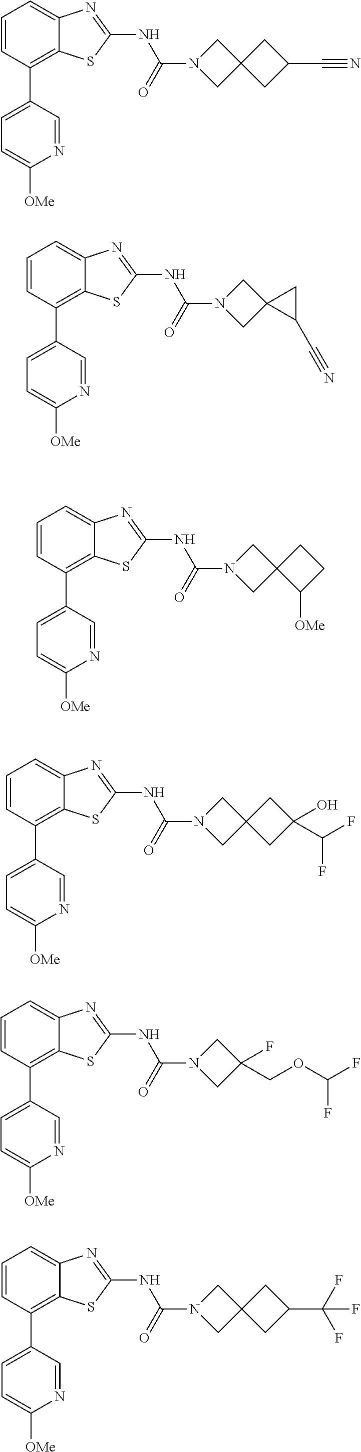 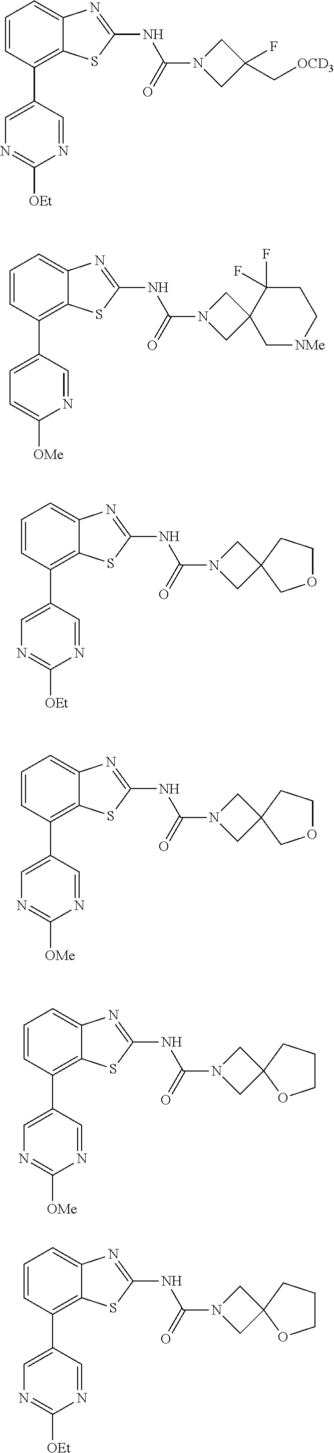 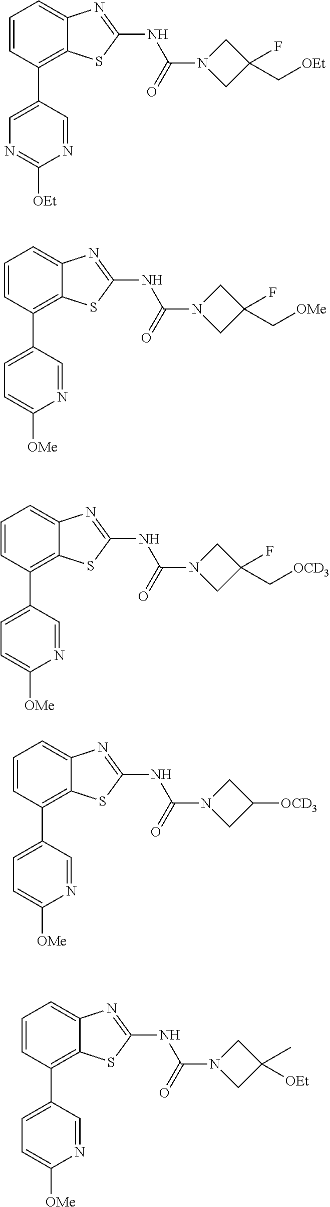 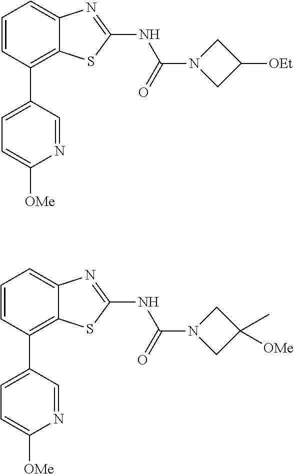 [SMILES: CCOC1CN(C(=O)Nc2nc3cccc(-c4ccc(OC)nc4)c3s2)C1 ; COc1ccc(-c2cccc3nc(NC(=O)N4CC(C)(OC)C4)sc23)cn1] 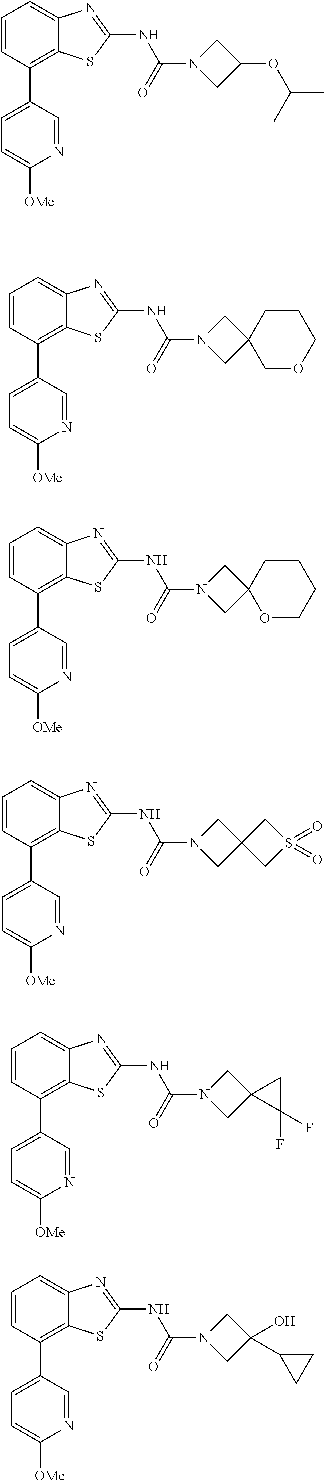 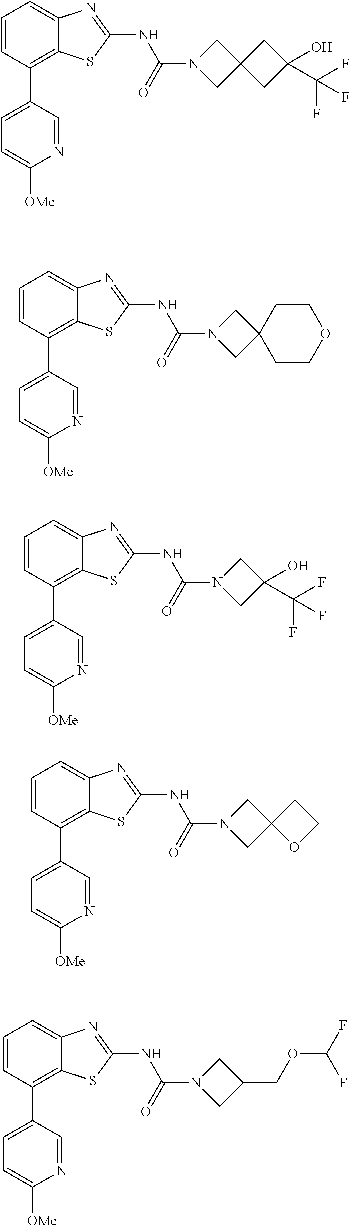 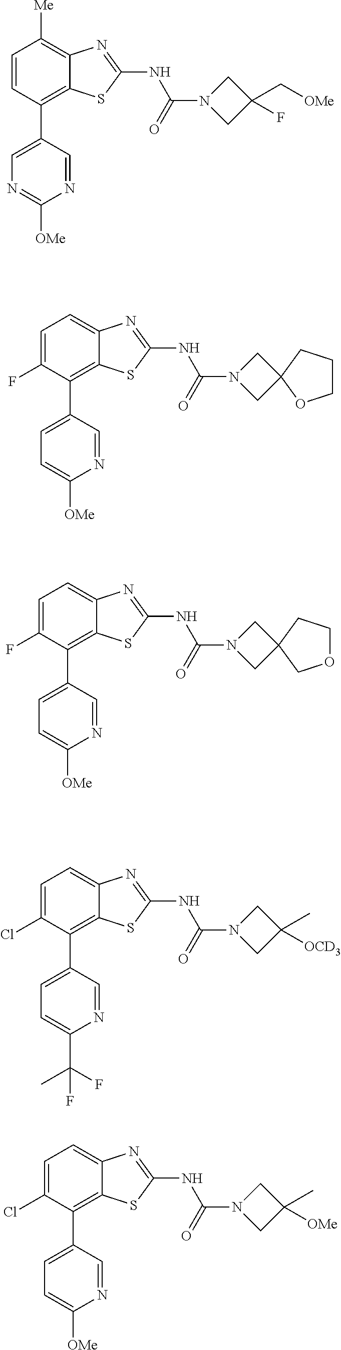 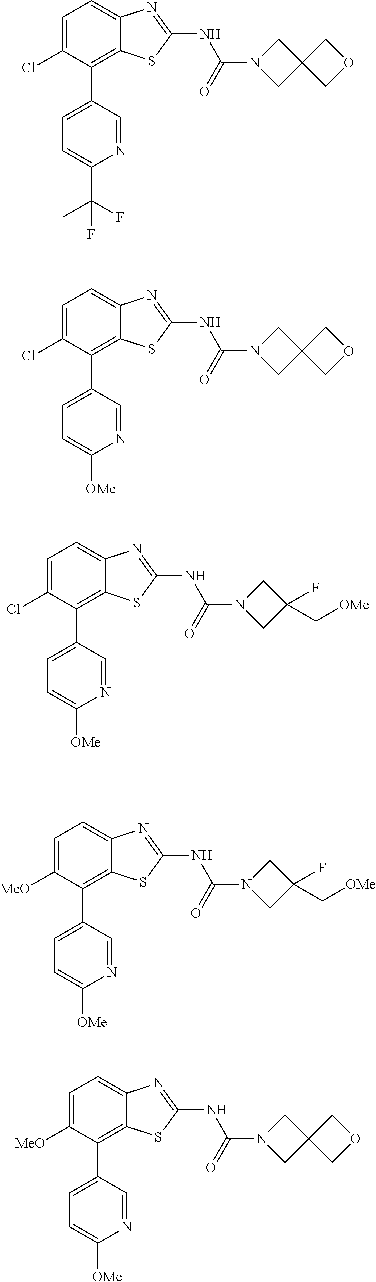 [SMILES: CC(F)(F)c1ccc(-c2c(Cl)ccc3nc(NC(=O)N4CC5(COC5)C4)sc23)cn1 ; COc1ccc(-c2c(Cl)ccc3nc(NC(=O)N4CC5(COC5)C4)sc23)cn1 ; COCC1(F)CN(C(=O)Nc2nc3ccc(Cl)c(-c4ccc(OC)nc4)c3s2)C1 ; COCC1(F)CN(C(=O)Nc2nc3ccc(OC)c(-c4ccc(OC)nc4)c3s2)C1 ; COc1ccc(-c2c(OC)ccc3nc(NC(=O)N4CC5(COC5)C4)sc23)cn1] 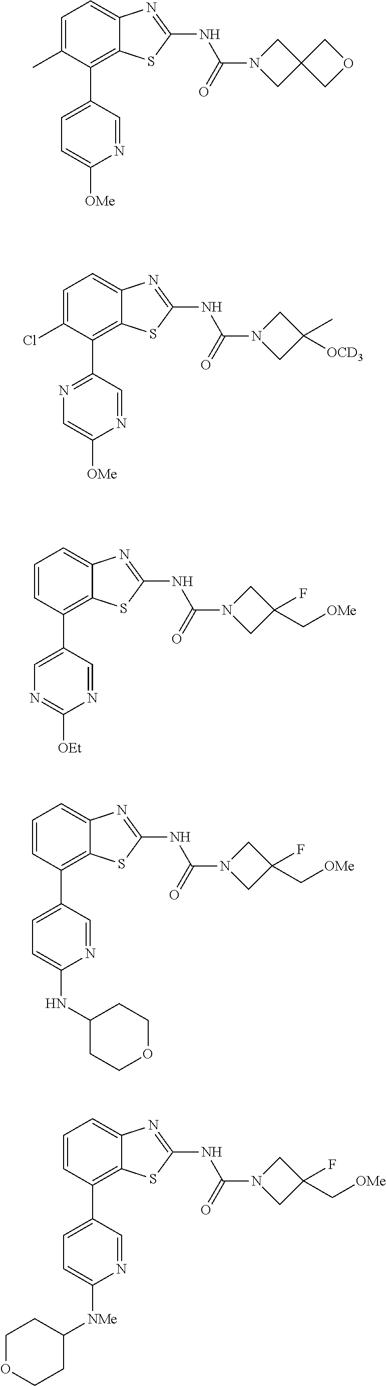 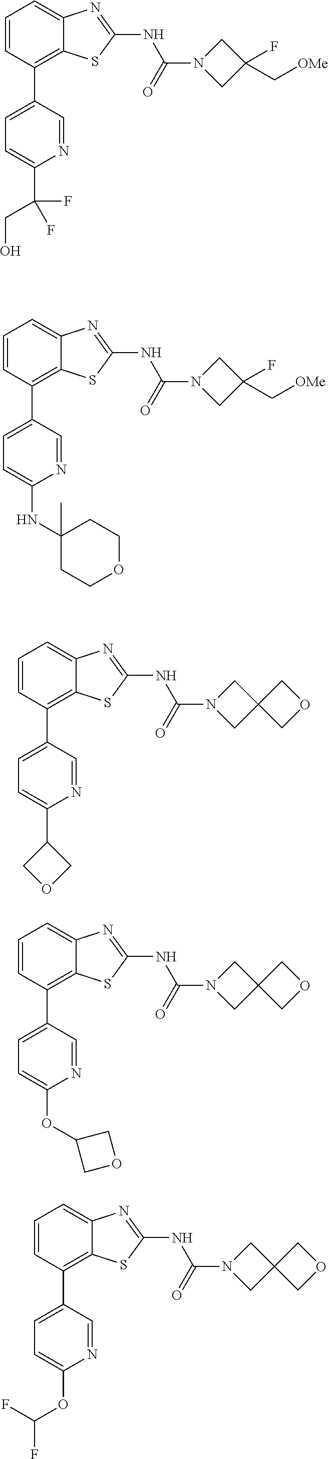 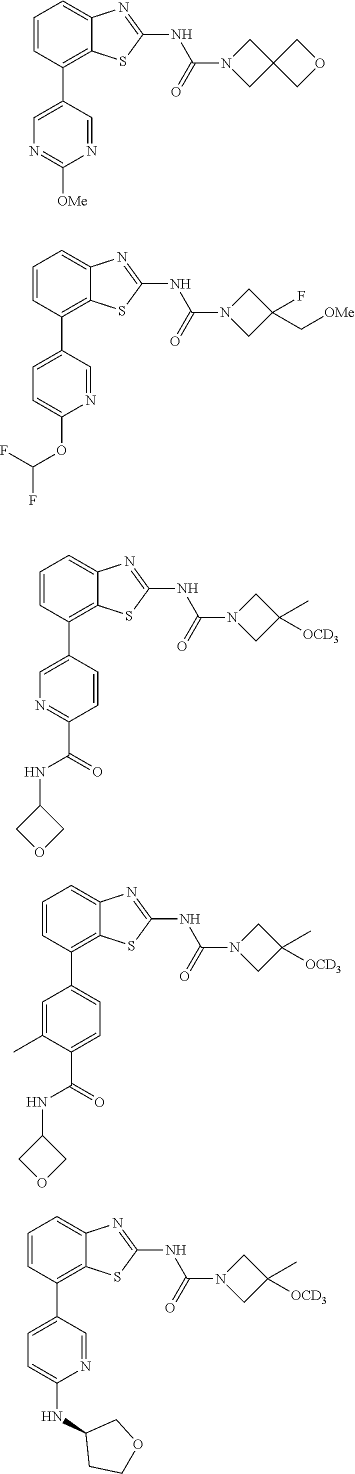 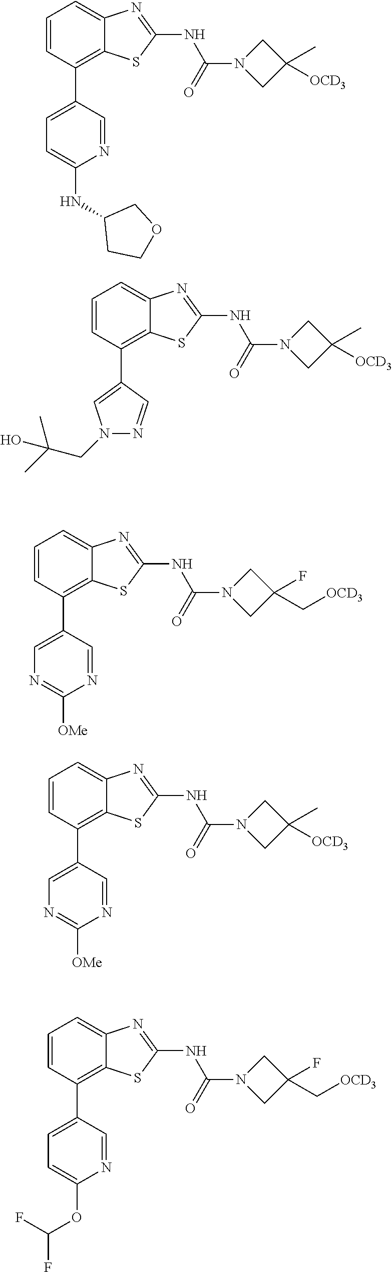 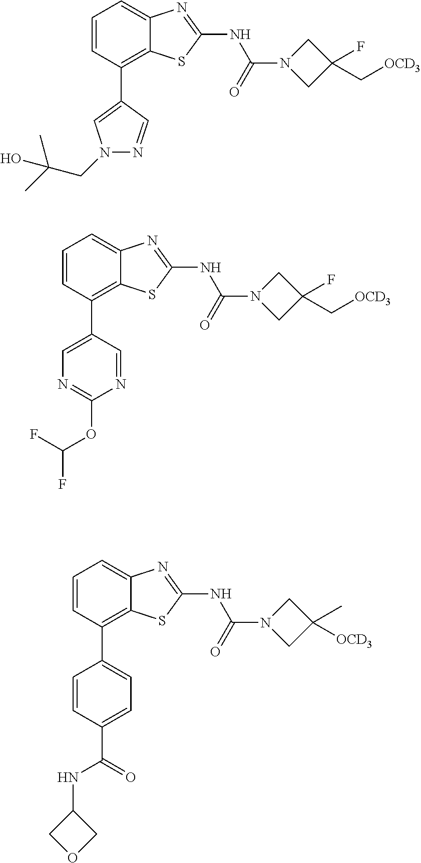 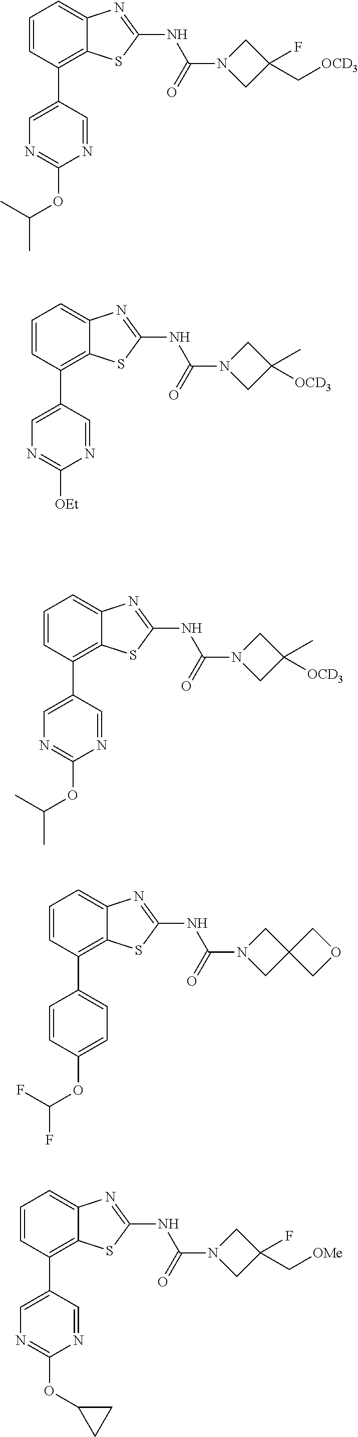 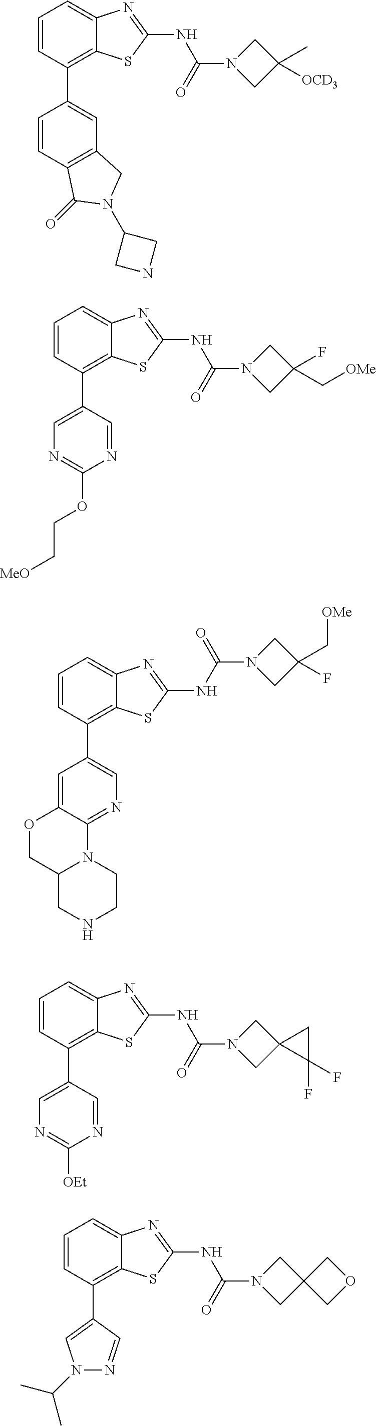 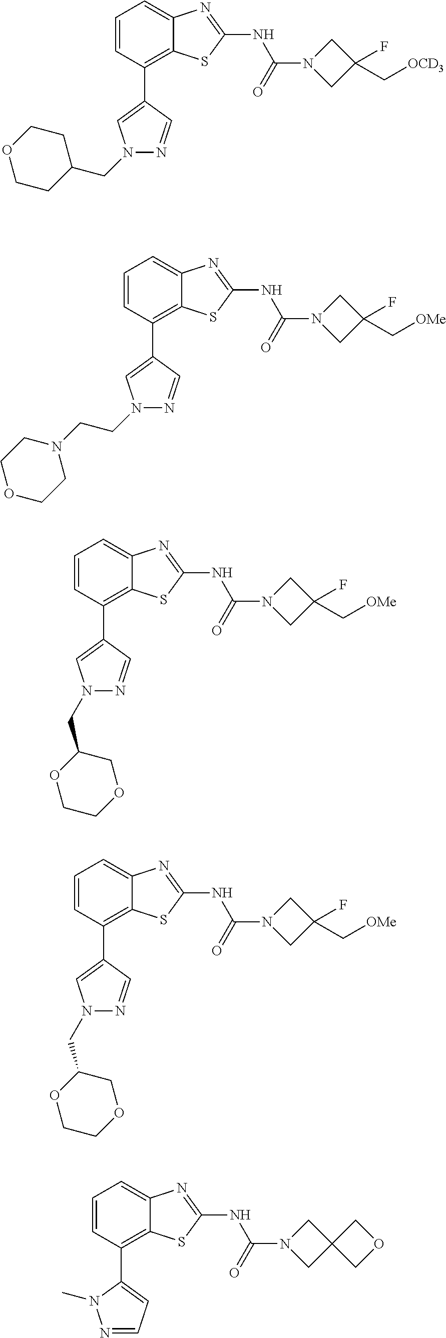 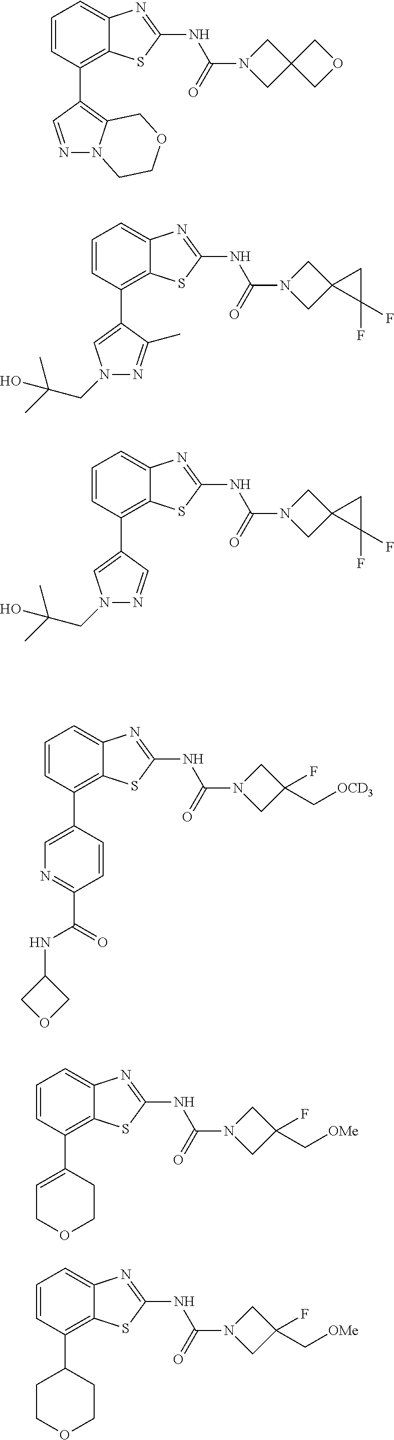 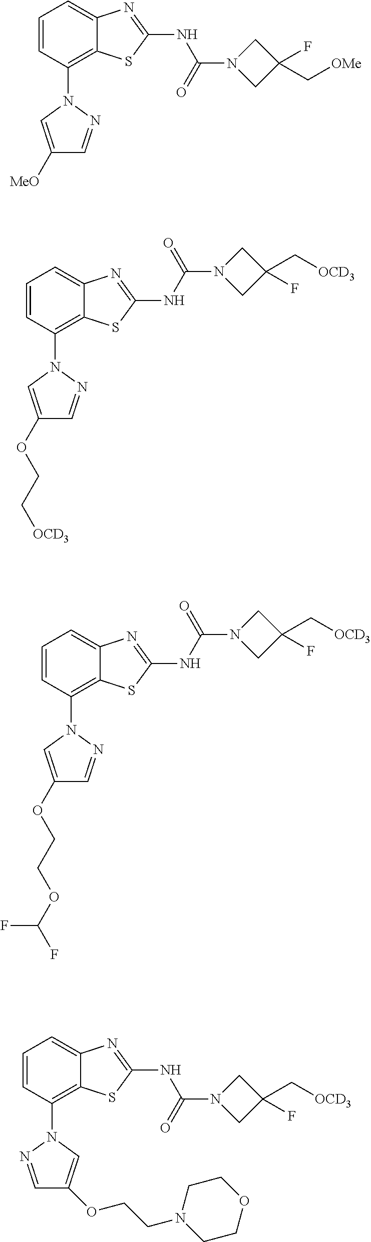 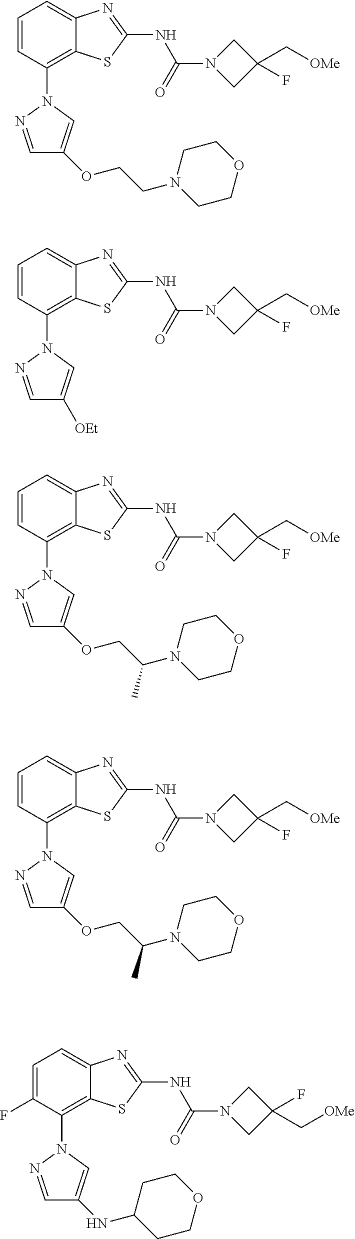 [SMILES: COCC1(F)CN(C(=O)Nc2nc3cccc(-n4cc(OCCN5CCOCC5)cn4)c3s2)C1 ; CCOc1cnn(-c2cccc3nc(NC(=O)N4CC(F)(COC)C4)sc23)c1 ; COCC1(F)CN(C(=O)Nc2nc3cccc(-n4cc(OC[C@@H](C)N5CCOCC5)cn4)c3s2)C1 ; COCC1(F)CN(C(=O)Nc2nc3cccc(-n4cc(OC[C@H](C)N5CCOCC5)cn4)c3s2)C1 ; COCC1(F)CN(C(=O)Nc2nc3ccc(F)c(-n4cc(NC5CCOCC5)cn4)c3s2)C1] 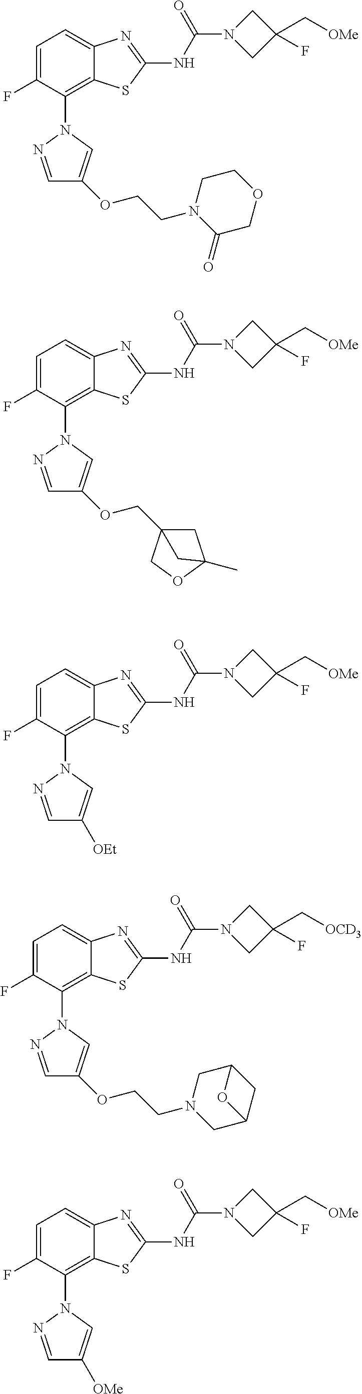 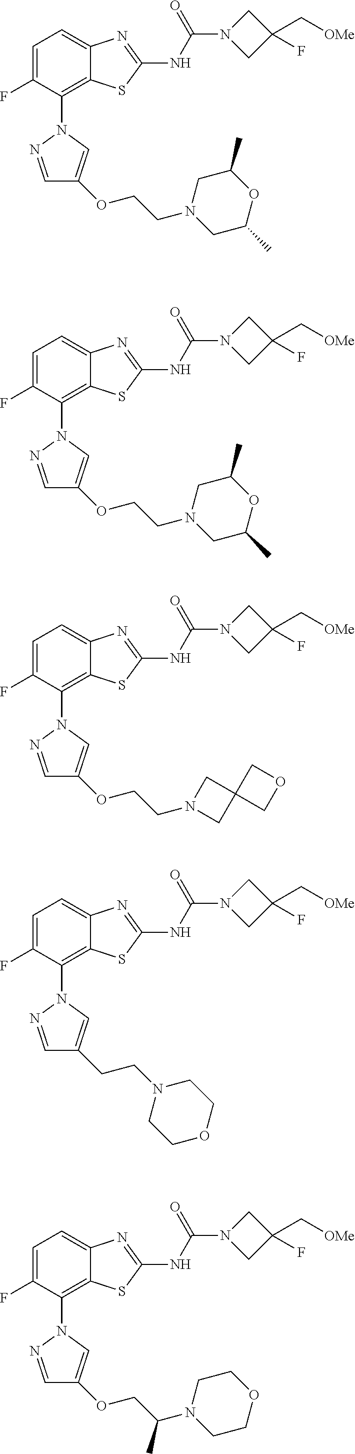 [SMILES: COCC1(F)CN(C(=O)Nc2nc3ccc(F)c(-n4cc(OCCN5C[C@@H](C)O[C@H](C)C5)cn4)c3s2)C1 ; COCC1(F)CN(C(=O)Nc2nc3ccc(F)c(-n4cc(OCCN5C[C@@H](C)O[C@@H](C)C5)cn4)c3s2)C1 ; COCC1(F)CN(C(=O)Nc2nc3ccc(F)c(-n4cc(OCCN5CC6(COC6)C5)cn4)c3s2)C1 ; COCC1(F)CN(C(=O)Nc2nc3ccc(F)c(-n4cc(CCN5CCOCC5)cn4)c3s2)C1 ; COCC1(F)CN(C(=O)Nc2nc3ccc(F)c(-n4cc(OC[C@H](C)N5CCOCC5)cn4)c3s2)C1] 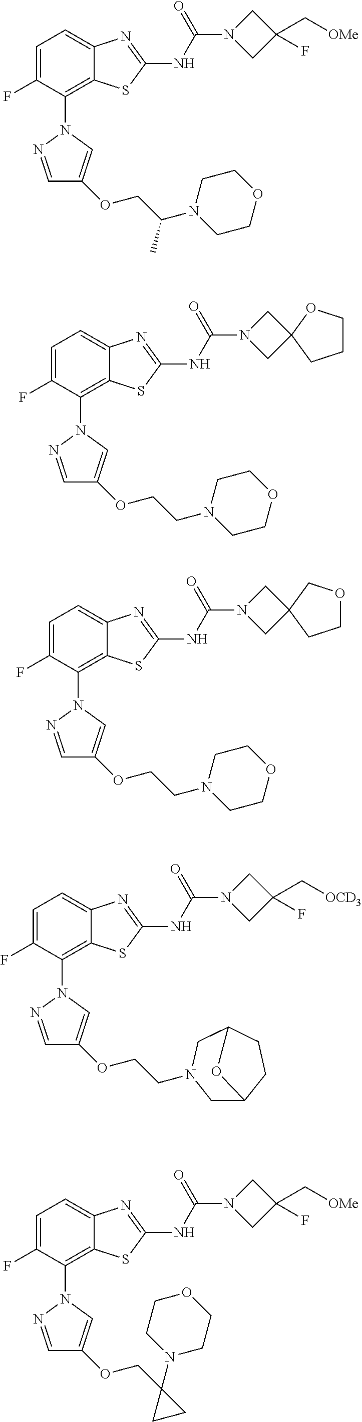 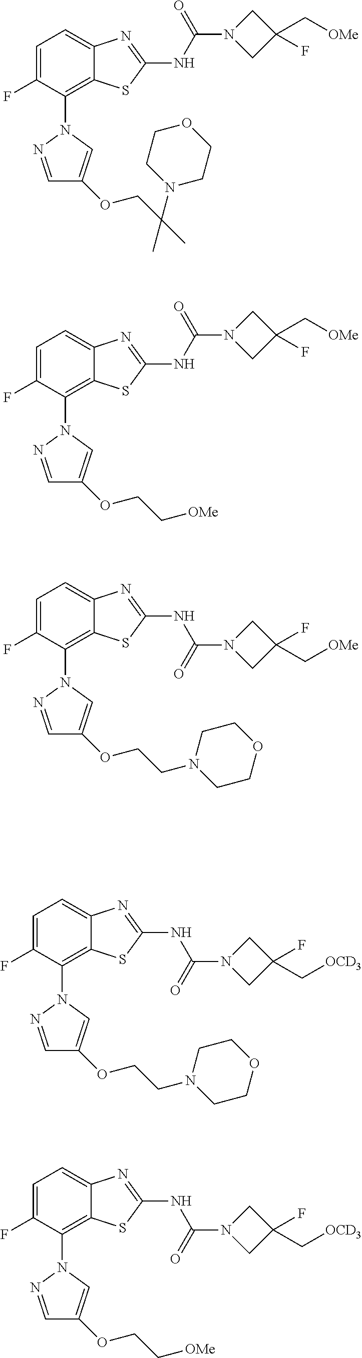 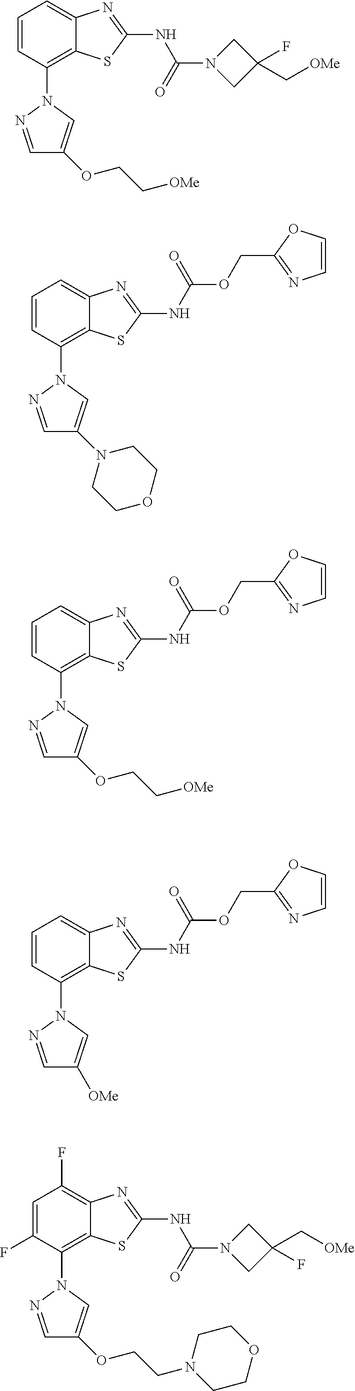 [SMILES: COCCOc1cnn(-c2cccc3nc(NC(=O)N4CC(F)(COC)C4)sc23)c1 ; O=C(Nc1nc2cccc(-n3cc(N4CCOCC4)cn3)c2s1)OCc1ncco1 ; COCCOc1cnn(-c2cccc3nc(NC(=O)OCc4ncco4)sc23)c1 ; COc1cnn(-c2cccc3nc(NC(=O)OCc4ncco4)sc23)c1 ; COCC1(F)CN(C(=O)Nc2nc3c(F)cc(F)c(-n4cc(OCCN5CCOCC5)cn4)c3s2)C1] 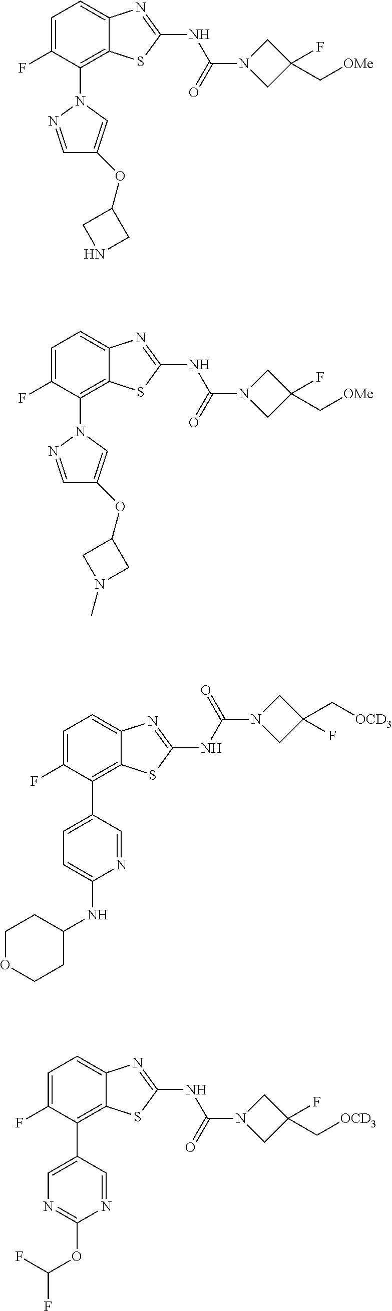 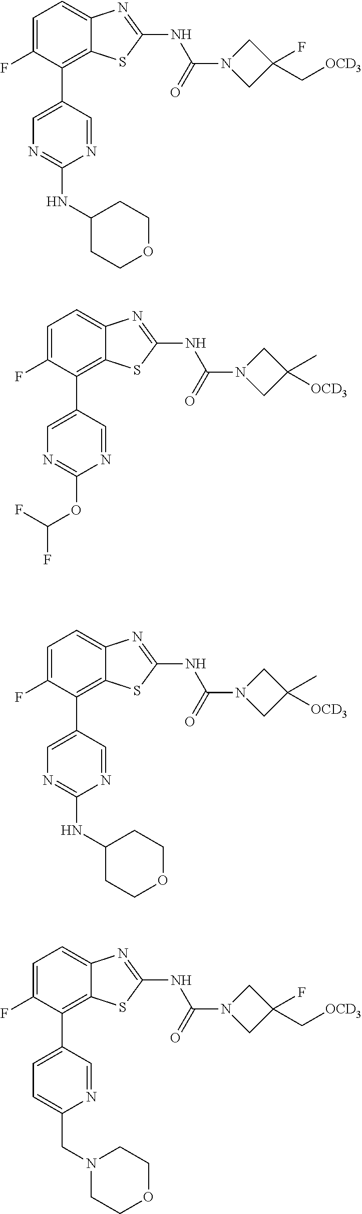 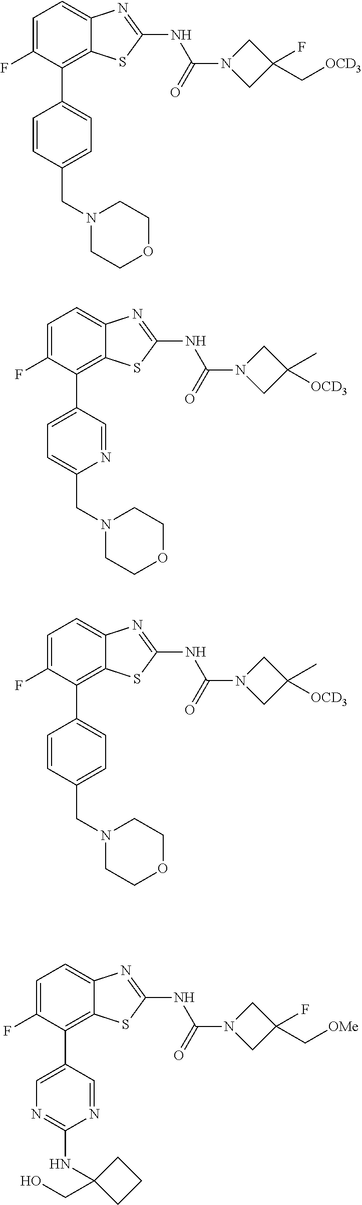 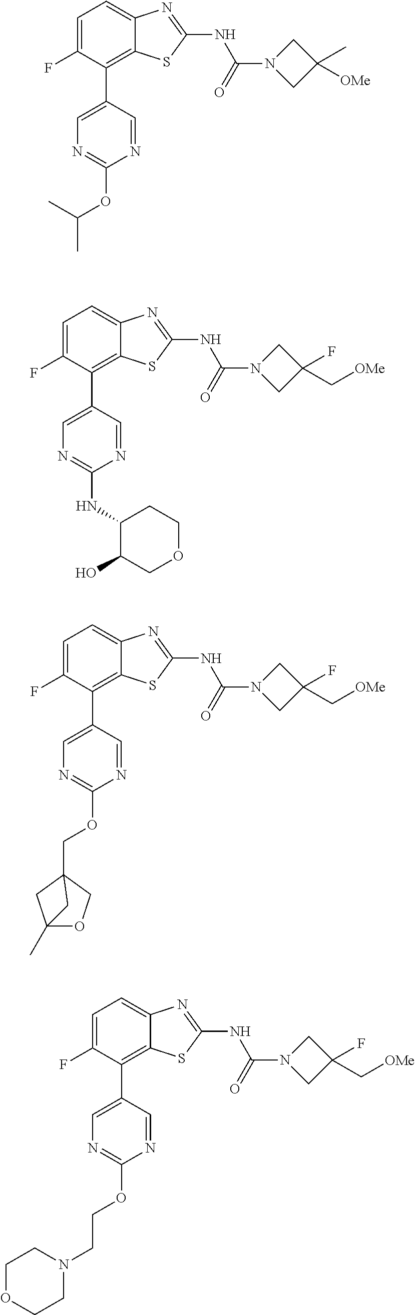 [SMILES: COC1(C)CN(C(=O)Nc2nc3ccc(F)c(-c4cnc(OC(C)C)nc4)c3s2)C1 ; COCC1(F)CN(C(=O)Nc2nc3ccc(F)c(-c4cnc(N[C@@H]5CCOC[C@H]5O)nc4)c3s2)C1 ; COCC1(F)CN(C(=O)Nc2nc3ccc(F)c(-c4cnc(OCC56COC(C)(C5)C6)nc4)c3s2)C1 ; COCC1(F)CN(C(=O)Nc2nc3ccc(F)c(-c4cnc(OCCN5CCOCC5)nc4)c3s2)C1] 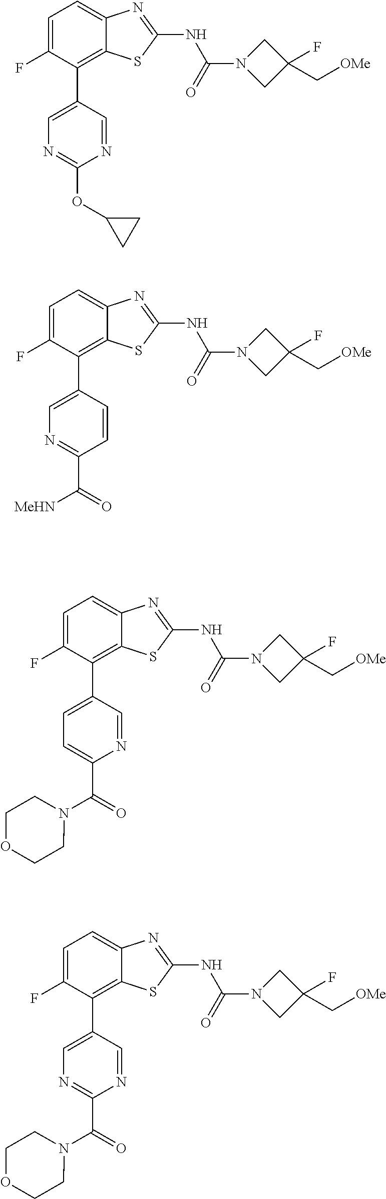 [SMILES: COCC1(F)CN(C(=O)Nc2nc3ccc(F)c(-c4cnc(OC5CC5)nc4)c3s2)C1 ; CNC(=O)c1ccc(-c2c(F)ccc3nc(NC(=O)N4CC(F)(COC)C4)sc23)cn1 ; COCC1(F)CN(C(=O)Nc2nc3ccc(F)c(-c4ccc(C(=O)N5CCOCC5)nc4)c3s2)C1 ; COCC1(F)CN(C(=O)Nc2nc3ccc(F)c(-c4cnc(C(=O)N5CCOCC5)nc4)c3s2)C1] 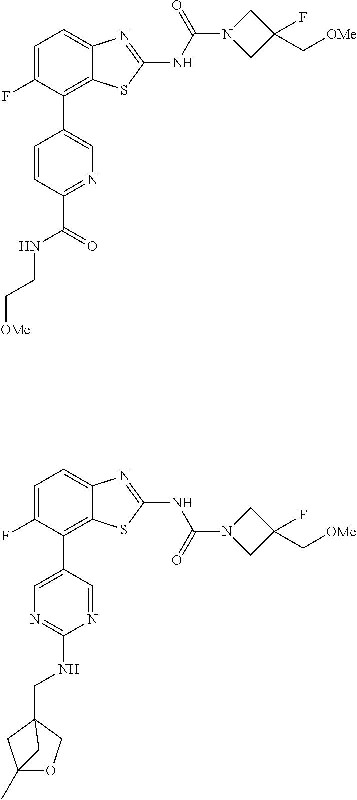 [SMILES: COCCNC(=O)c1ccc(-c2c(F)ccc3nc(NC(=O)N4CC(F)(COC)C4)sc23)cn1 ; COCC1(F)CN(C(=O)Nc2nc3ccc(F)c(-c4cnc(NCC56COC(C)(C5)C6)nc4)c3s2)C1] 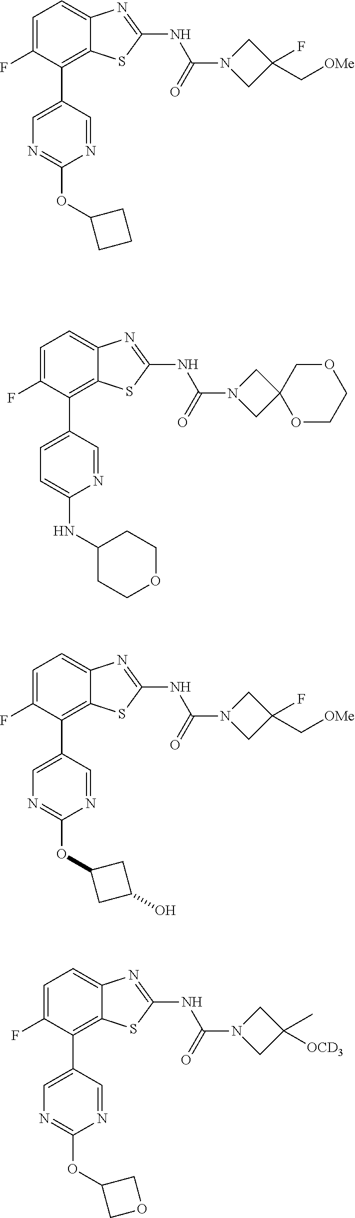 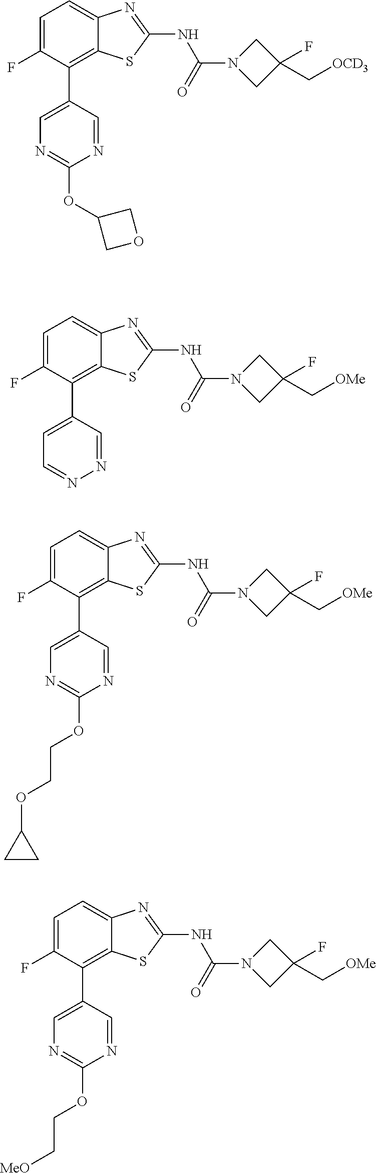 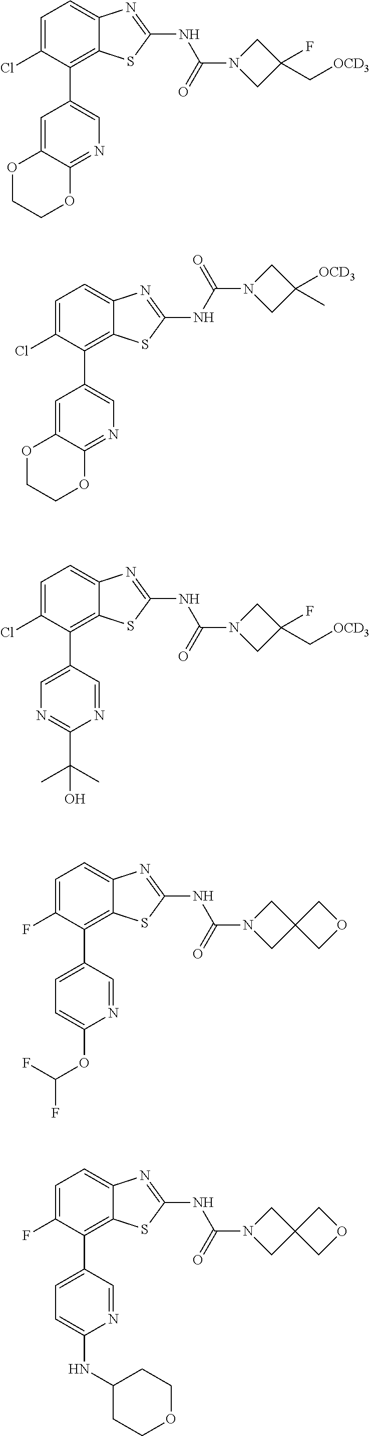 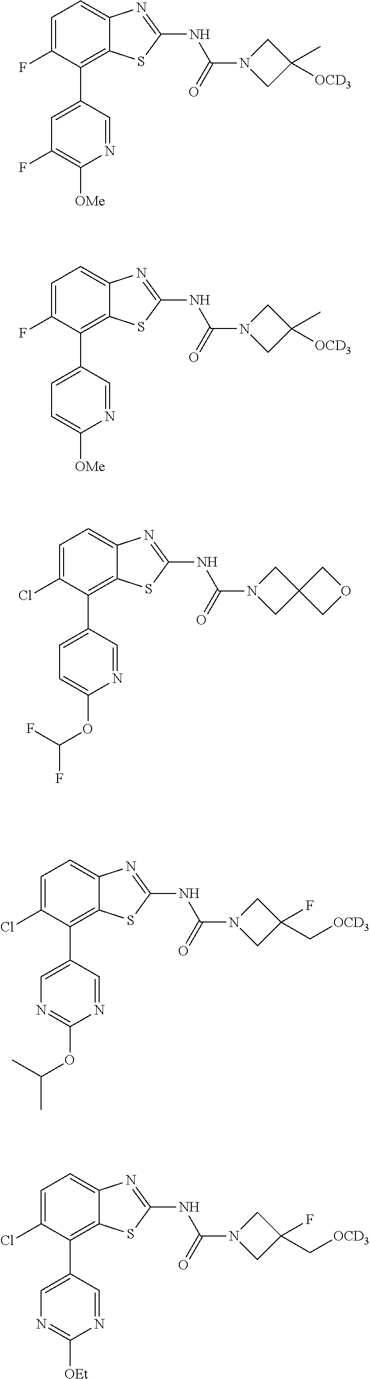 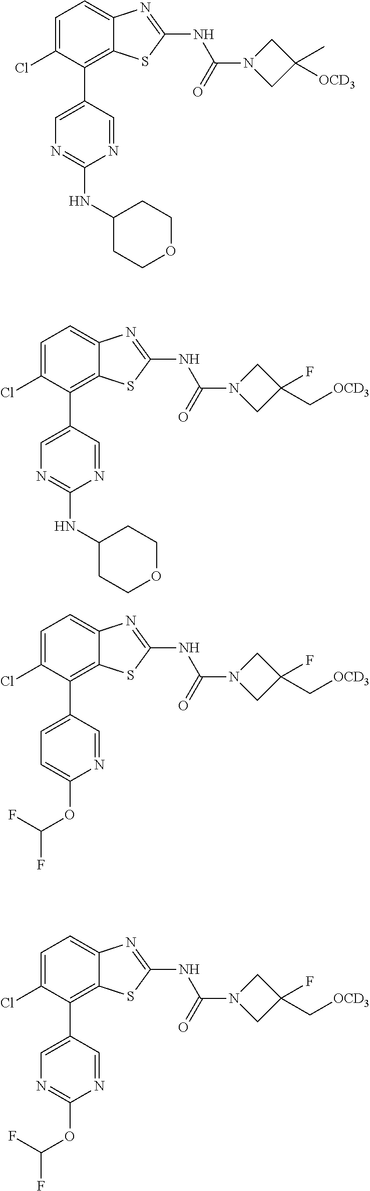 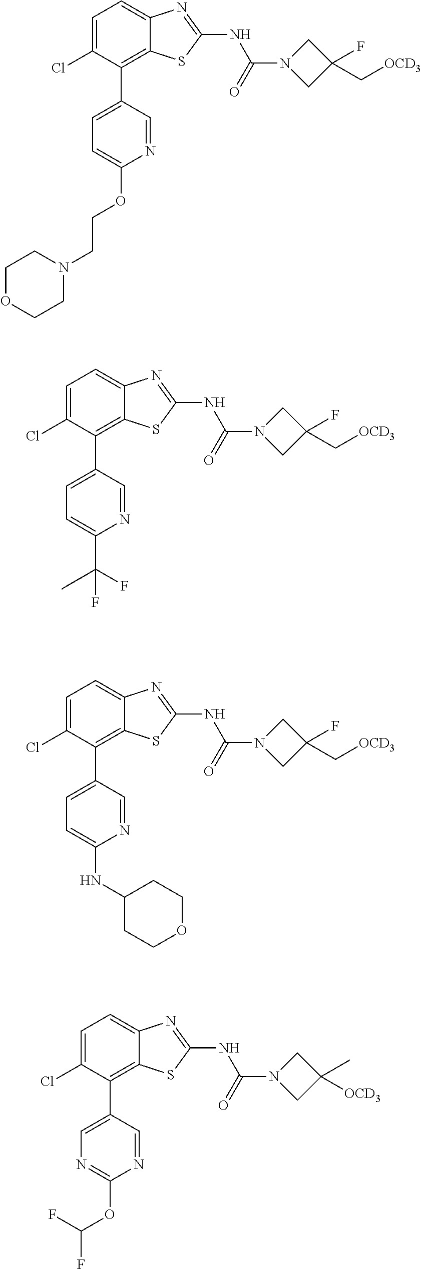 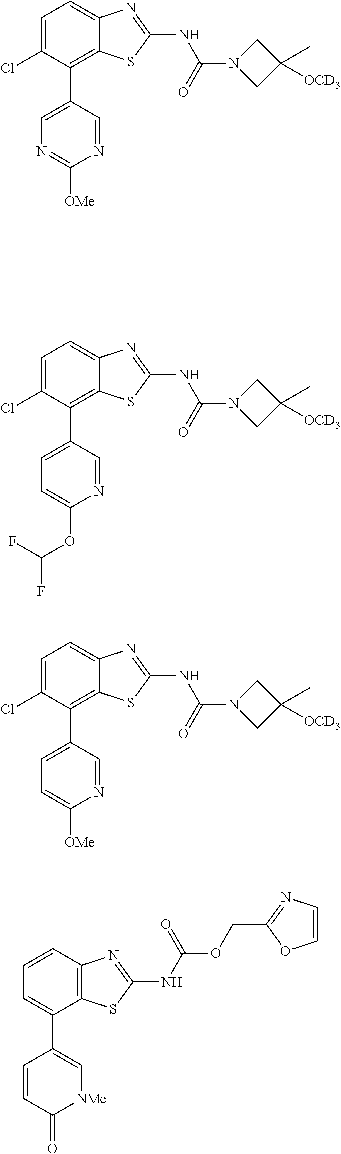 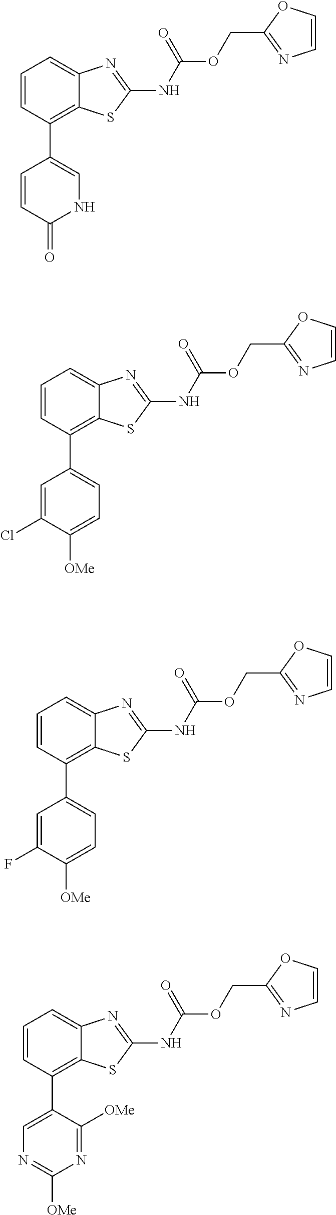 [SMILES: O=C(Nc1nc2cccc(-c3ccc(=O)[nH]c3)c2s1)OCc1ncco1 ; COc1ccc(-c2cccc3nc(NC(=O)OCc4ncco4)sc23)cc1Cl ; COc1ccc(-c2cccc3nc(NC(=O)OCc4ncco4)sc23)cc1F ; COc1ncc(-c2cccc3nc(NC(=O)OCc4ncco4)sc23)c(OC)n1] 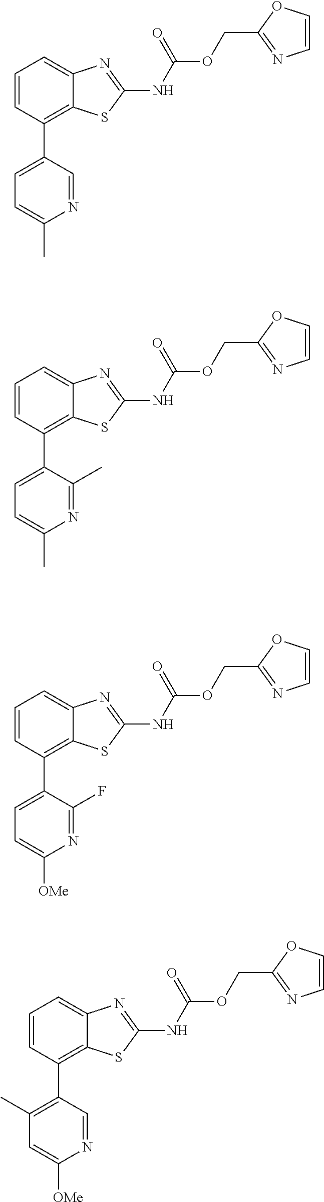 [SMILES: Cc1ccc(-c2cccc3nc(NC(=O)OCc4ncco4)sc23)cn1 ; Cc1ccc(-c2cccc3nc(NC(=O)OCc4ncco4)sc23)c(C)n1 ; COc1ccc(-c2cccc3nc(NC(=O)OCc4ncco4)sc23)c(F)n1 ; COc1cc(C)c(-c2cccc3nc(NC(=O)OCc4ncco4)sc23)cn1] 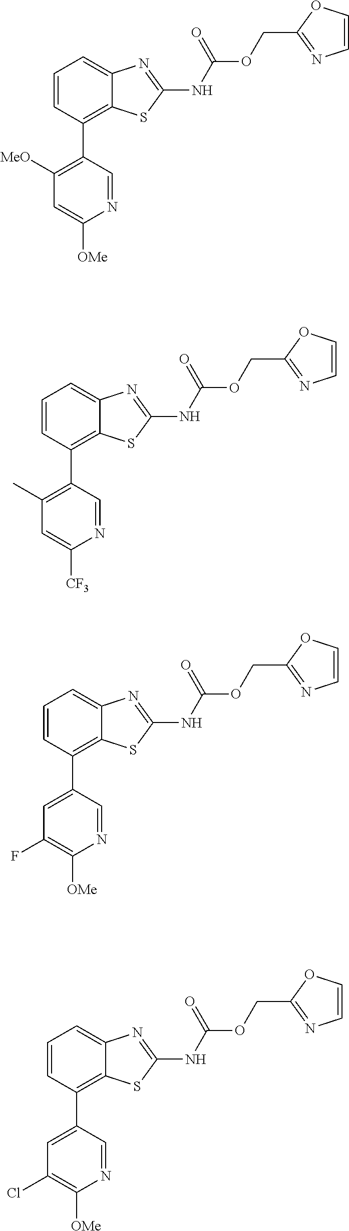 [SMILES: COc1cc(OC)c(-c2cccc3nc(NC(=O)OCc4ncco4)sc23)cn1 ; Cc1cc(C(F)(F)F)ncc1-c1cccc2nc(NC(=O)OCc3ncco3)sc12 ; COc1ncc(-c2cccc3nc(NC(=O)OCc4ncco4)sc23)cc1F ; COc1ncc(-c2cccc3nc(NC(=O)OCc4ncco4)sc23)cc1Cl] 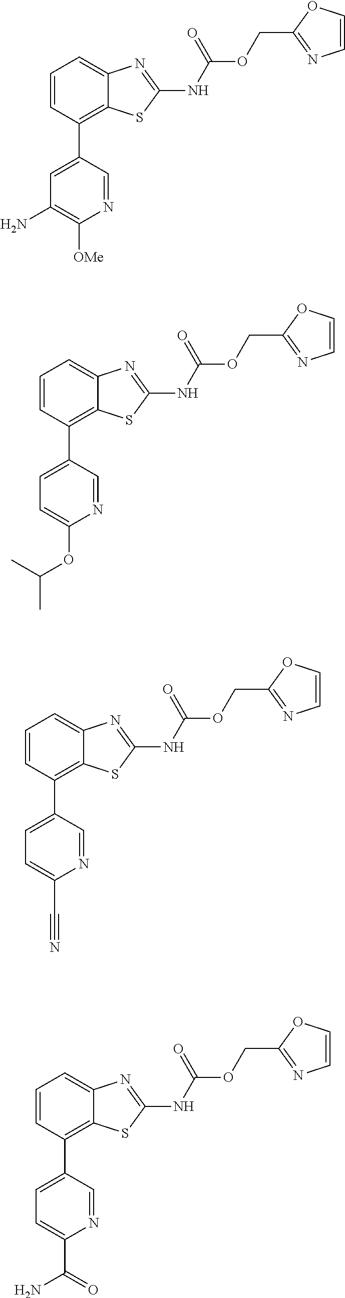 [SMILES: COc1ncc(-c2cccc3nc(NC(=O)OCc4ncco4)sc23)cc1N ; CC(C)Oc1ccc(-c2cccc3nc(NC(=O)OCc4ncco4)sc23)cn1 ; N#Cc1ccc(-c2cccc3nc(NC(=O)OCc4ncco4)sc23)cn1 ; NC(=O)c1ccc(-c2cccc3nc(NC(=O)OCc4ncco4)sc23)cn1] 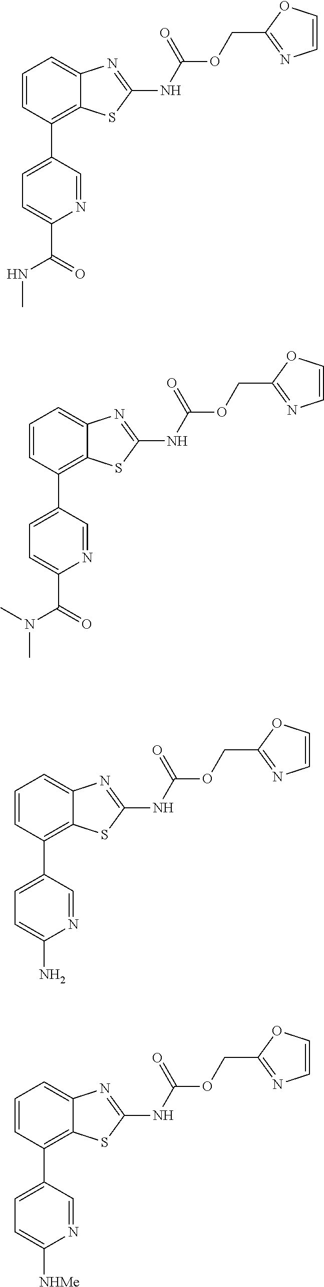 [SMILES: CNC(=O)c1ccc(-c2cccc3nc(NC(=O)OCc4ncco4)sc23)cn1 ; CN(C)C(=O)c1ccc(-c2cccc3nc(NC(=O)OCc4ncco4)sc23)cn1 ; Nc1ccc(-c2cccc3nc(NC(=O)OCc4ncco4)sc23)cn1 ; CNc1ccc(-c2cccc3nc(NC(=O)OCc4ncco4)sc23)cn1] 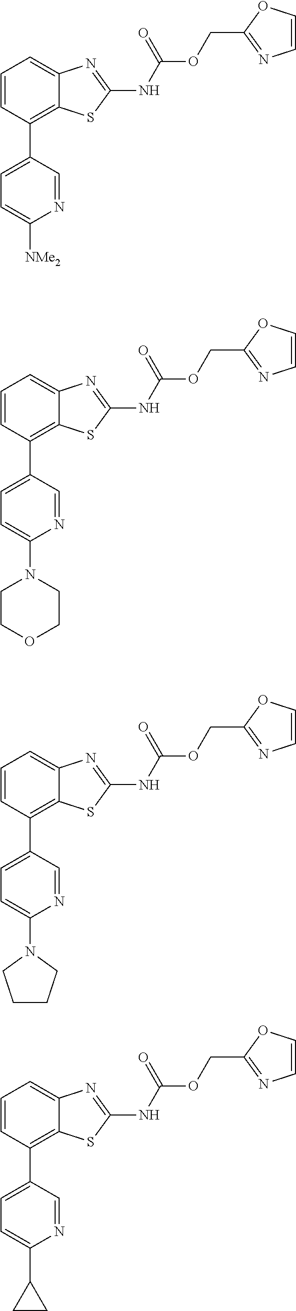 [SMILES: CN(C)c1ccc(-c2cccc3nc(NC(=O)OCc4ncco4)sc23)cn1 ; O=C(Nc1nc2cccc(-c3ccc(N4CCOCC4)nc3)c2s1)OCc1ncco1 ; O=C(Nc1nc2cccc(-c3ccc(N4CCCC4)nc3)c2s1)OCc1ncco1 ; O=C(Nc1nc2cccc(-c3ccc(C4CC4)nc3)c2s1)OCc1ncco1] 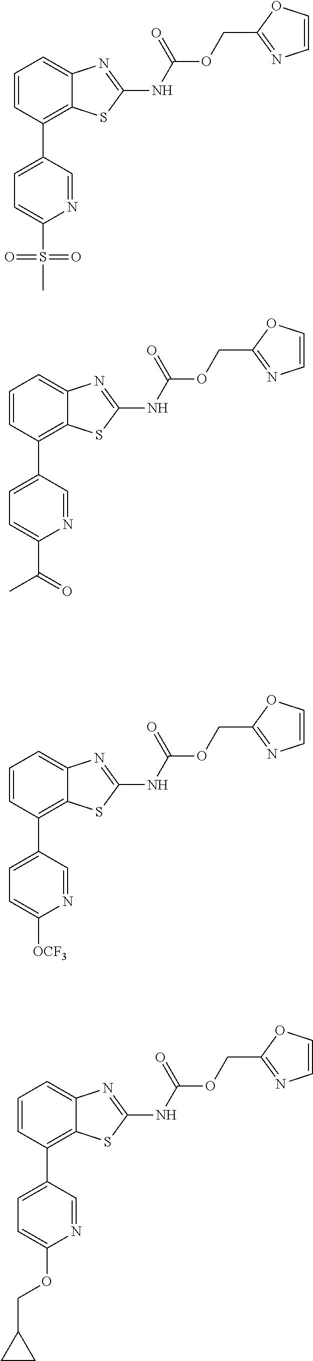 [SMILES: CS(=O)(=O)c1ccc(-c2cccc3nc(NC(=O)OCc4ncco4)sc23)cn1 ; CC(=O)c1ccc(-c2cccc3nc(NC(=O)OCc4ncco4)sc23)cn1 ; O=C(Nc1nc2cccc(-c3ccc(OC(F)(F)F)nc3)c2s1)OCc1ncco1 ; O=C(Nc1nc2cccc(-c3ccc(OCC4CC4)nc3)c2s1)OCc1ncco1] 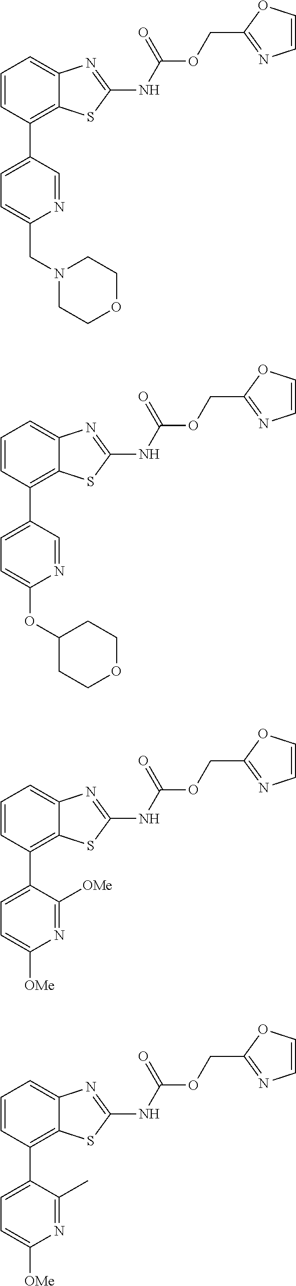 [SMILES: O=C(Nc1nc2cccc(-c3ccc(CN4CCOCC4)nc3)c2s1)OCc1ncco1 ; O=C(Nc1nc2cccc(-c3ccc(OC4CCOCC4)nc3)c2s1)OCc1ncco1 ; COc1ccc(-c2cccc3nc(NC(=O)OCc4ncco4)sc23)c(OC)n1 ; COc1ccc(-c2cccc3nc(NC(=O)OCc4ncco4)sc23)c(C)n1] 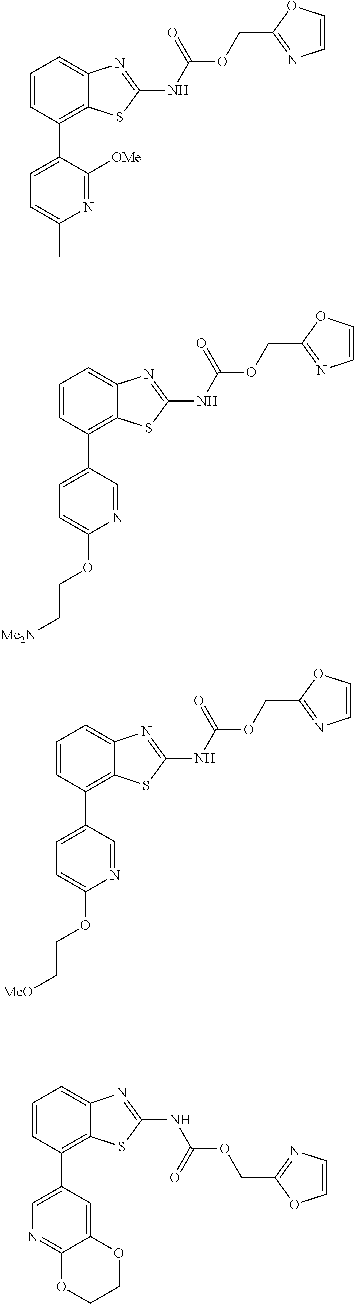 [SMILES: COc1nc(C)ccc1-c1cccc2nc(NC(=O)OCc3ncco3)sc12 ; CN(C)CCOc1ccc(-c2cccc3nc(NC(=O)OCc4ncco4)sc23)cn1 ; COCCOc1ccc(-c2cccc3nc(NC(=O)OCc4ncco4)sc23)cn1 ; O=C(Nc1nc2cccc(-c3cnc4c(c3)OCCO4)c2s1)OCc1ncco1] 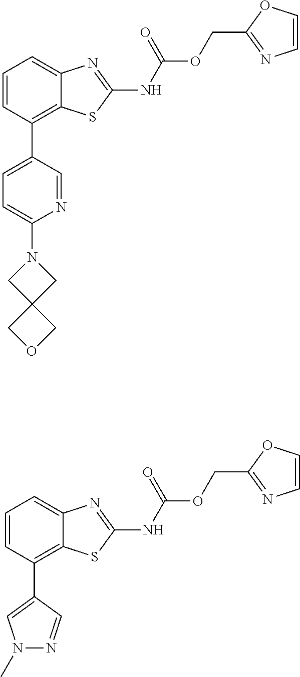 [SMILES: O=C(Nc1nc2cccc(-c3ccc(N4CC5(COC5)C4)nc3)c2s1)OCc1ncco1 ; Cn1cc(-c2cccc3nc(NC(=O)OCc4ncco4)sc23)cn1] 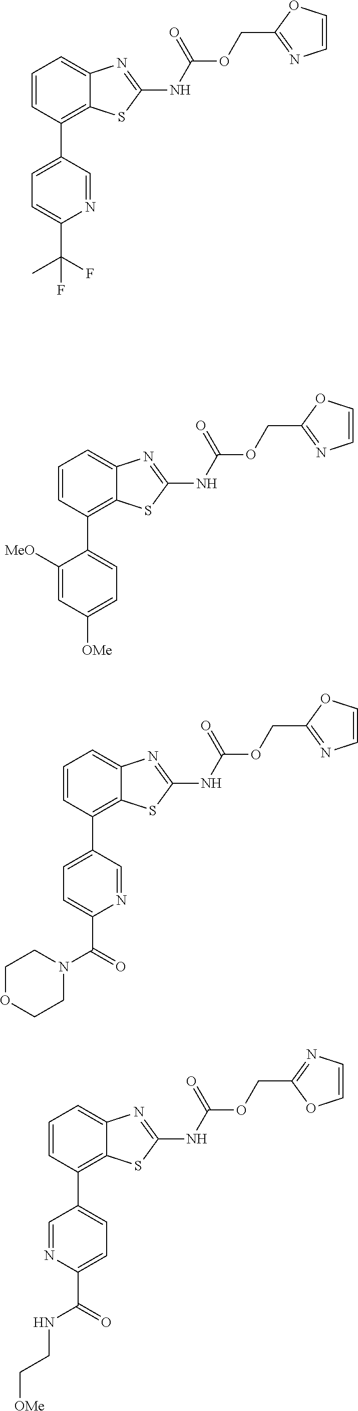 [SMILES: CC(F)(F)c1ccc(-c2cccc3nc(NC(=O)OCc4ncco4)sc23)cn1 ; COc1ccc(-c2cccc3nc(NC(=O)OCc4ncco4)sc23)c(OC)c1 ; O=C(Nc1nc2cccc(-c3ccc(C(=O)N4CCOCC4)nc3)c2s1)OCc1ncco1 ; COCCNC(=O)c1ccc(-c2cccc3nc(NC(=O)OCc4ncco4)sc23)cn1] 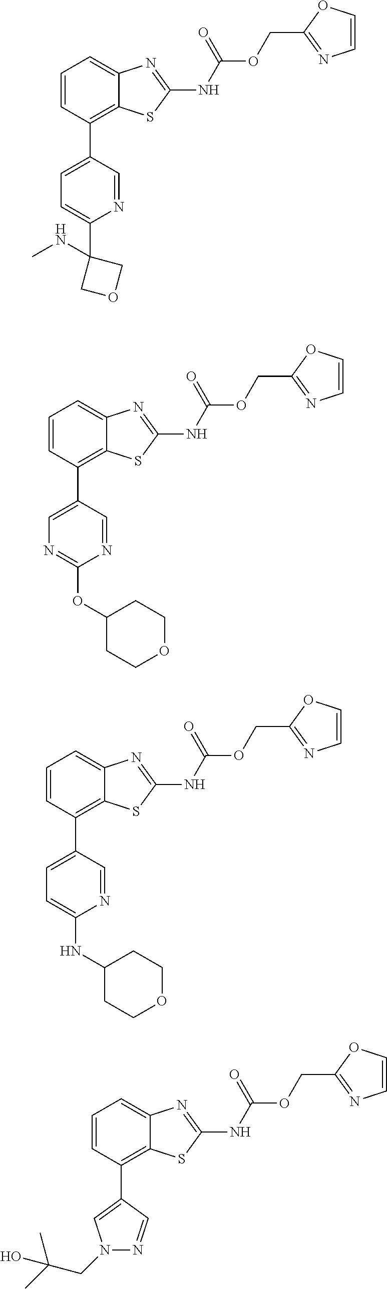 [SMILES: CNC1(c2ccc(-c3cccc4nc(NC(=O)OCc5ncco5)sc34)cn2)COC1 ; O=C(Nc1nc2cccc(-c3cnc(OC4CCOCC4)nc3)c2s1)OCc1ncco1 ; O=C(Nc1nc2cccc(-c3ccc(NC4CCOCC4)nc3)c2s1)OCc1ncco1 ; CC(C)(O)Cn1cc(-c2cccc3nc(NC(=O)OCc4ncco4)sc23)cn1] 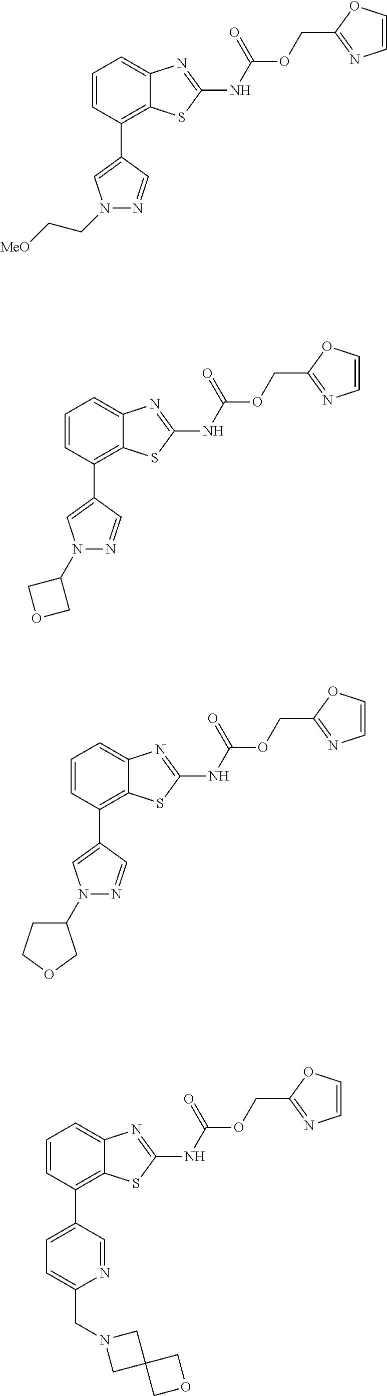 [SMILES: COCCn1cc(-c2cccc3nc(NC(=O)OCc4ncco4)sc23)cn1 ; O=C(Nc1nc2cccc(-c3cnn(C4COC4)c3)c2s1)OCc1ncco1 ; O=C(Nc1nc2cccc(-c3cnn(C4CCOC4)c3)c2s1)OCc1ncco1 ; O=C(Nc1nc2cccc(-c3ccc(CN4CC5(COC5)C4)nc3)c2s1)OCc1ncco1] 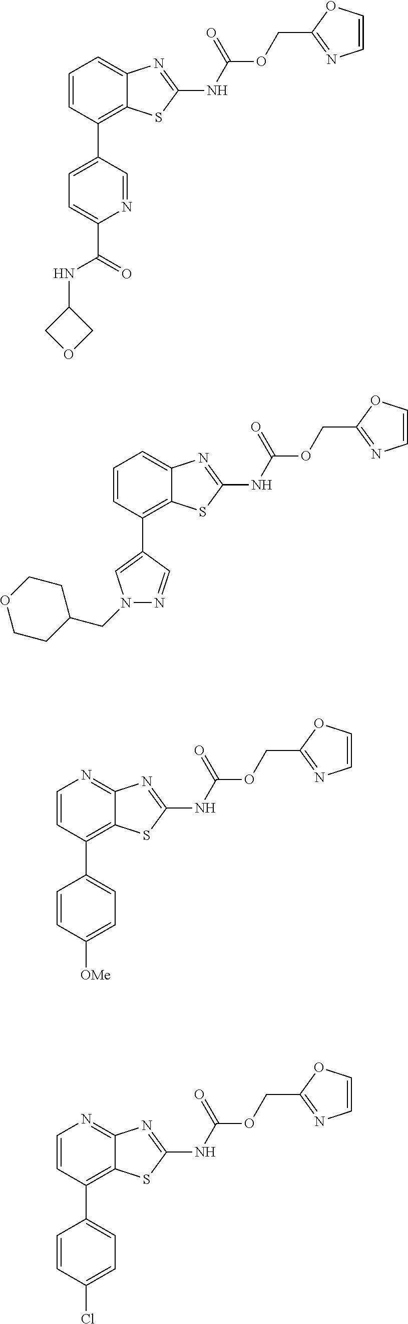 [SMILES: O=C(Nc1nc2cccc(-c3ccc(C(=O)NC4COC4)nc3)c2s1)OCc1ncco1 ; O=C(Nc1nc2cccc(-c3cnn(CC4CCOCC4)c3)c2s1)OCc1ncco1 ; COc1ccc(-c2ccnc3nc(NC(=O)OCc4ncco4)sc23)cc1 ; O=C(Nc1nc2nccc(-c3ccc(Cl)cc3)c2s1)OCc1ncco1] 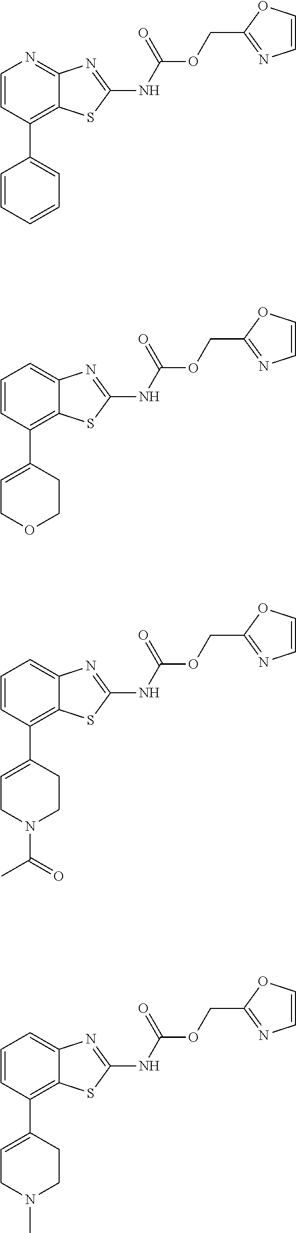 [SMILES: O=C(Nc1nc2nccc(-c3ccccc3)c2s1)OCc1ncco1 ; O=C(Nc1nc2cccc(C3=CCOCC3)c2s1)OCc1ncco1 ; CC(=O)N1CC=C(c2cccc3nc(NC(=O)OCc4ncco4)sc23)CC1 ; CN1CC=C(c2cccc3nc(NC(=O)OCc4ncco4)sc23)CC1] 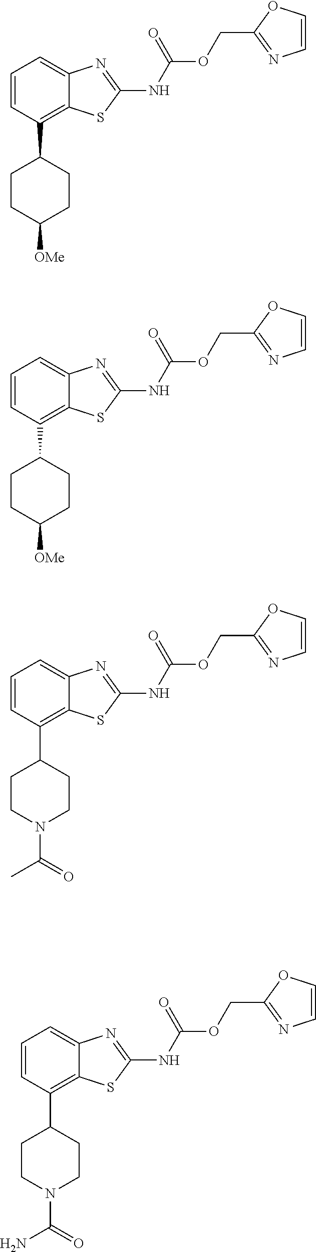 [SMILES: CO[C@H]1CC[C@@H](c2cccc3nc(NC(=O)OCc4ncco4)sc32)CC1 ; CO[C@H]1CC[C@H](c2cccc3nc(NC(=O)OCc4ncco4)sc32)CC1 ; CC(=O)N1CCC(c2cccc3nc(NC(=O)OCc4ncco4)sc23)CC1 ; NC(=O)N1CCC(c2cccc3nc(NC(=O)OCc4ncco4)sc23)CC1] 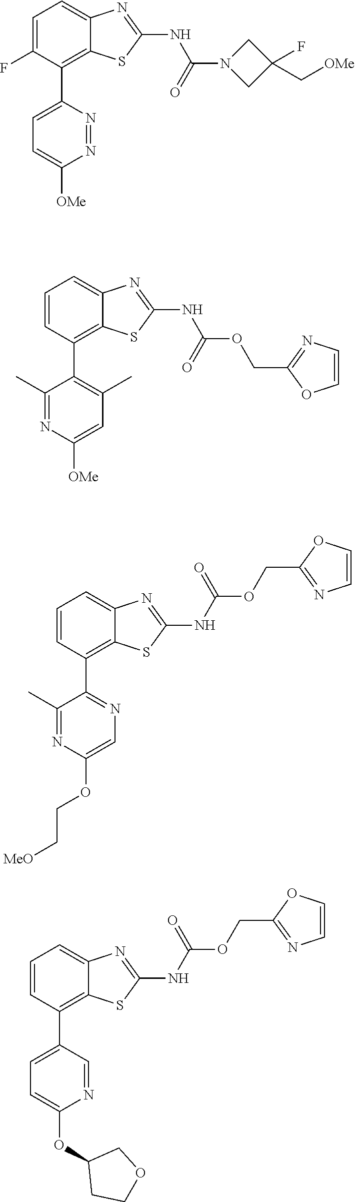 [SMILES: COCC1(F)CN(C(=O)Nc2nc3ccc(F)c(-c4ccc(OC)nn4)c3s2)C1 ; COc1cc(C)c(-c2cccc3nc(NC(=O)OCc4ncco4)sc23)c(C)n1 ; COCCOc1cnc(-c2cccc3nc(NC(=O)OCc4ncco4)sc23)c(C)n1 ; O=C(Nc1nc2cccc(-c3ccc(O[C@@H]4CCOC4)nc3)c2s1)OCc1ncco1] 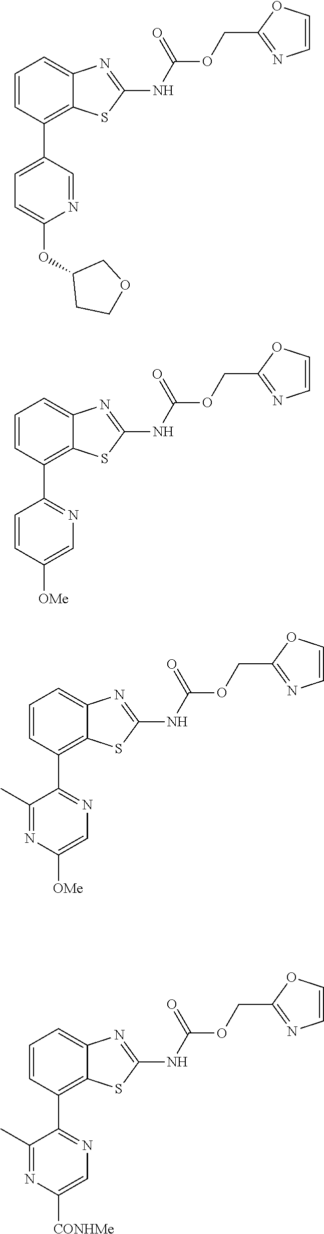 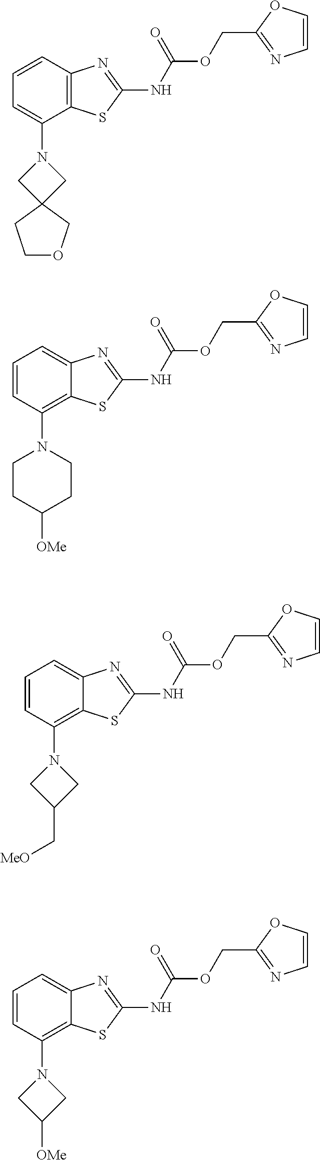 [SMILES: O=C(Nc1nc2cccc(N3CC4(CCOC4)C3)c2s1)OCc1ncco1 ; COC1CCN(c2cccc3nc(NC(=O)OCc4ncco4)sc23)CC1 ; COCC1CN(c2cccc3nc(NC(=O)OCc4ncco4)sc23)C1 ; COC1CN(c2cccc3nc(NC(=O)OCc4ncco4)sc23)C1] 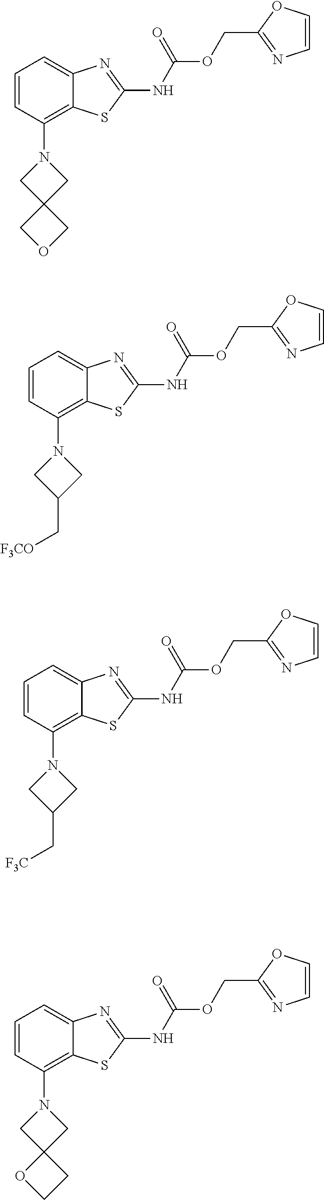 [SMILES: O=C(Nc1nc2cccc(N3CC4(COC4)C3)c2s1)OCc1ncco1 ; O=C(Nc1nc2cccc(N3CC(COC(F)(F)F)C3)c2s1)OCc1ncco1 ; O=C(Nc1nc2cccc(N3CC(CC(F)(F)F)C3)c2s1)OCc1ncco1 ; O=C(Nc1nc2cccc(N3CC4(CCO4)C3)c2s1)OCc1ncco1] 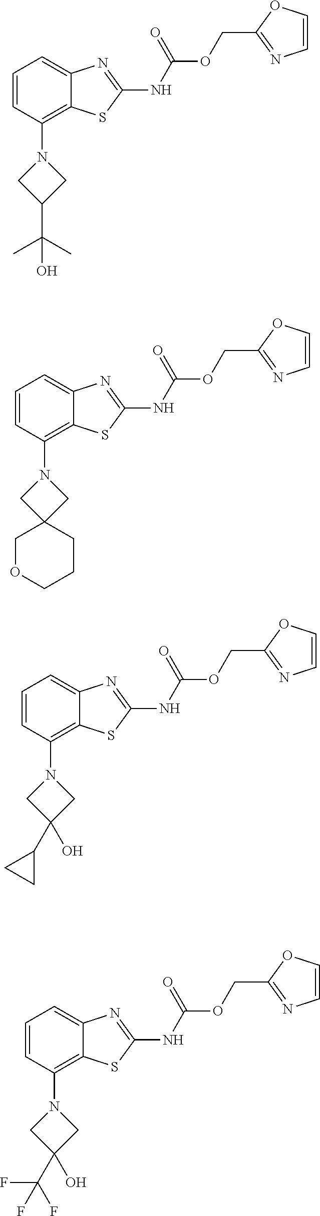 [SMILES: CC(C)(O)C1CN(c2cccc3nc(NC(=O)OCc4ncco4)sc23)C1 ; O=C(Nc1nc2cccc(N3CC4(CCCOC4)C3)c2s1)OCc1ncco1 ; O=C(Nc1nc2cccc(N3CC(O)(C4CC4)C3)c2s1)OCc1ncco1 ; O=C(Nc1nc2cccc(N3CC(O)(C(F)(F)F)C3)c2s1)OCc1ncco1] 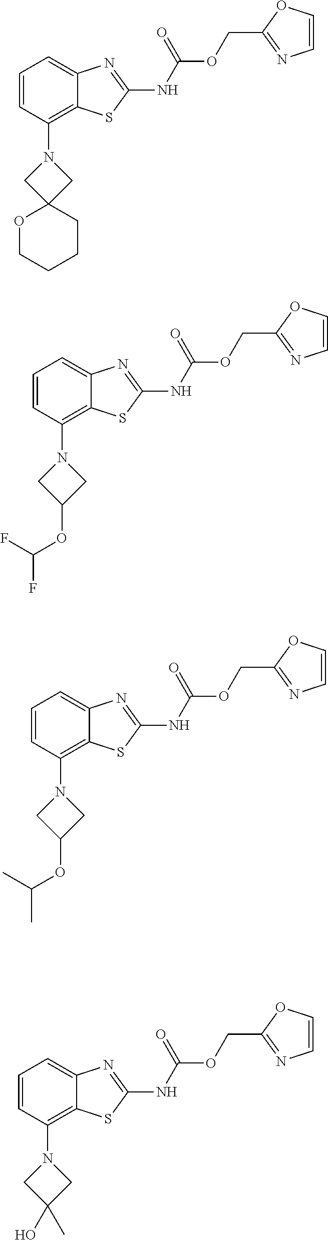 [SMILES: O=C(Nc1nc2cccc(N3CC4(CCCCO4)C3)c2s1)OCc1ncco1 ; O=C(Nc1nc2cccc(N3CC(OC(F)F)C3)c2s1)OCc1ncco1 ; CC(C)OC1CN(c2cccc3nc(NC(=O)OCc4ncco4)sc23)C1 ; CC1(O)CN(c2cccc3nc(NC(=O)OCc4ncco4)sc23)C1] 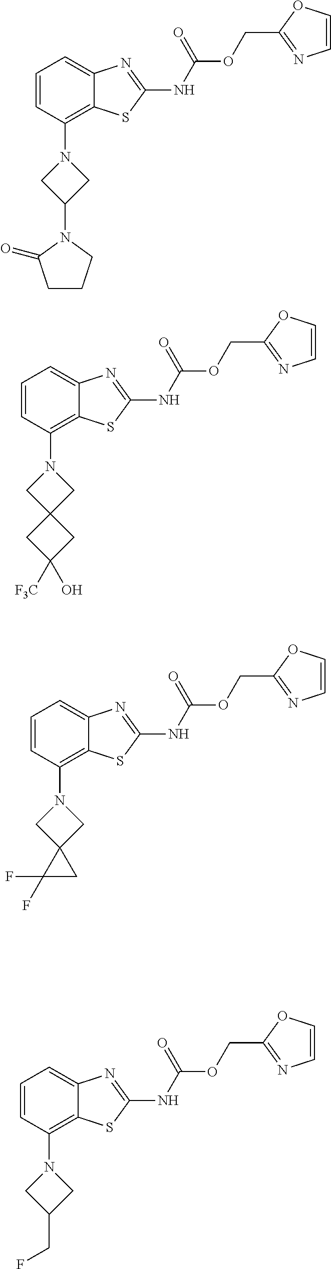 [SMILES: O=C(Nc1nc2cccc(N3CC(N4CCCC4=O)C3)c2s1)OCc1ncco1 ; O=C(Nc1nc2cccc(N3CC4(C3)CC(O)(C(F)(F)F)C4)c2s1)OCc1ncco1 ; O=C(Nc1nc2cccc(N3CC4(C3)CC4(F)F)c2s1)OCc1ncco1 ; O=C(Nc1nc2cccc(N3CC(CF)C3)c2s1)OCc1ncco1] 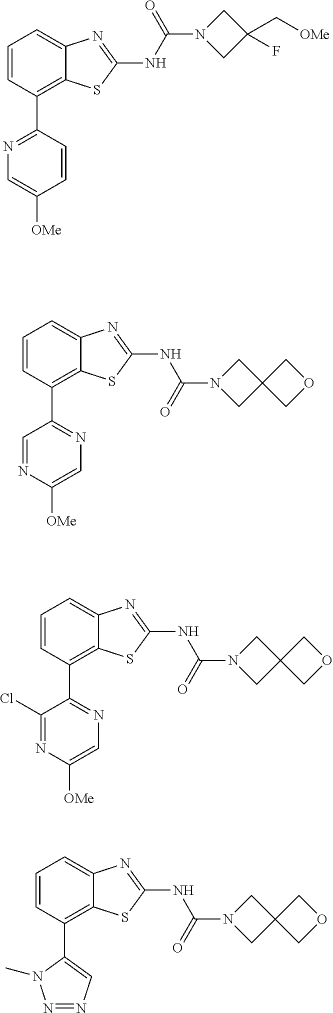 [SMILES: COCC1(F)CN(C(=O)Nc2nc3cccc(-c4ccc(OC)cn4)c3s2)C1 ; COc1cnc(-c2cccc3nc(NC(=O)N4CC5(COC5)C4)sc23)cn1 ; COc1cnc(-c2cccc3nc(NC(=O)N4CC5(COC5)C4)sc23)c(Cl)n1 ; Cn1nncc1-c1cccc2nc(NC(=O)N3CC4(COC4)C3)sc12] 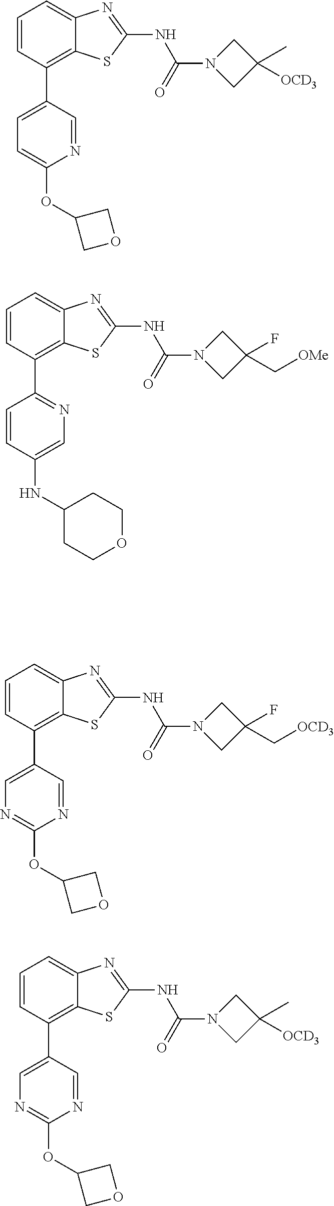 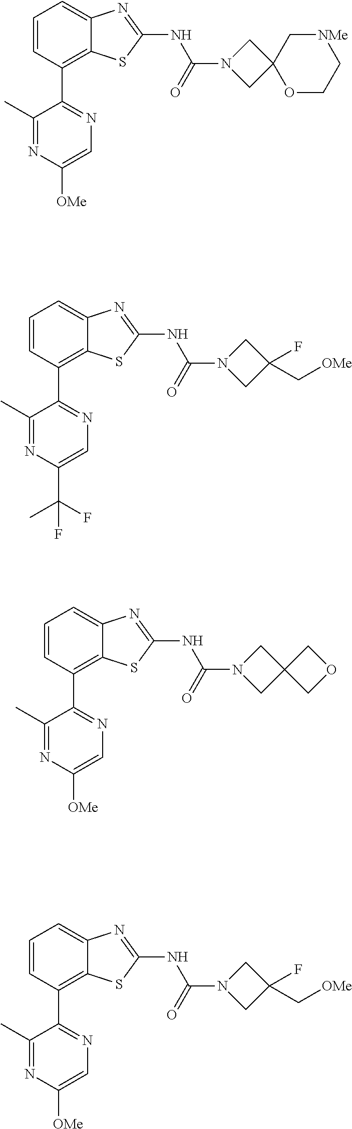 [SMILES: COc1cnc(-c2cccc3nc(NC(=O)N4CC5(CN(C)CCO5)C4)sc23)c(C)n1 ; COCC1(F)CN(C(=O)Nc2nc3cccc(-c4ncc(C(C)(F)F)nc4C)c3s2)C1 ; COc1cnc(-c2cccc3nc(NC(=O)N4CC5(COC5)C4)sc23)c(C)n1 ; COCC1(F)CN(C(=O)Nc2nc3cccc(-c4ncc(OC)nc4C)c3s2)C1] 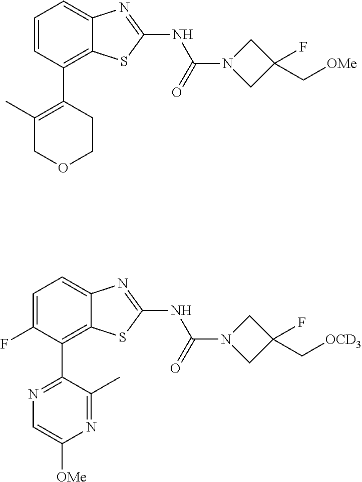 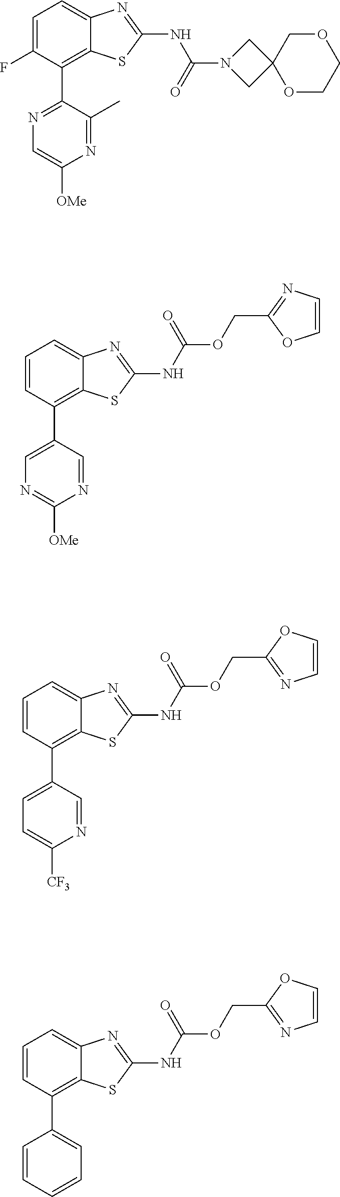 [SMILES: COc1cnc(-c2c(F)ccc3nc(NC(=O)N4CC5(COCCO5)C4)sc23)c(C)n1 ; COc1ncc(-c2cccc3nc(NC(=O)OCc4ncco4)sc23)cn1 ; O=C(Nc1nc2cccc(-c3ccc(C(F)(F)F)nc3)c2s1)OCc1ncco1 ; O=C(Nc1nc2cccc(-c3ccccc3)c2s1)OCc1ncco1] 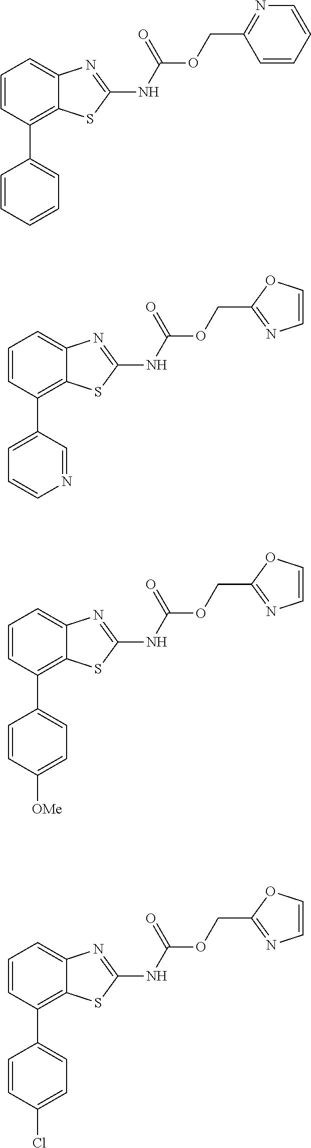 [SMILES: O=C(Nc1nc2cccc(-c3ccccc3)c2s1)OCc1ccccn1 ; O=C(Nc1nc2cccc(-c3cccnc3)c2s1)OCc1ncco1 ; COc1ccc(-c2cccc3nc(NC(=O)OCc4ncco4)sc23)cc1 ; O=C(Nc1nc2cccc(-c3ccc(Cl)cc3)c2s1)OCc1ncco1] 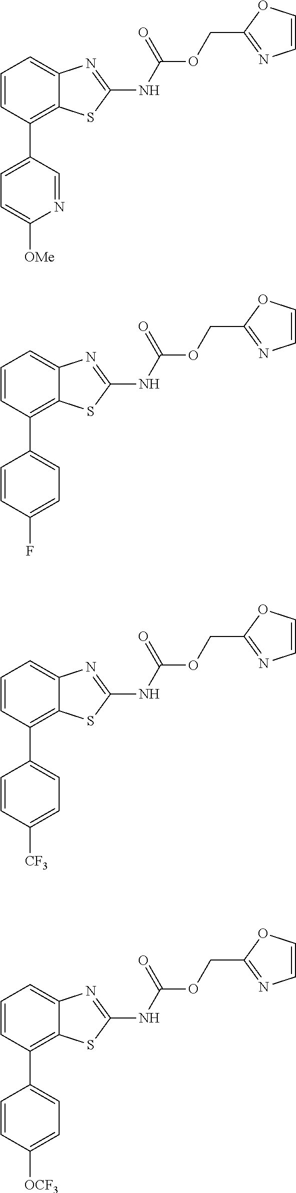 [SMILES: COc1ccc(-c2cccc3nc(NC(=O)OCc4ncco4)sc23)cn1 ; O=C(Nc1nc2cccc(-c3ccc(F)cc3)c2s1)OCc1ncco1 ; O=C(Nc1nc2cccc(-c3ccc(C(F)(F)F)cc3)c2s1)OCc1ncco1 ; O=C(Nc1nc2cccc(-c3ccc(OC(F)(F)F)cc3)c2s1)OCc1ncco1] 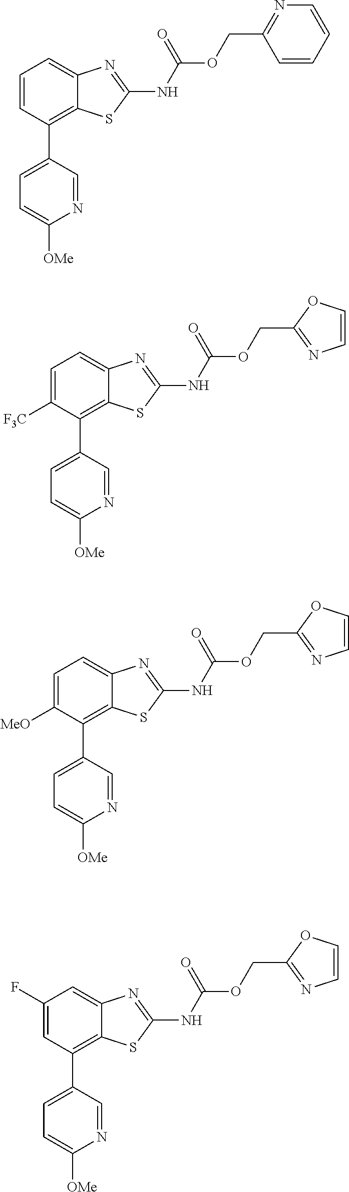 [SMILES: COc1ccc(-c2cccc3nc(NC(=O)OCc4ccccn4)sc23)cn1 ; COc1ccc(-c2c(C(F)(F)F)ccc3nc(NC(=O)OCc4ncco4)sc23)cn1 ; COc1ccc(-c2c(OC)ccc3nc(NC(=O)OCc4ncco4)sc23)cn1 ; COc1ccc(-c2cc(F)cc3nc(NC(=O)OCc4ncco4)sc23)cn1] 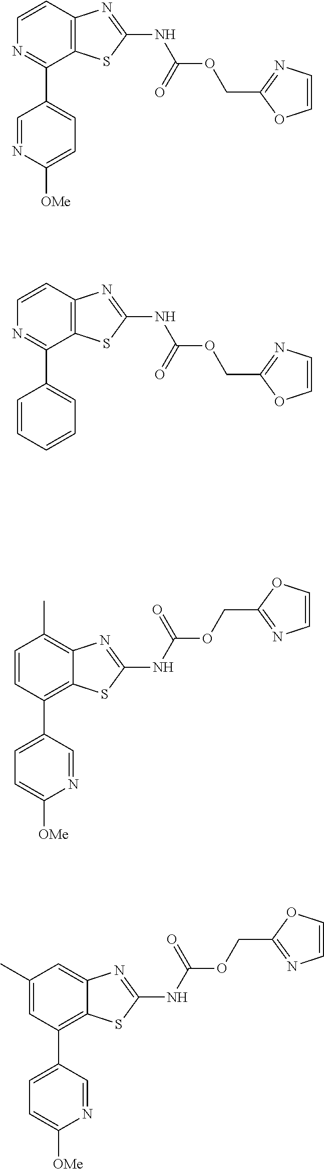 [SMILES: COc1ccc(-c2nccc3nc(NC(=O)OCc4ncco4)sc23)cn1 ; O=C(Nc1nc2ccnc(-c3ccccc3)c2s1)OCc1ncco1 ; COc1ccc(-c2ccc(C)c3nc(NC(=O)OCc4ncco4)sc23)cn1 ; COc1ccc(-c2cc(C)cc3nc(NC(=O)OCc4ncco4)sc23)cn1] 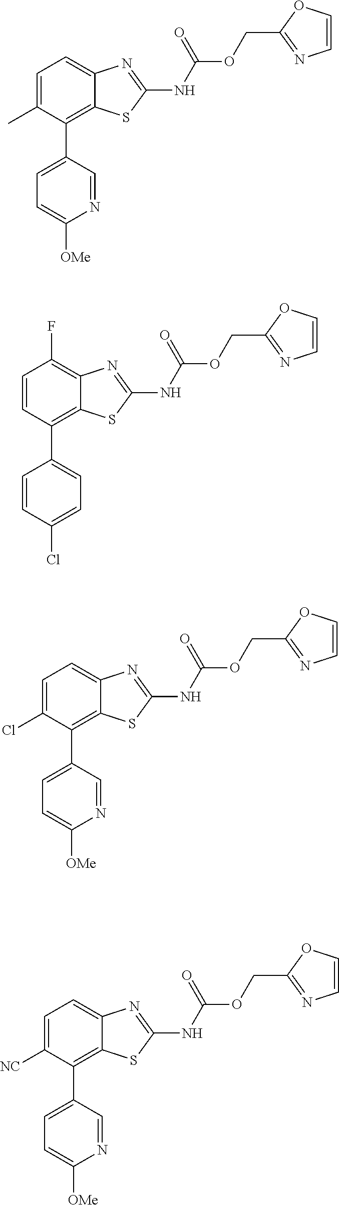 [SMILES: COc1ccc(-c2c(C)ccc3nc(NC(=O)OCc4ncco4)sc23)cn1 ; O=C(Nc1nc2c(F)ccc(-c3ccc(Cl)cc3)c2s1)OCc1ncco1 ; COc1ccc(-c2c(Cl)ccc3nc(NC(=O)OCc4ncco4)sc23)cn1 ; COc1ccc(-c2c(C#N)ccc3nc(NC(=O)OCc4ncco4)sc23)cn1] 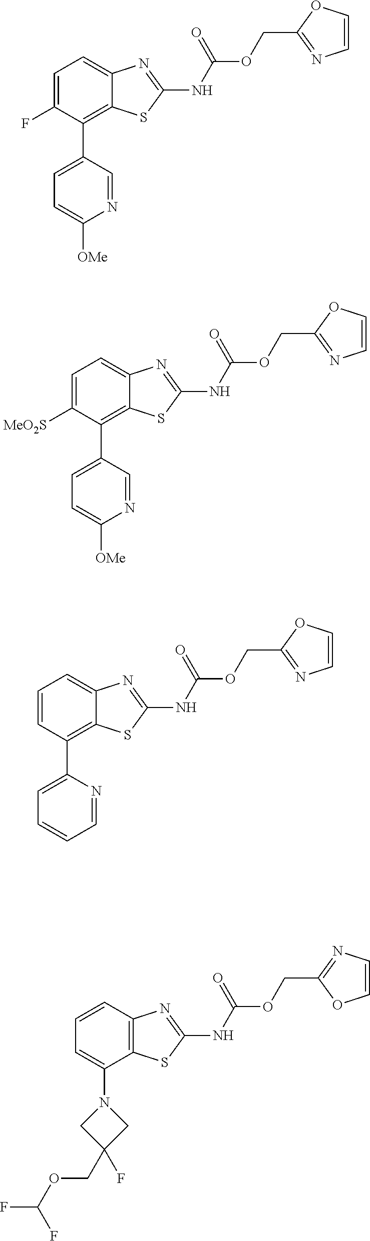 [SMILES: COc1ccc(-c2c(F)ccc3nc(NC(=O)OCc4ncco4)sc23)cn1 ; COc1ccc(-c2c(S(C)(=O)=O)ccc3nc(NC(=O)OCc4ncco4)sc23)cn1 ; O=C(Nc1nc2cccc(-c3ccccn3)c2s1)OCc1ncco1 ; O=C(Nc1nc2cccc(N3CC(F)(COC(F)F)C3)c2s1)OCc1ncco1] 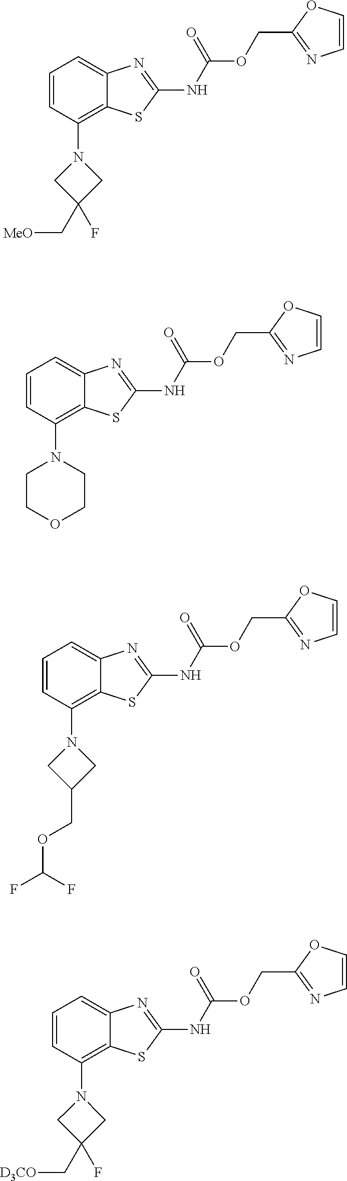 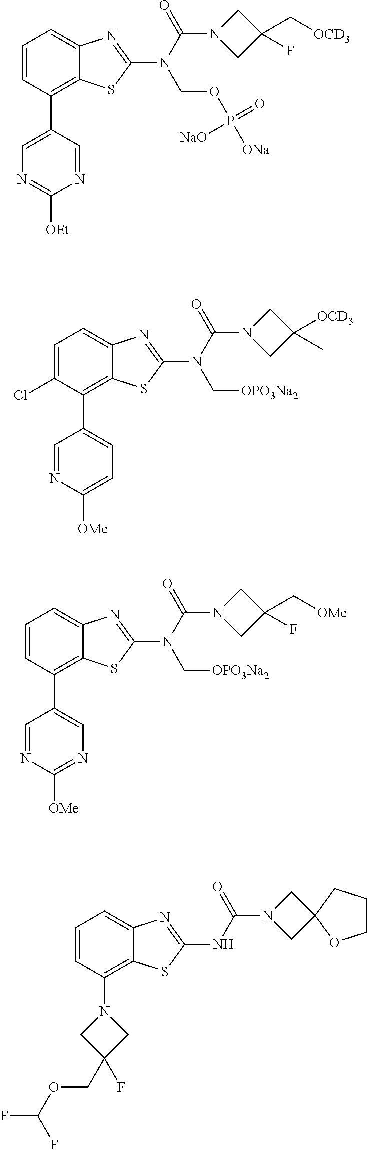 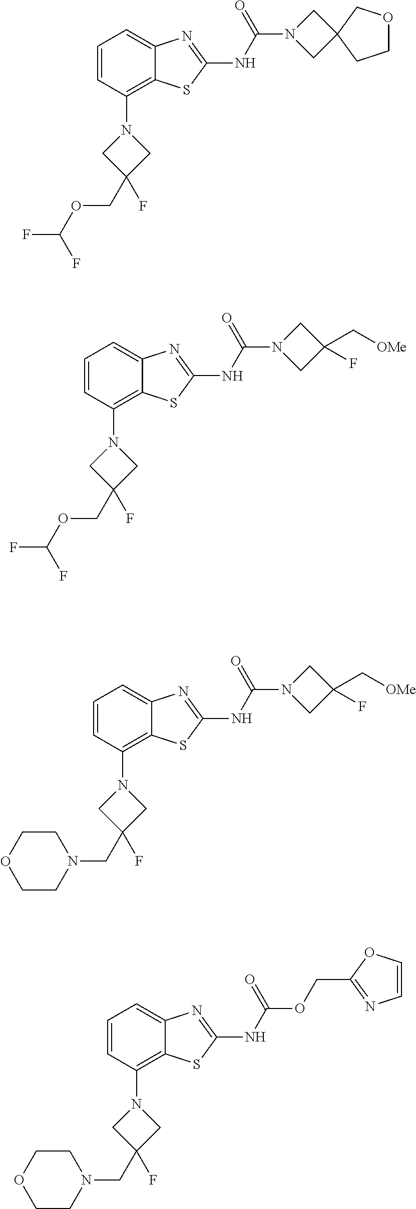 [SMILES: O=C(Nc1nc2cccc(N3CC(F)(COC(F)F)C3)c2s1)N1CC2(CCOC2)C1 ; COCC1(F)CN(C(=O)Nc2nc3cccc(N4CC(F)(COC(F)F)C4)c3s2)C1 ; COCC1(F)CN(C(=O)Nc2nc3cccc(N4CC(F)(CN5CCOCC5)C4)c3s2)C1 ; O=C(Nc1nc2cccc(N3CC(F)(CN4CCOCC4)C3)c2s1)OCc1ncco1] 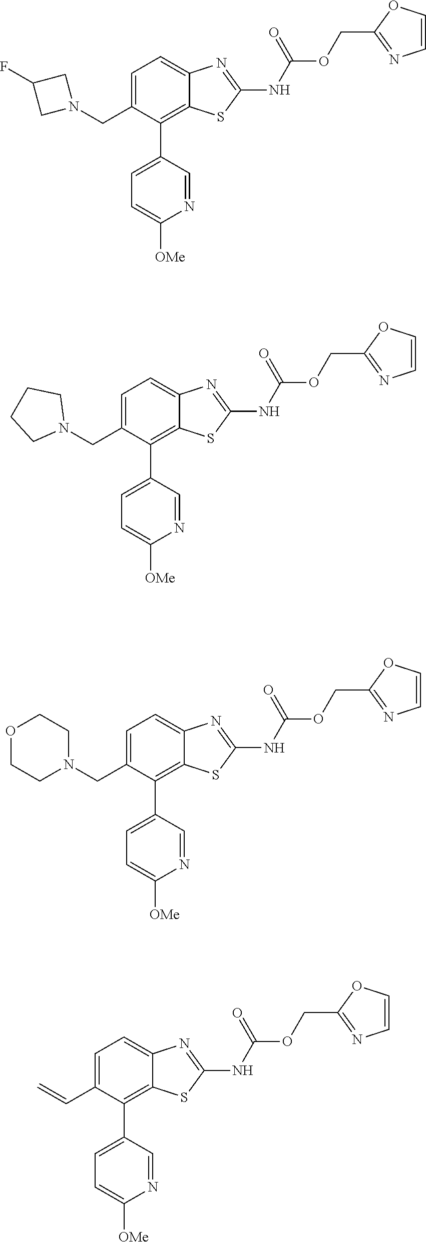 [SMILES: COc1ccc(-c2c(CN3CC(F)C3)ccc3nc(NC(=O)OCc4ncco4)sc23)cn1 ; COc1ccc(-c2c(CN3CCCC3)ccc3nc(NC(=O)OCc4ncco4)sc23)cn1 ; COc1ccc(-c2c(CN3CCOCC3)ccc3nc(NC(=O)OCc4ncco4)sc23)cn1 ; C=Cc1ccc2nc(NC(=O)OCc3ncco3)sc2c1-c1ccc(OC)nc1] 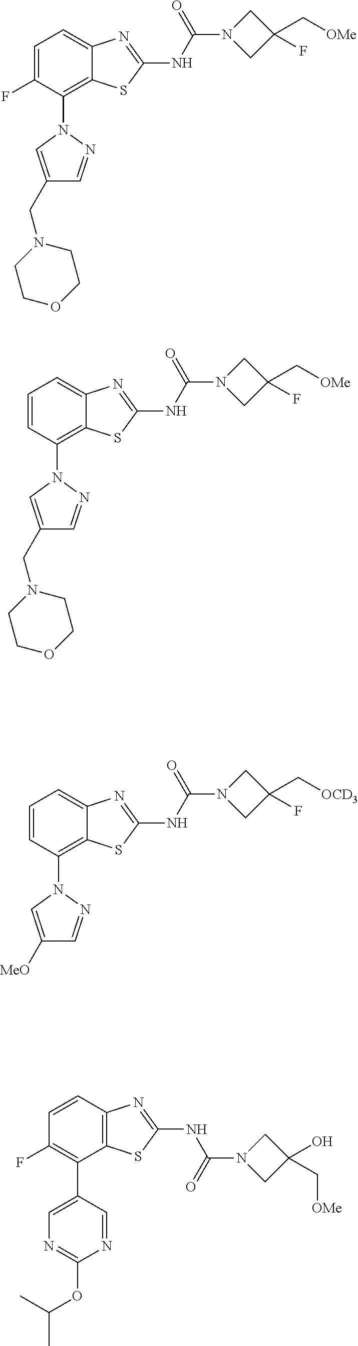 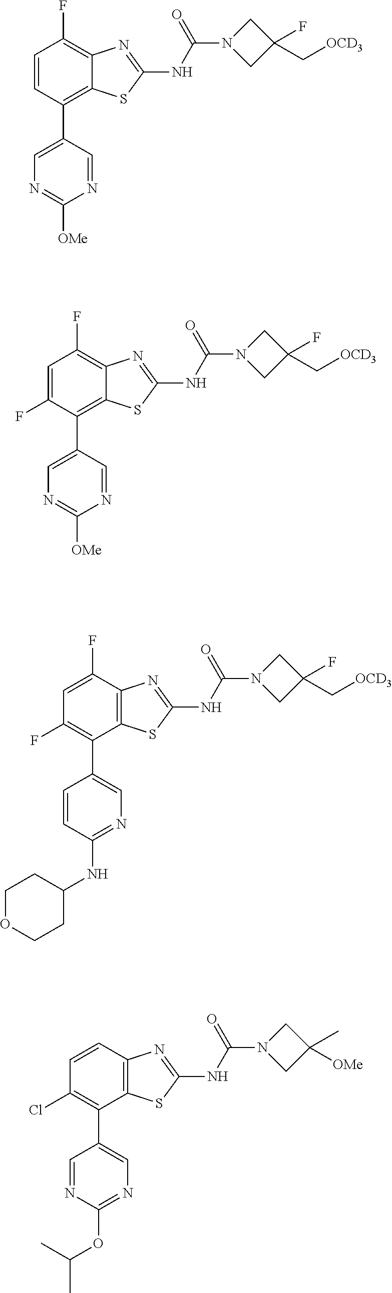 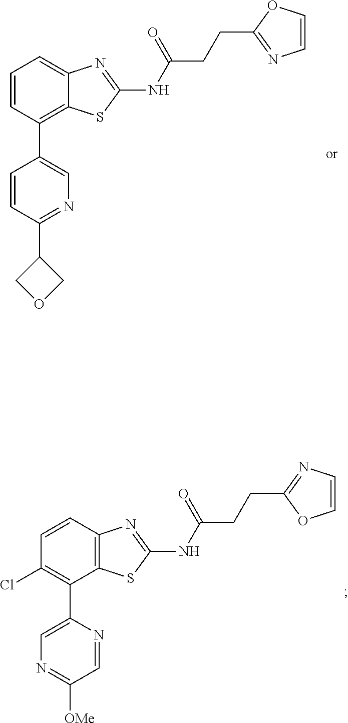 [SMILES: O=C(CCc1ncco1)Nc1nc2cccc(-c3ccc(C4COC4)nc3)c2s1 ; COc1cnc(-c2c(Cl)ccc3nc(NC(=O)CCc4ncco4)sc23)cn1]
or a pharmaceutically acceptable salt thereof.

40. A pharmaceutical composition comprising a compound according to claim 1, or a pharmaceutically acceptable salt thereof, and a pharmaceutically acceptable carrier.

41. A method of treating a disease or condition responsive to the modulation of beta catenin condensates, the method comprising administering to a subject in need an effective amount of a compound according to claim 1, or a pharmaceutically acceptable salt thereof, or a pharmaceutical composition comprising a compound according to claim 1, or a pharmaceutically acceptable salt thereof, and a pharmaceutically acceptable carrier.

42. The method of claim 41, wherein the disease or condition is:
(i) cancer;
(ii) bladder cancer, breast cancer, colorectal cancer, endometrial cancer, gastric cancer, head and neck cancer, leukemia, liver cancer, lung cancer, ovarian cancer, pancreatic cancer, or a solid tumor; or
(iii) triple negative breast cancer, chronic lymphocytic leukemia, acute myeloid leukemia, or non-small cell lung cancer.

43.-44. (canceled)
